(19) United States
(12) Patent Application Publication
(10) Pub. No.: US 2009/0226500 A1
(43) Pub. Date: Sep. 10, 2009

(54) SUTURES AND ANTI-SCARRING AGENTS

(75) Inventors: Rui Avelar, Vancouver (CA); Arpita Maiti, Vancouver (CA); Philip M. Toleikis, Vancouver (CA); Johanne Diane Cashman, Vancouver (CA); David M. Gravett, Mountain View, CA (US)

Correspondence Address: SEED INTELLECTUAL PROPERTY LAW GROUP PLLC, 701 FIFTH AVENUE, SUITE 5400, SEATTLE, WA, 98104-7092, US

(73) Assignee: Angiotech Pharmaceuticals, Inc, Vancouver (CA)

(21) Appl. No.: 12/162,572

(22) PCT Filed: Jan. 31, 2007
(86) PCT No.: PCT/US07/02714

§ 371 Date: May 6, 2009

Related U.S. Application Data
(60) Provisional application No. 60/763,945, filed on Jan. 31, 2006.

(51) Int. Cl. A61F 2/04; A61K 31/135; A61K 31/553; A61K 31/496; A61K 31/4164; A61K 31/395; A61K 31/426

(52) U.S. Cl. 424/423; 514/649; 514/211.08; 514/254.07; 514/399; 514/183; 514/370

(57) ABSTRACT

Sutures are used in combination with anti-scarring agents to inhibit fibrosis between the sutures and the host tissues into which the sutures are inserted. Compositions and methods are described for use in reducing excessive scarring, surgical adhesion, and other disorders.

### BACKGROUND OF THE INVENTION

1. Field of the Invention

The present invention relates generally to pharmaceutical agents and compositions for administration in association with sutures. More specifically, the present invention relates to compositions and methods for preparing sutures that inhibits a fibrotic response between the sutures and the tissue in contact with the suture material.

2. Description of the Related Art

Surgical adhesions are abnormal, fibrous bands of scar tissue that can form inside the body as a result of the healing process that follows any open or minimally invasive surgical procedure including abdominal, gynecologic, cardiothoracic, spinal, plastic, vascular, ENT, opthalmologic, urologic, neuro, or orthopedic surgery. Surgical adhesions are typically connective tissue structures that form between adjacent injured areas within the body. Briefly, localized areas of injury trigger an inflammatory and healing response that culminates in healing and scar tissue formation. If scarring results in the formation of fibrous tissue bands or adherence of adjacent anatomical structures (that should be separate), surgical adhesion formation is said to have occurred. Adhesions can range from flimsy, easily separable structures to dense, tenacious fibrous structures that can only be separated by surgical dissection. While many adhesions are benign, some can cause significant clinical problems and are a leading cause of repeat surgical intervention. Surgery to breakdown adhesions (adhesiolysis) often results in failure and recurrence because the surgical trauma involved in breaking down the adhesion triggers the entire process to repeat itself. Surgical breakdown of adhesions is a significant clinical problem and it is estimated that there were 473,000 adhesiolysis procedures in the US in 2002 According to the Diagnosis-Related Groups (DRGs), the total hospital charges for these procedures is likely to be at least US $10 billion annually.

Since all interventions involve a certain degree of trauma to the operative tissues, virtually any procedure (no matter how well executed) has the potential to result in the formation of clinically significant adhesion formation. Adhesions can be triggered by surgical trauma such as cutting, manipulation, retraction or suturing, as well as from inflammation, infection (e.g., fungal or mycobacterium), bleeding or the presence of a foreign body. Surgical trauma may also result from tissue drying, ischemia, or thermal injury. Due to the diverse etiology of surgical adhesions, the potential for formation exists regardless of whether the surgery is done in a so-called minimally invasive fashion (e.g., catheter-based therapies, laparoscopy) or in a standard open technique involving one or more relatively large incisions. Although a potential complication of any surgical intervention, surgical adhesions are particularly problematic in GI surgery (causing bowel obstruction), gynecological surgery (causing pain and/or infertility), tendon repairs (causing shortening and flexion deformities), joint capsule procedures (causing capsular contractures), and nerve and muscle repair procedures (causing diminished or lost function).

Surgical adhesions may cause various, often serious and unpredictable clinical complications; some of which manifest themselves only years after completion of the original procedure. Complications from surgical adhesions are a major cause of failed surgical therapy and are the leading cause of bowel obstruction and infertility. Other adhesion-related complications include chronic back or pelvic pain, intestinal obstruction, urethral obstruction and voiding dysfunction. Relieving the post-surgical complications caused by adhesions generally requires another surgery. However, the subsequent surgery is further complicated by adhesions formed as a result of the previous surgery. In addition, the second surgery is likely to result in further adhesions and a continuing cycle of additional surgical complications

Adhesions generally begin to form within the first several days after surgery Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation may be inhibited.

Various modes of adhesion prevention have been examined, including (1) prevention of fibrin deposition, (2) reduction of local tissue inflammation and (3) removal of fibrin deposits. Fibrin deposition is prevented through the use of physical barriers that are either mechanical or comprised of viscous solutions. Barriers have the added advantage of physically preventing adjacent tissues from contacting each other and thereby reducing the probability that they will scar together. Although many investigators and commercial products utilize adhesion prevention barriers, a number of technical difficulties exist and significant failure rates have been reported. Inflammation is reduced by the administration of drugs such as corticosteroids and non-steroidal anti-inflammatory drugs. However, the results from the use of these drugs in animal models have not been encouraging due to the extent of the inflammatory response and dose restriction due to systemic side effects. Finally, the removal of fibrin deposits has been investigated using proteolytic and fibrinolytic enzymes. A potential complication to the clinical use of these enzymes is the possibility for post-surgical excessive bleeding (surgical hemostasis is critical for procedural success).

Accordingly, there is need for developing new prevention or treatment methods for surgical adhesion. The present invention fulfils this need and also provides additional related advantages.

### SUMMARY OF THE INVENTION

Briefly stated, the present invention provides sutures (including plain and self-retaining sutures) that comprise an anti-scarring agent, as well as methods for making and using such sutures In addition, the present invention also provides compositions that comprise anti-scarring agents and methods for using such compositions in combination with sutures (including plain and self-retaining sutures) in various applications (e.g., reducing excessive scarring or surgical adhesion).

In one aspect, the present invention provides an anti-scarring suture comprising a suture and an anti-scarring agent. In certain embodiments, the suture is a self-retaining suture. In certain embodiments, the anti-scarring suture further comprises a polymer, including a polymeric carrier for the anti-scarring agent.

In a related aspect, the present invention provides an anti-scarring suture connector comprising a suture connector and an anti-scarring agent. In another related aspect, the present invention provides an anti-scarring suture anchor that comprises a suture and an anti-scarring agent.

In another aspect, the present invention provides a method for making an anti-scarring suture comprising: combining a suture with an anti-scarring agent or a composition comprising an anti-scarring agent.

In another aspect, the present invention provides a method for reducing, or reducing the risk of, excessive scarring, comprising: infiltrating a tissue at which a suture (e.g., a plain suture or a self-retaining suture) has been, is being, or to be implanted with an effective amount of an anti-scarring agent or a composition comprising an anti-scarring agent.

In another aspect, the present invention provides a method for reducing, or reducing the risk of, excessive scarring, comprising: implanting an anti-scarring suture provided herein into a tissue.

In certain embodiments of the methods for reducing, or reducing the risk of, excessive scarring, the excessive scarring may be at a wound closure site a site of attachment of a foreign element to a tissue or a site of a tissue repositioning surgery In certain embodiments, the excessive scarring results in a keloid or hypertrophic scar or in surgical adhesion

The anti-scarring agent useful in combination with a suture according to the present invention may be any anti-scarring agent disclosed herein. In certain embodiments, the anti-scarring agent is cerivastatin, terbinafine, radicicol, trichostatin A, mithramycin A, 5-fluorouracil, doxorubicin, mitoxantrone, etoposide, paclitaxel, rapamycin, halofuginone hydrobromide, pitavastatin, or pirarubicin. In certain embodiments, the anti-scarring agent inhibits inflammation; collagen production in, or release from, cells; or is an anti-infective or antifungal agent. In certain embodiments, the anti-scarring agent is an mTOR inhibitor, HMGCoA reductase inhibitor, DNA intercalator or reversible DNA binder, heat shock protein 90 (HSP90) inhibitor, or histone deacetylase (HDAC) inhibitor. In certain embodiments, the anti-scarring agent is hydrophobic.

These and other aspects of the present invention will become evident upon reference to the following detailed description and attached drawings. In addition, various references are set forth herein which describe in more detail certain procedures and/or compositions (e.g., polymers), and are therefore incorporated by reference in the entirety.

### BRIEF DESCRIPTION OF THE DRAWINGS

FIG. 1 is a graph showing the results for the screening assay for assessing the effect of mitoxantrone on nitric oxide production by macrophages.

FIG. 2 is a graph showing the results for the screening assay for assessing the effect of various therapeutic agents on TNF-alpha production by macrophages.

FIG. 3 is a graph showing the results for the screening assay for assessing the effect of rapamycin concentration for TNFα production by THP 1 cells.

FIG. 4 is graph showing the results of a screening assay for assessing the effect of mitoxantrone (mitoxantrone IC₅₀=20 nM) on proliferation of human fibroblasts

FIG. 5 is graph showing the results of a screening assay for assessing the effect of rapamycin on cell proliferation of human fibroblasts.

FIG. 6 is graph showing the results of a screening assay for assessing the effect of paclitaxel on cell proliferation of human fibroblasts.

FIG. 7 is a picture that shows an uninjured carotid artery from a rat balloon injury model.

FIG. 8 is a picture that shows an injured carotid artery from a rat balloon injury model.

FIG. 9 is a picture that shows a paclitaxel/mesh treated carotid artery in a rat balloon injury model (345 μg paclitaxel in a 50:50 PLG coating on a 10:90 PLG mesh).

FIG. 10A schematically depicts the transcriptional regulation of matrix metalloproteinases.

FIG. 10B is a blot that demonstrates that IL-1 stimulates AP-1 transcriptional activity.

FIG. 10C is a graph that shows that IL-1 induced binding activity decreased in lysates from chondrocytes that were pretreated with paclitaxel.

FIG. 10D is a blot which shows that IL-1 induction increases collagenase and stromelysin in RNA levels in chondrocytes, and that this induction can be inhibited by pretreatment with paclitaxel.

FIGS. 11A-H are blots that show the effect of various anti-microtubule agents in inhibiting collagenase expression.

FIG. 12 is a graph showing the results of a screening assay for assessing the effect of paclitaxel on smooth muscle cell migration (paclitaxel IC₅₀=0.76 nM)

FIG. 13 is a graph showing the results of a screening assay for assessing the effect of geldanamycin on IL-1β production by macrophages (IC₅₀=20 nM for IL-1β production by THP-1 cells)

FIG. 14 is a graph showing the results of a screening assay for assessing the effect of geldanamycin on IL-8 production by macrophages (IC₅₀=27 nM for IL-8 production by THP-1 cells).

FIG. 15 is a graph showing the results of a screening assay for assessing the effect of geldanamycin on MCP-1 production by macrophages (IC₅₀=7 nM for MCP-1 production by THP-1 cells).

FIG. 16 is graph showing the results of a screening assay for assessing the effect of paclitaxel on proliferation of smooth muscle cells.

FIG. 17 is graph showing the results of a screening assay for assessing the effect of paclitaxel (IC₅₀=134 nM) for proliferation of the murine RAW 264.7 macrophage cell line.

FIG. 18 is a graph showing the effect of PDGF-BB on smooth muscle cell migration.

FIG. 19 is a graph showing the results of the TGF-β analysis in silicone disk implanted compared to sham treated rats.

### DETAILED DESCRIPTION OF THE INVENTION

The present invention provides sutures and self-retaining sutures that comprise an anti-scarring agent, as well as methods for making and using such sutures. In addition, the present invention also provides compositions that comprise anti-scarring agents and methods for using such compositions in combination with sutures in various applications (e.g., surgical adhesion).

### DEFINITIONS

Prior to setting forth the invention, it may be helpful to an understanding thereof to first set forth definitions of certain terms that is used hereinafter.

“Fibrosis,” or “scarring,” or “fibrotic response” refers to the formation of fibrous (scar) tissue in response to injury or medical intervention. Therapeutic agents which inhibit fibrosis or scarring are referred to herein as “fibrosis-inhibiting agents,” “fibrosis-inhibitors,” “anti-scarring agents,” “anti-fibrotic agents,” “anti-fibrosis agents,” and the like, where these agents inhibit fibrosis through one or more mechanisms including: inhibiting inflammation or the acute inflammatory response, inhibiting migration or proliferation of connective tissue cells (such as fibroblasts, smooth muscle cells, vascular smooth muscle cells), inhibiting angiogenesis, reducing extracellular matrix (ECM) production or promoting ECM breakdown, and/or inhibiting tissue remodeling.

“Host”, “person”, “subject”, “patient” and the like are used synonymously to refer to the living being into which a suture of the present invention is implanted.

“Inhibit fibrosis”, “reduce fibrosis”, “inhibits scarring” and the like are used synonymously to refer to the action of agents or compositions which result in a statistically significant decrease in the formation of fibrous tissue that can be expected to occur in the absence of the agent or composition.

“Inhibitor” refers to an agent that prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent that prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent that stimulates a biological process or rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or other molecule that impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995).

“Release of an agent” from a suture refers to a statistically significant presence of an agent, or a subcomponent thereof, which has dissociated from a suture that comprises the agent.

“Suture” refers to the fine thread or other material used to join two surfaces or edges together (e.g., closing a wound, joining tissues, or performing repositioning procedures). Sutures include both plain sutures and self-retaining sutures, and may comprise bioabsorbable or nonabsorbable material.

“Plain suture” refers to a suture without any barbs or other retainers located along the body of the suture.

“Self-retaining suture” refers to a suture with one or more retainers are located along the suture. The retainers are of sufficient size and appropriate geometry for fastening to, or gripping, the tissue through which the self-retaining suture is inserted and achieving closure of an incision or wound (or repositioning tissue) with superior attachment or without the need for tying knots. Retainers may be configured to have tissue insertion points (such as barbs), tissue insertion edges (such as conical or frusto-conical structures), and so forth.

“One-directional self-retaining suture” (also referred to as “one-directional suture,” “one-way self-retaining suture,” “one-way suture,” “uni-directional self-retaining suture,” or “uni-directional suture”) refers to a suture having retainers on its exterior surface and facing towards one end of the suture. Such arrangement of retainers on the suture allows the suture to be drawn in only only one direction through tissue, but not in the opposite direction.

“Two-way self-retaining suture” (also referred to “two-way suture,” “two-directional self-retaining suture,” “two-directional suture,” “bi-directional self-retaining suture,” or “bi-directional suture”) refers to a suture that has retainers facing toward one end of the suture for about half the suture length and retainers facing the opposite direction toward the other end of the suture for the other half of the suture length. This arrangement allows the retainers to move in the same direction as each respective suture end is inserted into host tissue.

“Localized delivery” refers to administration of a therapeutic agent from a suture or composition into or near a tissue in need of the therapeutic agent and provides a high local (regional) concentration of the therapeutic agent at or near the site of suture implantation. In certain aspects, the anti-scarring agent or composition that comprises the anti-scarring agent is released from a suture locally into or in the vicinity of the site where the suture is implanted. In other aspects, “localized delivery” is achieved by direct contact between the surface of a suture and the surface of the tissue in contact with the suture.

“Release of an agent from a suture” refers to any statistically significant dissociation of an agent, or a subcomponent thereof from a suture.

“Biodegradable” (used interchangeably with “degradable” or “absorbable”) refers to materials for which the degradation process is at least partially mediated by, or performed in, a biological system. “Degradation” refers to a chain scission process by which a polymer chain is cleaved into oligomers and monomers. Chain scission may occur through various mechanisms, including, for example, by chemical reaction (e.g., hydrolysis, oxidation/reduction, enzymatic mechanisms or a combination or these) or by a thermal or photolytic process. Polymer degradation may be characterized, for example, using gel permeation chromatography (GPC), which monitors the polymer molecular mass changes during erosion and drug release. “Biodegradable” also refers to materials may be degraded by an erosion process at least partially mediated by, or performed in, a biological system. “Erosion” refers to a process in which material is lost from the bulk. In the case of a polymeric system, the material may be a monomer, an oligomer, a part of a polymer backbone, and/or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del Rev. (2001), 48, 229-247): (1) water-soluble polymers that have been insolubilized by covalent cross-links and that solubilize as the cross-links or the backbone undergo a hydrolytic cleavage, enzymatic cleavage or a combination of these; (2) polymers that are initially water insoluble are solubilized by hydrolysis, enzymatic cleavage, ionization, or pronation of a pendant group or a combination of these mechanisms; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance (EPR) spectroscopy, NMR imaging, and recording mass loss during an erosion experiment. For microspheres, photon correlation spectroscopy (PCS) and other particles size measurement techniques may be applied to monitor the size evolution of erodible devices versus time.

Any concentration ranges, percentage range, or ratio range recited herein are to be understood to include concentrations, percentages or ratios of any integer within that range and fractions thereof, such as one tenth and one hundredth of an integer, unless otherwise indicated Also, any number range recited herein relating to any physical feature, such as polymer subunits, size or thickness are to be understood to include any integer within the recited range, unless otherwise indicated. It should be understood that the terms “a” and “an” as used above and elsewhere herein refer to “one or more” of the enumerated components. As used herein, the term “about” means±15%

With regard to nomenclature pertinent to molecular structures, the following definitions apply:

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of one to six carbon atoms, preferably one to four carbon atoms. “Substituted alkyl” refers to alkyl substituted with one or more substituent groups. “Alkylene,” “lower alkylene” and “substituted alkylene” refer to divalent alkyl, lower alkyl, and substituted alkyl groups, respectively.

The term “aryl” as used herein, and unless otherwise specified, refers to an aromatic substituent containing a single aromatic ring (monocyclic) or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl in which at least one carbon atom is replaced with a heteroatom. The terms “arylene” and “substituted arylene” refer to divalent aryl and substituted aryl groups as lust defined.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon

“Hydrocarbyl” refers to univalent hydrocarbyl radicals containing 1 to about 30 carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. The term “hydrocarbylene” intends a divalent hydrocarbyl moiety containing 1 to about 30 carbon atoms, preferably 1 to about 24 carbon atoms, most preferably 1 to about 12 carbon atoms, including branched or unbranched, saturated or unsaturated species, or the like. The term “lower hydrocarbylene” intends a hydrocarbylene group of one to six carbon atoms, preferably one to four carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and “heterohydrocarbylene” refer to hydrocarbylene in which at least one carbon atom is replaced with a heteroatom. If not otherwise indicated, “hydrocarbyl” indicates both unsubstituted and substituted hydrocarbyls, “heteroatom-containing hydrocarbyl” indicates both unsubstituted and substituted heteroatom-containing hydrocarbyls and so forth.

By “substituted” as in “substituted hydrocarbyl,” “substituted alkyl,” and the like, as alluded to in some of the aforementioned definitions, is meant that in the hydrocarbyl, alkyl, or other moiety, at least one hydrogen atom bound to a carbon atom is replaced with one or more substituents that are functional groups such as alkoxy, hydroxy, halo, nitro, and the like. Unless otherwise indicated, it is to be understood that specified molecular segments can be substituted with one or more substituents that do not compromise a compound's utility. For example, “succinimidyl” is intended to include unsubstituted succinimidyl as well as sulfosuccinimidyl and other succinimidyl groups substituted on a ring carbon atom, e.g., with alkoxy substituents, polyether substituents, or the like.

### A. Anti-Scarring Agents

Numerous anti-scarring agents have been identified that can be used in combination with a suture according to the present invention. The agents may be further formulated with one or more other materials, such as another therapeutic agent (e.g., an anti-infective agent or an anti-inflammatory agent) and/or a polymeric carrier, which formulations are discussed below.

Many suitable anti-scarring agents are specifically identified herein (including those used in the Examples section), and others, depending on their mechanisms of action, may be readily determined based upon one or more in vitro and in vivo (animal) models including those provided in the examples and those otherwise known in the art. For example, anti-scarring agents useful in the present invention include those having an IC₅₀ value tested according to Example 32 between 0.1 nM and 10 nM, between 10 nM and 100 nM, between 100 nM and 600 nM, or between 600 nM and 1200 nM.

Anti-scarring agents useful in the present invention may inhibit one or more aspect of the fibrosis process. For example, in certain embodiments, the anti-scarring agent inhibits inflammation; collagen production in, or release from, cells; and/or is an anti-infective or antifungal agent.

Exemplary therapeutic compounds that may be useful in the invention include, but are not limited to:

1. Angiogenesis Inhibitors

In one embodiment, the pharmacologically active compound is an angiogenesis inhibitor (e.g., 2-ME (NSC-659853), PI-88 (D-mannose, O-6-O-phosphono-alpha-D-mannopyranosyl-(1-3)-O-alpha-D-mannopyranosyl-(1-3)-O-alpha-D-mannopyranosyl-(1-3)-O-alpha-D-mannopyranosyl-(1-2)-hydrogen sulphate), thalidomide (1H-isoindole-1,3(2H)-dione, 2-(2,6-dioxo-3-piperidinyl)-), CDC-394, CC-5079, ENMD-0995 (S-3-amino-phthalidoglutarimide), AVE-8062A, vatalanib, SH-268, halofuginone hydrobromide, atiprimod dimaleate (2-azaspivo[4.5]decane-2-propanamine, N,N-diethyl-8,8-dipropyl, dimaleate), ATN-224, CHIR-258, combretastatin A-4 (phenol, 2-methoxy-5-[2-(3,4,5-trimethoxyphenyl)ethenyl]-, (Z)-), GCS-100LE, or an analogue or derivative thereof).

Additional angiogenesis inhibitor include, but are not limited to, AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen). TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zenaca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (such as combretastatin A-1, A-2, A-3, A-4, A-5, A-6, B-1, B-2, B-3, B-4, D-1, D-2, and combretastatin A-4 phosphate (Oxigene)), Rebimastat (Bristol-Meyers Squibb), Dextrin 2-sulfate (ML Laboratories), Cilengitide (Merk KGaA), NSC-706704 (Phaminox) KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), or an analogue or derivative thereof). In other embodiments, the angiogenesis inhibitor may be a recombinant anti-angiogenic compound such as ANGIOCOL (available from Biostratum Inc., Durham, N.C.).

2. 5-Lipoxygenase Inhibitors and Antagonists

In another embodiment, the pharmacologically active compound is a 5-lipoxygenase inhibitor or antagonist (e.g., Wy-50295 (2-naphthaleneacetic acid, alpha-methyl-6-(2-quinolinylmethoxy)-, (S)—), ONO-LP-269 (2,11,14-eicosatrienamide, N-(4-hydroxy-2-(1H-tetrazol-5-yl)-8-quinolinyl)-, (E,Z,Z)-), licofelone (1H-pyrrolizine-5-acetic acid, 6-(4-chlorophenyl)-2,3-dihydro-2,2-dimethyl-7-phenyl-), CMI-568 (urea, N-butyl-N-hydroxy-N′-(4-(3-(methylsulfonyl)-2-propoxy-5-(tetrahydro-5-(3,4,5-trimethoxyphenyl)-2-furanyl)phenoxy)butyl)-,trans-), IP-751 ((3R,4R)-(delta 6)-THC-DMH-11-oic acid), PF-5901 (benzenemethanol, alpha-pentyl-3-(2-quinolinylmethoxy)-), LY-293111 (benzoic acid, 2-(3-(3-((5-ethyl-4′-fluoro-2-hydroxy(1,1′-biphenyl)-4-yl)oxy)propoxy)-2-propylphenoxy)-), RG-5901-A (benzenemethanol, alpha-pentyl-3-(2-quinolinylmethoxy)-, hydrochloride), rilopirox (2(1H)-pyridinone, 6-((4-(4-chlorophenoxy)phenoxy)methyl)-1-hydroxy-4-methyl-), L-674636 (acetic acid, ((4-(4-chlorophenyl)-1-(4-(2-quinolinylmethoxy)phenyl)butyl)thio)-AS)), 7-((3-(4-methoxy-tetrahydro-2H-pyran-4-yl)phenyl)methoxy)-4-phenylnaphtho(2,3-c)furan-1(3H)-one, MK-886 (1H-indole-2-propanoic acid, 1-((4-chlorophenyl)methyl)-3-((1,1-dimethylethyl)thio)-alpha,alpha-dimethyl-5-(1-methylethyl)-), quiflapon (1H-indole-2-propanoic acid, 1-((4-chlorophenyl)methyl)-3-((1,1-dimethylethyl)thio)-alpha,alpha-dimethyl-5-(2-quinolinylmethoxy)-), quiflapon (1H-Indole-2-propanoic acid, 1-((4-chlorophenyl)methyl)-3-((1,1-dimethylethyl)thio)-alpha,alpha-dimethyl-5-(2-quinolinylmethoxy)-), docebenone (2,5-cyclohexadiene-1,4-dione, 2-(12-hydroxy-5,10-dodecadiynyl)-3,5,6-trimethyl-), zileuton (urea, N-(1-benzo(b)thien-2-ylethyl)-N-hydroxy-), or an analogue or derivative thereof).

3. Chemokine Receptor Antagonists CCR (1, 3, and 5)

In another embodiment, the pharmacologically active compound is a chemokine receptor antagonist which inhibits one or more subtypes of CCR (1, 3, and 5) (e.g., ONO-4128 (1,4,9-triazaspiro(5.5)undecane-2,5-dione, 1-butyl-3-(cyclohexylmethyl)-9-((2,3-dihydro-1,4-benzodioxin-6-yl)methyl-), L-381, CT-112 (L-arginine, L-threonyl-L-threonyl-L-seryl-L-glutaminyl-L-valyl-L-arginyl-L-prolyl-), AS-900004, SCH-C, ZK-811752, PD-172084, UK-427857, SB-380732, vMIP II, SB-265610, DPC-168, TAK-779 (N,N-dimethyl-N-(4-(2-(4-methylphenyl)-6,7-dihydro-5H-benzocyclohepten-8-ylcarboxamido)benzyl)tetrahydro-2H-pyran-4-aminium chloride), TAK-220, KRH-1120), GSK766994, SSR-150106, or an analogue or derivative thereof). Other examples of chemokine receptor antagonists include a-Immunokine-NNS03, BX-471, CCX-282, Sch-350634; Sch-351125; Sch-417690; SCH-C, and analogues and derivatives thereof.

4. Cell Cycle Inhibitors

In another embodiment, the pharmacologically active compound is a cell cycle inhibitor. Representative examples of such agents include taxanes (eg. paclitaxel (discussed in more detail below) and docetaxel) (Schiff et al. Nature 277 665-667, 1979, Long and Fairchild. Cancer Research 54.4355-4361, 1994, Ringel and Horwitz, J Nat'l Cancer Inst 83(4):288-291, 1991, Pazdur et al., Cancer Treat. Rev. 19(40) 351-386, 1993), etanidazole, nimorazole (B. A. Chabner and D. L. Longo. Cancer Chemotherapy and Biotherapy—Principles and Practice. Lippincott-Raven Publishers, New York, 1996, p. 554), perfluorochemicals with hyperbaric oxygen, transfusion, erythropoietin, BW12C, nicotinamide, hydralazine, BSO, WR-2721, ludR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat. Stud. Phys., Chem. Med. 40(2):153-61, 1981), SR-2508 (Brown et al., Int. J. Radiat. Oncol., Biol. Phys. 7(6):695-703, 1981), 2H-isoindolediones (J. A. Myers, 2H-Isoindolediones, the synthesis and use as radiosensitizers. U.S. Pat. No. 4,494,547, Jan. 22, 1985), chiral (((2-bromoethyl)-amino)methyl)-nitro-1H-imidazole-1-ethanol (V. G. Beylin, et al., Process for preparing chiral (((2-bromoethyl)-amino)methyl)-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and the use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997. Process for Preparing 1,2,4 Benzotriazine oxides U.S. Pat. No. 5,175,287. Dec. 29, 1992), nitric oxide (J. B. Mitchell et al. Use of Nitric oxide releasing compounds as hypoxic cell radiation sensitizers U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient U.S. Pat. No. 5,270,330, Dec. 14, 1993; T Suzuki et al. 2-Nitroimidazole derivative, production thereof, and radiosensitizer containing the same as active ingredient. U.S. Pat. No. 5,270,330, Dec. 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W.W. Lee. Substituted Benzamide Radiosensitizers. U.S. Pat. No. 5,032,617, Jul. 16, 1991), autobiotics (L. G. Egyud. Autobiotics and the use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, Jun. 1, 1993), acridine-intercalator (M Papadopoulou-Rosenzweig Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183. Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T. Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T Kagitanil Nitrotriazole Compound Publication Number 07149737 A (Japan) Jun. 22, 1993), cisplatin, doxorubin, misonidazole, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12):3156-3174, 1996), camptothecin (Ewend M. G. et al., Local delivery of chemotherapy and concurrent external beam radiotherapy prolongs survival in metastatic brain tumor models. Cancer Research 56(22):5217-5223, 1996) and paclitaxel (Tishler R. B. et al. Taxol: a novel radiation sensitizer. International Journal of Radiation Oncology and Biological Physics 22(3):613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, fluorouracil, epirubicin, doxorubicin, vindesine and etoposide. Analogues and derivatives include (CPA)₂Pt(DOLYM) and (DACH)Pt(DOLYM) cisplatin (Choi et al., Arch. Pharmacal Res. 22(2):151-156, 1999), Cis-(PtCl₂(4,7-H-5-methyl-7-oxo) 1,2,4(triazolo(1,5-a)pyrimidine)₂) (Navarro et al., J. Med. Chem. 41(3):332-338, 1998), (Pt(cis-1,4-DACH)(trans-Cl₂)(CBDCA)).½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂(NHCHN(C(CH₂)(CH₃)))₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans,cis-(Pt(OAc)₂I₂(en)) (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1) 75, 1996), cis-1,4-diaminocyclohexane cisplatin analogues (Shamsuddin et al., J. Inorg. Biochem. 61(4) 291-301, 1996), 5′ orientational isomer of cis-(Pt(NH₃)(4-aminoTEMP-O){d(GpG)}) (Dunham & Lippard, J. Am. Chem. Soc 117(43): 10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diamminedichloroplatinum(II) and its analogues cis-1,1-cyclobutanedicarbosylato(2R)-2-methyl-1,4-butanediam-mineplatinum(II) and cis-diammine(glycolato)platinum (Claycamp & Zimbrick, J. Inorg. Biochem., 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyphenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexanemalonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J Labelled Compd Radiopharm. 25(4) 349-57, 1988), aminoalkylaminoanthraquinone-derived cisplatin analogues (Kitov et al. Eur J. Med. Chem. 23(4):381-3, 1988), spiroplatin, carboplatin, iproplatin and JM40 platinum analogues (Schroyen et al., Eur. J. Cancer Clin. Oncol. 24(8): 1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike DaxueXuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et alt, J. Chem. Soc., Chem. Commun. 6:443-5, 1987), aliphatic tricarboxylic acid platinum complexes (EPA 185225), cis-dichloro(amino acid) (tert-butylamine)platinum(II) complexes (Pasini & Bersanetti, Inorg. Chim. Acta 107(4):259-67, 1985); 4-hydroperoxycylcophosphamide (Ballard et al., Cancer Chemother. Pharmacol. 26(6):397-402, 1990), acyclouridine cyclophosphamide derivatives (Zakerinia et al., Helv. Chim. Acta 73(4):912-15, 1990), 1,3,2-dioxa- and -oxazaphosphorinane cyclophosphamide analogues (Yang et al., Tetrahedron 44(20):6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1):31-7, 1989), phenylketophosphamide cyclophosphamide analogues (Ludeman et al., J. Med. Chem. 29(5):716-27, 1986), ASTA Z-7557 cyclophosphamide analogues (Evans et al., Int. J. Cancer 34(6):883-90, 1984), 3-(1-oxy-2,2,6,6-tetramethyl-4-piperidinyl)cyclophosphamide (Tsui et al., J. Med. Chem. 25(9):1106-10, 1982), 2-oxobis(2-β-chloroethylamino)-4-,6-dimethyl-1,3,2-oxazaphosphorinane cyclophosphamide (Carpenter et al., Phosphorus Sulfur 12(3):287-93, 1982), 5-fluoro- and 5-chlorocyclophosphamide (Foster et al., J. Med. Chem. 24(12) 1399-403, 1981), cis- and trans-4-phenylcyclophosphamide (Boyd et al., J Med. Chem. 23(4):372-5, 1980). 5-bromocyclophosphamide, 3,5-dehydrocyclophosphamide (Ludeman et al, J. Med. Chem. 22(2):151-8, 1979), 4-ethoxycarbonyl cyclophosphamide analogues (Foster. J. Pharm. Sci. 67(5):709-10, 1978), arylaminotetrahydro-2H-1,3,2-oxazaphosphorine 2-oxide cyclophosphamide analogues (Hamacher, Arch. Pharm. (Weinheim, Ger.) 310(5):J.428-34, 1977), NSC-26271 cyclophosphamide analogues (Montgomery & Struck, Cancer Treat. Rep. 60(4); J381-93, 1976), benzo annulated cyclophosphamide analogues (Ludeman & Zon, J. Med. Chem. 18(12):J1251-3, 1975), 6-trifluoromethylcyclophosphamide (Farmer & Cox, J. Med. Chem. 18(11):J 1106-10, 1975), 4-methycyclophosphamide and 6-methycyclophosphamide analogues (Cox et al., Biochem. Pharmacol. 24(5):J599-606, 1975); FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al. Cancer Chemother Pharmacol. 33(1):10-16, 1993), (6-maleimidocaproyl)hydrazone doxorubicin derivative (Willner et al., Bioconjugate Chem 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80, 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoeizel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″ cyano-4″-morpholinyl doxorubicin; 3′-deamino-3′-(3″-cyano-4″-morpholinyl)-13-dihydroxorubicin, (3′-deamino-3′-(3″-cyano-4″-morpholinyl) daunorubicin; 3′-deamino-3′-(3″-cyano-4″-morpholinyl)-3-dihydrodaunorubicin, and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43(6):1337-44, 1992), azo and azoxy misonidazole derivatives (Gattavecchia & Tonelli, Int. J. Radiat. Biol. Relat. Stud. Phys., Chem. Med. 45(5):469-77, 1984); RB90740 (Wardman et al., Br. J. Cancer, 74 Suppl. (27):S70-S74, 1996); 6-bromo and 6-chloro-2,3-dihydro-1,4-benzothiazines nitrosourea derivatives (Rai et al., Heterocycl. Commun. 2(6):587-592, 1996), diamino acid nitrosourea derivatives (Dulude et al., Bioorg. Med. Chem. Lett. 4(22):2697-700, 1994, Dulude et al., Bioorg. Med. Chem. 3(2):151-60, 1995), amino acid nitrosourea derivatives (Zheleva et al., Pharmazie 50(11):25-6, 1995), 3′,4′-didemethoxy-3′,4′-dioxo-4-deoxypodophyllotoxin nitrosourea derivatives (Miyahara et al., Heterocycles 39(1):361-9, 1994), ACNU (Matsunaga et al., Immunopharmacology 23(3):199-204, 1992), tertiary phosphine oxide nitrosourea derivatives (Guguva et al., Pharmazie 46(8):603, 1991), sulfamerizine and sulfamethizole nitrosourea derivatives (Chiang et al., Zhonghua Yaozue Zazhi 43(5):401-6, 1991), thymidine nitrosourea analogues (Zhang et al., Cancer Commun. 3(4):119-26, 1991), 1,3-bis(2-chloroethyl)-1-nitrosourea (August et al., Cancer Res. 51(6):1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9):1311-16, 1989), pyrimidine (II) nitrosourea derivatives (Wei et al., Chung-hua Yao Hsuch Tsa Chih 41(1):19-26, 1989), CGP 6809 (Schieweck et al., Cancer Chemother. Pharmacol 23(6) 341-7, 1989), B-3839 (Prajda et al., In Vivo 2(2) 151-4, 1988), 5-halogenocytosine nitrosourea derivatives (Chiang & Tseng, T'ai-wan Yao Hsuch Tsa Chih 38(1); 37-43, 1986), 1-(2-chloroethyl)-3-isobutyl-3-(β-maltosyl)-1-nitrosourea (Fujimoto & Ogawa, J. Pharmacobio-Dyn. 10(7):341-5, 1987), sulfur-containing nitrosoureas (Tang et al., Yaoxue Xuebao 21(7):502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33(11):969-77, 1985), CNCC, RFCNU and chlorozotocin (Mena et al., Chemotherapy (Basel) 32(2):131-7, 1986), CNUA (Edanami et al., Chemotherapy (Tokyo) 33(5):455-61, 1985), 1-(2-chloroethyl)-3-isobutyl-3-(D-maltosyl)-1-nitrosourea (Fujimoto & Ogawa, Jpn. J. Cancer Res. (Gann) 76(7):651-6, 1985), choline-like nitrosoalkylureas (Belyaev et al., Izv. Akad NAUK SSSR, Ser Khim. 3:553-7, 1985), sucrose nitrosourea derivatives (JP 84219300), sulfa drug nitrosourea analogues (Chiang et al., Proc. Nat'l Sci. Counc., Repub. China, Part A 8(1):18-22, 1984), DONU (Asanuma et al., J. Jpn. Soc. Cancer Ther. 17(8):2035-43, 1982), N,N′-bis(N-(2-chloroethyl)-N-nitrosocarbamoyl)cystamine (CNCC) (Blazsek et al., Toxicol. Appl. Pharmacol. 74(2):250-7, 1984), dimethylnitrosourea (Krutova et al., Izv. Akad. NAUK SSSR, Ser. Biol. 3:439-45, 1984), GANU (Sava & Giraldi, Cancer Chemother. Pharmacol. 10(3):167-9, 1983), CCNU (Capelli et al., Med., Biol., Environ. 11(1): 111-16, 1983), 5-aminomethyl-2′-deoxyuridine nitrosourea analogues (Shiau, Shih Ta Hsuch Pao (Taipei) 27:681-9, 1982), TA-077 (Fujimoto & Ogawa, Cancer Chemother. Pharmacol. 9(3):134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.):165-74, 1981), thiocolchicine nitrosourea analogues (George, Shih Ta Hsuch Pao (Taipei) 25:355-62, 1980), 2-chloroethyl-nitrosourea (Zeller & Eisenbrand, Oncology 38(1):39-42, 1981), ACNU, (1-(4-amino-2-methyl-5-pyrimidinyl)methyl-3 (2-chloroethyl)-3-nitrosourea hydrochloride) (Shibuya et al., Gan To Kagaku Ryoho 7(8):1393-401, 1980), N-deacetylmethyl thiocolchicine nitrosourea analogues (Lin et al., J. Med. Chem. 23(12):1440-2, 1980), pyridine and piperidine nitrosourea derivatives (Crider et al., J. Med. Chem. 23(8):848-51, 1980), methyl-CCNU (Zimber & Perk, Refu. Vet 35(1):28, 1978), phensuzimide nitrosourea derivatives (Crider et al., J. Med. Chem. 23(3):324-6, 1980), ergoline nitrosourea derivatives (Crider et al., J. Med. Chem. 22(1):32-5, 1979), glucopyranose nitrosourea derivatives (JP 78 95917), 1-(2-chloroethyl)-3-cyclohexyl-1-nitrosourea (Farmer et al., J. Med. Chem. 21(6):514-20, 1978), 4-(3-(2-chloroethyl)-3-nitrosoureid-o)-cis-cyclohexanecarboxylic acid (Drewinko et al., Cancer Treat. Rep. 61(8):J1513-18, 1977), RPCNU (ICIG 1163) (Larnicol et al., Biomedicine 26(3):J176-81, 1977), IOB-252 (Sorodoc et al., Rev. Roum. Med., Virol. 28(1):J 55-61, 1977), 1,3-bis(2-chloroethyl)-1-nitrosourea (BCNU) (Siebert & Eisenbrand, Mutat. Res. 42(11):J45-50, 1977), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaminopterin analogues (DeGraw et al., J. Med. Chem. 40(3) 370-376, 1997), 5-deazaminopterin and 5,10-dideazaminopterin methotrexate analogues (Piper et al, J Med Chem, 40(3):377-384, 1997), indoline moiety-bearing methotrexate derivatives (Matsuoka et al., Chem. Pharm Bull 44(7):1332-1337, 1996), lipophilic amide methotrexate derivatives (Pignatello et al., World Meet. Pharm., Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2), 101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8) 1463-9, 1987), polymeric platinol methotrexate derivative (Carraher et al., Polym Sci Technol. (Plenum), 35(Adv. Biomed. Polym) 311-24, 1987), methotrexate-γ-dimyristoylphophatidylethanolamine (Kinsky et al. Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122 (Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly(L-lysine) methotrexate conjugates (Rosowsky et al., J. Med. Chem. 27(7):888-93, 1984), dilysine and trilysine methotrexate derivates' (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv Exp Med Biol, 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al. J. Med. Chem. 25(7):877-80, 1982), lectin derivatives of methotrexate (Lin et al., JNCI (3):523-8, 1981), polyglutamate methotrexate derivatives (Galivan, Mol. Pharmacol. 17(1): 105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al. Mol Pharmacol 31(3):301-6, 1987), doxifluridine (Matuura et al., Oyo Yakun 29(5) 803-31, 1985), 5′-deoxy-5-fluorouridine (Bollag & Hartmann. Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22(4):391-400, 1979); and Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

Within one preferred embodiment of the invention, the cell cycle inhibitor is paclitaxel, a compound that disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325, 1971) that has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL (Bristol Myers Squibb, New York, N.Y., TAXOTERE (Aventis Pharmaceuticals, France), docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, eg., Schiff et al., Nature 277:665-667, 1979; Long and Fairchild, Cancer Research 54:4355-4361, 1994; Ringel and Horwitz, J. Nat'l Cancer Inst. 83(4):288-291, 1991, Pazdur et al., Cancer Treat. Rev. 19(4):351-386, 1993, WO 94/07882, WO 94/07881; WO 94/07880; WO 94/07876; WO 93/23555, WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxeis, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol, 2′,7-diacetyltaxol; 2′succinyltaxol, 2′-(beta-alanyl)-taxol), 2′gamma-aminobutyryltaxol formate, ethylene glycol derivatives of 2′-succinyltaxol, 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′, 7-di(L-isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′, 7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol, taxol analogues with modified phenylisoserine side chains, TAXOTERE, (N-debenzoyl-N-tert-(butoxycaronyl)-10-deacetyltaxol, and taxanes (e.g., baccatin III, cephalomannine, 10-deacety(baccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfonamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the cell cycle inhibitor is a taxane having the formula (C1):

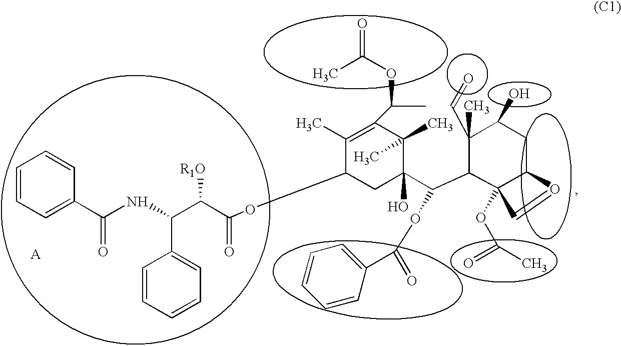

where the gray-highlighted portions may be substituted and the non-highlighted portion is the taxane core A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2):

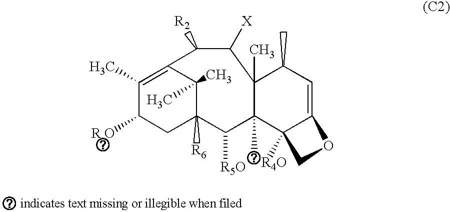

wherein X may be oxygen (paclitaxel), hydrogen (9-deoxy derivatives), thioacyl, or dihydroxyl precursors; R₁ is selected from paclitaxel or TAXOTERE side chains or alkanoyl of the formula (C3)

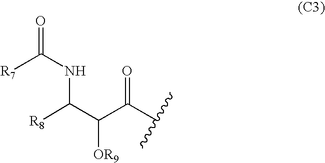

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as cell cycle inhibitors are disclosed in PCT International Patent Application No. WO 93/10076 As disclosed in this publication, the analogue or derivative should have a side chain attached to the taxane nucleus at C₁₃, as shown in the structure below (formula C4), in order to confer antitumor activity to the taxane.

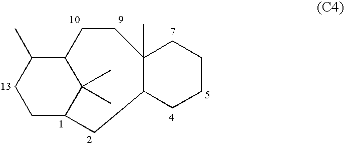

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In one aspect, the taxane-based cell cycle inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a cell cycle inhibitor by acting as an anti-microtubule agent, and more specifically as a stabilizer These compounds have been shown useful in the treatment of proliferative disorders, including: non-small cell (NSC) lung, small cell lung, breast, prostate, cervical, endometrial, head and neck cancers

In another aspect, the anti-microtuble agent (microtubule inhibitor) is albendazole (carbamic acid, [5-(propylthio)-1H-benzimidazol-2-yl]-, methyl ester), LY-355703 (1,4-dioxa-8,11-diazacyclohexadec-13-ene-2,5,9,12-tetrone, 10-[(3-chloro-4-methoxyphenyl)methyl]-6,6-dimethyl-3-(2-methylpropyl)-16-[(1S)-1-[(2S,3R)-3-phenyloxiranyl]ethyl]-, (3S,10R,13E,16S)—), vindesine (vincaleukoblastine, 3-(aminocarbonyl)-O4-deacetyl-3-de(methoxycarbonyl)-), or WAY-174286

In another aspect, the cell cycle inhibitor is a vinca alkaloid. Vinca alkaloids have the following general structure. They are indole-dihydroindole dimers.

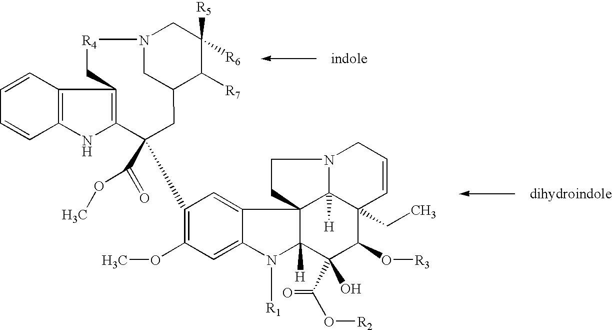

As disclosed in U.S. Pat. Nos. 4,841,045 and 5,030,620, R₁ can be a formyl or methyl group or alternately H. R₁ can also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group, such as maleoyl amino acid. R₃ can also be substituted to form an alkyl ester which may be further substituted R₄ may be —CH₂— or a single bond R₅ and R₆ may be H, OH or a lower alkyl, typically —CH₂CH₃ Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids are vinblastine, vincristine, vincristine sulfate, vindesine, and vinorelbine, having the structures:

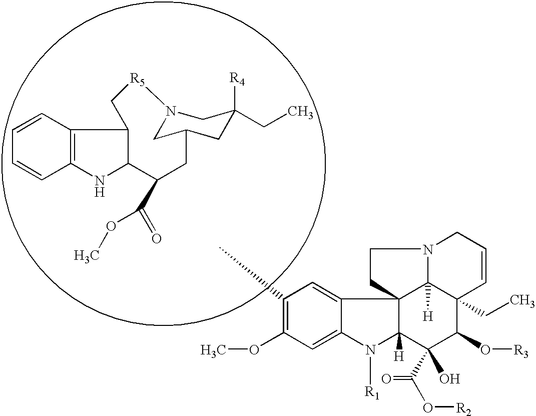

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₂ | CH₃ | H | single bond |

Analogues typically require the side group (shaded area) in order to have activity. These compounds are thought to act as cell cycle inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the cell cycle inhibitor is a camptothecin, or an analog or derivative thereof. Camptothecins have the following general structure

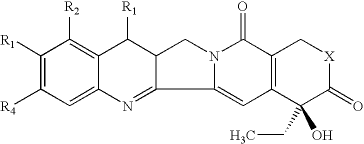

In this structure, X is typically 0, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

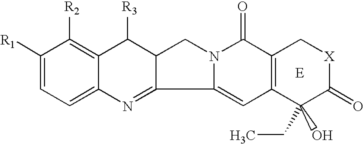

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as cell cycle inhibitors, where they can function as topoisomerase I inhibitors and/or DNA cleavage agents They have been shown useful in the treatment of proliferative disorders, including, for example, NSC lung: small cell lung: and cervical cancers.

In another aspect, the cell cycle inhibitor is a podophyllotoxin, or a derivative or an analogue thereof. Exemplary compounds of this type are etoposide or teniposide, which have the following structures:

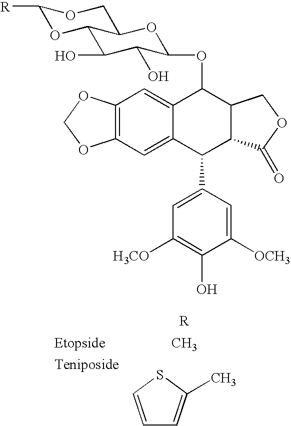

These compounds are thought to function as cell cycle inhibitors by being topoisomerase II inhibitors and/or by DNA cleaving agents. They have been shown useful as antiproliferative agents in, e.g., small cell lung, prostate, and brain cancers, and in retinoblastoma.

Another example of a DNA topoisomerase inhibitor is lurtotecan dihydrochloride (11H-1,4-dioxino[2,3-g]pyrano[3′,4′:6,7]indolizino[1,2-b]quinoline-9,12(8H,14H)-dione, 8-ethyl-2,3-dihydro-8-hydroxy-15-[(4-methyl-1-piperazinyl)methyl]-, dihydrochloride, (S)—).

In another aspect, the cell cycle inhibitor is an anthracycline. Anthracyclines have the following general structure, where the R groups may be a variety of organic groups:

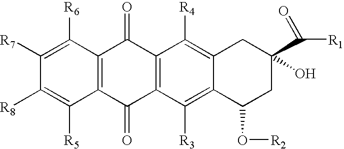

According to U.S. Pat. No. 5,594,158, suitable R groups are R₁ is CH₃ or CH₂OH, R₂ is daunosamine or H, R₃ and R₄ are independently one of OH, NO₂, NH₂, F, Cl, Br, I, CN, H or groups derived from these, R₅₋₇ are all H or R₅ and R₆ are H and R₇ and R₈ are alkyl or halogen, or vice versa R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

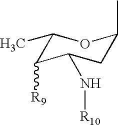

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

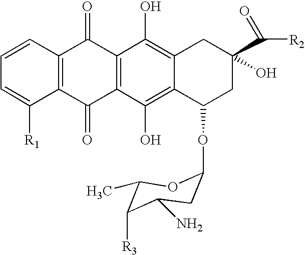

| | R₂ | R₂ | R₃ |
|---|---|---|---|
| Doxorubicin | OCH₃ | CH₂OH | OH out of ring plane |
| Epirubicin | OCH₃ | CH₂OH | OH in ring plane |
| (4 epimer of | | | |
| doxorubicin) | | | |
| Daunorubicin | OCH₃ | CH₃ | OH out of ring plane |
| Idarubicin | H | CH₃ | OH out of ring plane |
| Pirarubicin | OCH₃ | OH | A |
| Zorubicin | OCH₃ | ═N—NHC(O)C₆H₃ | B |
| Carubicin | OH | CH₃ | B |

A

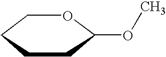

B

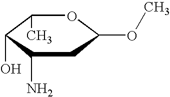

Other suitable anthracyclines are anthramycin, mitoxantrone, menogaril, nogalamycin, aclacinomycin A, olivomycin A, chromomycin A₃, and plicamycin having the structures:

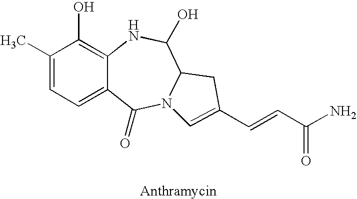

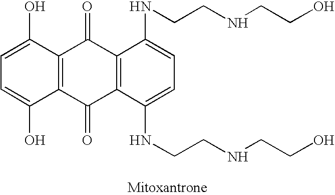

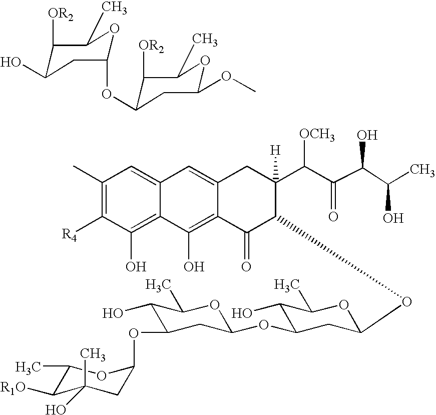

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

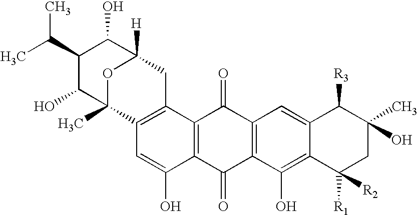

| | R₁ | R₂ | R₃ |
|---|---|---|---|
| Menogaril | H | OCH₃ | H |
| Nogalamycin | O-sugar | H | COOCH₃ |

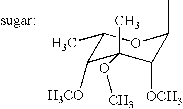

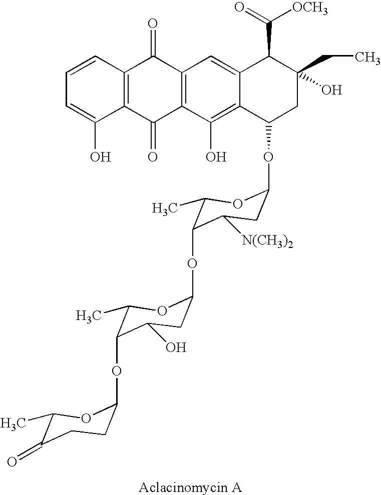

These compounds are thought to function as cell cycle inhibitors by being topoisomerase inhibitors and/or by DNA cleaving agents They have been shown useful in the treatment of proliferative disorders, including small cell lung; breast; endometrial; head and neck; retinoblastoma; liver; bile duct; islet cell; and bladder cancers; and soft tissue sarcoma

In another aspect, the cell cycle inhibitor is a platinum compound. In general, suitable platinum complexes may be of Pt(II) or Pt(IV) and have this basic structure:

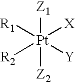

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

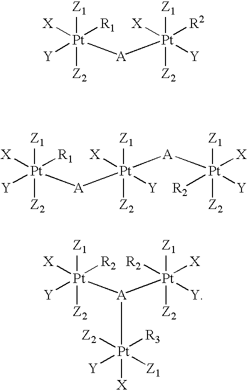

Exemplary platinum compounds are cisplatin, carboplatin, oxaliplatin, and miboplatin having the structures:

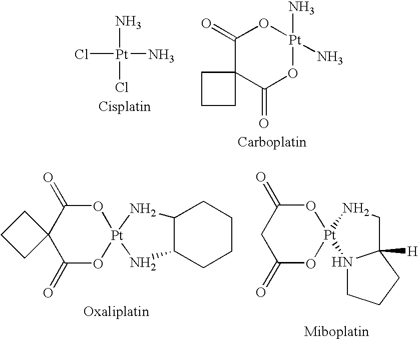

These compounds are thought to function as cell cycle inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal, retinoblastom, liver, bile duct, bladder; penile; and vulvar cancers, and soft tissue sarcoma

In another aspect, the cell cycle inhibitor is a nitrosourea Nitrosourease have the following general structure (C5), where typical R groups are shown below.

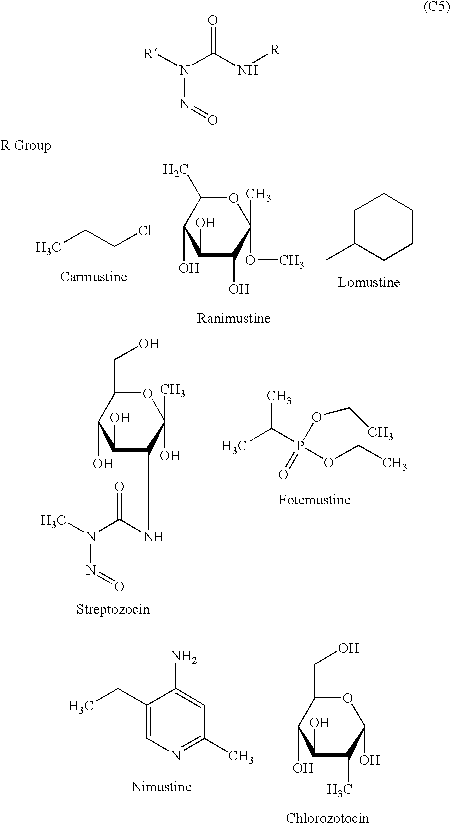

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: -alkylene-N—R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., methyl 2′-(N—(N-(2-chloroethyl)-N-nitroso-carbamoyl)-glycyl)amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylic acid or CONH₂ group.

Exemplary nitrosoureas are BCNU (carmustine), methyl-CCNU (semustine), CCNU (lomustine), ranimustine, nimustine, chlorozotocin, fotemustine, and streptozocin, having the structures:

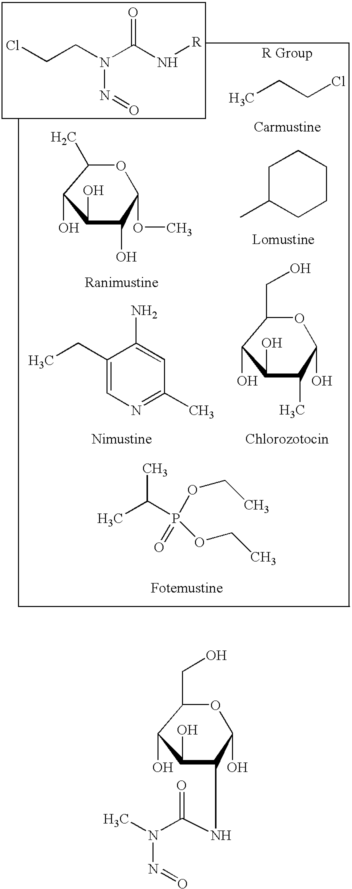

These nitrosourea compounds are thought to function as cell cycle inhibitors by binding to DNA, that is, by functioning as DNA alkylating agents. These cell cycle inhibitors have been shown useful in treating cell proliferative disorders such as, for example, islet cell; small cell lung; melanoma; and brain cancers.

In another aspect, the cell cycle inhibitor is a nitroimidazole, where exemplary nitroimidazoles are metronidazole, benznidazole, etanidazole, and misonidazole, having the structures:

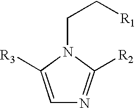

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂OH | NO₂ | H |

Suitable nitroimidazole compounds are disclosed in, e.g., U.S. Pat. Nos. 4,371,540 and 4,462,992.

In another aspect, the cell cycle inhibitor is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

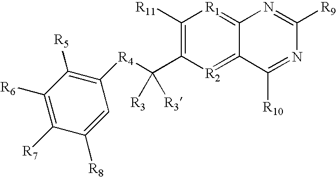

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, eg. CH₃, R₄ may be a single bond or NR, where R is H or alkyl group R_{5,6,8} may be H, OCH₃, or alternately they can be halogens or hydro groups. R₇ is a side chain of the general structure:

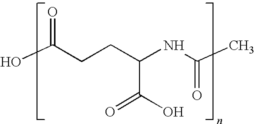

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

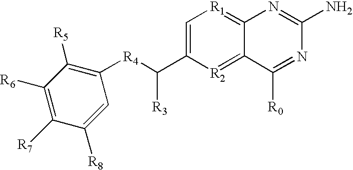

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | N(CH₂CH₃) | H | H | A (n = 1) | H |
| Trimetrexate | NH₂ | N | C(CH₃) | H | NH | H | OCH₃ | OCH₃ | OCH₃ |
| Pteropterin | NH₂ | N | N | H | N(CH₃) | H | H | A (n = 3) | H |
| Denopterin | OH | N | N | CH₃ | N(CH₃) | H | H | A (n = 1) | H |
| Piritrexim | NH₂ | N | C(CH₃)H | single | OCH₃ | H | H | OCH₃ | H |
| | | | | bond | | | | | |

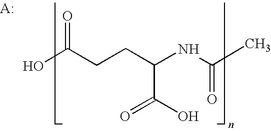

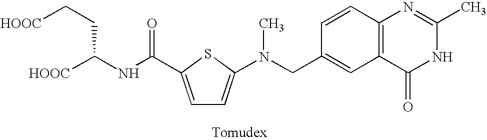

These compounds are thought to function as cell cycle inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the cell cycle inhibitor is a cytidine analogue, such as cytarabine or derivatives or analogues thereof, including enocitabine, FMdC ((E(-2′-deoxy-2′-(fluoromethylene)cytidine), gemcitabine, 5-azacitidine, ancitabine, and 6-azauridine. Exemplary compounds have the structures:

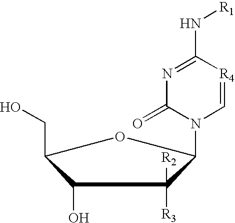

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

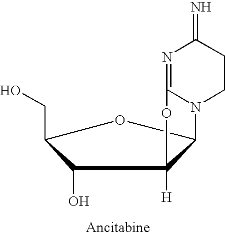

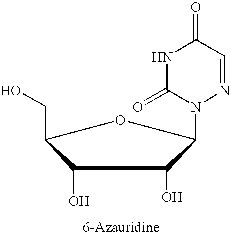

These compounds are thought to function as cell cycle inhibitors as acting as antimetabolites of pyrimidine. These compounds have been shown useful in the treatment of cell proliferative disorders including, for example, pancreatic, breast, cervical, NSC lung, and bile duct cancers.

In another aspect, the cell cycle inhibitor is a pyrimidine analogue. In one aspect, the pyrimidine analogues have the general structure:

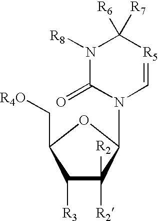

wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆=—NH—R₁ and R₇═H. R₈ is H or R₇ and R₈ together can form a double bond or R₈ can be X, where X is:

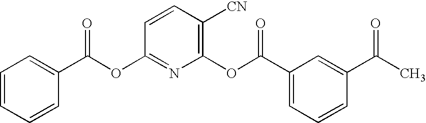

Specific pyrimidine analogues are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the cell cycle inhibitor is a fluoropyrimidine analogue, such as 5-fluorouracil, or an analogue or derivative thereof, including carmofur, doxifluridene, emitefur, tegafur, and floxuridine Exemplary compounds have the structures:

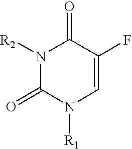

| | R₁ | R₂ |
|---|---|---|
| 5 Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

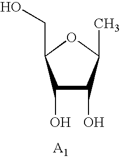

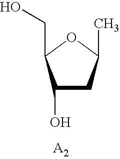

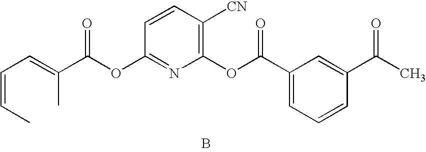

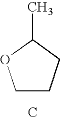

Other suitable fluoropyrimidine analogues include 5-FudR (5-fluoro-deoxyuridine), or an analogue or derivative thereof, including 5-iododeoxyuridine (5-IudR),5-bromodeoxyuridine (5-BudR), fluorouridine triphosphate (5-FUTP), and fluorodeoxyuridine monophosphate (5-dFUMP). Exemplary compounds have the structures:

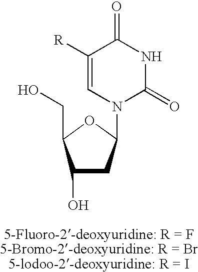

These compounds are thought to function as cell cycle inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the cell cycle inhibitor is a purine analogue. Purine analogues have the following general structure.

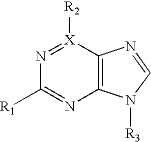

wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R2 is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogues of the type shown in the formula.

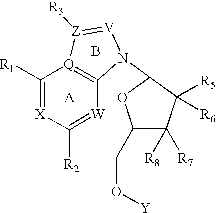

wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogues include 6-mercaptopurine, thiguanosine, thiamiprine, cladribine, fludaribine, tubercidin, puromycin, pentoxyfilline; where these compounds may optionally be phosphorylated. Exemplary compounds have the structures:

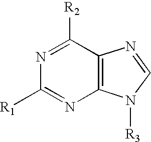

| | R₁ | R₂ | R₃ |
|---|---|---|---|
| 6-Mercaptopurine | H | SH | H |
| Thioguanosine | NH₂ | SH | B₁ |
| Thiamiprine | NH₂ | A | H |
| Cladribine | Cl | NH₂ | B₂ |
| Fludarabine | F | NH₂ | B₃ |
| Puromycin | H | N(CH₃)₂ | B₄ |
| Tubercidin | H | NH₂ | B₁ |

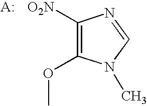

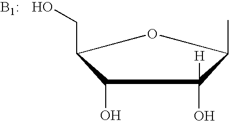

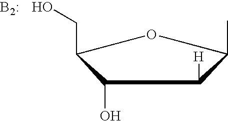

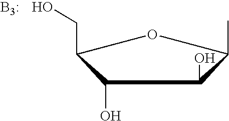

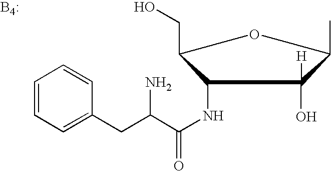

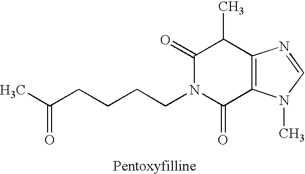

These compounds are thought to function as cell cycle inhibitors by serving as antimetabolites of purine.

In another aspect, the cell cycle inhibitor is a nitrogen mustard. Many suitable nitrogen mustards are known and are suitably used as a cell cycle inhibitor in the present invention. Suitable nitrogen mustards are also known as cyclophosphamides.

A preferred nitrogen mustard has the general structure:

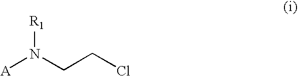

### Where A is

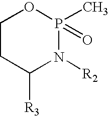

or —CH₃ or other alkane, or chloronated alkane, typically CH₂CH(CH₃)Cl, or a polycyclic group such as B, or a substituted phenyl such as C or a heterocyclic group such as D.

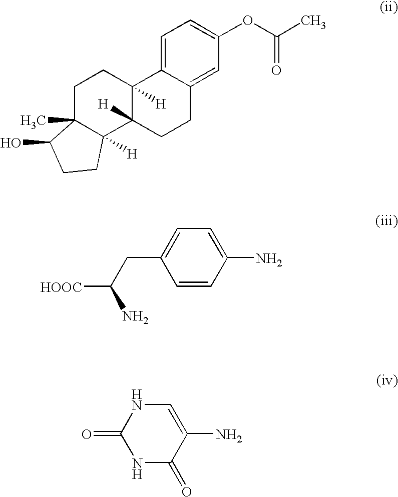

Examples of suitable nitrogen mustards are disclosed in U.S. Pat. No. 3,808,297, wherein A is:

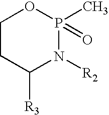

R₁₋₂ are H or CH₂CH₂Cl R₃ is H or oxygen-containing groups such as hydroperoxy; and R₄ can be alkyl, aryl, heterocyclic

The cyclic moiety need not be intact See, e.g., U.S. Pat. Nos. 5,472,956, 4,908,356, 4,841,085 that describe the following type of structure.

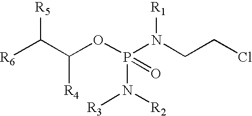

wherein R₁ is H or CH₂CH₂Cl, and R₂₋₆ are various substituent groups.

Exemplary nitrogen mustards include methylchloroethamine, and analogues or derivatives thereof, including methylchloroethamine oxide hydrochloride, novembichin, and mannomustine (a halogenated sugar). Exemplary compounds have the structures:

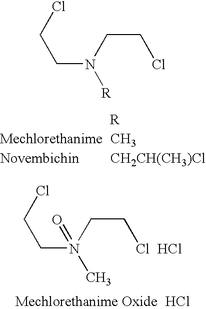

The nitrogen mustard may be cyclophosphamide, ifosfamide, perfosfamide, or torofosfamide, where these compounds have the structures:

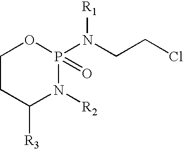

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The nitrogen mustard may be estramustine or an analogue or derivative thereof, including phenesterine, prednimustine, and estramustine PO₄ Thus, suitable nitrogen mustard type cell cycle inhibitors of the present invention have the structures.

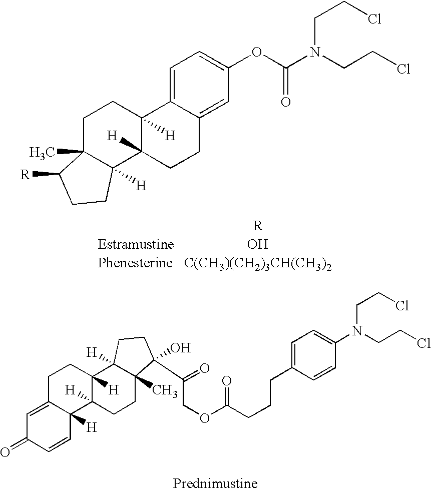

The nitrogen mustard may be chlorambucil, or an analogue or derivative thereof, including melphalan and chlormaphazine. Thus, suitable nitrogen mustard type cell cycle inhibitors of the present invention have the structures:

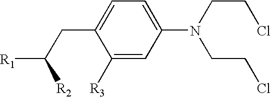

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The nitrogen mustard may be uracil mustard, which has the structure:

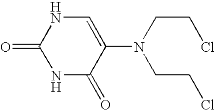

The nitrogen mustards are thought to function as cell cycle inhibitors by serving as alkylating agents for DNA. Nitrogen mustards have been shown useful in the treatment of cell proliferative disorders including, for example, small cell lung, breast, cervical, head and neck, prostate, retinoblastoma, and soft tissue sarcoma.

The cell cycle inhibitor of the present invention may be a hydroxyurea. Hydroxyureas have the following general structure:

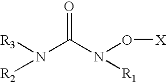

Suitable hydroxyureas are disclosed in, for example, U.S. Pat. No. 6,080,874, wherein R₁ is:

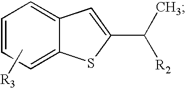

and R₂ is an alkyl group having 14 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable hydroxyureas are disclosed in, eg. U.S. Pat. No. 5,066,658, wherein R₂ and R₃ together with the adjacent nitrogen form:

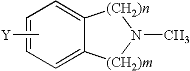

wherein m is 1 or 2, n is 0-2 and Y is an alkyl group.

In one aspect, the hydroxy urea has the structure:

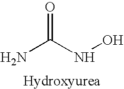

Hydroxyureas are thought to function as cell cycle inhibitors by serving to inhibit DNA synthesis.

In another aspect, the cell cycle inhibitor is a mytomicin, such as mitomycin C, or an analogue or derivative thereof, such as porphyromycin. Exemplary compounds have the structures:

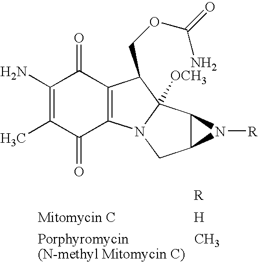

These compounds are thought to function as cell cycle inhibitors by serving as DNA alkylating agents. Mitomycins have been shown useful in the treatment of cell proliferative disorders such as, for example, esophageal, liver, bladder, and breast cancers.

In another aspect, the cell cycle inhibitor is an alkyl sulfonate, such as busulfan, or an analogue or derivative thereof, such as treosulfan, improsulfan, piposultan, and pipobroman Exemplary compounds have the structures

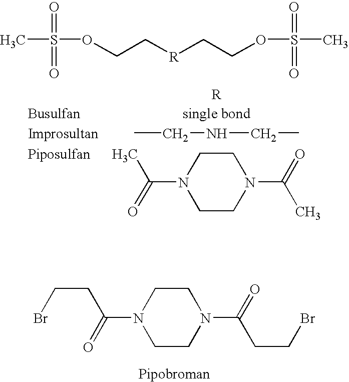

These compounds are thought to function as cell cycle inhibitors by serving as DNA alkylating agents.

In another aspect, the cell cycle inhibitor is a benzamide. In yet another aspect, the cell cycle inhibitor is a nicotinamide. These compounds have the basic structure:

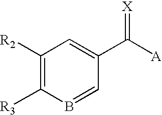

wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-ethyl (see, e.g., U.S. Pat. No. 5,215,738).

Suitable benzamide compounds have the structures

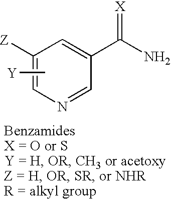

where additional compounds are disclosed in U.S. Pat. No. 5,215,738, (listing some 32 compounds).

Suitable nicotinamide compounds have the structures:

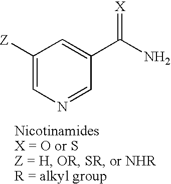

where additional compounds are disclosed in U.S. Pat. No. 5,215,738,

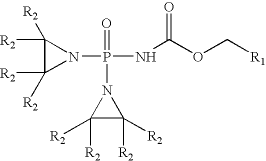

| | R₁ | R₂ |
|---|---|---|
| Benzodepa | phenyl | H |
| Meturedepa | CH₃ | CH₃ |
| Uredepa | CH₃ | H |

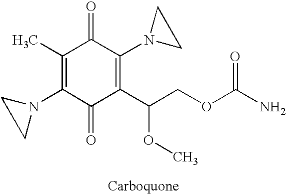

In another aspect, the cell cycle inhibitor is a halogenated sugar, such as mitolactol, or an analogue or derivative thereof, including mitobronitol and mannomustine. Exemplary compounds have the structures:

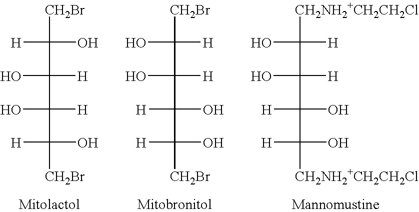

In another aspect, the cell cycle inhibitor is a diazo compound, such as azaserine, or an analogue or derivative thereof, including 6-diazo-5-oxo-L-norleucine and 5-diazouracil (also a pyrimidine analog). Exemplary compounds have the structures:

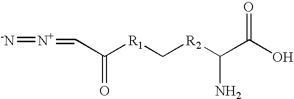

| | R₁ | R₂ |
|---|---|---|
| Azaserine | O | single bond |
| 6-diazo-5-oxo- | single bond | CH₂ |
| L-norleucine | | |

Other compounds that may serve as cell cycle inhibitors according to the present invention are pazelliptine; wortmannin; metoclopramide; RSU; buthionine sulfoxime; tumeric; curcumin; AG337, a thymidylate synthase inhibitor; levamisole; lentinan, a polysaccharide; razoxane, an EDTA analogue; indomethacin; chlorpromazine; α and β interferon; MnBOPP; gadolinium texaphyrin; 4-amino-1,8-naphthalimide; staurosporine derivative of CGP; and SR-2508.

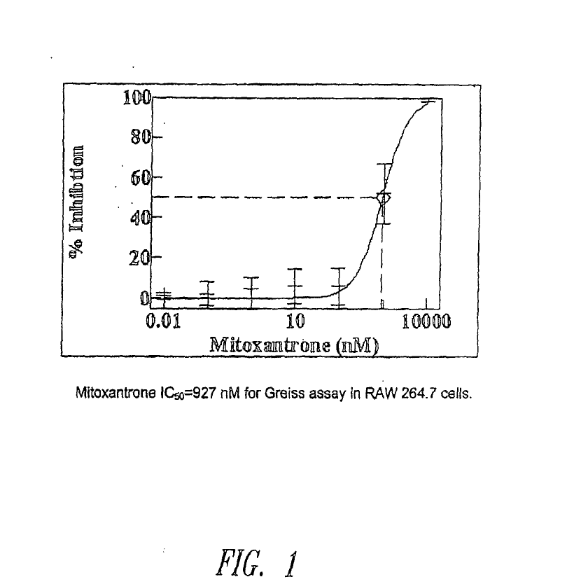

Thus, in one aspect, the cell cycle inhibitor is a DNA alylating agent. In another aspect, the cell cycle inhibitor is an anti-microtubule agent. In another aspect, the cell cycle inhibitor is a topoisomerase inhibitor. In another aspect, the cell cycle inhibitor is a DNA cleaving agent. In another aspect, the cell cycle inhibitor is an antimetabolite. In another aspect, the cell cycle inhibitor functions by inhibiting adenosine deaminase (e.g., as a purine analogue). In another aspect, the cell cycle inhibitor functions by inhibiting purine ring synthesis and/or as a nucleotide interconversion inhibitor (eg. as a purine analogue such as mercaptopurine) In another aspect, the cell cycle inhibitor functions by inhibiting dihydrofolate reduction and/or as a thymidine monophosphate block (e.g., methotrexate). In another aspect, the cell cycle inhibitor functions by causing DNA damage (e.g. bleomycin). In another aspect, the cell cycle inhibitor functions as a DNA intercalation agent and/or RNA synthesis inhibition (e.g., doxorubicin, aclarubicin, or detorubicin (acetic acid, diethoxy-, 2-[4-[(3-amino-2,3,6-trideoxy-alpha-L-lyxo-hexopyranosyl)oxy]-1,2,3,4,6,11-hexahydro-2,5,12-trihydroxy-7-methoxy-6,11-dioxo-2-naphthacenyl]-2-oxoethyl ester, (2S-cis)-)). In another aspect, the cell cycle inhibitor functions by inhibiting pyrimidine synthesis (e.g., N-phosphonoacetyl-L-aspartate). In another aspect, the cell cycle inhibitor functions by inhibiting ribonucleotides (e.g., hydroxyurea). In another aspect, the cell cycle inhibitor functions by inhibiting thymidine monophosphate (e.g., 5-fluorouracil). In another aspect, the cell cycle inhibitor functions by inhibiting DNA synthesis (e.g., cytarabine). In another aspect, the cell cycle inhibitor functions by causing DNA adduct formation (e.g., platinum compounds). In another aspect, the cell cycle inhibitor functions by inhibiting protein synthesis (e.g., L-asparginase). In another aspect, the cell cycle inhibitor functions by inhibiting microtubule function (e.g., taxanes). In another aspect, the cell cycle inhibitor acts at one or more of the steps in the biological pathway shown in FIG. 1.

Additional cell cycle inhibitors useful in the present invention, as well as a discussion of the mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066.658; 5,166,149; 5,190,929; 5,215,738; 5,292,731; 5,380.897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561.

In another embodiment, the cell-cycle inhibitor is camptothecin, mitoxantrone, etoposide, 5-fluorouracil, doxorubicin, methotrexate, peloruside A, mitomycin C, or a CDK-2 inhibitor or an analogue or derivative of any member of the class of listed compounds.

In another embodiment, the cell-cycle inhibitor is HTI-286, plicamycin; or mithramycin, or an analogue or derivative thereof.

Other examples of cell cycle inhibitors also include, e.g., 7-hexanoyltaxol (QP-2), cytochalasin A, lantrunculin D, actinomycin-D, Ro-31-7453 (3-(6-nitro-1-methyl-3-indolyl)-4-(1-methyl-3-indolyl)pyrrole-2,5-dione), PNU-151807, brostallicin, C2-ceramide, cytarabine ocfosfate (2(1H)-pyrimidinone, 4-amino-1-(5-O-(hydroxy(octadecyloxy)phosphinyl)-β-D-arabinofuranosyl)-, monosodium salt), paclitaxel (5β,20-epoxy-1,2 alpha,4,7β,10β,13 alpha-hexahydroxytax-11-en-9-one-4,10-diacetate-2-benzoate-13-(alpha-phenylhippurate)), doxorubicin (5,12-naphthacenedione, 10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-, (8S)-cis-), daunorubicin (5,12-naphthacenedione, 8-acetyl-10-((3-amino-2,3,6-trideoxy-alpha-L-lyxo-hexopyranosyl)oxy)-7,8,9,10-tetrahydro-6,8,11-trihydroxy-1-methoxy-, (8S-cis)-), gemcitabine hydrochloride (cytidine, 2′-deoxy-2′,2′-difluoro-,monohydrochloride), nitacrine (1,3-propanediamine, N,N-dimethyl-N′-(1-nitro-9-acridinyl)-), carboplatin (platinum, diammine(1,1-cyclobutanedicarboxylato(2-))-, (SP-4-2)-), altretamine (1,3,5-triazine-2,4,6-triamine, N,N,N′,N′,N″,N″-hexamethyl-), teniposide (furo(3′,4′:6,7)naphtho(2,3-d)-1,3-dioxol-6(5aH)-one, 5,8,8a,9-tetrahydro-5-(4-hydroxy-3,5-dimethoxyphenyl)-9-((4,6-O-(2-thienylmethylene)-β-D-glucopyranosyl)oxy)-, (5R-(5alpha,5aβ,8aAlpha,9β(R*)))-), eptaplatin (platinum, ((4R,5R)-2-(1-methylethyl)-1,3-dioxolane-4,5-dimethanamine-kappa N4,kappa N5)(propanedioato(2-)-kappa O1, kappa O3)-, (SP-4-2)-), amrubicin hydrochloride (5,12-naphthacenedione, 9-acetyl-9-amino-7-((2-deoxy-βD-erythro-pentopyranosyl)oxy)-7,8,9,10-tetrahydro-6,11-dihydroxy-, hydrochloride, (7S-cis)-), ifosfamide (2H-1,3,2-oxazaphosphorin-2-amine, N,3-bis(2-chloroethyl)tetrahydro-,2-oxide), cladribine (adenosine, 2-chloro-2′-deoxy-), mitobronitol (D-mannitol, 1,6-dibromo-1,6-dideoxy-), fludaribine phosphate (9H-purin-6-amine, 2-fluoro-9-(5-O-phosphono-β-D-arabinofuranosyl)-), enocitabine (docosanamide, N-(1-β-D-arabinofuranosyl-1,2-dihydro-2-oxo-4-pyrimidinyl)-), vindesine (vincaleukoblastine, 3-(aminocarbonyl)-O4-deacetyl-3-de(methoxycarbonyl)-), idarubicin (5,12-naphthacenedione, 9-acetyl-7-((3-amino-2,3,6-trideoxy-alpha-L-lyxo-hexopyranosyl)oxy)-7,8,9,10-tetrahydro-6,9,11-trihydroxy-, (7S-cis)-), zinostatin (neocarzinostatin), vincristine (vincaleukoblastine, 22-oxo-), tegafur (2,4(1H,3H)-pyrimidinedione, 5-fluoro-1-(tetrahydro-2-furanyl)-), razoxane (2,6-piperazinedione, 4,4′-(1-methyl-1,2-ethanediyl)bis-), methotrexate (L-glutamic acid, N-(4-(((2,4-diamino-6-pteridinyl)methyl)methylamino)benzoyl)-), raltitrexed (L-glutamic acid, N-((5-(((1,4-dihydro-2-methyl-4-oxo-6-quinazolinyl)methyl)methylamino)-2-thienyl)carbonyl)-), oxaliplatin (platinum, (1,2-cyclohexanediamine-N,N′)(ethanedioato(2-)-O,O′)-, (SP-4-2-(1R-trans))-), doxifluridine (uridine, 5′-deoxy-5-fluoro-), mitolactol (galactitol, 1,6-dibromo-1,6-dideoxy-), piraubicin (5,12-naphthacenedione, 10-((3-amino-2,3,6-trideoxy-4-O-(tetrahydro-2H-pyran-2-yl)-alpha-L-lyxo-hexopyranosyl)oxy)-7,8,9,10-tetrahydro-6,8,11-trihydroxy-8-(hydroxyacetyl)-1-methoxy-, (8S-(8 alpha, 10 alpha(S*)))-), docetaxel ((2R,3S)—N-carboxy-3-phenylisoserine, N-tert-butyl ester, 13-ester with 5β,20-epoxy-1,2 alpha,4,7β,10β,13 alpha-hexahydroxytax-11-en-9-one 4-acetate 2-benzoate-), capecitabine (cytidine, 5-deoxy-5-fluoro-N-((pentyloxy)carbonyl)-), cytarabine (2(1H)-pyrimidone, 4-amino-1-β-D-arabino furanosyl-), valrubicin (pentanoic acid, 2-(1,2,3,4,6,11-hexahydro-2,5,12-trihydroxy-7-methoxy-6,11-dioxo-4-((2,3,6-trideoxy-3-((trifluoroacetyl)amino)-alpha-L-lyxo-hexopyranosyl)oxy)-2-naphthacenyl)-2-oxoethyl ester (2S-cis)-), trofosfamide (3-2-(chloroethyl)-2-(bis(2-chloroethyl)amino)tetrahydro-2H-1,3,2-oxazaphosphorin 2-oxide), prednimustine (pregna-1,4-diene-3,20-dione, 21-(4-(4-(bis(2-chloroethyl)amino)phenyl)-1-oxobutoxy)-11,17-dihydroxy-, (11β)-), lomustine (Urea, N-(2-chloroethyl)-N′-cyclohexyl-N-nitroso-), epirubicin (5,12-naphthacenedione, 10-((3-amino-2,3,6-trideoxy-alpha-L-arabino-hexopyranosyl)oxy)-7,8,9,10-tetrahydro-6,8,11-trihydroxy-8-(hydroxyacetyl)-1-methoxy-, (8S-cis)-), or an analogue or derivative thereof).

In certain embodiments, the fibrosis-inhibiting compound is a cell cycle inhibitor (e.g., SNS-595 (Sunesis) or an analogue or derivative thereof).

In certain embodiments, the cell cycle inhibitor is an anti-microtubule agent (e.g., synthadotin, or an analogue or derivative thereof).

In certain embodiments, cell cycle inhibitor is a microtubule stimulant (e.g., KRX-0403, or an analogue or derivative thereof).

5. Cyclin Dependent Protein Kinase Inhibitors

In another embodiment, the pharmacologically active compound is a cyclin dependent protein kinase inhibitor (e.g., R-roscovitine, CYC-101, CYC-103, CYC-400, MX-7065, alvocidib (4H-1-Benzopyran-4-one, 2-(2-chlorophenyl)-5,7-dihydroxy-8-(3-hydroxy-1-methyl-4-piperidinyl)-, cis-(−)-), SU-9516, AG-12275, PD-0166285, CGP-79807, fascaplysin, GW-8510 (benzenesulfonamide, 4-(((Z)-(6,7-dihydro-7-oxo-8H-pyrrolo[2,3-g]benzothiazol-8-ylidene)methyl)amino)-N-(3-hydroxy-2,2-dimethylpropyl)-), GW491619, Indirubin 3′ monoxime, GW8510, AZD-5438, ZK-CDK or an analogue or derivative thereof).

In another embodiment, the fibrosis-inhibiting compound is a cyclin dependent kinase (CDK) inhibitor. In certain embodiments, the cyclin dependent kinase inhibitor is a CDK-1 inhibitor. In certain embodiments, the cyclin dependent kinase inhibitor is a CDK-2 inhibitor. In certain embodiments, the cyclin dependent kinase inhibitor is a CDK-4 inhibitor In certain embodiments, the cyclin dependent kinase inhibitor is a CDK-6 inhibitor Representative examples of cyclin dependent kinase inhibitors include CAK1 inhibitors from GPC Biotech and Bristol-Myers Squibb. RGB-286199 (GPC Biotech), or an analogue or derivative thereof.

Additional exemplary cyclin dependent protein kinase inhibitors include an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No 199986-75-9) (CV Therapeutics), and an analogue or derivative thereof.

6. EGF (Epidermal Growth Factor) Receptor Kinase Inhibitors

In another embodiment, the pharmacologically active compound is an EGF (epidermal growth factor) kinase inhibitor (e.g., erlotinib (4-quinazolinamine, N-(3-ethynylphenyl)-6,7-bis(2-methoxyethoxy)-, monohydrochloride), erbstatin, BIBX-1382, gefitinib (4-quinazolinamine, N-(3-chloro-4-fluorophenyl)-7-methoxy-6-(3-(4-morpholinyl)propoxy)), or an analogue or derivative thereof).

7. Elastase Inhibitors

In another embodiment, the pharmacologically active compound is an elastase inhibitor (e.g., ONO-6818, sivelestat sodium hydrate (glycine, N-(2-(((4-(2,2-dimethyl-1-oxopropoxy)phenyl)sulfonyl)amino)benzoyl)-), erdosteine (acetic acid, ((2-oxo-2-((tetrahydro-2-oxo-3-thienyl)amino)ethyl)thio)-), MDL-100948A, MDL-104238 (N-(4-(4-morpholinylcarbonyl)benzoyl)-L-valyl-N′-(3,3,4,4,4-pentafluoro-1-(1-methylethyl)-2-oxobutyl)-L-2-azetamide), MDL-27324 (L-prolinamide, N-((5-(dimethylamino)-1-naphthalenyl)sulfonyl)-L-alanyl-L-alanyl-N-(3,3,3-trifluoro-1-(1-methylethyl)-2-oxopropyl)-, (S)). SR-26831 (thieno(3,2-c)pyridinium, 5-((2-chlorophenyl)methyl)-2-(2,2-dimethyl-1-oxopropoxy)-4,5,6,7-tetrahydro-5-hydroxy-), Win-68794, Win-63110, SSR-69071 (2-(9(2-piperidinoethoxy)-4-oxo-4H-pyrido(1,2-a)pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (N alpha-(1-adamantanesulphonyl)-N-(4-carboxybenzoyl)-L-lysyl-alanyl-L-valinal), R-665, FCE-28204, ((6R,7R)-2-(benzoyloxy)-7-methoxy-3-methyl-4-pivaloyl-3-cephem 1,1-dioxide), 1,2-benzisothiazol-3(2H)-one, 2-(2,4-dinitrophenyl)-, 1,1-dioxide, L-658758 (L-proline, 1-((3-((acetyloxy)methyl)-7-methoxy-8-oxo-5-thia-1-azabicyclo(4.2.0)oct-2-en-2-yl)carbonyl)-, S,S-dioxide, (6R-cis)-), L-659286 (pyrrolidine, 1-((7-methoxy-8-oxo-3-(((1,2,5,6-tetrahydro-2-methyl-5,6-dioxo-1,2,4-triazin-3-yl)thio)methyl)-5-thia-1-azabicyclo(4.2.0)oct-2-en-2-yl)carbonyl)-, S,S-dioxide, (6R-cis)-), L-680833 (benzeneacetic acid, 4-((3,3-diethyl-1-(((1-(4-methylphenyl)butyl)amino)carbonyl)-4-oxo-2-azetidinyl)oxy)-, (S—(R*,S*))-), FK-706 (L-prolinamide, N-[4-[[(carboxymethyl)amino]carbonyl]benzoyl]-L-valyl-N-[3,3,3-trifluoro-1-(1-methylethyl)-2-oxopropyl]-, monosodium salt), Roche R-665, or an analogue or derivative thereof).

8. Factor Xa Inhibitors

In another embodiment, the pharmacologically active compound is a factor Xa inhibitor (e.g., CY-222, fondaparinux sodium (alpha-D-g lucopyranoside, methyl O-2-deoxy-6-O-sulfo-2-(sulfoamino)-alpha-D-glucopyranosyl-(1-4)-O-β-D-glucopyranuronosyl-(1-4)-O-2-deoxy-3,6-di-O-sulfo-2-(sulfoamino)-alpha-D-glucopyranosyl-(1-4)-O-2-O-sulfo-alpha-L-idopyranuronosyl-(14)-2-deoxy-2-(sulfoamino)-, 6-(hydrogen sulfate)), danaparoid sodium, or an analogue or derivative thereof).

9. Farnesyltransferase Inhibitors

In another embodiment, the pharmacologically active compound is a farnesyltransferase inhibitor (e.g., dichlorobenzoprim (2,4-diamino-5-(4-(3,4-dichlorobenzylamino)-3-nitrophenyl)-6-ethylpyrimidine), B-581, B-956 (N-(8(R)-amino-2(S)-benzyl-5(S)-isopropyl-9-sulfanyl-3(Z),6(E)-nonadienoyl)-L-methionine), OSI-754, perillyl alcohol (1-cyclohexene-1-methanol, 4-(1-methylethenyl)-, RPR-114334, lonafarnib (1-piperidinecarboxamide, 4-(2-(4-((11R)-3,10-dibromo-8-chloro-6,11-dihydro-5H-benzo(5,6)cyclohepta(1,2-b)pyridin-11-yl)-1-piperidinyl)-2-oxoethyl)-), Sch-48755, Sch-226374, (7,8-dichloro-5H-dibenzo(b,e)(1,4)diazepin-11-yl)-pyridin-3-ylmethylamine, J-104126, L-639749, L-731734 (pentanamide, 2-((2-((2-amino-3-mercaptopropyl)amino)-3-methylpentyl)amino)-3-methyl-N-(tetrahydro-2-oxo-3-furanyl)-, (3S-(3R*(2R*(2R*(S*),3S*),3R*)))-), L-744832 (butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))-), L-745631 (1-piperazinepropanethiol, 1-amino-2-(2-methoxyethyl)-4-(1-naphthalenylcarbonyl)-, (βR,2S)—), N-acetyl-N-naphthylmethyl-2(S)-((1-(4-cyanobenzyl)-1H-imidazol-5-yl)acetyl)amino-3(S)-methylpentamine, (2alpha)-2-hydroxy-24,25-dihydroxylanost-8-en-3-one, BMS-316810, UCF-1-C (2,4-decadienamide, N-(5-hydroxy-5-(7-((2-hydroxy-5-oxo-1-cyclopenten-1-yl)amino-oxo-1,3,5-heptatrienyl)-2-oxo-7-oxabicyclo(4.1.0)hept-3-en-3-yl)-2,4,6-trimethyl-, (1S-(1alpha,3(2E,4E,6S*),5 alpha, 5(1E,3E,5E), 6 alpha))-), UCF-116-B, ARGLABIN (3H-oxireno[8,8a]azuleno[4,5-b]furan-8(4aH)-one, 5,6,6a,7,9a,9b-hexahydro-1,4-a-dimethyl-7-methylene-, (3aR,4aS,6aS,9aS,9b)-) from ARGLABIN—Paracure, Inc. (Virginia Beach, Va.), or an analogue or derivative thereof).

10. Fibrinogen Antagonists

In another embodiment, the pharmacologically active compound is a fibrinogen antagonist (e.g., 2(S)-((p-toluenesulfonyl)amino)-3-(((5,6,7,8,-tetrahydro-4-oxo-5-(2-(piperidin-4-yl)ethyl)-4H-pyrazolo-(1,5-a)(1,4)diazepin-2-yl)carbonyl)-amino)propionic acid, streptokinase (kinase (enzyme-activating), strepto-), urokinase (kinase (enzyme-activating), uro-), plasminogen activator, pamiteplase, monteplase, heberkinase, anistreplase, alteplase, pro-urokinase, picotamide (1,3-benzenedicarboxamide, 4-methoxy-N,N′-bis(3-pyridinylmethyl)-), or an analogue or derivative thereof).

11. Guanylate Cyclase Stimulants

In another embodiment, the pharmacologically active compound is a guanylate cyclase stimulant (e.g., isosorbide-5-mononitrate (D-glucitol, 1,4:3,6-dianhydro-, 5-nitrate), or an analogue or derivative thereof).

12. Heat Shock Protein 90 Antagonists

In another embodiment, the pharmacologically active compound is a heat shock protein 90 antagonist (e.g., geldanamycin; NSC-33050 (17-allylaminogeldanamycin), rifabutin (rifamycin XIV, 1′,4-didehydro-1-deoxy-1,4-dihydro-5′-(2-methylpropyl)-1-oxo-), 17AAG, or an analogue or derivative thereof).

13. HMGCoA Reductase Inhibitors

In another embodiment, the pharmacologically active compound is an HMGCoA reductase inhibitor (e.g., BCP-671, BB-476, fluvastatin (6-heptenoic acid, 7-(3-(4-fluorophenyl)-1-(1-methylethyl)-1H-indol-2-yl)-3,5-dihydroxy-, monosodium salt, (R*,S*-(E))-(±)-), dalvastatin (2H-pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4alpha,6β(E))-(+/−)-), glenvastatin (2H-pyran-2-one, 6-(2-(4-(4-fluorophenyl)-2-(1-methylethyl)-6-phenyl-3-pyridinyl)ethenyl)tetrahydro-4-hydroxy-, (4R-(4alpha,6β(E)))-), S-2468, N-(1-oxododecyl)-4Alpha,10-dimethyl-8-aza-trans-decal-3′-ol, atorvastatin calcium (1H-Pyrrole-1-heptanoic acid, 2-(4-fluorophenyl)-β,delta-dihydroxy-5-(1-methylethyl)-3-phenyl-4-((phenylamino)carbonyl)-, calcium salt (R—(R*,R*))-). CP-83101 (6,8-nonadienoic acid, 3,5-dihydroxy-9,9-diphenyl-, methyl ester, (R*,S*-(E))-(+/−)-), pravastatin (1-naphthaleneheptanoic acid, 1,2,6,7,8,8a-hexahydro-β,delta,6-trihydroxy-2-methyl-8-(2-methyl-1-oxobutoxy)-, monosodium salt, (1S-(1 alpha(βS*,deltaS*),2 alpha.6 alpha,8β(R*),8a alpha))-), U-20685, pitavastatin (6-heptenoic acid, 7-(2-cyclopropyl-4-(4-fluorophenyl)-3-quinolinyl)-3,5-dihydroxy-, calcium salt (2.1), (S—(R*,S*-(E)))-), N-((1-methylpropyl)carbonyl)-8-(2-(tetrahydro-4-hydroxy-6-oxo-2H-pyran-2-yl)ethyl)-perhydro-isoquinoline, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha, 4a alpha,7β,8β(2S*,4S*),8aβ))-), HBS-107, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyL ester(1 alpha(R*), 3 alpha,4a alpha,7β,8β(2S*,4S*),8aβ))-), L-669262 (butanoic acid, 2,2-dimethyl-, 1,2,6,7,8,8a-hexahydro-3,7-dimethyl-6-oxo-8-(2-(tetrahydro-4-hydroxy-6-oxo-2H-pyran-2-yl)ethyl)-1-naphthalenyl(1S-(1Alpha,7β,8β(2S*,4S*),8aβ))-), simvastatin (butanoic acid, 2,2-dimethyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1alpha, 3alpha,7β,8β(2S*,4S*),8aβ))-), rosuvastatin calcium (6-heptenoic acid, 7-(4-(4-fluorophenyl)-6-(1-methylethyl)-2-(methyl(methylsulfonyl)amino)-5-pyrimdinyl)-3,5-dihydroxy-calcium salt (2:1) (S—(R*, S*-(E)))), meglutol (2-hydroxy-2-methyl-1,3-propandicarboxylic acid), lovastatin (butanoic acid, 2-methyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1 alpha.(R*),3 alpha,7β,8β(2S*,4S*),8aβ))-), or an analogue or derivative thereof).

14. Hydroorotate Dehydrogenase Inhibitors

In another embodiment, the pharmacologically active compound is a hydroorotate dehydrogenase inhibitor (e.g., leflunomide (4-isoxazolecarboxamide, 5-methyl-N (4-(trlfluoromethyl)phenyl)-), laflunimus (2-propenamide, 2-cyano-3-cyclopropyl-3-hydroxy-N-(3-methyl-4(trifluoromethyl)phenyl)-, (Z)-), or atovaquone (1,4-naphthalenedione, 2-[4-(4-chlorophenyl)cyclohexyl]-3-hydroxy-, trans-, or an analogue or derivative thereof).

15. IKK2 Inhibitors

In another embodiment, the pharmacologically active compound is an IKK2 inhibitor (e.g., MLN-120B, SPC-839, or an analogue or derivative thereof).

16. IL-1, ICE and IRAK Antagonists

In another embodiment, the pharmacologically active compound is an IL-1, ICE or an IRAK antagonist (e.g., E-5090 (2-propenoic acid, 3-(5-ethyl-4-hydroxy-3-methoxy-1-naphthalenyl)-2-methyl-, (Z)-), CH-164, CH-172, CH-490, AMG-719, iguratimod (N-(3-(formylamino)-4-oxo-6-phenoxy-4H-chromen-7-yl)methanesuffonamide), AV94-88, pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)—), (2S-cis)-5-(benzyloxycarbonylamino-1,2,4,5,6,7-hexahydro-4-(oxoazepino(3,2,1-hi)indole-2-carbonyl)-amino)-4-oxobutanoic acid, AVE-9488, esonarimod (benzenebutanoic acid, alpha-((acetylthio)methyl)-4-methyl-gamma-oxo-), pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)—), tranexamic acid (cyclohexanecarboxylic acid, 4-(aminomethyl)-, trans-), Win-72052, romazarit (Ro-31-3948) (propanoic acid, 2-((2-(4-chlorophenyl)-4-methyl-5-oxazolyl)methoxy)-2-methyl-), PD-163594, SDZ-224-015 (L-alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-), L-709049 (L-alaninamide, N-acetyl-L-tyrosyl-L-valyl-N-(2-carboxy-1-formylethyl)-(S)—), TA-383 (1H-imidazole, 2-(4-chlorophenyl)-4,5-dihydro-4,5-diphenyl-, monohydrochloride, c's-), EI-1507-1 (6a,12a-epoxybenz(a)anthracen-1,12(2H,7H)-dione, 3,4-dihydro-3,7-dihydroxy-8-methoxy-3-methyl-), ethyl 4-(3,4-dimethoxyphenyl)-6,7-dimethoxy-2-(1,2,4-triazol-1-ylmethyl)quinoline-3-carboxylate, EI-1941-1, TJ-114, anakinra (interleukin 1 receptor antagonist (human isoform x reduced), N2-L-methionyl-), IX-207-887 (acetic acid, (10-methoxy-4H-benzo[4,5]cyclohepta[1,2-b]thien-4-ylidene)-), K-832, or an analogue or derivative thereof).

17. IL-4 Agonists

In another embodiment, the pharmacologically active compound is an IL-4 agonist (e.g., glatiramir acetate (L-glutamic acid, polymer with L-alanine, L-lysine and L-tyrosine, acetate (salt)), or an analogue or derivative thereof).

18. Immunomodulatory Agents

In another embodiment, the pharmacologically active compound is an immunomodulatory agent (e.g., biolimus, ABT-578, methylsulfamic acid 3-(2-methoxyphenoxy)-2-(((methylamino)sulfonyl)oxy)propyl ester, sirolimus (also referred to as rapamycin or RAPAMUNE (American Home Products, Inc., Madison, N.J.)), CCI-779 (rapamycin 42-(3-hydroxy-2-(hydroxymethyl)-2-methylpropanoate)), LF-15-0195, NPC15669 (L-leucine, N-(((2,7-dimethyl-9H-fluoren-9-yl)methoxy)carbonyl)-), NPC-15670 (L-leucine, N-(((4,5-dimethyl-9H-fluoren-9-yl)methoxy)carbonyl)-), NPC-16570 (4-(2-(fluoren-9-yl)ethyloxy-carbonyl)aminobenzoic acid), sufosfamide (ethanol, 2-((3-(2-chloroethyl)tetrahydro-2H-1,3,2-oxazaphosphorin-2-yl)amino)-, methanesulfonate (ester), P-oxide), tresperimus (2-(N-(4-(3-aminopropylamino)butyl)carbamoyloxy)-N-(6-guanidinohexyl)acetamide), 4-(2-(fluoren-9-yl)ethoxycarbonylamino)-benzo-hydroxamic acid, iaquinimod, PBI-1411, azathioprine (6-((1-Methyl-4-nitro-1H-imidazol-5-yl)thio)-1H-purine), PBI0032, beclometasone, MDL-28842 (9H-purin-6-amine, 9-(5-deoxy-5-fluoro-9-D-threo-pent-4-enofuranosyl)-, (Z)-), FK-788, AVE-1726. ZK-90695, ZK-90695, Ro-54864, didemnin-B, Illinois (didemnin A, N-(1-(2-hydroxy-1-oxopropyl)-L-prolyl)-, (S)—), SDZ-62-826 (ethanaminium, 2-((hydroxy((1-((octadecyloxy)carbonyl)-3-piperidinyl)methoxy)phosphinyl)oxy)-N,N,N-trimethyl-, inner salt), argyrin B ((4S,7S,13R,22R)-13-Ethyl-4-(1H-indol-3-ylmethyl)-7-(4-methoxy-1H-indol-3-ylmethyl) 18,22-dimethyl-16-methyl-ene-24-thia-3,6,9,12,15,18,21,26-octaazabicyclo(21.2.1)-hexacosa-1(25),23(26)-diene-2,5,8,11,14,17,20-heptaone), everolimus (rapamycin, 42-O-(2-hydroxyethyl)-), SAR-943, L-687795, 6-((4-chlorophenyl)sulfinyl)-2,3-dihydro-2-(4-methoxy-phenyl)-5-methyl-3-oxo-4-pyridazinecarbonitrile, 91 Y78 (1H-imidazo[4,5-c]pyridin-4-amine, 1-β-D-ribofuranosyl-), auranofin (gold, (1-thio-β-D-glucopyranose 2,3,4,6-tetraacetato-S) (triethylphosphine)-), 27-O-demethylrapamycin, tipredane (and rosta-1,4-dien-3-one, 17-(ethylthio)-9-fluoro-11-hydroxy-17-(methylthio)-, (11β,17 alpha)-), AI-402, LY-178002 (4-thiazolidinone, 5-((3,5-bis(1,1-dimethylethyl)-4-hydroxyphenyl)methylene)-), SM-8849 (2-thiazolamine, 4-(1-(2-fluoro(1,1′-biphenyl)-4-yl)ethyl)-N-methyl-), piceatannol, resveratrol, triamcinolone acetonide (pregna-1,4-diene-3,20-dione, 9-fluoro-11,21-dihydroxy-16,17-((1-methylethylidene)bis(oxy))-, (11β,16 alpha)-), ciclosporin (cyclosporin A), tacrolimus (15,19-epoxy-3H-pyrido(2,1-c)(1,4)oxaazacyclotricosine-1,7,20,21 (4H,23H)-tetrone, 5,6,8,11,12,13,14,15,16,17,18,19,24,25,26,26a-hexadecahydro-5,1,9-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1 S*,3S*,4S*)),4S*,5R*,8S*,9E,12R*,14R*,15S*,16R*,18S*,19S*,26aR*))-), gusperimus (heptanamide, 7-((aminoiminomethyl)amino)-N-(2-((4-((3-aminopropyl)amino)butyl)amino)-1-hydroxy-2-oxoethyl)-, (+/−)-), tixocortol pivalate (pregn-4-ene-3,20-dione, 21-((2,2-dimethyl-1-oxopropyl)thio)-11,17-dihydroxy-, (11β)-), alefacept (1-92 LFA-3 (antigen) (human) fusion protein with immunoglobulin G1 (human hinge-CH2-CH3 gamma1-chain), dimer), halobetasol propionate (pregna-1,4-diene-3,20-dione, 21-chloro-6,9-difluoro-11-hydroxy-16-methyl-17-(1-oxopropoxy)-, (6Alpha, 11β,16β)-), iloprost trometamol (pentanoic acid, 5-(hexahydro-5-hydroxy-4-(3-hydroxy-4-methyl-1-octen-6-ynyl)-2(1H)-pentalenylidene)-), beraprost (1H-cyclopenta(b)benzofuran-5-butanoic acid, 2,3,3a,8b-tetrahydro-2-hydroxy-1-(3-hydroxy-4-methyl-1-octen-6-ynyl)-), rimexolone (androsta-1,4-dien-3-one, 11-hydroxy-16,17-dimethyl-17-(1-oxopropyl)-, (11β,16Alpha, 17β)-), dexamethasone (pregna-1,4-diene-3,20-dione,9-fluoro-11,17,21-trihydroxy-16-methyl-, (11β,16alpha)-), sulindac (cis-5-fluoro-2-methyl-1-((p-methylsulfinyl)benzylidene)indene-3-acetic acid), proglumetacin (1H-Indole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (+/−)-), alclometasone dipropionate (pregna-1,4-diene-3,20-dione, 7-chloro-11-hydroxy-16-methyl-17,21-bis(1-oxopropoxy)-, (7alpha, 11β,16alpha)-), pimecrolimus (15,19-epoxy-3H-pyrido(2,1-c)(1,4)oxaazacyclotricosine-1,7,20,21 (4H,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*,15S*,16R*,18S*,19S*,26aR*))-), hydrocortisone-17-butyrate (pregn-4-ene-3,20-dione, 11,21-dihydroxy-17-(1-oxobutoxy)-, (11β)-), mitoxantrone (9,10-anthracenedione, 1,4-dihydroxy-5,8-bis((2-((2-hydroxyethyl)amino)ethyl)amino)-), mizoribine (1H-imidazole-4-carboxamide, 5-hydroxy 1-β-D-ribofuranosyl-), prednicarbate (pregna-1,4-diene-3,20-dione, 17-((ethoxycarbonyl)oxy)-11-hydroxy-21(1-oxopropoxy)-, (11β)-), iobenzarit (benzoic acid, 2-((2-carboxyphenyl)amino)-4-chloro-), glucametacin (D-glucose, 2-(((1-(4-chlorobenzoyl)-5-methoxy-2-methyl-1H-indol-3-yl)acetyl)amino)-2-deoxy-), fluocortolone monohydrate ((6 alpha)-fluoro-16alpha-methylpregna-1,4-dien-11β,21-diol-3,20-dione), fluocortin butyl (pregna-1,4-dien-21-oic acid, 6-fluoro-11-hydroxy-16-methyl-3,20-dioxo-, butyl ester, (6alpha, 11β,16alpha)-), difluprednate (pregna-1,4-diene-3,20-dione, 21-(acetyloxy)-6,9-difluoro-11-hydroxy-17-(1-oxobutoxy)-, (6 alpha, 11β)-), diflorasone diacetate (pregna-1,4-diene-3,20-dione, 17,21-bis(acetyloxy)-6,9-difluoro-11-hydroxy-16-methyl-, (6Alpha, 11β,16β)-), dexamethasone valerate (pregna-1,4-diene-3,20-dione, 9-fluoro-11,21-dihydroxy-16-methyl-17-((1-oxopentyl)oxy)-, (11β,16Alpha)-), methylprednisolone, deprodone propionate (pregna-1,4-diene-3,20-dione, 11-hydroxy-17-(1-oxopropoxy)-, (11.beta.)-), bucillamine (L-cysteine, N-(2-mercapto-2-methyl-1-oxopropyl)-), amcinonide (benzeneacetic acid, 2-amino-3-benzoyl-, monosodium salt, monohydrate), acemetacin (1H-indole-3-acetic acid, 1-(4-chlorobenzoyl)-5-methoxy-2-methyl-, carboxymethyl ester), or an analogue or derivative thereof).

Further, analogues of rapamycin include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| RAPAMUNE | NSC-226080 | | |
| Rapamycin | | | |
| Tacrolimus | FK506 | Fujusawa | See below |

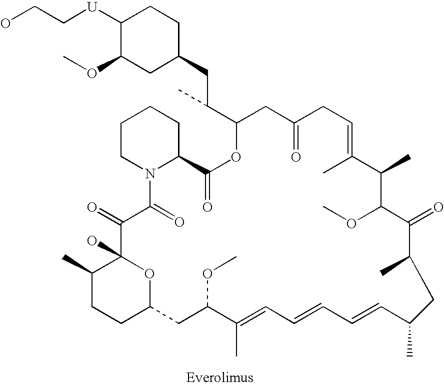

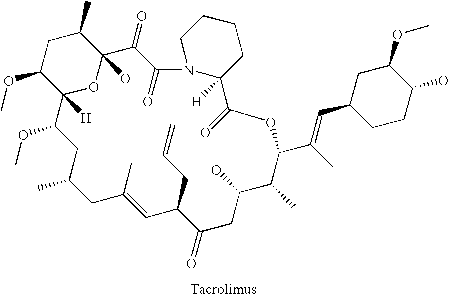

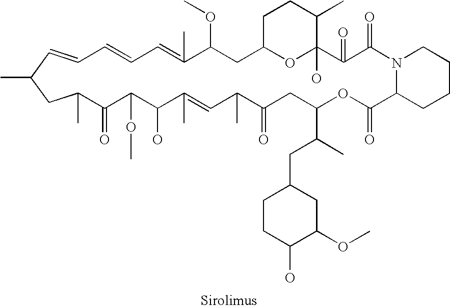

Further sirolimus analogues and derivatives include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives include ABT-578 and others may be found in PCT Publication Nos. WO 97/10502. WO 96/41807, WO 96/35423, WO 96/03430, WO 9600282, WO 95/16691, WO 95/5328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234, 5,597,715; 5,583,139; 5,563,172; 5,561,228; 5,561,137; 5,541,193; 5,541,189, 5,534,632; 5,527,907; 5,484,799; 5,457,194, 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

In one aspect, the fibrosis-inhibiting agent may be, e.g., rapamycin (sirolimus), everolimus, biolimus, tresperimus, auranofin, 27-O-demethylrapamycin, tacrolimus, gusperimus, pimecrolimus, or ABT-578.

19. Inosine Monophosphate Dehydrogenase Inhibitors

In another embodiment, the pharmacologically active compound is an inosine monophosphate dehydrogenase (IMPDH) inhibitor (e.g., mycophenolic acid, mycophenolate mofetil (4-hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-), ribavirin (1H-1,2,4-triazole-3-carboxamide, 1-β-D-ribofuranosyl-), tiazofurin (4-thiazolecarboxamide, 2-β-D-ribofuranosyl-), viramidine, aminothiadiazole, thiophenfurin, tiazofurin) or an analogue or derivative thereof. Additional representative examples are included in U.S. Pat. Nos. 5,536,747, 5,807,876, 5,932,600, 6,054,472, 6,128,582, 6,344,465, 6,395,763, 6,399,773, 6,420,403, 6,479,628, 6,498,178, 6,514,979, 6,518,291, 6,541,496, 6,596,747, 6,617,323, 6,624,184, Patent Application Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, 2003/0195202A1, and PCT Publication Nos. WO 0024725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 2051814A1, WO 2057287A2, WO2057425A2, WO 2060875A1, WO 2060896A1, WO 2060898A1, WO 2068058A2, WO 3020298A1, WO 3037349A1, WO 3039548A1, WO 3045901A2, WO 3047512A2, WO 3053958A1, WO 3055447A2, WO 3059269A2, WO 3063573A2, WO 3087071A1, WO 90/01545A1, WO 97/40028A1, WO 97/41211A1, WO 98/40381A1, and WO 99/55663A1).

Leukotriene Inhibitors

In another embodiment, the pharmacologically active compound is a leukotreine inhibitor (e.g., ONO-4057(benzenepropanoic acid, 2-(4-carboxybutoxy)-6-((6-(4-methoxyphenyl)-5-hexenyl)oxy)-, (E)-), ONO-LB-448, pirodomast 1,8-naphthyridin-2(1H)-one, 4-hydroxy-1-phenyl-3-(1-pyrrolidinyl)-, Sch-40120 (benzo(b)(1,8)naphthyridin-5(7H)-one, 10-(3-chlorophenyl)-6,8,9,10-tetrahydro-), L-656224 (4-benzofuranol, 7-chloro-2-((4-methoxyphenyl)methyl)-3-methyl-5-propyl-), MAFP (methyl arachidonyl fluorophosphonate), ontazolast (2-benzoxazolamine, N-(2-cyclohexyl-1-(2-pyridinyl)ethyl)-5-methyl-, (S)—), amelubant (carbamic acid, ((4-((3-((4-(1-(4-hydroxyphenyl)-1-methylethyl)phenoxy)methyl)phenyl)methoxy)phenyl)iminomethyl)-ethyl ester), SB-201993 (benzoic acid, 3-((((6-((1E)-2-carboxyethenyl)-5-((8-(4-methoxyphenyl)octyl)oxy)-2-pyridinyl)methyl)thio)methyl)-), LY-203647 (ethanone, 1-(2-hydroxy-3-propyl-4-(4-(2-(4-(1H-tetrazol-5-yl)butyl)-2H-tetrazol-5-yl)butoxy)phenyl)-), LY-210073, LY-223982 (benzenepropanoic acid, 5-(3-carboxybenzoyl)-2-((6-(4-methoxyphenyl)-5-hexenyl)oxy)-, (E)-), LY-293111 (benzoic acid, 2-(3-(3-((5-ethyl-4′-fluoro-2-hydroxy(1,1′-biphenyl)-4-yl)oxy)propoxy)-2-propylphenoxy)-), SM-9064 (pyrrolidine, 1-(4,11-dihydroxy-13-(4-methoxyphenyl)-1-oxo-5,7,9-tridecatrienyl)-, (E,E,E)-), T-0757 (2,6-octadienamide, N-(4-hydroxy-3,5-dimethylphenyl)-3,7-dimethyl-, (2E)-), or an analogue or derivative thereof).

21. MCP-1 Antagonists

In another embodiment, the pharmacologically active compound is a MCP-1 antagonist (e.g., nitronaproxen (2-napthaleneacetic acid, 6-methoxy-alpha-methyl 4-(nitrooxy)butyl ester (alpha S)—), bindarit (2-(1-benzylindazol-3-ylmethoxy)-2-methylpropanoic acid), 1-alpha-25 dihydroxy vitamin D₃ or an analogue or derivative thereof).

22. MMP Inhibitors

In another embodiment, the pharmacologically active compound is a matrix metalloproteinase (MMP) inhibitor (e.g., D-9120, doxycycline (2-naphthacenecarboxamide, 4-(dimethylamino)-1,4,4a,5,5a,6,11,12a-octahydro-3,5,10,12,12a-pentahydroxy-6-methyl-1,11-dioxo-(4S-(4 alpha, 4a alpha, 5 alpha, 5a alpha, 6 alpha, 12a alpha))-), BB-2827, BB-1101 (2S-allyl-N-1-hydroxy-3R-isobutyl-N4-(1S-methylcarbamoyl-2-phenylethyl)-succinamide), BB-2983, solimastat (N′-(2,2-dimethyl-1(S)—(N-(2-pyridyl)carbamoyl)propyl)-N4-hydroxy-2(R)-isobutyl-3(S)-methoxysuccinamide), batimastat (butanediamide, N4-hydroxy-N1-(2-(methylamino)-2-oxo-1-(phenylmethyl)ethyl)-2-(2-methylpropyl)-3-((2-thienylthio)methyl)-, (2R-(1(S*),2R*,3S*))-), CH-138, CH-5902, D-1927, D-5410, EF-13 (gamma-linolenic acid lithium salt), CMT-3 (2-naphthacenecarboxamide, 1,4,4a,5,5a,6,11,12a-octahydro-3,10,12,12a-tetrahydroxy-1,11-dioxo-, (4aS,5aR,12aS)—), marimastat (N-(2,2-dimethyl-1 (S)—(N-methylcarbamoyl)propyl)-N,3(S)-dihydroxy-2(R)-isobutylsuccinamide), TIMP'S, ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-), PS-508, CH-715, nimesulide (methanesulfonamide, N-(4-nitro-2 phenoxyphenyl)-), hexahydro-2-(2(R)-(1 (RS)-(hydroxycarbamoyl)-4-phenylbutyl)nonanoyl)-N-(2,2,6,6-etramethyl-4-piperidinyl)-3(S)-pyridazine carboxamide, Rs-113-080, Ro-1130830, cipemastat (1-piperidinebutanamide β-(cyclopentylmethyl)-N-hydroxy-gamma-oxo-alpha-((3,4,4-trimethyl-2,5-dioxo-1-imidazolidinyl)methyl)-,(alpha R,βR)—), 5-(4′-biphenyl)-5-(N-(4-nitrophenyl)piperazinyl)barbituric acid, 6-methoxy-1,2,3,4-tetrahydro-norharman-1-carboxylic acid, Ro-31-4724 (L-alanine, N-(2-(2-(hydroxyamino)-2-oxoethyl)-4-methyl-1-oxopentyl)-L-leucyl-, ethyl ester), prinomastat (3-thiomorpholinecarboxamide, N-hydroxy-2,2-dimethyl-4-((4-(4-pyridinyloxy) phenyl)sulfonyl)-, (3R)—), AG-3433 (1H-pyrrole-3-propanic acid, 1-(4′-cyano(1,1′-biphenyl)-4-yl)-b-((((3S)-tetrahydro-4,4-dimethyl-2-oxo-3-furanyl)amino)carbonyl)-, phenylmethyl ester, (bS)—), PNU-142769 (2H-Isoindole-2-butanamide, 1,3-dihydro-N-hydroxy-alpha-((3S)-3-(2-methylpropyl)-2-oxo-1-(2-phenylethyl)-3-pyrrolidinyl)-1,3-dioxo-, (alpha R)—), (S)-1-(2-((((4,5-dihydro-5-thioxo-1,3,4-thiadiazol-2-yl)amino)-carbonyl)amino)-1-oxo-3-(pentafluorophenyl)propyl)-4-(2-pyridinyl)piperazine, SU-5402 (1H-pyrrole-3-propanoic acid, 2-((1,2-dihydro-2-oxo-3H-indol-3-ylidene)methyl)-4-methyl-), SC-77964, PNU-171829, CGS-27023A, N-hydroxy-2(R)-((4-methoxybenzene-sulfonyl)(4-picolyl)amino)-2-(2-tetrahydrofuranyl)-acetamide, L-758354 ((1,1′-biphenyl)-4-hexanoic acid, alpha-butyl-gamma-(((2,2-dimethyl-1-((methylamino)carbonyl)propyl)amino)carbonyl)-4′-fluoro-, (alpha S-(alpha R*, gammaS*(R*)))-, GI-155704A, CPA-926, TMI-005, XL-784, or an analogue or derivative thereof). Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696.082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437.177; 6,376,665; 5,268,384; 5,183,900; 5,189,178; 6,511,993; 6,617,354; 6,331,563, 5,962,466; 5,861,427; 5,830,869, and 6,087,359.

23. NF Kappa B Inhibitors

In another embodiment, the pharmacologically active compound is a NF kappa B (NFKB) inhibitor (e.g., AVE-0545, Oxi-104 (benzamide, 4-amino-3-chloro-N-(2-(diethylamino)ethyl)-), dexlipotam, R-flurbiprofen ((1,1′-biphenyl)-4-acetic acid, 2-fluoro-alpha-methyl), SP100030 (2-chloro-N-(3,5-di(trifluoromethyl)phenyl)-4-(trifluoromethyl)pyrimidine-5-carboxamide), AVE-0545, Viatris, AVE-0547, Bay 11-7082, Bay 11-7085, 15 deoxy-prostaylandin J2, bortezomib (boronic acid, ((1R)-3-methyl-1-(((2S)-1-oxo-3-phenyl-2-((pyrazinylcarbonyl)amino)propyl)amino)butyl)-, benzamide and nicotinamide derivatives that inhibit NF-kappaB, such as those described in U.S. Pat. Nos. 5,561,161 and 5,340,565 (OxiGene), PG490-88Na, or an analogue or derivative thereof).

24. NO Agonists

In another embodiment, the pharmacologically active compound is a NO antagonist (e.g., NCX-4016 (benzoic acid, 2-(acetyloxy)-, 3-((nitrooxy)methyl)phenyl ester, NCX-2216, L-arginine or an analogue or derivative thereof).

25. P38 MAP Kinase Inhibitors

In another embodiment, the pharmacologically active compound is a p38 MAP kinase inhibitor (e.g., GW-2286, CGP-52411, BIRB-798, SB220025, Ro-320-1195, RWJ-67657, RWJ-68354, SCIO-469, SCIO-323, AMG-548, CMC-146, SD-31145, CC-8866, Ro-320-1195, PD-98059 (4H-1-benzopyran-4-one, 2-(2-amino-3-methoxyphenyl)-), CGH-2466, doramapimod, SB-203580 (pyridine, 4-(5-(4-fluorophenyl)-2-(4-(methylsulfinyl)phenyl)-1H-imidazol-4-yl)-), SB-220025 ((5-(2-amino-4-pyrimidinyl)-4-(4-fluorophenyl) 1-(4 piperidinyl)imidazole), SB-281832, PD169316, SB202190, GSK-681323, EO-1606, GSK-681323, or an analogue or derivative thereof). Additional representative examples are included in U.S. Pat. Nos. 6,300,347, 6,316,464; 6,316,466; 6,376,527; 6,444,696; 6,479,507; 6,509,361; 6,579,874; 6,630,485, US Patent Application Publication Nos. 2001/0044538A1; 2002/0013354A1; 2002/0049220A1; 2002/0103245A1; 2002/0151491A1; 2002/0156114A1; 2003/0018051A1; 2003/0073832A1; 2003/0130257A1; 2003/0130273A1; 2003/0130319A1; 2003/0139388A1; 20030139462A1; 2003/0149031A1; 2003/0166647A1; 2003/0181411A1; and PCT Publication Nos. WO 00/63204A2; WO 01/21591A1; WO 01/35959A1; WO 01/7481 A2; WO 02/18379A2; WO 2064594A2; WO 2083622A2; WO 2094842A2; WO 2096426A1; WO 2101015A2; WO 2103000A2; WO 3008413A1; WO 3016248A2; WO 3020715A1; WO 3024899A2; WO 3031431A1; WO3040103A1; WO 3053940A1; WO 3053941A2; WO 3063799A2, WO 3079986A2; WO 3080024A2; WO 3082287A1; WO 97/44467A1; WO 99/01449A1; and WO 99/58523A1.

26. Phosphodiesterase Inhibitors

In another embodiment, the pharmacologically active compound is a phosphodiesterase inhibitor (e.g., CDP-840 (pyridine, 4-((2R)-2-(3-(cyclopentyloxy)-4-methoxyphenyl)-2-phenylethyl)-), CH-3697, CT-2820, D-22888 (imidazo[1,5-a]pyrido(3,2-e)pyrazin-6(5H)-one, 9-ethyl-2-methoxy-7-methyl-5-propyl-), D-4418 (8-methoxyquinoline-5-(N-(2,5-dichloropyridin-3-yl))carboxamide), 1-(3-cyclopentyloxy-4-methoxyphenyl)-2-(2,6-dichloro-4-pyridyl)ethanone oxime, D-4396, ONO-6126, CDC-998, CDC-801, V-11294A (3-(3-(cyclopentyloxy)-4-methoxybenzyl)-6-(ethylamino)-8-isopropyl-3H-purine hydrochloride), S,S′-methylene-bis(2-(8-cyclopropyl-3-propyl-6-(4-pyridylmethylamino)-2-thio-3H-purine)) tetrahyrochloride, rolipram (2-pyrrolidinone, 4-(3-(cyclopentyloxy)-4-methoxyphenyl)-), CP-293121, CP-353164 (5-(3-cyclopentyloxy-4-methoxyphenyl)pyridine-2-carboxamide), oxagrelate (6-phthalazinecarboxylic acid, 3,4-dihydro-1-(hydroxymethyl)-5,7-dimethyl-4-oxo-, ethyl ester), PD-168787, ibudilast (1-propanone, 2-methyl-1-(2-(1-methylethyl)pyrazolo(1,5-a)pyridin-3-yl)-), oxagrelate (6-phthalazinecarboxylic acid, 3,4-dihydro-1-(hydroxymethyl)-5,7-dimethyl-4-oxo-, ethyl ester), griseolic acid (alpha-L-talo-oct-4-enofuranuronic acid, 1-(6-amino-9H-purin-9-yl)-3,6-anhydro-6-C-carboxy-1,5-dideoxy-), KW-4490, KS-506, T-440, roflumilast (benzamide, 3-(cyclopropylmethoxy)-N-(3,5-dichloro-4-pyridinyl)-4-(difluoromethoxy)-), rolipram, milrinone, triflusinal (benzoic acid, 2-(acetyloxy)-4-(trifluoromethyl)-), anagrelide hydrochloride (imidazo(2,1-b)quinazolin-2(3H)-one, 6,7-dichloro-1,5-dihydro-, monohydrochloride), cilostazol (2(1H)-quinolinone, 6-(4-(1-cyclohexyl-1H-tetrazol-5-yl)butoxy)-3,4-dihydro-), propentofylline (1H-purine-2,6-dione, 3,7-dihydro-3-methyl-1-(5-oxohexyl)-7-propyl-), sildenafil citrate (piperazine, 1-((3-(4,7-dihydro-1-methyl-7-oxo-3-propyl-1H-pyrazolo(4,3-d) pyrimidin-5-yl)-4-ethoxyphenyl)sulfonyl)-4-methyl, 2-hydroxy-1,2,3-propanetricarboxylate-(1:1)), tadalafil (pyrazino(1′,2′:1,6)pyrido(3,4-b)indole1,4-dione, 6-(1,3-benzodioxol-5-yl)-2,3,6,7,12,12a-hexahydro-2-methyl-, (6R-trans)), vardenafil (piperazine, 1-(3-(1,4-dihydro-5-methyl(-4-oxo-7-propylimidazo(5,1-f)(1,2,4)-triazin-2-yl)-4-ethoxyphenyl)sulfonyl)-4-ethyl-), milrinone ((3,4′-bipyridine)-5-carbonitrile, 1,6-dihydro-2-methyl-6-oxo-), enoximone (2H-imidazol-2-one, 1,3-dihydro-4-methyl-5-(4-(methylthio)benzoyl)-), theophylline (1H-purine-2,6-dione, 3,7-dihydro-1,3-dimethyl-), ibudilast (1-propanone, 2-methyl-1-(2-(1-methylethyl)pyrazolo(1,5-a)pyridin-3-yl)-), aminophylline (1H-purine-2,6-dione, 3,7-dihydro-1,3-dimethyl-, compound with 1,2-ethanediamine (2:1)-), acebrophylline (7H-purine-7-acetic acid, 1,2,3,6-tetrahydro-1,3-dimethyl-2,6-dioxo-, compd. with trans-4-(((2-amino-3,5-dibromophenyl)methyl)amino)cyclohexanol (1:1)), plafibride (propanamide, 2-(4-chlorophenoxy)-2-methyl-N-(((4-morpholinylmethyl)amino)carbonyl)-), ioprinone hydrochloride (3-pyridinecarbonitrile, 1,2-dihydro-5-imidazo[1,2-a]pyridin-6-yl-6-methyl-2-oxo-, monohydrochloride-), fosfosal (benzoic acid, 2-(phosphonooxy)-), aminone ((3,4′-bipyridin)-6(1H)-one, 5-amino-, or an analogue or derivative thereof)

Other examples of phosphodiesterase inhibitors include denbufylline (1H-purine-2,6-dione, 1,3-dibutyl-3,7-dihydro-7-(2-oxopropyl)-), propentofylline (1H-purine-2,6-dione, 3,7-dihydro-3-methyl-1-(5-oxohexyl)-7-propyl-) and pelrinone (5-pyrimidinecarbonitrile, 1,4-dihydro-2-methyl-4-oxo-6-[(3-pyridinylmethyl)amino]-).

Other examples of phosphodiesterase III inhibitors include enoximone (2H-imidazol-2-one, 1,3-dihydro-4-methyl-5-[4-(methylthio)benzoyl]-), and saterinone (3-pyridinecarbonitrile, 1,2-dihydro-5-[4-[2-hydroxy-3-[(4-(2-methoxyphenyl)-1-piperazinyl]propoxy]phenyl]-6-methyl-2-oxo-).

Other examples of phosphodiesterase IV inhibitors include AWD-12-281, 3-auinolinecarboxylic acid, 1-ethyl-6-fluoro-1,4-dihydro-7-(4-methyl-1-piperazinyl)-4-oxo-), tadalafil (pyrazino(1′,2′:1,6)pyrido(3,4-b)indole1,4-dione, 6-(1,3-benzodioxol-5-yl)-2,3,6,7,12,12a-hexahydro-2-methyl-, (6R-trans)), and filaminast (ethanone, 1-[3-(cyclopentyloxy)-4-methoxyphenyl]-, O-(aminocarbonyl)oxime, (1E)-)

Another example of a phosphodiesterase V inhibitor is vardenafil (piperazine, 1-(3-(1,4-dihydro-5-methyl(-4-oxo-7-propylimidazo(5,1-f)(1,2,4)-triazin-2-yl)-4-ethoxyphenyl)sulfonyl)-4-ethyl-).

27. TGF beta Inhibitors

In another embodiment, the pharmacologically active compound is a TGF beta Inhibitor (e.g., mannose-6-phosphate, LF-984, tamoxifen (ethanamine, 2-(4-(1,2-diphenyl-1-butenyl)phenoxy)-N,N-dimethyl-, (Z)-), tranilast, or an analogue or derivative thereof).

28. Thromboxane A2 Antagonists

In another embodiment, the pharmacologically active compound is a thromboxane A2 antagonist (e.g., CGS-22652 (3-pyridineheptanoic acid, γ-(4-(((4-chlorophenyl)sulfonyl)amino)butyl)-.(+−)-), ozagrel (2-propenoic acid 3-(4-(1H-imidazol-1-ylmethyl)phenyl)-, (E)-), argatroban (2-piperidinecarboxylic acid, 1-(5-((aminoiminomethyl)amino)-1-oxo-2-(((1,2,3,4-tetrahydro-3-methyl-8-quinolinyl)sulfonyl)amino)pentyl)-4-methyl-), ramatroban (9H-carbazole-9-propanoic acid, 3-(((4-fluorophenyl)sulfonyl)amino)-1,2,3,4-tetrahydro-, (R)—), torasemide (3-pyridinesulfonamide, N-(((1-methylethyl)amino)carbonyl)-4-((3-methylphenyl)amino)-), gamma linoleic acid ((Z,Z,Z)-6,9,12-octadecatrienoic acid), seratrodast (benzeneheptanoic acid, zeta-(2,4,5-trimethyl-3,6-dioxo-1,4-cyclohexadien-1-yl)-, (+/−)-, or an analogue or derivative thereof).

29. TNFa Antagonists and TACE Inhibitors

In another embodiment, the pharmacologically active compound is a TNFa antagonist or TACE inhibitor (e.g., E-5531 (2-deoxy-6-O-(2-deoxy-3-0-(3(R)-(5(Z)-dodecenoyloxy)-decyl)-6-0-methyl-2-(3-oxotetradecanamido)-4-O-phosphono-β-D-glucopyranosyl)-3-0-(3(R)-hydroxydecyl)-2-(3-oxotetradecanamido)-alpha-D-glucopyranose-1-O-phosphate), AZD-4717, glycophosphopeptical, UR-12715 (B=benzoic acid, 2-hydroxy-5-((4-(3-(4-(2-methyl-1H-imidazol(4,5-c)pyridin-1-yl)methyl)-1-piperidinyl)-3-oxo-1-phenyl-1-propenyl)phenyl)azo) (Z)), PMS-601, AM-87, xyloadenosine (9H-purin-6-amine, 9-β-D-xylofuranosyl-), RDP-58, RDP-59, BB2275, benzydamine, E-3330 (undecanoic acid, 2-((4,5-dimethoxy-2-methyl-3,6-dioxo-1,4-cyclohexadien-1-yl)methylene)-, (E)-), N-(D, L-2-(hydroxyaminocarbonyl)methyl-4-methylpentanoyl)-L-3-(2′-naphthyl)alanyl-L-alanine, 2-aminoethyl amide, CP-564959, MLN-608, SPC-839, ENMD-0997, Sch-23863 ((2-(10,11-dihydro-5-ethoxy-5H-dibenzo (a,d) cyclohepten-S-yl)-N,N-dimethyl-ethanamine), SH-636, PKF-241-466, PKF-242-484, TNF-484A, cilomilast (cis-4-cyano-4-(3-(cyclopentyloxy)-4-methoxyphenyl)cyclohexane-1-carboxylic acid), GW-3333, GW4459, BMS-561392, AM-87, cloricromene (acetic acid, ((8-chloro-3-(2-(diethylamino)ethyl)-4-methyl-2-oxo-2H-1-benzopyran-7-yl)oxy)-, ethyl ester), thalidomide (1H-Isoindole-1,3(2H)-dione, 2-(2,6-dioxo-3-piperidinyl)-), vesnarinone (piperazine, 1-(3,4-dimethoxybenzoyl)-4-(1,2,3,4-tetrahydro-2-oxo-6-quinolinyl)-), infliximab, lentinan, etanercept (1-235-tumor necrosis factor receptor (human) fusion protein with 236-467-Immunoglobulin G1 (human gamma1-chain Fc fragment)), diacerein (2-anthracenecarboxylic acid, 4,5-bis(acetyloxy)-9,10-dihydro-9,10-dioxo-, or an analogue or derivative thereof).

30. Tyrosine Kinase Inhibitors

In another embodiment, the pharmacologically active compound is a tyrosine kinase inhibitor (e.g., SKI-606, ER-068224, SD-208, N-(6-benzothiazolyl)-4-(2-(1-piperazinyl)pyrid-5-yl)-2-pyrimidineamine, celastrol (24,25,26-trinoroleana-1(10),3,5,7-tetraen-29-oic acid, 3-hydroxy-9,13-dimethyl-2-oxo-, (9 beta-,13alpha,14β,20 alpha)-), CP-127374 (geldanamycin, 17-demethoxy-17-(2-propenylamino)-), CP-564959, PD-171026, CGP-52411 (1H-Isoindole-1,3(2H)-dione, 4,5-bis(phenylamino)-), CGP-53716 (benzamide, N-(4-methyl-3-((4-(3-pyridinyl)-2-pyrimidinyl)amino)phenyl)-), imatinib (4-((methyl-1-piperazinyl)methyl)-N-(4-methyl-3-((4-(3-pyridinyl)-2-pyrimidinyl)amino)-phenyl)benzamide methanesulfonate), NVP-AAK980-NX, KF-250706 (13-chloro,5(R),6(S)-epoxy-14,16-dihydroxy-11-(hydroyimino)-3(R)-methyl-3,4,5,6,11,12-hexahydro-1H-2-benzoxacyclotetradecin-1-one), 5-(3-(3-methoxy-4-(2-((E)-2-phenylethenyl)-4-oxazolyl methoxy)phenyl)propyl)-3-(2-((E)-2-phenylethenyl)-4-oxazolylmethyl)-2,4-oxazolidinedione, genistein, NV-06, or an analogue or derivative thereof).

31. Vitronectin Inhibitors

In another embodiment, the pharmacologically active compound is a vitronectin inhibitor (e.g., O-(9,10-dimethoxy-1,2,3,4,5,6-hexahydro-4-((1,4,5,6-tetrahydro-2-pyrimidinyl)hydrazono)-8-benz(e)azulenyl)-N-((phenylmethoxy)carbonyl)-DL-homoserine 2,3-dihydroxypropyl ester, (2S)-benzoylcarbonylamino-3-(2-((4S)-(3-(4,5-dihydro-1H-imidazol-2-ylamino)-propyl)-2,5-dioxo-imidazolidin-1-yl)-acetylamino)-propionate, Sch-221153, S-836, SC-68448 (β-((2-2-(((3-((aminoiminomethyl)amino)-phenyl)carbonyl)amino)acetyl)amino)-3,5-dichlorobenzenepropanoic acid), SD-7784, S-247, or an analogue or derivative thereof)

32. Fibroblast Growth Factor Inhibitors

In another embodiment, the pharmacologically active compound is a fibroblast growth factor inhibitor (e.g., CT-052923 (((2H-benzo(d)1,3-dioxalan-5-methyl)amino)(4-(6,7-dimethoxyquinazolin-4-yl)piperazinyl)methane-1-thione), or an analogue or derivative thereof).

33. Protein Kinase Inhibitors

In another embodiment, the pharmacologically active compound is a protein kinase inhibitor (e.g., KP-0201448, NPC15437 (hexanamide, 2,6-diamino-N-((1-(1-oxotridecyl)-2-piperidinyl)methyl)-), fasudil (1H-1,4-diazepine, hexahydro-1-(5-isoquinolinylsulfonyl)-), midostaurin (benzamide, N-(2,3,10,11,12,13-hexahydro-10-methoxy-9-methyl-1-oxo-9,13-epoxy-1H,9H-diindolo(1,2,3-gh:3′,2′,1′-lm)pyrrolo(3,4-j)(1,7)benzodiazonin-11-yl)-N-methyl-, (9Alpha, 10β,11β,13Alpha)-),fasudil (1H-1,4-diazepine, hexahydro-1-(5-isoquinolinylsulfonyl)-, dexniguldipine (3,5-pyridinedicarboxylic acid, 1,4-dihydro-2,6-dimethyl-4-(3-nitrophenyl)-, 3-(4,4-diphenyl-1-piperidinyl)propyl methyl ester, monohydrochloride, (R)—), LY-317615 (1H-pyrole-2,5-dione, 3-(1-methyl-1H-indol-3-yl)-4-[1-[1-(2-pyridinylmethyl)-4-piperidinyl]-1H-indol-3-yl]-, monohydrochloride), perifosine (piperidinium, 4-[[hydroxy(octadecyloxy)phosphinyl]oxy]-1,1-dimethyl-, inner salt), LY-333531 (9H, 18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)—), Kynac; SPC-100270 (1,3-octadecanediol, 2-amino-, [S—(R*,R*)]-), Kynacyte, or an analogue or derivative thereof).

34. PDGF Receptor Kinase Inhibitors

In another embodiment, the pharmacologically active compound is a PDGF: receptor kinase inhibitor (e.g., RPR-127963E, or an analogue or derivative thereof)

35. Endothelial Growth Factor Receptor Kinase Inhibitors

In another embodiment, the pharmacologically active compound is an endothelial growth factor receptor kinase inhibitor (e.g., CEP-7055, SU-0879 ((E)-3-(3,5-di-tert-butyl-4-hydroxyphenyl)-2-(aminothiocarbonyl)acrylonitrile), BIBF-1000, AG-013736 (CP-868596), AMG-706, AVE-0005, NM-3 (3-(2-methylcarboxymethyl)-6-methoxy-8-hydroxy-isocoumarin), Bay-43-9006, SU-011248, or an analogue or derivative thereof).

36. Retinoic Acid Receptor Antagonists

In another embodiment, the pharmacologically active compound is a retinoic acid receptor antagonist (e.g., etarotene (Ro-15-1570) (naphthalene, 6-(2-(4-(ethylsulfonyl)phenyl)-1-methylethenyl)-1,2,3,4-tetrahydro-1,1,4,4-tetramethyl-, (E)-), (2E,4E)-3-methyl-5-(2-((E)-2-(2,6,6-trimethyl-1-cyclohexen-1-yl)ethenyl)-1-cyclohexen-1-yl)-2,4-pentadienoic acid, tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))—(O)—), aliretinoin (retinoic acid, cis-9, trans-13-), bexarotene (benzoic acid, 4-(1-(5,6,7,8-tetrahydro-3,5,5,8,8-pentamethyl-2-naphthalenyl)ethenyl)-), tocoretinate (retinoic acid, 3,4-dihydro-2,5,7,8-tetramethyl-2-(4,8,12-trimethyltridecyl)-2H-1-benzopyran-6-yl ester, [2R*(4R*,8R*)]-(O)—, or an analogue or derivative thereof).

37. Platelet Derived Growth Factor Receptor Kinase Inhibitors

In another embodiment, the pharmacologically active compound is a platelet derived growth factor receptor kinase inhibitor (e.g., leflunomide (4-isoxazolecarhoxamide, 5-methyl-N-(4-(trifluoromethyl)phenyl)-, or an analogue or derivative thereof).

38. Fibronogin Antagonists

In another embodiment, the pharmacologically active compound is a fibrinogin antagonist (e.g., picotamide (1,3-benzenedicarboxamide, 4-methoxy-N,N′-bis(3-pyridinylmethyl)-, or an analogue or derivative thereof).

39. Antimycotic Agents

In another embodiment, the pharmacologically active compound is an antimycotic agent (e.g., miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (piperazine, 1-(4-((2-(2,4-dichlorophenyl)-2-(1H-1,2,4-triazol-1-ylmethyl)-1,3-dioxolan-4-yl)methoxy)phenyl)-4-(1-methylethyl)-, cis-), isoconazole (1-(2-(2-6-dichlorobenzyloxy)-2-(2-,4-dichlorophenyl)ethyl)), griseofulvin (spiro(benzofuran-2(3H), 1′-(2)cyclohexane)-3,4′-dione, 7-chloro-2′,4,6-trimeth-oxy-6′methyl-, (1′S-trans)-), bifonazole (1H-imidazole, 1-((1,1′-biphenyl)-4-ylphenylmethyl)-), econazole nitrate (1-(2-((4-chlorophenyl)methoxy)-2-(2,4-dichlorophenyl)ethyl)-1H-imidazole nitrate), croconazole (1H-imidazole, 1-(1-(2-((3-chlorophenyl)methoxy)phenyl)ethenyl)-), sertaconazole (1H-imidazole, 1-(2-((7-chlorobenzo(b)thien-3-yl)methoxy)-2-(2,4-dichlorophenyl)ethyl)-), omoconazole (1H-imidazole, 1-(2-(2-(4-chlorophenoxy)ethoxy)-2-(2,4-dichlorophenyl)-1-methylethenyl)-, (Z)-), flutrimazole (1H-imidazole, 1-((2-fluorophenyl)(4-fluorophenyl)phenylmethyl)-), fluconazole (1H-1,2,4-triazole-1-ethanol, alpha-(2,4-difluorophenyl)-alpha-(1H-1,2,4-triazol-1-ylmethyl)-), neticonazole (1H-Imidazole, 1-(2-(methylthio)-1-(2-(pentyloxy)phenyl)ethenyl)-, monohydrochloride, (E)-), butoconazole (1H-imidazole, 1-(4-(4-chlorophenyl)-2-((2,6-dichlorophenyl)thio)butyl)-, (+/−)-), clotrimazole (1-((2-chlorophenyl)diphenylmethyl)-1H-imidazole, or an analogue or derivative thereof).

40. Bisphosphonates

In another embodiment, the pharmacologically active compound is a bisphosphonate (e.g. clodronate, alendronate, pamidronate, zoledronate, or an analogue or derivative thereof).

41. Phospholipase A1 Inhibitors

In another embodiment, the pharmacologically active compound is a phospholipase A1 inhibitor (e.g., ioteprednol etabonate (androsta-1,4-diene-17-carboxylic acid, 17-((ethoxycarbonyl)oxy)-11-hydroxy-3-oxo-, chloromethyl ester, (11β,17 alpha)-, or an analogue or derivative thereof).

42. Histamine H1/H2/H3 Receptor Antagonists

In another embodiment, the pharmacologically active compound is a histamine H1, H2, or H3 receptor antagonist (e.g., ranitidine (1,1-ethenediamine, N-(2-(((5-((dimethylamino)methyl)-2-furanyl)methyl)thio)ethyl)-N′-methyl-2-nitro-), niperotidine (N-(2-((5-((dimethylamino)methyl)furfuryl)thio)ethyl)-2-nitro-N′-piperonyl-1,1-ethenediamine), famotidine (propanimidamide, 3-(((2-((aminoiminomethyl)amino)-4-thiazolyl)methyl)thio)-N-(aminosulfonyl)-), roxitadine acetate HCl (acetamide, 2-(acetyloxy)-N-(3-(3-(1-piperidinylmethyl)phenoxy)propyl)-, monohydrochloride), lafutidine (acetamide, 2-((2-furanylmethyl)sulfinyl)-N-(4-((4-(1-piperidinylmethyl)-2-pyridinyl)oxy)-2-butenyl)-, (Z)-), nizatadine (1,1-ethenediamine, N-(2-(((2-((dimethylamino)methyl)-4-thiazolyl)methyl)thio)ethyl)-N′-methyl-2-nitro-), ebrotidine (benzenesulfonamide, N-(((2-(((2-((aminoiminomethyl)amino)-4-thiazoly)methyl)thio)ethyl)amino)methylene)-4-bromo-), rupatadine (5H-benzo(5,6)cyclo hepta(1,2-b)pyridine, 8-chloro-6,11-dihydro-11-(1-((5-methyl-3-pyridinyl)methyl)-4-piperidinylidene)-, trihydrochloride-), fexofenadine HCl (benzeneacetic acid, 4-(1-hydroxy-4-(4(hydroxydiphenylmethyl)-1-piperidinyl)butyl)-alpha,alpha-dimethyl-, hydrochloride, or an analogue or derivative thereof)

43. Macrolide Antibiotics

In another embodiment, the pharmacologically active compound is a macrolide antibiotic (e.g., dirithromycin (erythromycin, 9-deoxo-11-deoxy-9,11-(imino(2-(2-methoxyethoxy)ethylidene)oxy)-, (9S(R))-), flurithromycin ethylsuccinate (erythromycin, 8-fluoro-mono(ethyl butanedioate) (ester)-), erythromycin stinoprate (erythromycin, 2′-propanoate, compound with N-acetyl-L-cysteine (1:1)), clarithromycin (erythromycin, 6-O-methyl-), azithromycin (9-deoxo-9a-aza-9a-methyl-9a-homoerythromycin-A), telithromycin (3-de((2,6-dideoxy-3-C-methyl-3-O-methyl-alpha-L-ribo-hexopyranosyl)oxy)-11,12-dideoxy-6-O-methyl-3-oxo-12,11-(oxycarbonyl((4-(4-(3-pyridinyl)-1H-imidazol-1-yl)butyl)imino))-), roxithromycin (erythromycin, 9-(0-((2-methoxyethoxy)methyl)oxime)), rokitamycin (leucomycin V, 4B-butanoate 3B-propanoate), RV-11 (erythromycin monopropionate mercaptosuccinate), midecamycin acetate (leucomycin V, 3B,9-diacetate 3,4B-dipropanoate), midecamycin (leucomycin V, 3,4B-dipropanoate), josamycin (leucomycin V, 3-acetate 4B-(3-methylbutanoate), or an analogue or derivative thereof).

44. GPIIb IIIa Receptor Antagonists

In another embodiment, the pharmacologically active compound is a GPIIb IIIa receptor antagonist (e.g., tirofiban hydrochloride (L-tyrosine, N-(butylsulfonyl)-O-(4-(4-piperidinyl)butyl)-, monohydrochloride-), eptifibatide (L-cysteinamide, N6-(aminoiminomethyl)-N2-(3-mercapto-1-oxopropyl)-L-lysylglycyl-L-alpha-aspartyl-L-tryptophyl-L-prolyl-, cyclic(1->6)-disulfide), xemilofiban hydrochloride, or an analogue or derivative thereof).

45. Endothelin Receptor Antagonists

In another embodiment, the pharmacologically active compound is an endothelin receptor antagonist (e.g., bosentan (benzenesulfonamide, 4-(1,1-dimethylethyl)-N-(6-(2-hydroxyethoxy)-5-(2-methoxyphenoxy)(2,2′-bipyrimidin)-4-yl)-, or an analogue or derivative thereof).

46. Peroxisome Proliferator-Activated Receptor Agonists

In another embodiment, the pharmacologically active compound is a peroxisome proliferator-activated receptor agonist (e.g., gemfibrozil (pentanoic acid, 5-(2,5-dimethylphenoxy)-2,2-dimethyl-), fenofibrate (propanoic acid, 2-(4-(4-chlorobenzoyl)phenoxy)-2-methyl-, 1-methylethyl ester), ciprofibrate (propanoic acid, 2-(4-(2,2-dichlorocyclopropyl)phenoxy)-2-methyl-), rosiglitazone maleate (2,4-thiazolidinedione, 5-((4-(2-(methyl-2-pyridinylamino)ethoxy)phenyl)methyl)-, (Z)-2-butenedioate (1:1)), pioglitazone hydrochloride (2,4-thiazolidinedione, 5-((4-(2-(5-ethyl-2-pyridinyl)ethoxy)phenyl)methyl)-, monohydrochloride (+/−)-), etofylline clofibrate (propanoic acid, 2-(4-chlorophenoxy)-2-methyl-, 2-(1,2,3,6-tetrahydro-1,3-dimethyl-2,6-dioxo-7H-purin-7-yl)ethyl ester), etofibrate (3-pyridinecarboxylic acid, 2-(2-(4-chlorophenoxy)-2-methyl-1-oxopropoxy)ethyl ester), clinofibrate (butanoic acid, 2,2′-(cyclohexylidenebis(4,1-phenyleneoxy))bis(2-methyl-)), bezafibrate (propanoic acid, 2-(4-(2-((4-chlorobenzoyl)amino)ethyl)phenoxy)-2-methyl-), binifibrate (3-pyridinecarboxylic acid, 2-(2-(4-chlorophenoxy)-2-methyl-1-oxopropoxy)-1,3-propanediyl ester), or an analogue or derivative thereof).

In one aspect, the pharmacologically active compound is a peroxisome proliferator-activated receptor alpha agonist, such as GW-590735, GSK-677954, GSK501516, pioglitazone hydrochloride (2,4-thiazolidinedione, 5-[[4-[2-(5-ethyl-2-pyridinyl)ethoxy]phenyl]methyl]-, monohydrochloride (+/−)-, or an analogue or derivative thereof).

47. Estrogen Receptor Agents

In another embodiment, the pharmacologically active compound is an estrogen receptor agent (eg. estradiol, 17-β-estradiol, or an analogue or derivative thereof).

48. Somatostatin Analogues

In another embodiment, the pharmacologically active compound is a somatostatin analogue (e.g., angiopeptin, or an analogue or derivative thereof).

49. Neurokinin 1 Antagonists

In another embodiment, the pharmacologically active compound is a neurokinin 1 antagonist (e.g., GW-597599, lanepitant ((1,4′-bipiperidine)-1′-acetamide, N-(2-(acetyl((2-methoxyphenyl)methyl)amino)-1-(1H-indol-3-ylmethyl)ethyl)-(R)—), nolpitantium chloride (1-azoniabicyclo[22,2]octane, 1-[2-[3-(3,4-dichlorophenyl)-1-[[3-(1-methylethoxy)phenyl]acetyl]-3-piperidinyl]ethyl]-4-phenyl-, chloride, (S)—), or saredutant (benzamide, N-[4-[4-(acetylamino)-4-phenyl-1-piperidinyl]-2-(3,4-dichlorophenyl)butyl]-N-methyl-, (S)—), or vofopitant (3-piperidinamine, N-[[2-methoxy-5-[5-(trifluoromethyl)-1H-tetrazol-1-yl]phenyl]methyl]-2-phenyl-, (2S,3S)—, or an analogue or derivative thereof).

50. Neurokinin 3 Antagonist

In another embodiment, the pharmacologically active compound is a neurokinin 3 antagonist (e.g., talnetant (4-quinolinecarboxamide, 3-hydroxy-2-phenyl-N-[(1S)-1-phenylpropyl]-, or an analogue or derivative thereof).

51. Neurokinin Antagonist

In another embodiment, the pharmacologically active compound is a neurokinin antagonist (e.g., GSK-679769, GSK-823296, SR-489686 (benzamide N-[4-[4-(acetylamino)-4-phenyl-1-piperidinyl]-2-(3,4-dichlorophenyl)butyl]-N-methyl-, (S)—), SB-223412, SB-235375 (4-quinolinecarboxamide, 3-hydroxy-2-phenyl-N-[(1S)-1-phenylpropyl]-), UK-226471, or an analogue or derivative thereof)

52. VLA-4 Antagonist

In another embodiment, the pharmacologically active compound is a VLA-4 antagonist (e.g., GSK683699, or an analogue or derivative thereof).

53. Osteoclast Inhibitor

In another embodiment, the pharmacologically active compound is a osteoclast inhibitor (e.g., ibandronic acid (phosphonic acid, [1-hydroxy-3-(methylpentylamino)propylidene]bis-), alendronate sodium, or an analogue or derivative thereof).

54. DNA topoisomerase ATP Hydrolysing Inhibitor

In another embodiment, the pharmacologically active compound is a DNA topoisomerase ATP hydrolysing inhibitor (e.g., enoxacin (1,8-naphthyridine-3-carboxylic acid, 1-ethyl-6-fluoro-1,4-dihydro-4-oxo-7-(1-piperazinyl)-), levofloxacin (7H-Pyrido[1,2,3-de]-1,4-benzoxazine-6-carboxylic acid, 9-fluoro-2,3-dihydro-3-methyl-10-(4-methyl-1-piperazinyl)-7-oxo-, (S)—), ofloxacin (7H-pyrido[1,2,3-de]-1,4-benzoxazine-6-carboxylic acid, 9-fluoro-2,3-dihydro-3-methyl-10-(4-methyl-1-piperazinyl)-7-oxo-, (+/−)-), pefloxacin (3-quinolinecarboxylic acid, 1-ethyl-6-fluoro-1,4-dihydro-7-(4-methyl-1-piperazinyl)-4-oxo-), pipemidic acid (pyrido[2,3-d]pyrimidine-6-carboxylic acid, 8-ethyl-5,8-dihydro-5-oxo-2-(1-piperazinyl)-), pirarubicin (5,12-naphthacenedione, 10-[[3-amino-2,3,6-trideoxy-4-O-(tetrahydro-2H-pyran-2-yl)-alpha-L-lyxo-hexopyranosyl]oxy]-7,8,9,10-tetrahydro-6,8,11-trihydroxy-8-(hydroxyacetyl)-1-methoxy-, [8S-[8 alpha,10 alpha(S*)]]-), sparfloxacin (3-quinolinecarboxylic acid, 5-amino-1-cyclopropyl-7-(3,5-dimethyl-1-piperazinyl)-6,8-difluoro-1,4-dihydro-4-oxo-, cis-), AVE-6971, cinoxacin ([1,3]dioxolo[4,5-g]cinnoline-3-carboxylic acid, 1-ethyl-1,4-dihydro-4-oxo-), or an analogue or derivative thereof)

55. Angiotensin I Converting Enzyme Inhibitor

In another embodiment, the pharmacologically active compound is an angiotensin I converting enzyme inhibitor (e.g., ramipril (cyclopenta[b]pyrrole-2-carboxylic acid, 1-[2-[[1-(ethoxycarbonyl)-3-phenylpropyl]amino]-1-oxopropyl]octahydro-, [2S-[1-[R*(R*)],2 alpha, 3aβ,6aβ]]-), trandolapril (1H-indole-2-carboxylic acid, 1-[2-[(1-carboxy-3-phenylpropyl)amino]-1-oxopropyl]octahydro-, [2S-[1[R*(R*)],2 alpha,3a alpha, 7aβ]]-), fasidotril (L-alanine, N-[(2S)-3-(acetylthio)-2-(1,3-benzodioxol-5-ylmethyl)-1-oxopropyl]-, phenylmethyl ester), cilazapril (6H-pyridazino[1,2-a][1,2]diazepine-1-carboxylic acid, 9-[[1-(ethoxycarbonyl)-3-phenylpropyl]amino]octahydro-10-oxo-, [1S-[1 alpha, 9 alpha(R*)]]-), ramipril (cyclopenta[b]pyrrole-2-carboxylic acid, 1-[2-[[1-(ethoxycarbonyl)-3-phenylpropyl]amino]-1-oxopropyl]octahydro-, [2S-[1[R*(R*)], 2 alpha,3aβ,3aβ,6aβ]]-, or an analogue or derivative thereof).

56. Angiotensin II Antagonist

In another embodiment, the pharmacologically active compound is an angiotensin II antagonist (e.g., HR-720 (1H-imidazole-5-carboxylic acid, 2-butyl-4-(methylthio)-1-[[2′-[[[(propylamino)carbonyl]amino]sulfonyl][1,1′-biphenyl]-4-yl]methyl]-, dipotassium salt, or an analogue or derivative thereof).

57. Enkephalinase Inhibitor

In another embodiment, the pharmacologically active compound is an enkephalinase inhibitor (e.g., Aventis 100240 (pyrido[2,1-a][2]benzazepine-4-carboxylic acid, 7-[[2-(acetylthio)-1-oxo-3-phenylpropyl]amino]-1,2,3,4,6,7,8,12b-octahydro-6-oxo-[4S-[4 alpha, 7 alpha(R*),12bβ]]-), AVE-7688, or an analogue or derivative thereof)

58. Peroxisome Proliferator-Activated Receptor Gamma Agonist Insulin Sensitizer

In another embodiment, the pharmacologically active compound is peroxisome proliferator-activated receptor gamma agonist insulin sensitizer (e.g., rosiglitazone maleate (2,4-thiazolidinedione, 5-((4-(2-(methyl-2-pyridinylamino)ethoxy)phenyl)methyl)-, (Z)-2-butenedioate (1:1), farglitazar (GI-262570, GW-2570, GW-3995, GW-5393, GW-9765), LY-929, LY-519818, LY-674, or LSN-862), or an analogue or derivative thereof).

59. Protein Kinase C Inhibitor

In another embodiment, the pharmacologically active compound is a protein kinase C inhibitor, such as ruboxistaurin mesylate (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)—), safingol (1,3-octadecanediol, 2-amino-, [S—(R*,R*)]-), or enzastaurin hydrochloride (1H-pyrole-2,5-dione, 3-(1-methyl-1H-indol-3-yl)-4-[1-[1-(2-pyridinylmethyl)-4-piperidinyl]-1H-indol-3-yl]-, monohydrochloride), or an analogue or derivative thereof.

60. ROCK (rho-Associated Kinase) Inhibitors

In another embodiment, the pharmacologically active compound is a ROCK (rho-associated kinase) inhibitor, such as Y-27632, HA-1077, H-1152 and 4-1-(aminoalkyl)-N-(4-pyridyl)cyclohexanecarboxamide or an analogue or derivative thereof.

61. CXCR3 Inhibitors

In another embodiment, the pharmacologically active compound is a CXCR3 inhibitor such as T487, T0906487 or analogue or derivative thereof

62. Itk Inhibitors

In another embodiment, the pharmacologically active compound is an Itk inhibitor such as BMS-509744 or an analogue or derivative thereof.

63. Cytosolic Phospholipase A₂-alpha Inhibitors

In another embodiment, the pharmacologically active compound is a cytosolic phospholipase A₂-alpha inhibitor such as efipladib (PLA-902) or analogue or derivative thereof.

64. PPAR Agonist

In another embodiment, the pharmacologically active compound is a PPAR Agonist (e.g., Metabolex ((−)-benzeneacetic acid, 4-chloro-alpha-[3-(trifluoromethyl)-phenoxy]-, 2-(acetylamino)ethyl ester), balaglitazone (5-(4-(3-methyl-4-oxo-3,4-dihydro-quinazolin-2-yl-methoxy)-benzyl)-thiazolidine-2,4-dione), ciglitazone (2,4-thiazolidinedione, 5-[[4-[(1-methylcyclohexyl)methoxy]phenyl]methyl]-), DRF-10945, farglitazar, GS K-677954, GW-409544, GW-501516, GW-590735, GW-590735, K-111, KRP-101, LSN-862, LY-519818, LY-674, LY-929, muraglitazar; BMS-298585 (Glycine, N-[(4-methoxyphenoxy)carbonyl]-N—[[4-[2-(5-methyl-2-phenyl-4-oxazolyl)ethoxy]phenyl]methyl]-), netoglitazone; isaglitazone (2,4-thiazolidinedione, 5-[[6-[(2-fluorophenyl) methoxy]-2-naphthalenyl]methyl]-), Actos AD-4833; U-72107A (2,4-thiazolidinedione, 5-[[4-[2-(5-ethyl-2-pyridinyl)ethoxy]phenyl]methyl]-, monohydrochloride (+/−)-), JTT-501; PNU-182716 (3,5-Isoxazolidinedione, 4-[[4-[2-(5-methyl-2-phenyl-4-oxazolyl)ethoxy]phenyl]methyl]-), AVANDIA (from SB Pharmco Puerto Rico, Inc. (Puerto Rico), BRL-48482, BRL-49653, BRL-49653c, NYRACTA and Venvia (both from (SmithKline Beecham (United Kingdom)), tesaglitazar ((2S)-2-ethoxy-3-[4-[2-[4-[(methylsulfonyl)oxy]phenyl]ethoxy]phenyl]propanoic acid), troglitazone (2,4-Thiazolidinedione, 5-[[4-[(3,4-dihydro-6-hydroxy-2,5,7,8-tetramethyl-2H-1-benzopyran-2-yl)methoxy]phenyl]methyl]-), and analogues and derivatives thereof).

65. Immunosuppressants

In another embodiment, the pharmacologically active compound is an immunosuppressant (e.g., batebulast (cyclohexanecarboxylic acid, 4-[[(aminoiminomethyl)amino]methyl]-, 4-(1,1-dimethylethyl)phenyl ester, trans-), cyclomunine, exalamide (benzamide, 2-(hexyloxy)-), LYN-001, CCI-779 (rapamycin 42-(3-hydroxy-2-(hydroxymethyl)-2-methylpropanoate)),1726; 1726-D; AVE-1726, or an analogue or derivative thereof).

66. Erb Inhibitor

In another embodiment, the pharmacologically active compound is an Erb inhibitor (e.g., canertinib dihydrochloride (N-[4-(3-(chloro-4-fluoro-phenylamino)-7-(3-morpholin-4-yl-propoxy)-quinazolin-6-yl]-acrylamide dihydrochloride), CP-724714, or an analogue or derivative thereof).

67. Apoptosis Agonist

In another embodiment, the pharmacologically active compound is an apoptosis agonist (e.g., CEFLATONIN (CGX-635) (from Chemgenex Therapeutics, Inc., Menlo Park, Calif.), CHML, LBH-589, metoclopramide (benzamide, 4-amino-5-chloro-N-[2-(diethylamino)ethyl]-2-methoxy-), patupilone (4,17-dioxabicyclo(14.1.0)heptadecane-5,9-dione, 7,11-dihydroxy-8,8,10,12,16-pentamethyl-3-(1-methyl-2-(2-methyl-4-thiazolyl)ethenyl, (1R,3S,7S,10R,11S,12S,16R)), AN-9; pivanex (butanoic acid, (2,2-dimethyl-1-oxopropoxy)methyl ester), SL-100, SL-102; SL-11093; SL-11098. SL-11099; SL-93; SL-98. SL-99, or an analogue or derivative thereof)

68. Lipocortin Agonist

In another embodiment, the pharmacologically active compound is an lipocortin agonist (e.g., CGP-13774 (9Alpha-chloro-6Alpha-fluoro-11β,17alpha-dihydroxy-16Alpha-methyl-3-oxo-1,4-androstadiene-17′-carboxylic acid-methylester-17-propionate), or analogue or derivative thereof).

69. VCAM-1 Antagonist

In another embodiment, the pharmacologically active compound is a VCAM-1 antagonist (e.g., DW-908e, or an analogue or derivative thereof).

70. Collagen Antagonist

In another embodiment, the pharmacologically active compound is a collagen antagonist (e.g., E-5050 (Benzenepropanamide, 4-(2,6-dimethylheptyl)-N-(2-hydroxyethyl)-β-methyl-), lufironil (2,4-Pyridinedicarboxamide, N,N′-bis(2-methoxyethyl)-), or an analogue or derivative thereof).

71. Alpha 2 Integrin Antagonist

In another embodiment, the pharmacologically active compound is an alpha 2 integrin antagonist (e.g., E-7820, or an analogue or derivative thereof).

72. TNF Alpha Inhibitor

In another embodiment, the pharmacologically active compound is a TNF alpha inhibitor (e.g., ethyl pyruvate, Genz-29155, lentinan (Ajinomoto Co., Inc. (Japan)), linomide (3-quinolinecarboxamide, 1,2-dihydro-4-hydroxy-N,1-dimethyl-2-oxo-N-phenyl-), UR-1505, or an analogue or derivative thereof).

73. Nitric Oxide Inhibitor

In another embodiment, the pharmacologically active compound is a nitric oxide inhibitor (e.g., guanidioethyldisulfide, or an analogue or derivative thereof).

74. Cathepsin Inhibitor

In another embodiment, the pharmacologically active compound is a cathepsin inhibitor (e.g., SB-462795 or an analogue or derivative thereof).

75. Adensosine A2A Receptor Antagonist

In another embodiment, the fibrosis-inhibiting compound is an adensosineA2A receptor antagonist (e.g., Sch-63390 (Schering-Plough) or an A2A receptor antagonists from Almirall-Prodesfarma, SCH-58261 (CAS No. 160098-96-4), or an analogue or derivative thereof).

76. AKT Inhibitor

In another embodiment, the fibrosis-inhibiting compound is an AKT inhibitor (e.g., PKB inhibitors from DeveloGen, AKT inhibitors from Array BioPharma, Celgene, Merck & Co, Amphora, NeoGenesis Pharmaceuticals, A-443654 (Abbott Laboratories), erucylphosphocholine (AEterna Zentaris), KRX-401 (Keryx), protein kinase B inhibitors from Astex Technology, PX-316 (ProIX), or an analogue or derivative thereof).

77. Alpha 2 Integrin Antagonist

In another embodiment, the fibrosis-inhibiting compound is an alpha 2 integrin antagonist (e.g., Pharmaprojects No. 5754 (Merck KGaA), or an analogue or derivative thereof).

78. Alpha 4 Integrin Antagonist

In another embodiment, the fibrosis-inhibiting compound is an alpha 4 integrin antagonist (e.g. T 0047 (Tanabe Seiyaku), VLA-4 antagonists from Sanofi-Aventis, Merck & Co Biogen Idec, Uriach, and Molecumetics, alpha 4 integrin antagonists from Genentech), BIO-2421 (Biogenidec), cell adhesion inhibitors from Kaken Pharmaceuticals, CT-737 (Wyeth), CT-767 (Elan), CY-9652 (Epimmune), CY-9701 (Epimmune), fibronectin antagonists from Uriach, integrin alpha4β7 antagonists frin Wilex, Pharmaprojects No. 5972 (UCB), Pharmaprojects No. 6603 (Wyeth), TBC-3342, TBC-772, and TBC-3486 (Encysive Pharmaceuticals), TBC-4746 (Schering-Plough), or a VLA4/VCAM inhibitor (Elan Pharmaceuticals), ZD-7349 (AstraZeneca), or an analogue or derivative thereof).

79. Alpha 7 Nicotinic Receptor Agonist

In another embodiment, the fibrosis-inhibiting compound is an alpha 7 nicotinic receptor agonist (e.g., AZD-0328 (AstraZeneca), galantamine (CAS No. 357-70-0) (Synaptec), MEM-3454 or nicotinic alpha-7 agonist (Memory Pharmaceuticals and Critical Therapeutics), Pharmaprojects No. 4779 (AstraZeneca), PNU-282987 (Pfizer), SSR-180711 (Sanofi-Aventis), TC-1698 or TC-5280 (Targacept), or an analogue or derivative thereof).

80. Angiogenesis Inhibitors

In one embodiment, the fibrosis-inhibiting compound is an angiogenesis inhibitor (e.g., AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Aftenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zenaca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto). Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (such as combretastatin A-1, A-2, A-3, A-4, A-5, A-6, B-1, B-2, B-3, B-4, D-1, D-2, and combretastatin A-4 phosphate (Oxigene)), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), or an analogue or derivative thereof). In other embodiments, the angiogenesis inhibitor may be a recombinant anti-angiogenic compound such as ANGIOCOL (available from Biostratum Inc., Durham, N.C.).

81. Apoptosis Antagonists

In another embodiment, the fibrosis-inhibiting compound is an apoptosis antagonist (e.g., didemnin B, RGB-286199 (GPC Biotech), 5F-DF-203 (Cancer Research Technology), aplidine, bongkrekic acid, triammonium salt, [6]-gingerol (CAS No. 23513-14-6), or an analogue or derivative thereof).

### 82. Apoptosis Activators

In another embodiment, the fibrosis-inhibiting compound is an apoptosis activator (e.g., aplidine (CAS No. 137219-37-5) (PharmaMar), canfosfamide hydrochloride (CAS No. 58382-37-74 and 39943-59-6) (Telik), idronoxil (CAS No. 81267-65-4) (Novogen), OSI-461 (OSI Pharmaceuticals), DE-098 (Santen), ARQ-550RP (ArQule), ABJ-879 (Novartis), adaphostin (NIH), anticancer agents from Apogenix Biotechnology and Momenta Pharmaceuticals, anti-PARP-1 or anti-PARP-2 (Octamer), BA-1037 (BioAxone), CP-248 (CAS No. 200803-37-8) (OSI Pharmaceuticals), EM-1421 (Erimos), IPI-504 (Infinity Pharmaceuticals), KP-372-1 (QLT), MPC-6827 (Maxim), MT-103 (Medisyn Technologies), MX-116407 or MX-126374 (Maxim), NPI-0052 (Nereus Pharmaceuticals), NVP-AEW541 (Novartis), PARP inhibitor from Agouron (Pfizer), R-306465 (Johnson & Johnson), TG-100-33 (TargeGen), a XIAP inhibitor from AEgera, ZEN-011 (AEterna Zentaris), canertinib dihydrochloride (CAS No. 289499-45-2) (Pfizer), BH31-1,3-BAABE, or an analogue or derivative thereof).

83. Beta 1 Integrin Antagonist

In another embodiment, the fibrosis-inhibiting compound is a beta 1 integrin antagonist (e.g., β-1 integrin antagonists, Berkeley Lab, or an analogue or derivative thereof).

84. Beta Tubulin Inhibitor

In another embodiment, the fibrosis-inhibiting compound is a beta tubulin inhibitor (e.g., ZEN-017 (AEterna Zentaris), laulimalide (Kosan Biosciences), or an analogue or derivative thereof).

85. Blockers of Enzyme Production in Hepatitis C

In another embodiment, the fibrosis-inhibiting compound is an agent that blocks enzyme production in hepatitis C (eg, merimepodib (Vertex Pharmaceuticals), or an analogue or derivative thereof).

86. Bruton's Tyrosine Kinase Inhibitor

In another embodiment, the fibrosis-inhibiting compound is a Bruton's tyrosine kinase inhibitor (e.g., a Btk inhibitor from Cellular Genomics, or an analogue or derivative thereof).

87. Calcineurin Inhibitors

In another embodiment, the fibrosis-inhibiting compound is a calcineurin inhibitor (e.g., tacrolimus (LifeCycle Pharma), or an analogue or derivative thereof).

88. Caspase 3 Inhibitors

In another embodiment, the fibrosis-inhibiting compound is a caspase 3 inhibitor (e.g., NM-3 (Mercian), or an analogue or derivative thereof).

89. CC Chemokine Receptor Antagonists

In another embodiment, the fibrosis-inhibiting compound is a CC chemokine receptor antagonist (e.g., a chemokine receptor 3 antagonist, a chemokine receptor 6 antagonist, and a chemokine receptor 7 antagonist). Representative examples of CC chemokine receptor antagonists include chemokine antagonists such as the CCR7 antagonists from Neurocrine Biosciences.

In a related embodiment, the fibrosis-inhibiting compound is a CC chemokine receptor antagonist (CCR) 1, 3, & 5 (e.g., peptide T (Advanced Immuni T), a CCR3 antagonist from GlaxoSmithKline, a chemokine antagonist (Pharmaprojects No. 6322) from Neurocrine Biosciences or Merck & Co., an HIV therapy agent from ReceptoPharm (Nutra Pharma), Pharmaprojects No. 6129 (Sangamo BioSciences), or an analogue or derivative thereof)

In certain embodiments, the CCCR antagonist is a CCR2b chemokine receptor antagonist such as RS 102895 (CAS No. 300815-41-2)

90. Cell Cycle Inhibitors

In another embodiment, the fibrosis-inhibiting compound is a cell cycle inhibitor (e.g., SNS-595 (Sunesis), homoharringtonine, or an analogue or derivative thereof).

In certain embodiments, the cell cycle inhibitor is an anti-microtubule agent (e.g., synthadotin, or an analogue or derivative thereof).

In certain embodiments, cell cycle inhibitor is a microtubule stimulant (e.g., KRX-0403, or an analogue or derivative thereof).

91. Cathepsin B Inhibitor

In another embodiment, the fibrosis-inhibiting compound is a cathepsin B inhibitor (e.g., AM-4299A (Asahi Kasei Pharma), BDI-7800 (Biopharmacopae), a cathepsin B inhibitor from Axys (Celera Genomics), MDL-104903 (CAS No. 180799-56-8) (Sanofi-Aventis), NC-700 (Nippon Chemiphar), Pharmaprojects No. 2332 (Hoffmann-La Roche), Pharmaprojects No. 4884 (Takeda), Pharmaprojects No. 5134 (Nippon Chemiphar), or an analogue or derivative thereof).

92. Cathepsin K Inhibitor

In another embodiment, the fibrosis-inhibiting compound is a cathepsin K inhibitor (e.g., 462795 (GlaxoSmithKline), INPL-022-D6 (Amura Therapeutics), or an analogue or derivative thereof).

93. Cathepsin L Inhibitor

In another embodiment, the fibrosis-inhibiting compound is a cathepsin L Inhibitor (eg, a cathepsin L inhibitor from Takeda, INPL-022-E10 (Amura Therapeutics), Pharmaprojects No 5447 (Taiho), or an analogue or derivative thereof)

94. CD40 Antagonists

In another embodiment, the fibrosis-inhibiting compound is a CD40 antagonists (e.g., 5D12 (Chiron), ABI-793 (Novartis), an anticancer antibody from Chiron, anti-CD40 MAb-2 (Kirin Brewery), anti-CD40 (Eli Lilly), anti-CD40L antibody (UCB), a CD40 inhibitor from Apoxis, CD40 ligand inhibitor from Millennium Pharmaceuticals, a CD40/CAP inhibitor from Snow Brand, CGEN-40 (Compugen), CHIR-12.12 (Chiron), Pharmaprojects No. 5163 (Nippon Kayaku), ruplizumab (Biogen Idec), SGN-40 (Seattle Genetics), TNX-100 (Akzo Nobel), toralizumab (CAS No 252662-47-8) (Biogen Idec), or an analogue or derivative thereof).

95. Chemokine Receptor Agonists

In another embodiment, the fibrosis-inhibiting compound is a chemokine receptor agonist (e.g., a chemokine agonist from NeuroTarget, or an analogue or derivative thereof).

96. Chymase Inhibitors

In another embodiment, the fibrosis-inhibiting compound is a chymase inhibitor (e.g., BL-3875 (Dainippon), LEX-043 (SuperGen), NK-3201 (CAS No. 204460-24-2) (Nippon Kayaku), or an analogue or derivative thereof).

97. Collagenase (Interstitial) Antagonists

In another embodiment, the fibrosis-inhibiting compound is a collagenase (interstitial) antagonist (e.g., IBFB-212543 (IBFB Pharma), Pharmaprojects No 3762 (Sanofi-Aventis), S-0885 (CAS No 117517-22-3) (Sanofi-Aventis), SC-40827 (CAS No 101470-42-2) (Pfizer), or an analogue or derivative thereof)

98. CXCR (2, 4) Antagonists

In another embodiment, the fibrosis-inhibiting compound is a CXCR (24) antagonist (e.g., SB-656933 (GlaxoSmithKline), AMD3100 octahydrochloride (CAS No. 155148-31-5), or an analogue or derivative thereof).

99. Cyclin Dependent Kinase Inhibitors

In another embodiment, the fibrosis-inhibiting compound is a cyclin dependent kinase (CDK) inhibitor. In certain embodiments, the cyclin dependent kinase inhibitor is a CDK-1 inhibitor. In certain embodiments, the cyclin dependent kinase inhibitor is a CDK-2 inhibitor. In certain embodiments, the cyclin dependent kinase inhibitor is a CDK-4 inhibitor. In certain embodiments, the cyclin dependent kinase inhibitor is a CDK-6 inhibitor. Representative examples of cyclin dependent kinase inhibitors include CAK1 inhibitors from GPC Biotech and Bristol-Myers Squibb, RGB-286199 (GPC Biotech), or an analogue or derivative thereof.

Additional exemplary cyclin dependent protein kinase inhibitors include an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), CGP 74514A, bohemine, olomoucine (CAS No. 101622-51-9), indole-3-carbinol (CAS No. 700-06-1), and an analogue or derivative thereof.

100. Cyclooxygenase Inhibitors

In another embodiment, the fibrosis-inhibiting compound is a cyclooxygenase inhibitor (eg. NS-398 (CAS No. 123653-11-2), ketoprofen, or an analogue or derivative thereof) In some embodiments, the cyclooxygenase inhibitor is a COX-1 inhibitor such as triflusal, or an analogue or derivative thereof).

101. Dihydroorotate Dehydrogenase Inhibitor (DHFR) Inhibitors

In another embodiment, the fibrosis-inhibiting compound is a DHFR inhibitor (e.g., PDX (Allos Therapeutics), SC12267, sulfamerazine (CAS No. 127-79-7), or an analogue or derivative thereof).

102. Dual Integrin Inhibitor

In another embodiment, the fibrosis-inhibiting compound is a dual integrin inhibitor (e.g, R411 (Roche Pharmaceuticals), or an analogue or derivative thereof).

103. Elastase Inhibitors

In another embodiment, the fibrosis-inhibiting compound is an elastase inhibitor (e.g., orazipone, depelestat (CAS No. 506433-25-6) (Dyax), AE-3763 (Dainippon), or an analogue or derivative thereof).

104. Elongation Factor-1 Alpha Inhibitors

In another embodiment, the fibrosis-inhibiting compound is an elongation factor-1alpha inhibitor (e.g., aplidine, or an analogue or derivative thereof).

105. Endothelial Growth Factor Antagonists

In another embodiment, the fibrosis-inhibiting compound is an endothelial growth factor (EGF) antagonist (e.g., neovastat, NM-3 (Mercian), or an analogue or derivative thereof).

106. Endothelial Growth Factor Receptor Kinase Inhibitors

In another embodiment, the fibrosis-inhibiting compound is an endothelial growth factor receptor (EGF-R) kinase inhibitor (e.g., sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), lavendustin A (CAS No. 125697-92-9), a KDR inhibitor from LG Life Sciences, CT-6685 or CT-6729 (UCB), KRN-633 or KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGF-R inhibitor such as SU 1498, a VEGFR-2 kinase inhibitor (Bristol-Myers Squibb), XL-647 (Exelixis), a KDR inhibitor from Abbott Laboratories, or an analogue or derivative thereof).

In another embodiment, the fibrosis-inhibiting compound is an endothelial growth factor receptor 2 kinase inhibitor (e.g., sorafenib tosylate, or an analogue or derivative thereof).

107. Endotoxin Antagonists

In another embodiment, the fibrosis-inhibiting compound is an endotoxin antagonist (e.g., E5564 (Eisai Pharmaceuticals), or an analogue or derivative thereof).

108. Epothilone and Tubulin Binders

In another embodiment, the fibrosis-inhibiting compound is an epothilone or tubulin binder (e.g., ixabepilone (BMS), or an analogue or derivative thereof.

109. Estrogen Receptor Antagonists

In another embodiment the fibrosis-inhibiting compound is an estrogen receptor antagonist (e.g ERB-041 (Wyeth), or an analogue or derivative thereof).

110. FGF Inhibitors

In another embodiment, the fibrosis-inhibiting compound is a FGF inhibitor (e.g., IDN-5390 (Indena), or an analogue or derivative thereof).

111. Farnexyl Transferase Inhibitors

In another embodiment, the fibrosis-inhibiting compound is an inhibitor of farnexyl transferase (FTI). In certain embodiments, the FTI inhibits the RAS oncogene family Examples of FTI's include SARASAR (from Schering Corporation, Kenilworth, N.J.), or an analogue or derivative thereof.

112. Farnesyltransferase Inhibitors

In another embodiment, the fibrosis-inhibiting compound is a farnesyltransferase inhibitor (e.g., A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), or an analogue or derivative thereof).

113. FLT-3 Kinase Inhibitors

In another embodiment, the fibrosis-inhibiting compound is a FLT-3 kinase inhibitor (e.g., Amphora, or an analogue or derivative thereof).

114. FGF Receptor Kinase Inhibitors

In another embodiment, the fibrosis inhibiting compound is a FGF receptor kinase inhibitor (e.g., MED-A300 (Gerolymatos). SSR-128129 (Sanofi-Aventis), TBC-2250 (Encysive Pharmaceuticals), XL-999 (Exelixis), or a FGF receptor kinase inhibitor from Paradigm Therapeutics, or an analogue or derivative thereof).

115. Fibrinogen Antagonists

In another embodiment, the fibrosis-inhibiting compound is a fibrinogen antagonist (e.g., AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), mevastatin, or an analogue or derivative thereof).

116. Heat Shock Protein 90 Antagonists

In another embodiment, the fibrosis-inhibiting compound is a heat shock protein 90 antagonist (e.g., SRN-005 (Sirenade), geldanamycin or a derivative thereof, such as NSC-33050 (17-allylaminogeldanamycin; 17-AAG) or 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), radicicol, Humicola fuscoatra (CAS No. 12772-57-6), or an analogue or derivative thereof).

117. Histone Deacetylase Inhibitors

In another embodiment, the fibrosis-inhibiting compound is a histone deacetylase inhibitor (e.g., FK228 (Gloucester), trichostatin A from Streptomyces sp. (CAS No. 58880-19-6), or an analogue or derivative thereof).

118. HMGCoA Reductase Inhibitors

In another embodiment, the fibrosis-inhibiting compound is an HMGCoA reductase inhibitor (e.g., an atherosclerosis therapeutic from Lipid Sciences, ATI-16000 (ARYx Therapeutics), KS-01-019 (Kos Pharmaceuticals), Pharmaprojects No 2197 (Sanofi-Aventi), RP 61969 (Sanofi-Aventis), cerivastatin Na) CAS No. 143201-11-0), or an analogue or derivative thereof)

119. ICAM Inhibitors

In another embodiment, the fibrosis-inhibiting compound is an ICAM inhibitor (e.g., alicaforsen (CAS No. 185229-68-9) (ISIS Pharmaceuticals), an ICAM-5 modulator (such as ICAM-4 from ICOS), or an analogue or derivative thereof).

120. IL-1, ICE & IRAK Antagonists

In another embodiment, the fibrosis-inhibiting compound is an IL-1, ICE & IRAK antagonist (e.g., CJ-14877 or CP-424174 (Pfizer), NF-61 (Negma-Lerads), or an analogue or derivative thereof).

121. IL-2 Inhibitors

In another embodiment, the fibrosis-inhibiting compound is an IL-2 inhibitor (e.g., AVE 8062 (Sanofi-Aventis), or an analogue or derivative thereof).

122. Immunosuppressants

In another embodiment, the fibrosis-inhibiting compound is an immunosuppressant (e.g., teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (inflazyme), MDL-9563 (CAS No. 27086-86-8) (Sanofi-Aventis), Pharmaprojects No 2330 (Sanofi-Aventis), Pharmaprojects No. 6426 (Abgenix). PXS-25 (Pharmaxis), rosmarinic acid (CAS No 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), or an analogue or derivative thereof).

123. IMPDH (Inosine Monophosphate)

In another embodiment, the fibrosis-inhibiting compound is IMPDH (inosine monophosphate) (e.g., ribavirin (Hoffmann-La Roche) or an analogue or derivative thereof).

124. Integrin Antagonists

In another embodiment, the fibrosis-inhibiting compound is an integrin antagonist (e.g., 683699 from Glaxo Smith Kline, integrin antagonists from Jerina AG (Germany), or an analogue or derivative thereof).

125. Interleukin Antagonists

In another embodiment, the fibrosis-inhibiting compound is an interleukin antagonist (e.g., dersalazine, or an analogue or derivative thereof).

In another embodiment, the fibrosis-inhibiting compound is an interleukin 1 antagonist (e.g., NPI-1302a-3, or an analogue or derivative thereof).

126. Inhibitors of Type III Receptor Tyrosine Kinases

In another embodiment, the fibrosis-inhibiting compound is an inhibitor of type III receptor tyrosine kinase such as FLT3, PDGRF and c-KIT (e.g., MLN518 (Millenium Pharmaceuticals), or an analogue or derivative thereof).

127. Irreversible Inhibitors of Enzyme Methionine Aminopeptidase Type 2

In another embodiment, the fibrosis-inhibiting compound is an irreversible Inhibitor of enzyme methionine aminopeptidase type 2 (e.g., PPI-2458 (Praecis Pharmaceuticals), or analogue or derivative thereof).

128. Isozyme-Selective Delta Protein Kinase C Inhibitors

In another embodiment, the fibrosis-inhibiting compound is an isozyme-selective delta protein kinase C inhibitor (e.g., KAI-9803 (Kai Pharmaceuticals), or an analogue or derivative thereof).

129. JAK3 Enzyme Inhibitors

In another embodiment, the fibrosis-inhibiting compound is a JAK3 enzyme inhibitor (e.g., CP-690,550 (Pfizer), or an analogue or derivative thereof).

130. JNK Inhibitors

In another embodiment, the fibrosis-inhibiting compound is a JNK inhibitor (e.g., BF-67192 (BioFocus), XG-101 or XG-102 (Xigen), or an analogue or derivative thereof).

131. Kinase Inhibitors

In another embodiment, the fibrosis-inhibiting compound is a kinase inhibitor (e.g., a kinase inhibitors from EVOTEC, or an analogue or derivative thereof).

132. Kinesin Antagonist

In another embodiment, the fibrosis-inhibiting compound is a kinesin antagonist (e.g., SB-715992 and an antifungal from Cytokinetics, or an analogue or derivative thereof).

133. Leukotriene Inhibitors and Antagonists

In another embodiment, the fibrosis-inhibiting compound is a leukotriene Inhibitor or antagonist (e.g., ambicromil (CAS No 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 or 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), or RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1) or SC-53228 (CAS No. 153633-01-3) (Pfizer), spaglumic acid (ZY-15106) (CAS No. 3106-85-2) or 80619-64-3 (Novartis), tipredane (CAS No. 85197-77-9) (Bristol-Myers Squibb), U-75302 (CAS No. 119477-85-9) (Pfizer), or analogue or derivative thereof).

134. MAP Kinase Inhibitors

In another embodiment, the fibrosis-inhibiting compound is a MAP kinase inhibitor (e.g., SRN-003-556 (Sirenade), AEG-3482 (AEgera), ARRY-142886 (Array BioPharma), CDP-146 (UCB), or analogue or derivative thereof).

135. Matrix Metalloproteinase Inhibitors (MMPI)

In another embodiment, the fibrosis-inhibiting compound is a matrix metalloproteinase inhibitor. A variety of MMPI's may be used in the practice of the invention. In one embodiment, the MMPI is a MMP-1 inhibitor. In another embodiment, the MMPI is a MMP-2 inhibitor. In other embodiments, the MMPI is a MMP-4, MMP-5, MMP-6, MMP-7, or MMP-8 inhibitor. Representative examples of MMPI's include glucosamine sulfate, neovastat, GM1489 (CAS No 170905-75-6). XL784 (EXEL-01370784). TNF-a Protease Inhibitor-1 or 2 (TAPI-1 or TAPI-2), galardin, or an analogue or derivative thereof.

136. MCP-CCR2 Inhibitors

In another embodiment, the fibrosis-inhibiting compound is a MCP-CCR2 inhibitor (e.g., MLN1202 (Millennium Pharmaceuticals) or an analogue or derivative thereof).

137. mTOR Inhibitor

In another embodiment, the fibrosis-inhibiting compound is an mTOR inhibitor (e.g., temsirolimus (CAS No. 162635-04-3) (Wyeth), or an analogue or derivative thereof).

138. mTOR Kinase Inhibitor

In another embodiment, the fibrosis-inhibiting compound is an mTOR kinase inhibitor (e.g., ABT-578 (Abbott), temsirolimus (Wyeth), AP-23573 (Ariad), or an analogue or derivative thereof).

139. Microtubule Inhibitors

In another embodiment, the fibrosis-inhibiting compound is a microtubule inhibitor (e.g., antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4 or huMy9-6-DM1 (ImmunoGen), IDN-5404 (Indena), IMO-098 or IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine dolastatin 15 (CAS No 123884-00-4) or an analogue or derivative thereof)

In certain embodiments, the microtubule inhibitor is a microtubule polymerization inhibitor such as vincamine, or an analogue or derivative thereof).

140. MIF Inhibitors

In another embodiment, the fibrosis-inhibiting compound is a MIF inhibitor (e.g., AVP-13546 (Avanir), an MIF inhibitor from Genzyme, migration stimulation factor D, or an analogue or derivative thereof).

141. MMP (Stromolysin) Inhibitors

In another embodiment, the fibrosis-inhibiting compound is a MMP (stromolysin) inhibitor (e.g., anticancer tetracycline from Tetragenex, rhostatin (BioAxone), TIMP's from Sanofi-Aventis (CAS No. 86102-31-0), and MMP inhibitors form Cognosci and Tetragenex, or an analogue or derivative thereof).

142. Neurokinin (NK) Antagonist

In another embodiment, the fibrosis-inhibiting compound is a neurokinin (NK) antagonist (e.g., anthrotainin (CAS No. 148084-40-6) (Sanofi-Aventis), an IBS thereapeutic such as SLV-332 from ArQule, MDL-105212A (CAS No. 167261-60-1) (Ssanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600 or SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), or an analogue or derivative thereof).

143. NF kappa B Inhibitors

In another embodiment, the fibrosis-inhibiting compound is a NF kappa B (NFKB) inhibitor (e.g., emodin (CAS No. 518-82-1), AVE-0545 or AVE-0547 (Sanofi-Aventis), bortezomib (CAS No. 179324-69-7) (Millennium Pharmaceuticals), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (FO05) from Fulcrum Pharmaceuticals, P61 (Phytopharm), R-flurbiprofen (CAS No. 5104-49-4) (Encore Pharmaceuticals), Bay 11-7085, or an analogue or derivative thereof).

144. Nitric Oxide Agonists

In another embodiment, the fibrosis-inhibiting compound is a nitric oxide agonist (e.g., Acclaim, Angx-1039 or Angx-3227 (Angiogenix), CAS-1609 (CAS No. 158590-73-9) (Sanofi-Aventis), GCI-503 (Spear Therapeutics), HCT-3012 (CAS No. 163133-43-5) (NicOx), hydralazine+ISDN (NitroMed), isosorbide dinitrate, Diffutab (CAS No. 87-33-2) (Eurand), isosorbide mononitrate (CAS No. 16051-77-7) from AstraZeneca, Schering AGor Schwarz Pharma, LA-419 (Lacer), molsidomine (CAS No. 25717-80-0) (from Takeda and Therabel), NCX-1000, NCX-2057, or NCX4040 (NicOx), nitric oxide (ProStrakan), nitroglycerin in the form of a nitroglycerin patch, such as DERMATRANS from (Rottapharm), nitroglycerin (CAS No. 55-63-0) (from Cellegy Pharmaceuticals, Forest Laboratories, NovaDel, Schwarz Pharma, and Watson), NO-releasing prodrugs (Inotek), OM-294DP (OM PHARMA), oxdralazine (CAS No. 27464-23-9) (Sanofi-Aventis), pirsidomine (CAS No. 132722-74-8) (Sanofi-Aventis), prostaglandin and NO donor (Cellegy Pharmaceuticals), upidosin derivatives (Recordati), or an analogue or derivative thereof).

145. Ornithine Decarboxylase Inhibitors

In another embodiment, the fibrosis-inhibiting compound is an ornithine decarboxylase inhibitor (e.g. aplidine, or an analogue or derivative thereof).

146. p38 MAP Kinase Inhibitors

In another embodiment, the fibrosis-inhibiting compound is a p38 MAP kinase inhibitor (e.g., AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), SKF86002 (CAS No. 72873-74-6), RPR-200765A (Sanofi-Aventis), SD-282 (Johnson & Johnson), TAK-715 (Takeda), or an analogue or derivative thereof).

147. Palmitoyl-Protein Thioesterase Inhibitors

In another embodiment, the fibrosis-inhibiting compound is a palmitoyl-protein thioesterase inhibitor (e.g., aplidine, or an analogue or derivative thereof).

148. PDGF Receptor Kinase Inhibitors

In another embodiment, the fibrosis-inhibiting compound is a PDGF receptor kinase inhibitors (e.g., AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), or an analogue or derivative thereof).

149. Peroxisome Proliferator-Activated Receptor Agonists

In another embodiment, the fibrosis-inhibiting compound is a peroxisome proliferator-activated receptor (PPAR) agonists (e.g., (−)-halofenate (Metabolex), AMG-131 (Amgen), antidiabetics from Japan Tobacco, AZD-4619, AZD-8450, or AZD-8677 (AstraZeneca), DRF-10945 or balaglitazone (Dr Reddy's), CS-00088 or CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001 or MC-3002 (MaxoCore Pharmaceuticals), mefformin HCl+pioglitazone (CAS No. 1115-70-4 and 112529-15-4) (such as ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 11:1025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), such as AVANDARYL or rosiglitazone maleate/metformin extend (CAS No. 155141-29-0 and 657-24-9) such as AVANDAMET, or rosiglitazone maleate+metformin, such as AVANDAMET (GlaxoSmithKline), tesaglitazar (AstraZeneca), LBM642, WY-14,643 (CAS No. 50892-23-4), or an analogue or derivative thereof).

In certain embodiments, the PPAR Agonist is a PPARα agonist such as GW7647 orfenofibric acid (CAS No. 42017-89-0), a PPAR γ agonist such as MCC-555 (CAS No. 161600-01-7), GW9662 or GW1929, a PPAERδ agonist such as GW501516, a PPARβ and PPARδ agonist such L-165,041 (CAS No. 79558-09-1), or an analogue or derivative thereof.

150. Phosphatase Inhibitor

In another embodiment, the fibrosis-inhibiting compound is a phosphatase inhibitor (e.g., diabetes therapy such as SQMO3, SQDM38, SODM60 from Sequenom, Pharmaprojects No 4191 (Sanofi-Aventis), PRL-3 inhibitors from Genzyme, WIP1 inhibitors from Amgen, or an analogue or derivative thereof).

151. Phosphodiesterase (PDE) Inhibitors

In another embodiment, the fibrosis-inhibiting compound is a phosphodiesterase (PDE) inhibitor (e.g., avanafil (Tanabe Seiyaku), dasantafil (CAS No. 569351-91-3) (Schering-Plough), A-906119 (CAS No. 134072-58-5) or DL-850 (Sanofi-Aventis), GRC-3015, GRC-3566, or GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, or IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), theobromine (CAS No. 83-67-0), papverine hydrochloride (CAS No. 61-25-6), quercetin dehydrate (CAS No. 6151-25-3), YM 976 (CAS No. 191219-80-4), irsogladine (CAS No 57381-26-7), or an analogue or derivative thereof).

In one embodiment, the phosphodiesterase inhibitor is a phosphodiesterase III inhibitor (e.g., enoximone, or an analogue or derivative thereof). In other embodiments, the phosphodiesterase inhibitor is a phosphodiesterase IV inhibitor (e.g., fosfosal, Atopik (Barrier Therapeutics), triflusal or an analogue or derivative thereof). In other embodiments, the phosphodiesterase inhibitor is a phosphodiesterase V inhibitor

152. PKC Inhibitor

In another embodiment, the fibrosis-inhibiting compound is a PKC inhibitor (e.g., HMR-105509 or P-10050 (Sanofi-Aventis), JNJ-10164830 (Johnson & Johnson), Ro-31-8425 (CAS No. 131848-97-0), NPC-15437 dihydrochloride (CAS No. 136449-85-9), or an analogue or derivative thereof).

In one embodiment, the PKC inhibitor is an inhibitor of PKC beta (e.g., ruboxistaurin (Eli Lilly), or an analogue or derivative thereof).

153. Platelet Activating Factor Antagonists

In another embodiment, the fibrosis-inhibiting compound is a platelet activating factor antagonist (e.g., dersalazine, or an analogue or derivative thereof).

154. Platelet-Derived Growth Factor Receptor Kinase Inhibitors

In another embodiment, the fibrosis-inhibiting compound is a platelet-derived growth factor receptor kinase inhibitor (e.g., sorafenib tosylate, Raf or Ras inhibitors such as sorafenib tosylate from Bayer and Onyx Pharmaceuticals, or an analogue or derivative thereof).

155. Prolyl Hydroxylase Inhibitors

In another embodiment, the fibrosis-inhibiting compound is a prolyl hydroxylase inhibitor (e.g., FG-2216 (CAS No. 11096-26-7) or HIF agonists from FibroGen, or an analogue or derivative thereof).

156. Polymorphonuclear Neutrophil Inhibitors

In another embodiment, the fibrosis-inhibiting compound is a polymorphonuclear neutrophil inhibitor (e.g., orazipone, or an analogue or derivative thereof)

157. Protein Kinase B Inhibitors

In another embodiment, the fibrosis-inhibiting compound is a protein kinase B inhibitor (e.g., Akt-1 inhibitors from Amphora, or an analogue or derivative thereof).

158. Protein Kinase C Stimulants

In another embodiment, the fibrosis-inhibiting compound is a protein kinase C stimulant (e.g., bryostatin-1, or analogue or derivative thereof).

159. Purine Nucleoside Analogues

In another embodiment, the fibrosis-inhibiting compound is a purine nucleoside analogue (e.g., cladrinbine and formulations thereof, such as MYLINAX from Serone SA and IVAX Research Inc. (Miami, Fla.), or an analogue or derivative thereof).

160. Purinoreceptor P2X Antagonist

In another embodiment, the fibrosis-inhibiting compound is a purinoreceptor P2X antagonist (e.g., AZD-9056 (AstraZeneca), R-1554 (Hoffmann-La Roche), AR-C118925XX (AstraZeneca), suramin (CAS No. 129-46-4), P2Y4 receptor from Euroscreen, or an analogue or derivative thereof).

161. Raf Kinase Inhibitors

In another embodiment, the fibrosis-inhibiting compound is a Raf kinase inhibitor (e.g., sorafenib tosylate, or an analogue or derivative thereof).

162. Reversible Inhibitors of ErbB1 and ERbB2

In another embodiment, the fibrosis-inhibiting compound is a reversible inhibitor (e.g., lapatinib (GSK), or an analogue or derivative thereof)

163. Ribonucleoside Triphosphate Reductase Inhibitors

In another embodiment, the fibrosis-inhibiting compound is a cytoplasmic tyrosine kinase inhibitor such as a SRC inhibitor (e.g., SRN-004 (Sirenade), gallium maltolate (Titan Pharmaceutcals), or an analogue or derivative thereof), or an analogue or derivative thereof)

164. SDF-1 Antagonists

In another embodiment, the fibrosis-inhibiting compound is a SDF-1 antagonist (e.g., CTCE-9908 (Chemokine Therapeutics), or an analogue or derivative thereof).

165. Sheddase Inhibitor

In another embodiment, the fibrosis-inhibiting compound is a sheddase inhibitor (e.g., INCB-7839 (Incyte Corporation), or an analogue or derivative thereof).

166. SRC Inhibitors

In another embodiment, the fibrosis-inhibiting compound is a SRC inhibitor (e.g., SRN-004 (Sirenade), or an analogue or derivative thereof).

In certain embodiments, the SRC inhibitor is a SRC kinase inhibitor (e.g., AZD0530 (AstraZeneca), or an analogue or derivative thereof).

167. Stromelysin Inhibitors

In another embodiment, the fibrosis-inhibiting compound is a stromelysin inhibitor (e.g., glucosamine sulfate, or an analogue or derivative thereof).

168. Syk Kinase Inhibitors

In another embodiment, the fibrosis-inhibiting compound is a syk kinase inhibitor (eg, R406 (Rigel), or an analogue or derivative thereof)

169. Telomerase Inhibitors

In another embodiment, the fibrosis-inhibiting compound is a telomerase inhibitor (e.g., AS-1410 (Antisoma), or an analogue or derivative thereof).

170. TGF Beta Inhibitors

In another embodiment, the fibrosis-inhibiting compound is a TGF beta inhibitor (e.g., pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists (e.g., 1090 and 1091 from Sydney; non-industrial source), TGF-βI receptor kinase inhibitors from Eli Lilly, TGF-β receptor inhibitors from Johnson & Johnson, or an analogue or derivative thereof).

171. TNFα Antagonists and TACE Inhibitors

In another embodiment, the fibrosis-inhibiting compound is a TNFα antagonist or TACE inhibitors (e.g., adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celitech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g. humicade from UCB), IC-485 (ICOS), infliximab (CAS No 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (from Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-214) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, or an analogue or derivative thereof).

172. Tumor Necrosis Factor Antagonists

In another embodiment, the fibrosis-inhibiting compound is a tumor necrosis factor (TNF) antagonist (e g anti-inflammatory compounds from Biota Inc. or an analogue or derivative thereof).

173. Toll Receptor Antagonists

In another embodiment, the fibrosis-inhibiting compound is a Toll receptor antagonist (e.g., E5564 (Eisai Pharmaceuticals), or an analogue or derivative thereof).

174. Tubulin Antagonist

In another embodiment, the fibrosis-inhibiting compound is a tubulin antagonist (e.g., synthadotin, KRX-0403 (Keryx Biopharmaceuticals), or an analogue or derivative thereof).

175. Tyrosine Kinase Inhibitors

In another embodiment, the fibrosis-inhibiting compound is a tyrosine kinase inhibitor (e.g., SU-011248 (e.g., SUTENT from Pfizer Inc. (New York, N.Y.), BMS-354825, PN-355 (Paracelsian Pharmaceuticals), AGN-199659 (Allergan), (e.g., AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No 184475-35-2) (Astraeneca), GW-2286 or GW-654652 (GlaxoSmithKline), her2/neu antigen from AlphaVax, HER-21neu inhibitor from Generex, Herzyme (Medipad) (Sirna Therapeutics), HKI-272 (Wyeth), HuMax-EGFr (Genmab), idronoxil (CAS No 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, or U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), herbimycin A, or an analogue or derivative thereof).

In certain embodiments, the tyrosine kinase inhibitor is an EGFR tyrosine kinase inhibitor such as EKB-569 (Wyeth), or an analogue or derivative thereof).

176. VEGF Inhibitors

In another embodiment, the fibrosis-inhibiting compound is a VEGF Inhibitor (e.g., AZD2171 (AstraZeneca), or an analogue or derivative thereof)

177. Vitamin D Receptor Agonists

In another embodiment, the fibrosis-inhibiting compound is a vitamin D receptor agonist (e.g., BXL-628, BXL-922 (BioXell), or an analogue or derivative thereof).

178. Histamine Receptor Antagonists

In another embodiment, the fibrosis-inhibiting compound is an histamine receptor antagonist. Certain embodiments, the histamine receptor antagonists, such as H1, H2, and H3 histamine receptor antagonists, block the production of pro-inflammatory cytokines such as TNFa and IL-1 (e.g., IL-1β). In certain embodiments, the histamine receptor antagonist inhibit NFkB activation. Representative examples of H1 histamine receptor antagonists include phenothiazines, such as promethazine, and alkylamines, such as chlorpheniramine (CAS No. 7054-11-7), brompheniramine (CAS No. 980-71-2), fexofenadine hydrochloride, promethazine hydrochloride, loratadine, ketotifen fumarate salt, and acrivastine. Other examples of histamine receptor antagonists include broad spectrum histamine receptor antagonists such as methylxanthines (e.g., theophylline, theobromine, and caffeine). Representative examples of H2 receptor antagonists include those with a histamine-like structure including cimetidine (available under the tradename TAGAMET from SmithKline Beecham Phamaceutical Co., Wilmington, Del.), ranitidine (available under the tradename ZANTAC from Warner Lambert Company, Morris Plains, N.J.), famotidine (available under the tradename PEPCID from Merck & Co., Whitehouse Station, N.J.), nizatidine (available under the tradename AXID from Reliant Pharmaceuticals, Inc., Liberty Corner, N.J.), nizatidine, and roxatidine acetate (CAS No 78628-28-1) Additional examples include H3 receptor antagonists (e.g., thioperamide and thioperamide maleate salt) and anti-histamines such as tricyclic dibenozoxepins, ethanolamines, ethylenediamines, piperizines, piperidines, and pthalazinones.

179. Alpha Adrenergic Receptor Antagonists

In another embodiment, the fibrosis-inhibiting compound is an alpha adrenergic receptor antagonist. Alpha adrenergic receptor antagonists may inhibit the production of pro-inflammatory cytokines such as TNFa. The alpha adrenergic receptor antagonist may be an alpha-1 and/or an alpha-2 adrenergic receptor antagonist. Representative examples of alpha-1/alpha-2 antagonists include phenoxybenzamine. In certain embodiments, the alpha adrenergic receptor antagonist is a haloalkylamine compound or a catecholamine uptake inhibitor. Representative examples of alpha-1 adrenergic receptor antagonists include phenoxybenzamine hydrochloride and prazosin, a piperizinyl quinazoline. Representative examples of alpha-2 adrenergic receptor antagonists include imadazole based compounds such as idazoxan (CAS No. 79944-56-2), idazoxan hydrochloride, and loxapine succinate salt (CAS No. 27833-64-3). Additional examples of alpha adrenergic receptor antagonists include prazosin hydrochloride.

180. Anti-Psychotic Compounds

In another embodiment, the fibrosis-inhibiting compound is an anti-psychotic compound, such as a phenothiazine compound or an analogue or derivative thereof. In some embodiments, the fibrosis-inhibiting compound is a phenothiazine derivative capable of suppressing the production of pro-inflammatory cytokines such as TNFa and/or IL-1. Representative examples of phenothiazine compounds include chlorpromazine, fluphenazine, trifluorphenazine, mesoridazine, thioridazine, and perphenazine. Other examples of anti-psychotic compounds include thioxanthines such as chlorprothixene and thiothixene, clozapine, loxapine succinate, and olanzapine.

181. CaM Kinase II Inhibitor

In another embodiment, the fibrosis-inhibiting compound is CaM kinase II inhibitor, such as a lavendustin C, or an analogue or derivative thereof.

182. CaM Kinase II Inhibitor

In another embodiment, the fibrosis-inhibiting compound is CaM kinase II inhibitor, such as a lavendustin C, or an analogue or derivative thereof.

183. G Protein Agonist

In another embodiment, the fibrosis-inhibiting compound is G protein agonist, such as aluminum fluoride, or an analogue or derivative thereof.

184. Antibiotics and Anti-Microbials

In another embodiment, the fibrosis-inhibiting compound is an antibiotic, such as apigenin (Cas No. 520-36-5), ampicillin sodium salt (CAS No. 69-52-3), puromycin, or an analogue or derivative thereof.

In another embodiment, the fibrosis-inhibiting compound is an anti-microbial agent, such as brefeldin A (CAS No. 20350-15-6), terbinafine, benzoyl peroxide, pentamidine, ornidazole, tinidazole, ketocanazole, sulconazole nitrate salt, or an analogue or derivative thereof.

185. DNA Topoisomerase Inhibitors

In another embodiment, the fibrosis-inhibiting compound is DNA topoisomerase I inhibitor, such as β-lapachone (CAS No. 4707-32-8), or an analogue or derivative thereof.

In another embodiment, the fibrosis-inhibiting compound is DNA topoisomerase II inhibitor, such as (..)-arctigenin (CAS No 7770-78-7), aurintricarboxylic acid, or an analogue or derivative thereof

186. Thromboxane A2 Receptor Inhibitor

In another embodiment, the fibrosis-inhibiting compound is thromboxane A2 receptor inhibitor, such as BM-531 (CAS No. 284464-46-6), ozagrel hydrochloride (CAS No. 78712-43-3), or an analogue or derivative thereof.

187. D2-Dopamine Receptor Antagonist

In another embodiment, the fibrosis-inhibiting compound is a D2 dopamine receptor antagonist, such as clozapine (CAS No. 5786-21-0), mesoridazine benzenesulfonate, or an analogue or derivative thereof.

188. Peptidyl-Prolyl Cis/Trans Isomerase Inhibitor

In another embodiment, the fibrosis-inhibiting compound is a Peptidyl-Prolyl Cis/Trans Isomerase Inhibitor, such as juglone (CAS No. 481-39-0), or an analogue or derivative thereof.

189. Dopamine Antagonists

In another embodiment, the fibrosis-inhibiting compound is a dopamine antagonist, such as thiothixene, thioridazine hydrochloride, or an analogue or derivative thereof.

190. Anesthetics

In another embodiment, the fibrosis-inhibiting compound is an anesthetic compound, such as lidocaine (CAS No. 137-58-6), or an analogue or derivative thereof.

191. Clotting Factors

In another embodiment, the fibrosis-inhibiting compound is a clotting factor, such as menadione (CAS No. 58-27-5), or an analogue or derivative thereof

192. Lysyl Hydrolase Inhibitor

In another embodiment, the fibrosis-inhibiting compound is a lysyl hydrolase inhibitor, such as minoxidil (CAS No. 38304-91-5), or an analogue or derivative thereof.

193. Muscarinic Receptor Inhibitor

In another embodiment, the fibrosis-inhibiting compound is a muscarinic receptor inhibitor, such as perphenazine (CAS No. 58-39-9), or an analogue or derivative thereof.

194. Superoxide Anion Generator

In another embodiment, the fibrosis-inhibiting compound is a superoxide anion generator, such as plumbagin (CAS No 481-42-5), or an analogue or derivative thereof.

195. Steroids

In another embodiment, the fibrosis-inhibiting compound is a steroid, such as prednisolone, prednisolone 21-acetate (CAS No. 52-21-1), loteprednol etabonate, (CAS No. 82034-46-6), clobetasol propionate, or an analogue or derivative thereof.

196. Anti-Proliferative Agents

In another embodiment, the fibrosis-inhibiting compound is an anti-proliferative agent, such as silibinin (CAS No. 22888-70-6), silymarin (CAS No. 65666-07-1), 1,2-hexanediol, dioctyl phthalate (CAS No. 117-81-7), zirconium (IV) oxide, glycyrrhizic acid, spermidine trihydrochloride or tetrahydrochloride, CGP 74514A, spermine tetrahydrochloride, NG-methyl-L-arginine acetate salt, galardin, halofuginone hydrobromide (HBr), fascaplysin, or an analogue or derivative thereof.

197. Diuretics

In another embodiment, the fibrosis-inhibiting compound is a diuretic, such as spironolactone (CAS No. 52-01-7), or an analogue or derivative thereof.

198. Anti-Coagulants

In another embodiment, the fibrosis-inhibiting compound is an anti-coagulant, such as fucoidan from Fucus vesiculosus (CAS No. 9072-19-9), or an analogue or derivative thereof.

199. Cyclic GMP Agonists

In another embodiment, the fibrosis-inhibiting compound is a cyclic GMP agonist, such as sinitrodil (CAS No. 143248-63-9), or an analogue or derivative thereof.

200. Adenylate Cyclase Agonist

In another embodiment, the fibrosis-inhibiting compound is an adenylate cyclase agonist, such as histamine (CAS No. 51-45-6), or an analogue or derivative thereof.

201. Antioxidants

In another embodiment, the fibrosis-inhibiting compound is an antioxidant, such as morpholine, phytic acid dipotassium salt, (−)-epigallocatechin or (−)-epigallocatechin gallate from green tea (CAS Nos. 970-74-1 and 1257-08-5, respectively), (−)-epigallocatechin gallate (CAS No. 989-51-5), nobiletin (CAS No 478-01-3), probucol (CAS No 23288-49-5), phosphorous acid, hesperetin. L-ascorbyl-2-phosphate, magnesium salt (CAS No. 84309-23-9), catechin, (±)-naringenin (CAS No. 67604-48-2), (−)-epicatechin, (−)-epicatechin gallate, 3-hydroxyflavone, (−)-arctigenin, or an analogue or derivative thereof.

202. Nitric Oxide Synthase Inhibitors

In another embodiment, the fibrosis-inhibiting compound is a nitric oxide synthase inhibitor, such as ammonium pyrrolidinedithiocarbamate (CAS No. 5108-96-3), or an analogue or derivative thereof.

In another embodiment, the fibrosis-inhibiting compound is a reversible nitric oxide synthase inhibitor, such as NB-methyl-L-arginine acetate salt (L-NMMA) (CAS No. 53308-83-1), or an analogue or derivative thereof.

203. Anti-Neoplastic Agents

In another embodiment, the fibrosis-inhibiting compound is an anti-neoplastic agent, such as tirapazamine (CAS No. 27314-97-2), fludarabine (CAS No. 21679-14-1), cladribine, imatinib mesilate, or an analogue or derivative thereof.

204. DNA Synthesis Inhibitors

In another embodiment, the fibrosis-inhibiting compound is a DNA synthesis inhibitor, such as S-(2-hydroxy-5-nitrobenzyl)-6-thioguanosine or uracilfludarabine phosphate (CAS No. 75607-67-9), 6,11-dihydroxy-5,12-naphthacenedione, or an analogue or derivative thereof.

205. DNA Alkylating Agents

In another embodiment, the fibrosis-inhibiting compound is a DNA alkylating agent, such as dacarbazine (CAS No. 4342-03-4), temozolomide, procarbazine HCl, or an analogue or derivative thereof.

206. DNA Methylation Inhibitors

In another embodiment, the fibrosis-inhibiting compound is a DNA methylation inhibitor, such as decitabine (CAS No. 2353-33-5), or an analogue or derivative thereof.

207. NSAID Agents

In another embodiment, the fibrosis-inhibiting compound is a NSAID agent, such as nabumetone, benzydamine hydrochloride, or an analogue or derivative thereof.

208. Peptidylglycine Alpha-Hydroxylating Monooxygenase Inhibitors

In another embodiment, the fibrosis-inhibiting compound is a peptidylglycine alpha-hydroxylating monooxygenase inhibitor, such as trans-styrylacetic acid, or an analogue or derivative thereof.

209. MEK1/MEK2 Inhibitors

In another embodiment, the fibrosis-inhibiting compound is a MEK1/MEK 2 inhibitor, such as U0126 (CAS No. 109511-58-2), or an analogue or derivative thereof.

210. NO Synthase Inhibitors

In another embodiment, the fibrosis-inhibiting compound is an NO synthase inhibitor, such as L-NAME (CAS No. 53308-83-1), NG-Methyl-L-arginine acetate salt, or an analogue or derivative thereof.

211. Retinoic Acid Receptor Antagonists

In another embodiment, the fibrosis-inhibiting compound is retinoic acid receptor antagonist, such as isotretinoin (CAS No. 4759-48-2), or an analogue or derivative thereof.

212. ACE Inhibitors

In another embodiment, the fibrosis-inhibiting compound is an ACE inhibitor, such as quinapril hydrochloride (CAS No 85441-61-8), enalapril, or an analogue or derivative thereof

213. Glycosylation Inhibitors

In another embodiment, the fibrosis-inhibiting compound is a glycosylation inhibitor, such as aminoguanidine hydrochloride, castanospermine, or an analogue or derivative thereof

214. Intracellular Calcium Influx Inhibitors

In another embodiment, the fibrosis-inhibiting compound is an intracellular calcium influx inhibitor, such as TAS-301 (CAS No. 193620-69-8), or an analogue or derivative thereof.

215. Anti-Emetic Agents

In another embodiment, the fibrosis-inhibiting compound is an anti-emetic agent, such as amifostine (CAS No. 20537-88-6), or an analogue or derivative thereof.

216. Acetylcholinesterase Inhibitors

In another embodiment, the fibrosis-inhibiting compound is an acetylcholinesterase inhibitor, such as (−)-huperzine A (CAS No. 102518-79-6), or an analogue or derivative thereof.

217. ALK-5 Receptor Antagonists

In another embodiment, the fibrosis-inhibiting compound is an ALK-5 receptor antagonist, such as SB 431542 (CAS No. 301836-41-9), or an analogue or derivative thereof.

218. RAR/RXR Antagonists

In another embodiment, the fibrosis-inhibiting compound is a RAR/RXT antagonist, such as 9-cis-retinoic acid, or an analogue or derivative thereof

219. EIF-2a Inhibitors

In another embodiment, the fibrosis-inhibiting compound is a eIF-2a inhibitor, such as salubrinal, or an analogue or derivative thereof.

220. S-Adenosyl-L-Homocysteine Hydrolase Inhibitors

In another embodiment, the fibrosis-inhibiting compound is a S-adenosyl-L-homocysteine hydrolase inhibitor, such as 3-deazaadenosine, or an analogue or derivative thereof.

221. Estrogen Agonists

In another embodiment, the fibrosis-inhibiting compound is an estrogen agonist, such as coumestrol, bisphenol A, 1-linoleoyl-rac-glycerol (CAS No. 2277-28-3), daidzein (4,7-dihydroxy-iso-flavone), dihexyl phthalate, kaempferol, formononetin, or an analogue or derivative thereof.

222. Serotonin Receptor Inhibitors

In another embodiment, the fibrosis-inhibiting compound is a serotonin receptor inhibitor, such as amitriptyline hydrochloride, or an analogue or derivative thereof.

223. Anti-Thrombotic Agents

In another embodiment, the fibrosis-inhibiting compound is an anti-thrombotic agent, such as geniposidic acid, geniposide, or an analogue or derivative thereof.

224. Tryptase Inhibitors

In another embodiment, the fibrosis-inhibiting compound is a tryptase inhibitors, such as 2-azetidinone, or an analogue or derivative thereof.

225. Pesticides

In another embodiment, the fibrosis-inhibiting compound is a pesticide, such as allyl disulfide, or an analogue or derivative thereof.

226. Bone Mineralization Promotor

In another embodiment, the fibrosis-inhibiting compound is a bone mineralization promotor, such as glycerol 2-phosphate disodium salt hydrate, or an analogue or derivative thereof.

227. Bisphosphonate Compounds

In another embodiment, the fibrosis-inhibiting compound is a bisphosphonate compound, such as risedronate, or an analogue or derivative thereof.

228. Anti-Inflammatory Compounds

In another embodiment, the fibrosis-inhibiting compound is an anti-inflammatory compound, such as aucubin, cepharanthine, or an analogue or derivative thereof.

229. DNA Methylation Promotors

In another embodiment, the fibrosis-inhibiting compound is a DNA methylation promotor, such as 5-azacytidine, or an analogue or derivative thereof.

230. Anti-Spasmodic Agents

In another embodiment, the fibrosis-inhibiting compound is an anti-spasmodic agent, such as 2-hydroxy-4,6-dimethoxyacetophenone, or an analogue or derivative thereof.

231. Protein Synthesis Inhibitors

In another embodiment, the fibrosis-inhibiting compound is a protein synthesis inhibitor, such as oxytetracycline hydrochloride, or an analogue or derivative thereof.

232. α-Glucosidase Inhibitors

In another embodiment, the fibrosis-inhibiting compound is a α-glucosidase inhibitor, such as myricetin (CAS No. 529-44-2), or an analogue or derivative thereof.

233. Calcium Channel Blockers

In another embodiment, the fibrosis-inhibiting compound is a calcium channel blocker, such as verapamil, nitrendipine, or an analogue or derivative thereof.

In another embodiment, the fibrosis-inhibiting compound is a L-type calcium channel blocker, such as nifedipine (CAS No. 21829-25-4), (+)-cis-diltiazem hydrochloride, or an analogue or derivative thereof.

In another embodiment, the fibrosis-inhibiting compound is a T-type calcium channel blocker, such as penfluridol (CAS No. 26864-56-2), or an analogue or derivative thereof.

234. Pyruvate Dehydrogenase Activators

In another embodiment, the fibrosis-inhibiting compound is a pyruvate dehydrogenase activator, such as dichloroacetic acid, or an analogue or derivative thereof.

235. Prostaglandin Inhibitors

In another embodiment, the fibrosis-inhibiting compound is a prostaglandin inhibitor, such as betulinic acid, or an analogue or derivative thereof.

236. Sodium Channel Inhibitors

In another embodiment, the fibrosis-inhibiting compound is a sodium channel inhibitor, such as amiloride hydrochloride hydrate, or an analogue or derivative thereof.

237. Serine Protease Inhibitors

In another embodiment, the fibrosis-inhibiting compound is a serine protease inhibitor, such as gabexate mesylate, or an analogue or derivative thereof.

238. Intracellular Calcium Flux Inhibitors

In another embodiment, the fibrosis-inhibiting compound is an intracellular calcium flux inhibitor, such as thapsigargin, or an analogue or derivative thereof.

239. JAK2 Inhibitors

In another embodiment, the fibrosis-inhibiting compound is a JAK2 inhibitor (e.g., AG490 (CAS No. 134036-52-5), or an analogue or derivative thereof).

240. Androgen Inhibitors

In another embodiment, the fibrosis-inhibiting compound is an androgen inhibitor (e.g., tibolone (CAS No. 5630-53-5), or an analogue or derivative thereof).

241. Aromatase Inhibitors

In another embodiment the fibrosis-inhibiting compound is an aromatase inhibitor (e.g. Ietrozole, or an analogue or derivative thereof)

242. Anti-Viral Agents

In another embodiment, the fibrosis-inhibiting compound is an anti-viral agent, such as imiquimod, or an analogue or derivative thereof.

243. 5-HT Inhibitors

In another embodiment, the fibrosis-inhibiting compound is a 5-HT inhibitor, such as ketanserin tartrate, amoxapine, or an analogue or derivative thereof.

244. FXR Antagonists

In another embodiment, the fibrosis-inhibiting compound is a FXR antagonist, such as guggulsterone (CAS No. 95975-55-6), or an analogue or derivative thereof.

245. Actin Polymerization and Stabilization Promotors

In another embodiment, the fibrosis-inhibiting compound is an actin polymerization and stabilization promotor, such as jasplakinolide, or an analogue or derivative thereof.

246. AXOR12 Agonists

In another embodiment, the fibrosis-inhibiting compound is an AXOR12 agonist, such as metastin (KiSS-1 (112-121), or an analogue or derivative thereof.

247. Angiotensin II Receptor Antagonists

In another embodiment, the fibrosis-inhibiting compound is an angiotensin II receptor agonist, such as losartan potassium, or an analogue or derivative thereof.

248. Platelet Aggregation Inhibitors

In another embodiment, the fibrosis-inhibiting compound is a platelet aggregation inhibitor, such as clopidogrel, or an analogue or derivative thereof.

249. CB1/CB2 Receptor Agonists

In another embodiment, the fibrosis-inhibiting compound is a CB1/CB2 receptor agonist, such as HU-210 (CAS No. 112830-95-2), or an analogue or derivative thereof.

250. Norepinephrine Reuptake Inhibitors

In another embodiment, the fibrosis-inhibiting compound is a norepinephrine reuptake inhibitor, such as nortriptyline hydrochloride, or an analogue or derivative thereof.

251. Selective Serotonin Reuptake Inhibitors

In another embodiment, the fibrosis-inhibiting compound is a selective serotonin reuptake inhibitor, such as paroxetine maleate, or an analogue or derivative thereof.

252. Reducing Agents

In another embodiment, the fibrosis-inhibiting compound is a reducing agent such as Ww-85 (Inotek), or an analogue or derivative thereof.

253. Immuno-Modulators

In another embodiment, the fibrosis-inhibiting compound is an immunomodulators such as Bay 11-7085, (−)-arctigenin, idazoxan hydrochloride, or an analogue or derivative thereof.

In certain embodiments, two or more anti-scarring agents may be used in combination with sutures according to the present invention. The effectiveness of various combinations of anti-scaring agents in using in combination with sutures (e.g., reducing surgical adhesion) may be determined according to the methods described in the examples.

### B. Therapeutic Compositions

Anti-scarring agents as described above may be used in combination with other therapeutic agents or components to form therapeutic compositions. In certain embodiments, such compositions may be used in making sutures that comprise anti-scarring agents, such as used as a coating or dipping solution. In other embodiments, anti-scarring agent-containing therapeutic compositions may be used to infiltrate tissue into which a suture has been, is being, or is to be implanted.

1. Secondary Therapeutic Agents

Within various embodiments of the invention, a therapeutic composition may include an agent that inhibits fibrosis and a second composition or compound which acts to have an inhibitory effect on pathological processes in or around the site where a suture has been, is being, or is to be implanted. Representative examples of additional therapeutically active agents include, by way of example and not limitation, anti-thrombotic agents, anti-infective agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors, tyrosine kinase inhibitors, MMP inhibitors, p38 MAP kinase inhibitors, immunosuppressants, apoptosis antagonists, caspase inhibitors, and JNK inhibitors

In certain embodiments, the composition may include an anti-thrombotic agent and/or antiplatelet agent and/or a thrombolytic agent, which reduces the likelihood of thrombotic events upon implantation of a medical implant Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulfate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

Representative examples of secondary therapeutic agents include: anti-inflammatory agents (e.g., dexamethasone, cortisone, fludrocortisone, prednisone, prednisolone, 6α-methylprednisolone, triamcinolone, and betamethasone); matrix metalloproteinase (MMP) inhibitors (e.g., marimistat, batimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408, 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869, and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat. Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071A1, WO 99/001545A1, WO 97/40028A1, WO 97/41211A1, WO 98/40381A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376.527; 6,444,696; 6,479,507; 6,509,361; 6,579,874; and 6,630,485, and US Patent Application Publication Nos 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2, WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of therapeutic agents that may be included include tyrosine kinase inhibitors (e.g., imantinib, ZK-222584, CGP-52411, CGP-53716, NVP-AAK980-NX, CP-127374, CP-564959, PD-171026, PD-173956, PD-180970, SU-0879, and SKI-606), MMP inhibitors (e.g., nimesulide, PKF-241-466, PKF-242-484, CGS-27023A, SAR-943, primomastat, SC-77964, PNU-171829, AG-3433, PNU-142769, SU-5402, and dextipotam), p38 MAP kinase inhibitors (e.g., CGH-2466 and PD-98-59), immunosuppressants (e.g., argyrin B, macrocyclic lactone, ADZ-62-826, CCI-779, tilomisole, amcinonide, FK-778, AVE-1726, and MDL-28842), TNF-484A, PD-172084, CP-293121, CP-353164, and PD-168787, NFKB inhibitors, (e.g., AVE-0547, AVE-0545, and IPL-576092), HMGCOA reductase inhibitors (e.g., pravestatin, atorvastatin, fluvastatin, dalvastatin, glenvastatin, pitavastatin, CP-83101, U-20685), apoptosis antagonists (e.g., troloxamine, TCH-346 (N-methyl-N-propargyl-10-aminomethyl-dibenzo(b,f)oxepin), caspase inhibitors (e.g., PF-5901 (benzenemethanol, alpha-pentyl-3-(2-quinolinylmethoxy)-), and JNK inhibitor (e.g., AS-602801).

In certain embodiments, a polymeric composition comprising a fibrosis-inhibiting agent is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting agent (e.g., ZD-6474, AP-23573, Synthadotin, S-0885, Aplidine, Ixabepilone, IDN-5390, SB-2723005, ABT-518, Combretastatin, Anecortave acetate, SB-715992, Temsirolimus, Adalimumab, erucyiphosphocholine, alphastatin, BXT-51072, Etanercept, Humicade, Gefitinib, rapamycin, everolimus) and a CYP inhibitor, which may be combined (e.g., coated) with any of the sutures described herein. Representative examples of CYP inhibitors include flavones, azole antifungals, macrolide antibiotics, HIV protease inhibitors, and anti-sense oligomers.

In certain embodiments, the therapeutic composition may comprise one or more anti-infective agents, which may reduces the likelihood of infections at the site where a suture is implanted. An “anti-infective agent” refers to an agent that reduces the likelihood of an infection. An agent is demonstrated to be an active anti-infective agent toward a microorganism by assays routinely practiced by persons skilled in the art, for example, an in vitro assay determining inhibition of bacterial growth as indicated by the M.I.C. (minimimum inhibitory concentration). In certain embodiments, anti-infective agents are chemotherapeutic agents that have antimicrobial activity at low doses (e.g., anthracyclines, fluoropyrimidines, folic acid antagonists, podophylotoxins, camptothecins, hydroxyureas, and platinum complexes.

In certain embodiments, the anti-infective agent may be an anti-septic agent. An “anti-septic agent” refers to an agent or substance that is capable of effective antisepsis, that is, prevention of infection by inhibiting the growth of an infectious organism without necessarily killing the organism. Representative examples of anti-septic agents include chlorhexadine, triclosan, and chloroxylenol.

In certain other embodiments, the anti-infective agent may be an antibiotic. An “antibiotic” refers to an agent that kills or inhibits the growth of microorganisms. Antibiotics may have a narrow or wide range of activity against either one or both of Gram-positive and Gram-negative organisms. Antibiotic agents can be identified through in vitro inhibition of bacterial growth as shown in the M.I.C. assay described herein. Representative examples of antibiotics include gentamicin sulfate, amikacin sulfate, kanamycin sulfate, polymyxin B, neomycin sulfate, cephazolin sodium, metronidazole, Ciprofloxacin, piperacillin, Cefoxitin, Cefepime, Azithromycin, and Trimethoprom-sulfamethoxazole.

In certain embodiments, the anti-infective agent may be further combined with anti-thrombotic and/or antiplatelet agents (for example, heparin, dextran sulfate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy

a. Anti-Infective Agents—Antibiotics

Antibiotics and combinations of antibiotics that are used by those skilled in the medical art include the following exemplary antibiotics: fourth generation penicillins such as meziocillin and piperacillin (ureidopenicillins), carbenicillin and ticarcillin (carboxypenicillins), and analogues and derivatives thereof; first generation cephalosporins such as cephazolin, Cephazolin Sodium, Cephalexin (Keflex), Cefazolin (Ancef), Cephapirin (Cefadyl), and Cephalothin (Keflin), and analogues and derivatives thereof; Ticarcillin; second generation cephalosporins such as Cefuroxime (Ceftin (oral) and Zinocef), Cefotetan (Cefotan), and Cefoxitin (Mefoxin), and analogues and derivatives thereof; third generation cephalosporin such as Naxcel (Ceftiofur Sodium), Cefdinir (Omnicef), Cefoperazone (Cefobid), Ceftazidime (Fortaz), and Ceftriaxone (Rocephin), and Cefotaxime (Claforan), and analogues and derivatives thereof; and fourth generation cephalosporins such as Cefepime (Maxipime) and analogues and derivatives thereof; monobactams such as aztreonam and analogues and derivatives thereof; carbapenems such as imipenem, ertapenem and meropenem, and analogues and derivatives thereof. Also included are inhibitors of protein synthesis such as aminoglycosides including streptomycin, gentamicin, gentamicin sulfate, tobramycin, and amikacin, amikacin sulfate, and analogues and derivatives thereof; inhibitors of protein synthesis such as the MSL group including macrolides (Erythromycin), long acting macrolides (Azithromycin) and lincosamides (Clindamycin) and streptogramins (Syneroid), clarithromycin, kanamycin, kanamycin sulfate, and analogues and derivatives thereof. Other exemplary antibiotics include inhibitors of DNA snthesis such as the quinolones including ciprofloxacin, ofloxacin, gatifloxacin, moxifloxacin, levofloxacin, trovafloxacin, and analogues and derivatives thereof, as well as other inhibitors of DNA synthesis such as metronidazole and analogues and derivatives thereof. Other antibiotics include inhibitors of folate metabolism such as sulfonamides and trimethoprim, and analogues and derivatives thereof. Additional agents include but are not limited to cefixime, spectinomycin, tetracycline, nitrofurantoin, doxycycline, polymyxin B, neomycin, neomycin sulfate, and analogues and derivatives thereof. In certain embodiments, the anti-infective agent is gentamicin sulfate, amikacin sulfate, kanamycin sulfate, polymyxin B, neomycin sulfate, cephazolin sodium, metronidazole, ciprofloxacin, piperacillin, cefoxitin, cefepime, azithromycin, or trimethoprim-sulfamethoxazole.

Furthermore, additional therapeutic agents may be delivered in combinations. Such combinations include, by way of example, but are not limited to amoxicillin and clavulanate, ampicillin and sulbactam, trimethoprom-sulfamethoxazole, ampicillin and probenecid, amoxicillin and probenecid, penicillin G and probenecid, and penicillin and a penicillinase inhibitor.

b. Anti-Infective Agents—Chemotherapeutic Agents

In certain embodiments, anti-infective agents useful in the present invention may be chemotherapeutic agnets, which have potent antimicrobial activity at extremely low doses. Discussed in more detail below are several representative examples of such agents: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

i. Anthracyclines

Anthracyclines have the following general structure, where the R groups may be a variety of organic groups:

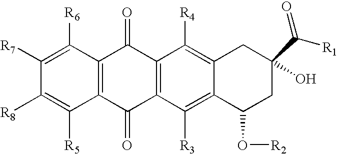

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group N—NHC(O)—Y, where Y is a group such as a phenyl or substituted phenyl ring. Alternately R₃ may have the following structure:

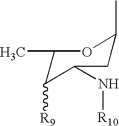

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl amino, hydroxyl mercapto, phenyl, benzyl or methylthio (see U.S. Pat. No. 4,296,105)

Exemplary anthracyclines are doxorubicin, daunorubicin, idarubicin epirubicin, pirarubicin, zorubicin, and carubicin. Suitable compounds have the structures:

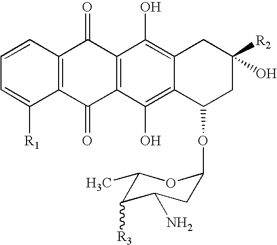

| | R₁ | R₂ | R₃ |
|---|---|---|---|
| Doxorubicin: | OCH₃ | C(O)CH₂OH | OH out of ring |
| | | | plane |
| Epirubicin: | OCH₃ | C(O)CH₂OH | OH in ring plane |
| (4′ epimer of | | | |
| doxorubicin) | | | |
| Daunorubicin: | OCH₃ | C(O)CH₃ | OH out of ring |
| | | | plane |
| Idarubicin: | H | C(O)CH₃ | OH out of ring |
| | | | plane |
| Pirarubicin: | OCH₃ | C(O)CH₂OH | 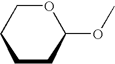 |
| Zorubicin: | OCH₃ | C(CH₃)(═N)NHC(O)C₆H₅ | OH |
| Carubicin: | OH | C(O)CH₃ | OH out of ring |
| | | | plane |

Other suitable anthracyclines are anthramycin, mitoxantrone, menogaril, nogalamycin, aclacinomycin A, olivomycin A, chromomycin A₃, and plicamycin having the structures:

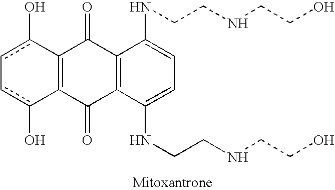

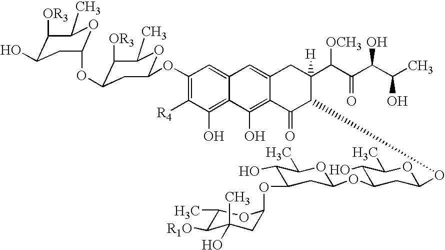

| | R₁ | R₂ | R₃ | R₄ |
|---|---|---|---|---|
| Olivomycin A | COCH(CH₃)₇ | CH₃ | COCH₃ | H |
| Chromomycin A₃ | COCH₃ | CH₃ | COCH₃ | CH₃ |
| Plicamycin | H | H | H | CH₃ |

| | R₁ | R₂ | R₃ |
|---|---|---|---|
| Menogaril | H | OCH₃ | H |
| Nogalamycin | O-sugar | H | COOCH₃ |

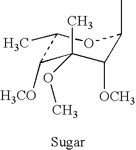

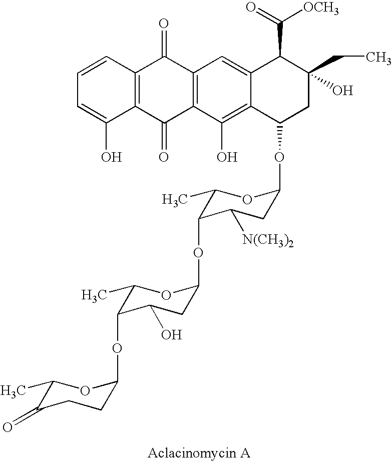

Other representative anthracyclines include, FCE 23762, a doxorubicin derivative (Quaglia et al., J. Liq. Chromatogr 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-dideoxy-4-O-(2,3,6-trideoxy-3-amino-α-L-lyxo-hexopyranosyl)-α-L-lyxo-hexopyranosyl]-adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9) 1413-16, 1995), cephalosporin doxorubicin derivatives (Vrudhula et al. J Med Chem 38(8):1380-5, 1995), hydroxyrubicin (Solary et al., Int. J. Cancer 56(1):85-94, 1994), methoxymorpholino doxorubicin derivative (Kuhl et al., Cancer Chemother. Pharmacol. 33(1):10-16, 1993), (6-mateimidocaproyl)hydrazone doxorubicin derivative (Willner et al., Bioconjugate Chem. 4(6):521-7, 1993), N-(5,5-diacetoxypent-1-yl) doxorubicin (Chemf & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80, 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci 67(12) 1748 52, 1978), SM 5887 (Pharma Japan 1468 20, 1995). MX-2 (Pharma Japan 1420 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-3′-dihydroxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

ii. Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

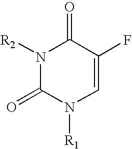

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

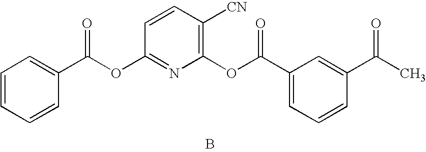

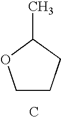

Other suitable fluoropyrimidine analogues include 5-FudR (5-fluoro-deoxyuridine), or an analogue or derivative thereof, including 5-iododeoxyuridine (5-ludR),5-bromodeoxyuridine (5-BudR), fluorouridine triphosphate (5-FUTP), and fluorodeoxyuridine monophosphate (5-dFUMP). Exemplary compounds have the structures:

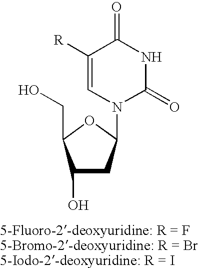

Other representative examples of fluoropyrimridine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al. J Pharmacobio-Dun 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3)-301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

iii. Folic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

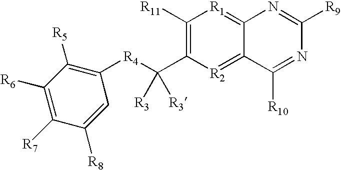

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure:

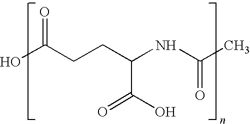

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

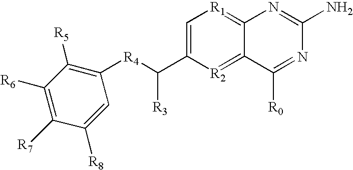

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Peritrexim | NH₂ | N | C(CH₃) | H | single bond | OCH₃ | H | H | OCH₃ |

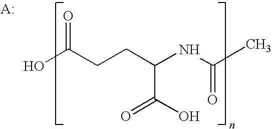

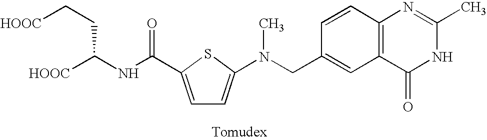

Other representative examples include 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,32-diazaphosphorines (Nilov et al. Mendeleev Commun 2:67, 1995), azathioprine (Chifotides et al., J Inorg Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8), 65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaminopterin analogues (DeGraw et al., J. Med. Chem. 40(3):370-376, 1997), 5-deazaminopterin and 5,10-dideazaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm. Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12) 2582, 1989), hydroxymethylmethotrexate (DE 267495). γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7): 1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,-omega-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al, J Med Chem 28(5):660-7, 1985), fluorinated methotrexate analogues (Tsushima et al., Heterocycles 23(1):45-9, 1985), folate methotrexate analogue (Trombe, J Bacteriol. 160(3):849-53, 1984), phosphonoglutamic acid analogues (Sturtz & Guillamot, Eur. J. Med Chem.-Chim. Ther. 19(3):267-73, 1984), poly(L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220);

These compounds are believed to act as antimetabolites of folic acid.

iv. Podophyllotoxins

In another aspect, the therapeutic agent is a Podophyllotoxin, or a derivative or an analogue thereof. Exemplary compounds of this type are etoposide or teniposide, which have the following structures:

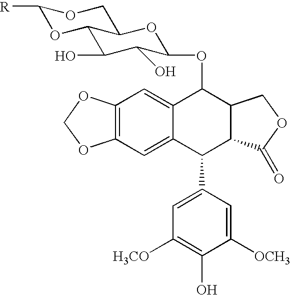

| | R |
|---|---|
| Etoposide | CH₃ |
| Temposide | 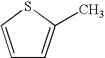 |

Other representative examples of podophyllotoxins include Cu(II)-VP-16 (etoposide) complex (Tawa et al., Bioorg Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

v. Camptothecins

In another aspect, the therapeutic agent is camptothecin, or an analogue or derivative thereof. Camptothecins have the following general structure.

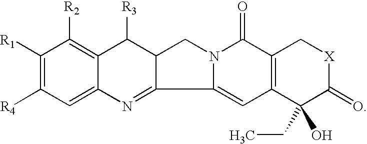

In this structure, X is typically 0, but can be other groups, eg., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

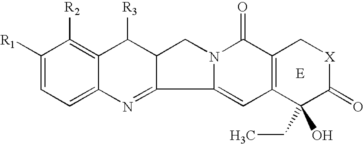

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

vi. Hydroxyureas

The therapeutic agent of the present invention may be a hydroxyurea. Hydroxyureas have the following general structure:

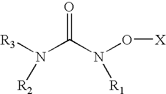

Suitable hydroxyureas are disclosed in, for example, U.S. Pat. No. 6,080,874, wherein R₁ is:

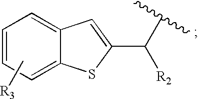

and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form:

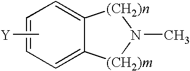

wherein m is 1 or 2, n is 0-2 and Y is an alkyl group.

In one aspect, the hydroxyurea has the structure:

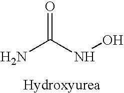

These compounds are thought to function by inhibiting DNA synthesis

vii. Platinum Complexes

In another aspect, the therapeutic agent is a platinum compound In general, suitable platinum complexes may be of Pt(II) or Pt(IV) and have this basic structure:

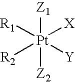

wherein X and Y are anionic leaving groups such as sulfate, phosphate, carboxylate, and halogen, R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxyl, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

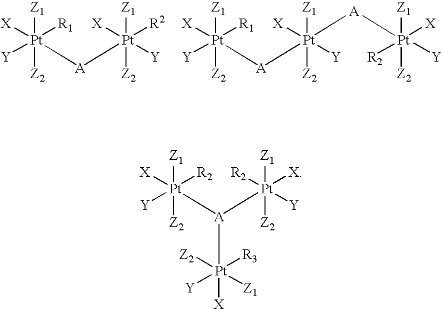

Exemplary platinum compounds are cisplatin, carboplatin, oxaliplatin, and miboplatin having the structures:

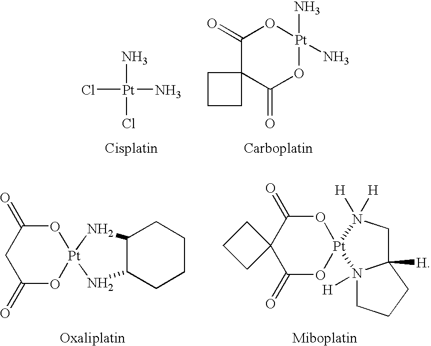

Other representative platinum compounds include (CPA)₂Pt[DOLYM] and (DACH)Pt[DOLYM] cisplatin (Choi et al., Arch. Pharmacal Res. 22(2):151-156, 1999), Cis-[PtCl₂(4,7-H-5-methyl-7-oxo]1,2,4-[triazolo[1,5-a]pyrimidine)₂] (Navarro et al., J. Med. Chem. 41(3):332-338, 1998), [Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxyl platinum (Tokunaga et al, Pharm Sci. 3(7):353-356, 1997), Pt(II) . . . Pt(II) (Pt₂[NHCHN(C(CH₂)(CH₃))]₄) (Navarro et al., Inorg Chem. 35(26).7829-7835, 1996), 254-S cisplatin analogue (Koga et al. Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(OaC)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43): 10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyphenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int Symp), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 13945, 1987), (NPr4)₂((PtCL4).cis-(PtCl2-(NH2Me)2)) (Brammer et al., J. Chem. Soc., Chem. Gommun. 6:443-5, 1987), aliphatic tricarboxylic acid platinum complexes (EPA 185225), and cis-dichloro(amino acid) (tert-butylamine)platinum(II) complexes (Pasini & Bersanetti, Inorg. Chim. Acta 107(4):259-67, 1985). These compounds are thought to function by binding to DNA, i.e., acting as alkylating agents of DNA.

viii. Combination Therapy

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the suture coating. Similarly anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be combined with traditional antibiotic and/or antifungal agents to enhance efficacy

Dosages

As sutures are made in a variety of configurations and sizes, the exact dose of anti-infective agent administered will vary with suture size, length, diameter, surface area, design and portions of the suture coated. However, certain principles can be applied in the application of this art. Drug dose can be calculated as a function of dose per unit area (of the portion of the suture being coated), or total drug dose. Total drug dose administered can be measured and appropriate surface concentrations of active drug can be determined. Regardless of the method of application of the drug to the suture, the preferred agents, used alone or in combination, should be administered under the following dosing guidelines:

Anti-infective agents are to be used at concentrations that range from several times more than, to 50%, 20%, 10%, 5%, or even less than 1% of the concentration typically used in a single anti-infective systemic dose application. In certain embodiments, the anti-infective agent is released from the composition in effective concentrations in a time period that may be measured from the time of infiltration into tissue adjacent to the suture, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-100 μg or about 100 μg-1 mg or about 1 mg to 10 mg, of about 10 mg-100 mg, of about 100 mg to 250 mg for coating a suture or a portion thereof or for infiltrating a tissue where a suture has been, is being, or is to be, implanted, or about 250 mg-1000 mg for infiltrating a tissue where a suture has been, is being, or is to be, implanted. The dose (amount) of anti-infective agent per unit area of suture or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm². As different compositions will release the anti-infective agent at differing rates, the above dosing parameters should be utilized in combination with the release rate of the drug from the composition such that a minimum concentration of about 10⁻⁸ M to 10⁻⁷ M, or about 10⁻⁷ M to 10⁻⁶ M or about 10⁻⁶ M to 10⁻⁵ M or about 10⁻⁵ M to 10⁻⁴ M of the agent is maintained in the vicinity of or on the tissue surface to maintain the desired therapeutic effect for the required period of time. The required minimum concentration is dependent on the potency of the agent under consideration and can be determined using standard tests such as the Minimum Inhibitory Concentration (M.I.C.) test.

(a) Anthracyclines Utilizing the anthracycline doxorubicin as an example, whether applied as a polymer coating, incorporated into the polymers which make up the suture (plain or self-retaining), or applied without a carrier polymer, the total dose of doxorubicin applied to the suture should not exceed 25 mg (range of 0.1 μg to 25 mg). In a particularly preferred embodiment, the total amount of drug applied should be in the range of 0.5 μg to 5 mg. The dose per unit area (i.e., the amount of drug as a function of the surface area of the portion of the suture to which drug is applied and/or incorporated) should fall within the range of 0.01 μg-100 μg per mm² of surface area. In a particularly preferred embodiment, doxorubicin should be applied to the suture surface at a dose of 0.1 μg/mm²-10 μg/mm². As different polymer and non-polymer coatings will release doxorubicin at differing rates, the above dosing parameters should be utilized in combination with the release rate of the drug from the suture surface such that a minimum concentration of 10⁻⁷-10⁻⁴ M of doxorubicin is maintained on the surface for the required duration of therapeutic effect It is necessary to insure that surface drug concentrations exceed concentrations of doxorubicin known to be lethal or inhibitory to the growth of multiple species of bacteria and/or fungi (i.e., are in excess of 10⁻⁴ M; although for some embodiments lower concentrations are sufficient). In a preferred embodiment, doxorubicin is released from the surface of the suture such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers that make up the suture (plain or self-retaining), or applied without a carrier polymer, the total dose of mitoxantrone applied should not exceed 5 mg (range of 0.01 μg to 5 mg). In a particularly preferred embodiment, the total amount of drug applied should be in the range of 0.05 μg to 1 mg. The dose per unit area (i.e., the amount of drug as a function of the surface area of the portion of the suture to which drug is applied and/or incorporated) should fall within the range of 0.01 μg-20 μg per mm² of surface area. In a particularly preferred embodiment, mitoxantrone should be applied to the suture surface at a dose of 0.05 μg/mm²-3 μg/mm². As different polymer and non-polymer coatings will release mitoxantrone at differing rates, the above dosing parameters should be utilized in combination with the release rate of the drug from the suture surface such that a minimum concentration of 10⁻⁵-10⁻⁶ M of mitoxantrone is maintained on the surface for the required duration of therapeutic effect. It is necessary to insure that drug concentrations on the suture surface exceed concentrations of mitoxantrone known to be lethal or inhibitory to the growth of multiple species of bacteria and/or fungi (i.e., are in excess of 10 M; although for some embodiments lower drug levels will be sufficient). In a preferred embodiment, mitoxantrone is released from the surface of the suture such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the suture (plain or self-retaining), or applied without a carrier polymer, the total dose of 5-fluorouracil applied should not exceed 20 mg (range of 0.1 μg to 20 mg). In a particularly preferred embodiment, the total amount of drug applied should be in the range of 10 μg to 10 mg. The dose per unit area (i.e., the amount of drug as a function of the surface area of the portion of the suture to which drug is applied and/or incorporated) should fall within the range of 0.01 μg-0.1 mg per mm², of surface area. In a particularly preferred embodiment, 5-fluorouracil should be applied to the suture surface at a dose of 0.1 μg/mm²-100 μg/mm². As different polymer and non-polymer coatings will release 5-fluorouracil at differing rates, the above dosing parameters should be utilized in combination with the release rate of the drug from the suture surface such that a minimum concentration of 10⁻⁴-10⁻⁷ M of 5-fluorouracil is maintained for the required duration of therapeutic effect. It is necessary to insure that surface drug concentrations exceed concentrations of 5-fluorouracil known to be lethal or inhibitory to the growth of numerous species of bacteria and/or fungi (i.e., are in excess of 10⁻⁴ M, although for some embodiments lower drug levels will be sufficient). In a preferred embodiment, 5-fluorouracil is released from the suture surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the suture (plain or self-retaining), or applied without a carrier polymer, the total dose of etoposide applied should not exceed 15 mg (range of 0.1 μg to 15 mg). In a particularly preferred embodiment, the total amount of drug applied should be in the range of 1 μg to 5 mg. The dose per unit area (i.e., the amount of drug as a function of the surface area of the portion of the suture to which drug is applied and/or incorporated) should fall within the range of 0.01 μg-100 μg per mm² of surface area. In a particularly preferred embodiment, etoposide should be applied to the suture surface at a dose of 0.1 μg/mm²-10 μg/mm². As different polymer and non-polymer coatings will release etoposide at differing rates, the above dosing parameters should be utilized in combination with the release rate of the drug from the suture surface such that a concentration of 10⁻⁵-10⁻⁶ M of etoposide is maintained for the required duration of therapeutic effect. It is necessary to insure that surface drug concentrations exceed concentrations of etoposide known to be lethal or inhibitory to the growth of a variety of bacteria and fungi (ie. are in excess of 10⁵ M; although for some embodiments lower drug levels will be sufficient). In a preferred embodiment, etoposide is released from the surface of the suture such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention, the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

2. Polymers

In certain embodiments, the compositions of the present invention may comprise a polymer that facilitates the delivery of a therapeutic agent or forms a sustained release formulation for a therapeutic agent. In certain embodiments, compositions that comprise polymers may further comprise additional agents (e.g., pharmaceutical excipents, echogenic agents, etc.).

For instance, the composition may be or include a hydrophilic polymer gel that has anti-thrombogenic properties. Such a composition can be in the form of a coating that can comprise a hydrophilic, biodegradable polymer that is physically removed from the surface of the suture over time, thus reducing adhesion of platelets to the suture surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA PLA-PEG-PLA, and the like) In one embodiment, the anti-thrombotic composition can include a crosslinked gel formed from a combination of molecules (e.g., PEG) having two or more terminal electrophilic groups and two or more nucleophilic groups.

Representative examples of biodegradable polymers suitable for the delivery of the anti-scarring agent (or other therapeutic agents) include albumin, collagen, gelatin, hyaluronic acid, starch, cellulose and cellulose derivatives (e.g., regenerated cellulose, methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, polyesters, poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X, Y—X—Y, R—(Y—X)ᵣ, or R—(X—Y)ₙ, where X is a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 15-dioxepan-2one (e.g., PLGA, PLA, PCL, polydioxanone and copolymers thereof) and R is a multifunctional initiator), and the copolymers as well as blends thereof (see generally, Illum, L., Davids, S. S. (eds.) “Polymers in Controlled Drug Delivery” Wright, Bristol, 1987: Arshady, J. Controlled Release 17:1-22, 1991; Pitt, Int. J. Phar. 59:173-196, 1990; Holland et al., J. Controlled Release 4:155-0180, 1986).

Representative examples of non-degradable polymers suitable for the delivery of fibrosis-inhibiting agents (or other therapeutic agents) include poly(ethylene-co-vinyl acetate) (“EVA”) copolymers, non-degradable polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), acrylic resin, polyethylene, polypropylene, polyamides (nylon 6,6), polyurethanes (e.g., CHRONOFLEX AR, CHRONOFLEX AL, BIONATE, and PELLETHANE), poly(ester urethanes), poly(ether urethanes), poly(ester-urea), cellulose esters (e.g., nitrocellulose), polyethers (poly(ethylene oxide), poly(propylene oxide), polyoxyalkylene ether block copolymers based on ethylene oxide and propylene oxide such as the PLURONIC polymers (e.g., F-127 or F87) from BASF Corporation (Mount Olive, N.J.), and poly(tetramethylene glycol), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends, copolymers and branched polymers thereof (see generally, Dunn et al., J. Applied Polymer Sci 50:353-365, 1993; Cascone et al., J. Materials Sci.: Materials in Medicine 5770-774, 1994; Shiraishi et al, Biol Pharm. Bull 16(11):1164-1168, 1993; Thacharodi and Rao, Int'l J. Pharm. 120:115-118, 1995; Miyazaki et al., Int'l J. Pharm. 118:257-263, 1995).

Some examples of preferred polymeric carriers include poly(ethylene-co-vinyl acetate), polyurethanes (e.g., CHRONOFLEX AR, CHRONOFLEX AL, BIONATE, and PELLETHANE), poly(D,L-lactic acid) oligomers and polymers, poly(L-lactic acid) oligomers and polymers, poly (glycolic acid), copolymers of lactic acid and glycolic acid, poly(caprolactone), poly(valerolactone), polyanhydrides, copolymers of poly(caprolactone) or poly (lactic acid) with a polyethylene glycol (e.g., MePEG), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.), R is a multifunctional initiator and copolymers as well as blends thereof), nitrocellulose, silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers, as well as blends thereof.

Other representative polymers capable of sustained localized delivery of fibrosis-inhibiting therapeutic agents (or other therapeutic agents) include carboxylic polymers, polyacetates, polycarbonates, polyethers, polyethylenes, polyvinylbutyrals, polysilanes, polyureas, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, natural and synthetic elastomers, rubber, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as the corresponding U.S. applications), U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611, 6,630,155, 6,528,080, RE37,950, 6,461,631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, 5,714,159, 5,612,052, and U.S. Patent Application Publication Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of fibrosis-inhibiting agents.

Polymeric carriers for fibrosis-inhibiting therapeutic agents (or other therapeutic agents) can be fashioned in a variety of forms, with desired release characteristics and/or with specific properties depending upon the composition being utilized. For example, polymeric carriers may be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide monomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer

Likewise, fibrosis-inhibiting therapeutic agents (or other therapeutic agents) can be delivered via polymeric carriers that are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Ret Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al. J Controlled Release 30:69-75, 1994, Yoshida et al. J Controlled Release 32:97-102, 1994; Okano et al., J. Controlled Release 36, 125-133, 1995, Chun and Kim, J. Controlled Release 38:39-47, 1996. D'Emanuele and Dinarvand, Int'l J Pharm. 118:237-242, 1995; Katono et al, J Controlled Release 16.215-228, 1991; Hoffman, “Thermally Reversible Hydrogels Containing Biologically Active Species,” in Migliaresi et al. (eds.), Polymers in Medicine III, Elsevier Science Publishers B.V, Amsterdam, 1988, pp. 161-167; Hoffman, “Applications of Thermally Reversible Polymers and Hydrogels in Therapeutics and Diagnostics,” in Third international Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and the gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C., hydroxypropylmethyl cellulose, 66° C., and ethylhydroxyethyl cellulose, polyalkylene oxide-polyester block copolymers of the structure X—Y, Y—X—Y and X—Y—X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977; U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975; U.S. Patent Application Publication Nos. 2002/012796A1, 2002/0127266A1, 2002/0151650A1, 2003/0104032A1, 2002/0091229A1, 2003/0059906A1; 2004/0219214, and 2004/0225077.

In certain embodiments, in situ formed polymers may be used in the therapeutic composition that comprises an anti-scarring agent (or another therapeutic agent). Such in situ formed polymers include those described in PCT Publication No. WO 05/051452.

In certain aspects, it is desirable to use compositions that can be administered as liquids, but subsequently form hydrogels at the site of administration. Such in situ hydrogel forming compositions can be administered as liquids from a variety of different devices, and are more adaptable for administration to any site, since they are not preformed. Examples of in situ forming hydrogels include photoactivatable mixtures of water-soluble co-polyester prepolymers and polyethylene glycol to create hydrogel barriers. Block copolymers of polyalkylene oxide polymers (e.g., PLURONIC compounds from BASF Corporation, Mount Olive, N.J.) and poloxamers have been designed that are soluble in cold water, but form insoluble hydrogels that adhere to tissues at body temperature (Leach, et al., Am. J. Obstet. Gynecol. 162:1317-1319 (1990)).

In certain embodiments, the present invention provides for polymeric crosslinked matrices, and polymeric carriers, that may be used to assist in the prevention of the formation or growth of fibrous connective tissue. The composition may contain and deliver fibrosis-inhibiting agents in the vicinity of the implanted suture. The following compositions are particularly useful when it is desired to infiltrate around the suture, with or without a fibrosis-inhibiting agent. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a delivery device (e.g., a syringe) in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery In a preferred aspect of the invention, the components of the matrix are delivered in a liquid state to the desired site in the body, whereupon in situ polymerization occurs.

Within further aspects of the present invention, polymeric carriers are provided which are adapted to contain and release a hydrophobic fibrosis-inhibiting compound (or another hydrophobic therapeutic agent), and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix that contains the hydrophobic therapeutic compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

It should be obvious to one of skill in the art that the polymers as described herein can also be blended or copolymerized in various compositions as required to deliver therapeutic doses of anti-scarring agents to sites of suture placement.

3. Additional Components

Within certain embodiments of the invention, the anti-scarring agents (or other therapeutic agents) can be delivered in association with suture placement using non-polymeric agents. These non-polymeric agents can include sucrose derivatives (e.g., sucrose acetate isobutyrate, sucrose oleate), sterols such as cholesterol, stigmasterol, β-sitosterol, and estradiol; cholesteryl esters such as cholesteryl stearate, C₁₂-C₂₄ fatty acids such as lauric acid, myristic acid, palmitic acid, stearic acid, arachidic acid, behenic acid, and lignoceric acid; C18-C36 mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Other carriers that may likewise be utilized to contain and deliver anti-scarring agents described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), nanoparticles (Violante and Lanzafame PAACR), nanoparticles-modified (U.S. Pat. No. 5,145,684), nanoparticies (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), and nanospheres (Hagan et al., Proc. Intern. Symp. Control Rel. Bioact. Mater. 22, 1995; Kwon et al., Pharm Res. 12(2): 192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

In certain embodiments of the invention, anti-scarring agents (or other therapeutic agents) can further comprise a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator), zeolites or cyclodextrins.

Within certain embodiments of the invention, the therapeutic compositions may also comprise additional ingredients such as surfactants (e.g., PLURONICS, such as F-127, L-122, L-101, L-92 L-81, and L-61), preservatives, and anti-oxidants.

Within certain embodiments of the invention, the anti-scarring agent-containing compositions can also comprise radio-opaque, echogenic materials and magnetic resonance imaging (MRI) responsive materials (i.e., MRI contrast agents) to aid in visualization of sutures under ultrasound, fluoroscopy and/or MRI. For example, a suture may be made with or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexyl, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a suture by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium (III) chelates or iron oxide compounds) may be incorporated into or onto the suture, such as a component in a coating. In some embodiments, a suture may include radio-opaque or MRI visible markers (e.g., bands) that may be used to orient and guide the suture during the implantation procedure.

In certain embodiments, sutures may be pre-attached to another device or element. For example, sutures may be pre-attached to a needle or an anchor member for securing its placement in soft or hard tissues. In such embodiments, sutures themselves, the other devices or elements (e.g., needles and anchor members), or both the sutures and the other devices or elements may be coated with a radio-opaque, echogenic, or magnetic resonance imaging (MRI) responsive material.

Sutures may, alternatively, or in addition, be visualized under visible light, using fluorescence, or by other spectroscopic means. Visualization agents that can be included for this purpose include dyes, pigments, and other colored agents In one aspect, the suture may further include a colorant to improve visualization of the suture in vivo and/or ex vivo Frequently, sutures can be difficult to visualize upon insertion, especially at the margins of suture. A coloring agent can be incorporated into a suture to reduce or eliminate the incidence or severity of this problem. The coloring agent provides a unique color, increased contrast, or unique fluorescence characteristics to the suture. In one aspect, a suture is provided that includes a colorant such that it is readily visible (under visible light or using a fluorescence technique) and easily differentiated from its implant site. In another aspect, a colorant can be included in a liquid or semi-solid composition. For example, a single component of a two-component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan. Derivatives, analogues, and isomers of any of the above colored compound also may be used. The method for incorporating a colorant into a suture or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity

In one aspect, the composition and sutures of the present invention include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into a suture or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of additional preservative include paraoxybenzoic acid esters, chlorobutanol, benzylalcohol, phenethyl alcohol, dehydroacetic acid, and sorbic acid. In one aspect, the compositions of the present invention include one or more bactericidal (also known as bacteriacidal) agents.

In one aspect, the compositions and sutures of the present invention include one or more antioxidants, present in an effective amount. Examples of the antioxidant include sulfites, alpha-tocopherol and ascorbic acid.

4. Physical Forms

The compositions of the present invention may be in various forms, such as microparticles or nanoparticles, microspheres, microcapsules, pastes, gels, sprays, and liquids. These can be applied to the surface of the suture or infiltrated into the tissues surrounding the suture. In certain embodiments, anti-scarring agents may be linked by occlusion in the matrices of a polymer, bound by covalent linkages, bound by ionic interactions, or encapsulated in microcapsules.

Within certain aspects of the present invention, therapeutic compositions may be fashioned in any size ranging from 20 nm to 1500 μm, depending upon the particular use. These compositions can be in the form of microspheres (porous or non-porous), microparticles and/or nanoparticles. These compositions can be formed by spray-drying methods, milling methods, coacervation methods, W/O (water-oil) emulsion methods, W/O/W emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a suture surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing anti-scarring agents are particularly useful for application to the surface of tissues that will be in contact with the suture.

The fibrosis-inhibiting therapeutic agent (or another therapeutic agent) may be delivered as a solution. The therapeutic agent can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethylcellulose (CMC), and the like). In another aspect of the invention, the solution can include a biocompatible solvent or liquid oligomers and/or polymers, such as ethanol, DMSO, glycerol, PEG-200, PEG-300 or NMP. These compositions may further comprise a polymer such a degradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, or block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from factide, lactic acid, glycolide, glycolic acid, e-caprolactone, gamma-caprolactone, hydroxyvaleric acid, hydroxybutyric, acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

C. Sutures that Comprise Anti-Scarring Agents

Various types of sutures (plain or self-retaining) may be used in combination with anti-scarring agents according to the present invention. In certain embodiments, sutures are absorbable (e.g those that are degraded by the body's enzymatic pathways and generally lose tensile strength by 60 days after implantation). In certain embodiments, the absorbable sutures are made of polymers or copolymers of glycolic and lactic acid. Exemplary absorbable sutures include catgut (both plain and chromic) (e.g., those with a trade name PROGUT from Dolphin Sutures, India), and those derived from polyglycolic acid with a trade name PETCRYL (Dolphin Sutures, India) and with a trade name DEXON™ (Sherwood Services AG, Schaffhausen, Switzerland), from poliglecaprone 25 with a trade name MONOCRYL® (copolymer of about 75% glycolide and about 25% caprolactone, Johnson & Johnson Co., New Brunswick, N.J.), from polyglactin 910 (such as VICRYL®, coated VICRYL®, coated VICRYL® Plus Antibacterial sutures that contain antibacterial triclosan, and Coated VICRYL RAPIDE® sutures, Johnson & Johnson Co., New Brunswick, N.J.), MULTIPASS® Needle Coating (Johnson & Johnson Co., New Brunswick, N.J.), copolymer of about 67% glycolide and about 33% trimethylene carbonate sold as MAXON™, Wyeth, Madison, N.J., and from polydioxanone with a trade name PDS II® (Johnson & Johnson Co., New Brunswick, N.J.).

In addition to the sutures described above, degradable sutures can be made from polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™ [glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). Other sutures that can be used in this invention include sutures composed of a polymer that comprises the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. These sutures can be in either a braided multifilament form or a monofilament form. The polymers used in the present invention can be linear polymers, branched polymers or multi-axial polymers. Examples of multi-axial polymers used in sutures are described in U.S. Patent Application Publication Nos. 20020161168, 20040024169, and 20040116620.

Absorbable sutures may be used below the surface of the skin to provide support to the skin closure. They may also be used in areas where suture removal might jeopardize the repair such as with small children who might not easily cooperate with suture removal.

In certain embodiments, sutures that may be used in combination with anti-scarring agents are non-absorbable, which may be of monofilament and braided types. Non-absorbable sutures are permanent and include sutures made of polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Exemplary non-absorbable sutures include coated polyester sutres with a trade name Procare (Dolphin Sutures, India), GORTEX™ (made of expanded polytetrafluoroethylene, sold by Gore), NOVAFIL™ (made of polybutester, Wyeth, Madison, N.J.), monofilament polyamide sutures with a trade name Linex (Dolphin Sutures, India), SUTURAE (black braided silk sutures, Sutura Inc., Fountain Valley, Calif.), monofilament polypropylene sutures with a trade name Duracare (Dolphin Sutures, India), MONOSOF® (monofilament nylon suture, United States Surgical Co., Norwalk, Conn.), DERMALON™ (monofilament nylon suture, Sherwood Services AG, Switzerland), SURGILON™ (braided nylon suture coated with silicone, Sherwood Services AG, Switzerland), Ethilon nylon suture (Ethicon, Inc., Somerville, N.J.), ETHIBOND EXCEL® (braided polyester suture from Johnson & Johnson Co., New Brunswick, N.J.), Pronova poly(hexafluoropropylene-VDF) suture (Ethicon, Inc. Somerville, N.J.), TEVDEK™ (braided polyester suture from J.A. Deknatel and Son, Inc. New York, N.Y.), PROLENE™ (polypropylene suture from Ethicon, Inc., Somerville, N.J.), FLUOROFIL™ (polypropylene suture from Pitman-Moore, Inc. Lake Forest, Ill.), and MERSILENE™ (polyester fiber suture from Ethicon, Inc., Somerville, N.J.).

Additional exemplary sutures that may be used in combination with anti-scarring agents according to the present invention are various sutures available from Surgical Specialities Co., Reading, Pa.), including monoderm undyed or dyed monofilament sutures, clear or dyed PCL monofilament sutures, dyed polypropylene monofilament sutures, undyed braided POLYSYN FA sutures, dyed or undyed braided PGA sutures, dyed or undyed braided polysyn suture, dyed monofilament polysyn sutures, dyed braided poloyester sutures, braided silk sutures, dyed braided polyviolene sutures, plain or chromic gut sutures, dyed or undyed monofilament nylon sutures, and dyed pliable nylon sutures. Additional exemplary sutures that may be used in combination with anti-scarring agents, according to the present invention are various sutures available from Tyco International Ltd., Bermuda or its companies. Such sutures include SURGITIE™ (single use ligating loops with delivery system) and SURGIWIP™ (single use sutrue ligatures with delivery system), absorbable sutures such as POLYSORB™ (suturtes composed of LACTOMER™ glycolide/lactide copolymer, a synthetic polyester composed of glycolide and lactide (derived from glycolic and lactic acids), DEXON™ II (synthetic suture composed of homopolymer of glycolic acid and coated with POLYCAPROLATE™, a copolymer of glycolide and epsilon-caprolactone), DEXON™ S (synthetic sutures composed of the homopolymer of glycolic acid), MAXON™ CV (polyglyconate synthetic sutures prepared from a copolymer of glycolic acid and trimethylene carbonate), plain, mild chromic, and chromic gut sutures composed of purified connective tissue (mostly collagen) derived from the serosal layer of beef intestines, and non-absorbable sutures such as DERMALON® (nylon), MONOSOF® (nylon), SURGILON® (nylon). SURGIDAC™ (polyethylene terephthalate) TI-CRON™ (sutures prepared from fibers of high molecular weight, long chain and linear polyesters having recurrent aromatic rings as an intergral component), SURGIPRO™ (sutures composed of an isotactic crystalline stereoisomer of polypropylene (a synthetic linear polyolefin) and polyethylene), SURGIPRO™ II (sutures composed of an isotactic crystalline stereoisomer of polypropylene (a synthetic linear polyolefin) and polyethylene), NOVAFIL™ (sutures composed of polybutester, a copolymer of butylenes terephthalate and polytetramethylene ether glycol), VASCUFIL™ (sutures composed of a copolymer of butylenes terephthalate and polytetramethylene ether glycol and coated with POLYTRIBOLATE™, an absorbable polymer of ε-caprolactone/glycolidelpoloxamer 188), FLEXON™ (twisted multistrand steel sutures coated with orange or white PTFE poly(tetrafloroproethylene) or clear FEP poly(tetrafluoroethylene-co-hexafluoropropylene), SOFSILK™ (sutures composed of natural proteinaceous silk fibers that are treated to remove the naturally-occurring sericin gum), and stainless steel sutures.

In certain embodiments, sutures that may be used in combination with anti-scarring agents are used in various dental procedures, i.e., oral and maxillofacial surgical procedures and thus may be referred to as “dental sutures.” The above-mentioned procedures include, but are not limited to, oral surgery (e.g., removal of impacted or broken teeth), surgery to provide bone augmentation, surgery to repair dentofacial deformities, repair following trauma (e.g., facial bone fractures and injuries), surgical treatment of odontogenic and non-odontogenic tumors, reconstructive surgeries, repair of cleft lip or cleft palate, congenital craniofacial deformities, and esthetic facial surgery. Many of the various sutures described above are used in such procedures and are available from many of the same commercial sources. As above, dental sutures may be degradable or non-degradable. Sutures used in oral and maxillofacial surgical procedures may typically range in size from USP 2-0 to USP 6-0. Dental sutures may have a surgical needle attached.

In certain embodiments, sutures that may be used in combination with anti-scarring agents are microsutures Microsutures are used in microsurgical procedures that are performed under a surgical microscope. Such surgical procedures include, but are not limited to, reattachment and repair of peripheral nerves, spinal microsurgery, microsurgery of the hand, various plastic microsurgical procedures (e.g., facial reconstruction), microsurgery of the male or female reproductive systems, and various types of reconstructive microsurgery. Microsurgical reconstruction is used for complex reconstructive surgery problems when other options such as primary closure, healing by secondary intention, skin grafting, local flap transfer, and distant flap transfer are not adequate. Microsutures are available from many of the commercial sources identified above and are made from the same materials described above. As above, microsutures may be degradable or non-degradable. Microsutures have a very small caliber, often as small as USP 9-0 or USP 10-0, and may have an attached needle of corresponding size.

Additional exemplary sutures that may be used in combination with anti-scarring agents are described in U.S. Pat. Nos. 5,766,188, 4,441,496, 6,692,516, 4,550,730, 4,052,988, and U.S. Patent Application Publication Nos. 2005267532, 2005240224, 2004111116, 2004088003, 2002095180.

In certain embodiments, the suture can further comprise a coating. The coating can comprise a degradable or a non-degradable polymer. Coatings can include but are not limited to polybutylene adipate (SURGIDAC™) to silicone (TI-CRON™), poly(glycolide-co-lactide) (e.g., polyglactin 370), copolymers of gylcolide, ε-caprolactone, and poloxamer 188, copolymers of glycolide and, ε-caprolactone (e.g., polycaprolate), poloxamer 188, calcium stearate, and calcium stearoyl lactylate, as well as blends and mixtures thereof. Coatings can also include polymers that can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Exemplary coatings that can be used are described in U.S. Pat. Nos. 4,047,533, 4,201,216, 4,470,416, 4,788,979, 4,857,602, 4,994,074, 5,037,950, 5,100,433, 5,102,420, 5,123,912, 5,522,842, 5,543,218, 6,703,035, and 6,703,035. In certain embodiments, the anti-scarring agent can be incorporated into or onto the suture coating polymers described above.

In certain embodiments, sutures that may be used in combination with anti-scarring agents are self-retaining sutures. Such sutures include but are not limited to: one-way sutures disclosed in U.S. Pat. Nos. 3,123,077, 5,053,047, 5,931,855, PCT Application Publication No. WO 98/52473, barbed suture described in McKenzie et al., J Bone Joint Surg [Br]49(3): 440-7, 1967, and bi-directional suture described in U.S. Pat. Nos. 5,342,376, 6,241,747, US 2003/0074023, and Dattilo et al., 2003 Society For Biomaterials 29^{th} Annual Meeting Transactions. Page 101. Additional description of self-retaining sutures useful in the present invention may be found in U.S. Pat. Nos. 6,599,310, 6,773,450, 6,848,152, published U.S. Application Nos. US 2004/0060410, US 2004/0060409, US 2004/0088003, and US 2004/0226427, and PCT Application Publication Nos. WO 03/001979, WO 2004/014236, WO 03/017850, WO 2004/030520, WO 2004/030704, WO 2004/030705, and WO 2007/005296.

In one embodiment, a commercially available suture that may be used in combination with an anti-scarring agent is CONTOUR THREADS™ (Quill Medical, Research Triangle Park, N.C.). CONTOUR THREADS™ are non-absorbable self-retaining suture product cleared by the FDA for the elevation and fixation of midface, brow and neck areas. They are made from clear polypropylene.

In another embodiment, the suture that may be used in combination with a fibrosing agent is a suture with a similar structure to the CONTOUR THREADS™ but is composed of a degradable polymer. These degradable polymers polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Examples include polydioxanone, poly(lactide-co-trimethylene carbonate) polymers, poly(lactide-co-glycolide) polymers and poly(lactide-co-glycolide-co-trimethylene carbonate) polymers.

In other embodiments, the suture that may be used in combination with an anti-scarring agent is Aptos Thread developed by Dr. Sulamandize of Moscow (available from I-LIFT TENSOR THREADS, Argentine). Description of such suture may be found in European Published Patent Application No. 1,075,843 A1 and WO 00/51658. The suture has conical retainers arranged sequentially along the length of a thread and oriented in a direction opposite to that of the thread tension, with the distance between retainers being no less than 1.5 times the thread diameter.

As indicated above, certain types of self-retaining sutures that may be used in combination with an anti-scarring agent according to the present invention are commercially available. In certain other embodiments, self-retaining sutures may be made using any suitable method, including injection molding, stamping, cuffing, laser, extrusion, separate manufacture and subsequent attachment of retainers, and the like. With respect to cutting, polymeric thread or filaments may be purchased, and the retainers are subsequently cut onto the filament body. In certain embodiments, barbed sutures may be produced according to U.S. Pat. No. 6,848,152 and U.S. Patent Application Publication Nos. US 2004/0226427 and US 2004/0060409.

In certain embodiments, sutures that may be used in combination with an anti-scarring agent according to the present invention are already attached to surgical needles. Attachment of sutures and surgical needles is described in U.S. Pat. Nos. 3,981,307, 5,084,063, 5,102,418, 5,123,911, 5,500,991, 5,722,991, 6,012,216, and 6,163,948, and U.S. Patent Application Publication No. US 2004/0088003. A method for the manufacture of surgical needles is described in U.S. Pat. No. 5,533,982, and a method for the manufacture of polymer-coated surgical needles is described in U.S. Pat. No. 5,258,013.

In certain embodiments, the sutures that may be used in combination with an anti-scarring agent according to the present invention are pointing at both ends (including suture connectors as described in U.S. Pat. No. 6,241,747) In certain other embodiments, the sutures may have one pointing end and an achor on the other end. The anchor may be used to secure the implantation of the suture in soft tissue (e.g., those described in U.S. Patent Application Publication No. US2005/0267531) or the attachment of sutures to the bone (e.g., those described in U.S. Pat. No. 6,773,450 and PCT Application Publication No. WO 2004/014236).

In certain other embodiments, the suture may be a relatively short suture with sharp pointing ends. Such a suture may function similar to a staple when used in connecting tissues and thus permits a surgeon to rapidly and securely attach the edges of a wond in a boldly tissue or reconfigure the tissue without the necessity for threading and tying numerous individual stitches or for the use of a complicated tool to insert the suture. This type of sutures may thus be referred to as “suture connector.” In certain embodiments, the suture connector may be a bi-directional self-retaining suture. In certain other embodiments, the suture connector may be found by linking two relatively short uni-directional self-retaining sutures together to form a bi-directional self-retaining suture (see, U.S. Pat. No. 6,241,747).

D. Methods for Making Sutures that Comprise Anti-Scarring Agents

Various methods may be used to make sutures that comprise anti-scarring agents. For example, such methods may comprise the step of coating (e.g., spraying or dipping) all or part of the sutures. Additionally, sutures themselves may be comprised at least in part of materials that inhibit fibrosis in or around the site where the sutures are implanted or inserted.

In certain embodiments, only selected portions (such as middle sections or the self-retaining sections) of sutures may be coated or otherwise comprise anti-scarring agents or anti-scarring agent-containing compositions. In certain further embodiments, portions of the sutures may be selectively left unassociated with anti-scarring agents or anti-scarring agent-containing compositions, for example, the suture surfaces between retainer and main suture body in which tissue may be gripped may be so selectively unassociated with anti-scarring agents or anti-scarring agent-containing compositions. In certain other embodiments, the suture surface may comprise one or more wells of anti-scarring agents or anti-scarring agent-containing compositions. In other embodiments, all sections of sutures may be coated or otherwise comprise anti-scarring agents or anti-scarring agent-containing compositions

1. Exemplary Methods for Combining Anti-scarring Agents with Sutures

Various exemplary methods for combining anti-scarring agents with sutures to produce sutures that comprise anti-scarring agents are described in more detail below

a. Coating of Sutures with Anti-Scarring Agents

Anti-scarring agents or compositions comprising anti-scarring agents may be coated onto or into a suture by various methods known in the art such as by dipping, spraying, electrospinning, painting or by vacuum deposition. In certain embodiments including self-retaining sutures, the sutures may be coated prior to the formation of retainers during the manufacturing process (thereby resulting in sutures having selectively uncoated portions). In certain other embodiments including self-retaining sutures, the sutures may be coated after or concurrent with the formation of retainers.

i. Dip Coating

Dip coating is one coating process that can be used to coat a suture In one embodiment, the anti-scarring agent is dissolved in a solvent for the anti-scarring agent and is then coated onto the suture.

Anti-Scarring Agent with an Inert-Solvent

In one embodiment, the solvent is an inert solvent for the suture such that the solvent does not dissolve the suture to any significant extent and is not absorbed by the suture to any significant extent. The suture can be immersed, either partially or completely, in the anti-scarring agent/solvent solution for a specific period of time. The rate of immersion into the anti-scarring agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the anti-scarring agent being coated on the surface of the suture.

Anti-Scarring Agent with a Swelling Solvent

In one embodiment, the solvent is one that will not dissolve the suture but will be absorbed by the suture. These solvents can thus swell the suture to some extent. The suture can be immersed, either partially or completely, in the anti-scarring agent/solvent solution for a specific period of time (seconds to days). The rate of immersion into the anti-scarring agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels This process will result in the anti-scarring agent being adsorbed into the suture The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

Anti-Scarring Agent with a Solvent

In one embodiment, the solvent is one that will be absorbed by a suture and that will dissolve the suture. The suture can be immersed, either partially or completely, in the anti-scarring agent/solvent solution for a specific period of time (seconds to hours). The rate of immersion into the anti-scarring agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the anti-scarring agent being adsorbed into the suture as well as being surface associated. In the preferred embodiment, the exposure time of the suture to the solvent would be such that the suture does not undergo significant permanent dimensional changes. The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

In the above description, the suture can be a suture that has not been modified as well as a suture that has been further modified by coating with a polymer (e.g., parylene), surface treated, surface etching, mechanical smoothing or roughening, or grafting prior to the coating process.

In one embodiment, the anti-scarring agent and a polymer are dissolved in a solvent, for both the polymer and the anti-scarring agent, and are then coated onto the suture

Anti-Scarring Agent/Polymer with an Inert-Solvent

In one embodiment, the solvent is an inert solvent for the suture such that the solvent does not dissolve the suture to any great extent and is not absorbed by the suture to any great extent. The suture can be immersed, either partially or completely, in the anti-scarring agent/polymer/solvent solution for a specific period of time. The rate of immersion into the anti-scarring agent/polymer/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the anti-scarring agent/polymer being coated on the surface of the suture.

Anti-Scarring Agent/Polymer with a Swelling Solvent

In one embodiment, the solvent is one that will not dissolve a suture but will be absorbed by the suture. These solvents can thus swell the suture to some extent. The suture can be immersed, either partially or completely, in the anti-scarring agent/polymer/solvent solution for a specific period of time (seconds to days). The rate of immersion into the anti-scarring agent/polymer/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the anti-scarring agent/polymer being coated onto the surface of the suture as well as the potential for the anti-scarring agent being adsorbed into the suture. The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

Anti-Scarring Agent/Polymer with a Solvent

In one embodiment, the solvent is one that will be absorbed by a suture and that will dissolve the suture. The suture can be immersed, either partially or completely, in the anti-scarring agent/solvent solution for a specific period of time (seconds to hours). The rate of immersion into the anti-scarring agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. In the preferred embodiment, the exposure time of the suture to the solvent would be such that there is not significant permanent dimensional change to the suture (other than those associated with the coating itself). The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

In the above description the suture can be a suture that has not been modified as well as a suture that has been further modified by coating with a polymer (e.g., parylene), surface treated, surface etching, mechanical smoothing or roughening, or grafting prior to the coating process.

In any one the above dip coating methods, the surface of the suture can be treated with a plasma polymerization method prior to coating of the anti-scarring agent or anti-scarring agent containing composition, such that a thin polymeric layer is deposited onto the suture surface. Examples of such methods include parylene coating of sutures and the use of various monomers such hydrocyclosiloxane monomers. Parylene coating may be especially advantageous if the suture, or portions of the suture, is composed of materials (e.g., stainless steel, nitinol) that do not allow incorporation of the therapeutic agent(s) into the surface layer using one of the above methods. A parylene primer layer may be deposited onto the electrical suture using a parylene coater (e.g., PDS 2010 LABCOTER2 from Cookson Electronics, Inc., Foxborough, Mass.) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and PARYLENE D, a dichlorinated derivative of PARYLENE N).

In another embodiment, a suspension of the anti-scarring agent in a polymer solution can be prepared. The suspension can be prepared by choosing a solvent that can dissolve the polymer but not the anti-scarring agent or a solvent that can dissolve the polymer and in which the anti-scarring agent is above its solubility limit. In similar processes described above, a suture can be dipped into the suspension of the anti-scarring agent and polymer solution such that the suture is coated with the suspension.

ii. Spray Coating

Spray coating is another coating process that can be used. In the spray coating process, a solution or suspension of the anti-scarring agent, with or without a polymeric or non-polymeric carrier, is nebulized and directed to the suture to be coated by a stream of gas. One can use spray sutures such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part #14545 and 14654, Alltech Associates, Inc Deerfield, Ill., and ultrasonic spray sutures (for example those available from Sono-Tek, Milton, N.Y.) One can also use powder sprayers and electrostatic sprayers.

In one embodiment, the anti-scarring agent is dissolved in a solvent for the anti-scarring agent and is then sprayed onto the suture.

Anti-Scarring Agent with an Inert-Solvent

In one embodiment, the solvent is an inert solvent for a suture such that the solvent does not dissolve the suture to any great extent and is not absorbed by the suture to any great extent. The suture can be held in place or the suture can be mounted onto a mandrel or rod that has the ability to move in an X, Y or Z plane or a combination of these planes. Using one of the above described spray sutures, the suture can be spray coated such that the suture is either partially (for example, coating of the self-retaining region only) or completely coated with the anti-scarring agent/solvent solution. The rate of spraying of the anti-scarring agent/solvent solution can be altered (e.g., 0.001 ml per sec to 10 ml per sec) to ensure that a good coating of the anti-scarring agent is obtained. The coated suture can be air-dried. The spray coating process can be repeated one or more times depending on the specific application. The suture can be dried under vacuum to reduce residual solvent levels. This process will result in the anti-scarring agent being coated on the surface of the suture.

Anti-Scarring Agent with a Swelling Solvent

In one embodiment, the solvent is one that will not dissolve a suture but will be absorbed by the suture. These solvents can thus swell the suture to some extent. The suture can be spray coated, either partially (for example, coating of the self-retaining region only) or completely, in the anti-scarring agent/solvent solution. The rate of spraying of the anti-scarring agent/solvent solution can be altered (e.g., 0.001 ml per sec to 10 ml per sec) to ensure that a good coating of the anti-scarring agent is obtained. The coated suture can be air-dried. The spray coating process can be repeated one or more times depending on the specific application. The suture can be dried under vacuum to reduce residual solvent levels. This process will result in the anti-scarring agent being adsorbed into the suture. The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

Anti-Scarring Agent with a Solvent

In one embodiment, the solvent is one that will be absorbed by a suture and that will dissolve the suture. The suture can be spray coated, either partially (for example, coating of the self-retaining region only) or completely, in the anti-scarring agent/solvent solution. The rate of spraying of the anti-scarring agent/solvent solution can be altered (e.g., 0.001 ml per sec to 10 ml per sec) to ensure that a good coating of the anti-scarring agent is obtained. The coated suture can be air-dried. The spray coating process can be repeated one or more times depending on the specific application. The suture can be dried under vacuum to reduce residual solvent levels. This process will result in the anti-scarring agent being adsorbed into the suture as well as being surface associated. In one embodiment, the exposure time of the suture to the solvent would be such that the suture would incur no significant permanent dimensional changes. The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

In the above description the suture can be a suture that has not been modified as well as a suture that has been further modified by coating with a polymer (eg, parylene), surface treated, surface etching, mechanical smoothing or roughening, or grafting prior to the coating process

In one embodiment, the anti-scarring agent and a polymer are dissolved in a solvent, for both the polymer and the anti-scarring agent, and are then spray coated onto the suture.

Anti-Scarring Agent/Polymer with an Inert-Solvent

In one embodiment, the solvent is an inert solvent for a suture such that the solvent does not dissolve the suture to any great extent and is not absorbed by the suture to any great extent. The suture can be spray coated, either partially (for example, coating of the self-retaining region only) or completely, in the anti-scarring agent/polymer/solvent solution for a specific period of time. The rate of spraying of the anti-scarring agent/solvent solution can be altered (e.g., 0.001 ml per sec to 10 ml per sec) to ensure that a good coating of the anti-scarring agent is obtained. The coated suture can be air-dried. The spray coating process can be repeated one or more times depending on the specific application. The suture can be dried under vacuum to reduce residual solvent levels. This process will result in the anti-scarring agent/polymer being coated on the surface of the suture.

Anti-Scarring Agent/Polymer with a Swelling Solvent

In one embodiment, the solvent is one that will not dissolve a suture but will be absorbed by the suture. These solvents can thus swell the suture to some extent. The suture can be spray coated, either partially (for example, coating of the self-retaining region only) or completely, in the anti-scarring agent/polymer/solvent solution. The rate of spraying of the anti-scarring agent/solvent solution can be altered (e.g., 0.001 ml per sec to 10 ml per sec) to ensure that a good coating of the anti-scarring agent is obtained. The coated suture can be air-dried. The spray coating process can be repeated one or more times depending on the specific application The suture can be dried under vacuum to reduce residual solvent levels This process will result in the anti-scarring agent/polymer being coated onto the surface of the suture as well as the potential for the anti-scarring agent being adsorbed into the suture. The anti-scarring agent may also be present on the surface of the suture. The amount of surface associated anti-scarring agent may be reduced by dipping the coated suture Into a solvent for the anti-scarring agent or by spraying the coated suture with a solvent for the anti-scarring agent.

Anti-Scarring Agent/Polymer with a Solvent

In one embodiment, the solvent is one that will be absorbed by a suture and that will dissolve the suture. The suture can be spray coated, either partially (for example, coating of the self-retaining region only) or completely, in the anti-scarring agent/solvent solution. The rate of spraying of the anti-scarring agent/solvent solution can be altered (e.g., 0.001 ml per sec to 10 ml per sec) to ensure that a good coating of the anti-scarring agent is obtained. The coated suture can be air-dried. The spray coating process can be repeated one or more times depending on the specific application. The suture can be dried under vacuum to reduce residual solvent levels. In the preferred embodiment, the exposure time of the suture to the solvent would be such that there are not significant permanent dimensional changes to the suture (other than those associated with the coating itself). The anti-scarring agent may also be present on the surface of the suture. The amount of surface associated anti-scarring agent may be reduced by dipping the coated suture into a solvent for the anti-scarring agent or by spraying the coated suture with a solvent for the anti-scarring agent.

In the above description the suture can be a suture that has not been modified as well as a suture that has been further modified by coating with a polymer (e.g., parylene), surface treated by plasma treatment, flame treatment, corona treatment, surface oxidation or reduction, surface etching, mechanical smoothing or roughening, or grafting prior to the coating process.

b. Other Exemplary Methods for Combining Anti-Scarring Agents with Sutures

In certain embodiment, a particulate form of the active agent (e.g., silk, wool, cyanoacrylate particles or chitosan) may be coated onto the suture. In one embodiment, the particulate form may be incorporated into a polymeric carrier (e.g., PLG, PLA, polyurethane, suture coatings as described above) Alternatively, or in addition, particles of the active agent can be applied onto a polymer-coated suture. For example, a suture can be coated with a polymer (e.g., a polyurethane) and then allowed to partially dry such that the surface is still tacky. A particulate form of the anti-scarring agent or an anti-scarring agent and secondary carrier, such as described above, can then be applied to all or a portion of the tacky coating after which the suture is dried.

In certain embodiments, a suture having a polymeric coating with or without an anti-scarring agent can be subjected to a thermal treatment process to soften the coating. An anti-scarring agent or an anti-scarring agent and secondary carrier then is applied to all or a portion of the softened coating.

Coated sutures may be further coated with an additional composition and/or be treated to alter the release characteristics of the coating composition and/or anti-scarring agent. For example, a suture having an anti-scarring agent or anti-scarring composition incorporated into or coated onto the suture may be further coated with a composition or compound which delays the onset of activity of the anti-scarring agent for a period of time after implantation. Protection of a biologically active surface can be achieved by coating the suture surface with an inert molecule that prevents access to the active site through steric hindrance. Representative examples of such compositions or compounds include biologically inert materials such as gelatin, PLGA/MePEG film, PLA, polyurethanes, suture coatings as described above, silicone rubbers, surfactants, lipids, or polyethylene glycol, as well as biologically active materials such as heparin (e.g., to induce coagulation). In one embodiment, the active agent (e.g., poly-L-lysine, fibronectin, chitosan, silk, wool, bleomycin, cyclosporine A, or CTGF) on the suture is top-coated with a physical barrier that does not contain an anti-scarring agent. The barrier layer can include non-degradable materials or biodegradable materials such as, e.g., gelatin PLGA/MePEG film, PLA, PLG, or polyethylene glycol. The barrier layer (e.g, dissolves slowly or degrades once implanted into the host As the top layer dissolves or degrades, the active agent becomes exposed to the surrounding tissue and/or can be released from the coating

Within yet another embodiment, the outer layer of the coated suture, which is capable of inducing an in vivo fibrotic response, is further treated to crosslink or functionalize the outer layer of the coating. Crosslinking of the coating (and/or additional surface modification) can be accomplished using a variety of methods, including, for example, subjecting the coated suture to a plasma treatment process. The degree of crosslinking and nature of the surface modification can be altered by changing the RF power setting, the location with respect to the plasma, the duration of treatment, as well as the gas composition introduced into the plasma chamber.

Protection of a biologically active surface can also be achieved by coating the surface with an inactive form of the anti-scarring agent, which is later activated. The anti-scarring suture may be activated before, during, or after deployment (e.g., an inactive agent on the suture may be first activated to one that induces or accelerates an in vivo fibrotic reaction).

In one embodiment, the suture can be coated with an inactive form of the anti-scarring agent, applied as described herein, which is then activated once the suture is deployed. Activation can be achieved by injecting an activating agent (e.g., an enzyme) or a composition that includes an activating agent into the tissue or area surrounding the suture after deployment of the suture or after the anti-scarring agent has been administered to the tissue (via drug delivery catheters or balloons).

In one embodiment, a suture includes a first coating layer that includes a biologically active anti-scarring agent and a first reactive component. In one embodiment, the first reactive component is capable of reaction with a polyethylene glycol The coated suture can be further coated with a second composition that includes a second reactive component (e.g., polyethylene glycol) that is capable of reaction with the first reactive component in the first coating layer. The reactive components of the first and second coating layers can be bonded via a condensation reaction through formation of ester bonds. Prior to the deployment of the intra-arterial segment of the suture, an esterase is injected into the treatment site around the outside of the suture, which can cleave the ester linkages, thus allowing the agent to become available to inhibit fibrosis

2. Anti-Scarring Agent Releasing Profiles

In certain embodiments, anti-scarring agents may be released from sutures that comprise anti-scarring agents. In other embodiments, the anti-scarring agents may be released from carriers that are applied to the surrounding tissue as liquids, gels, pastes, suspensions, microspheres, nanoparticles or other such delivery vehicles. In certain other embodiments, such agents become part of sutures permanently and no anti-scarring agents are released from the suture.

The sutures or compositions of the present invention may release the anti-scarring agent at one or more phases, the one or more phases having similar or different performance (e.g., release) profiles. The therapeutic agent may be made available to the tissue at amounts which may be sustainable, intermittent, or continuous; in one or more phases; and/or rates of delivery; effective to reduce or inhibit any one or more components of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing an anti-scarring agent at a time such that at least one of the components of fibrosis is inhibited or reduced. Moreover, the predetermined release rate may reduce agent loading and/or concentration as well as potentially providing minimal drug washout and thus, increases efficiency of drug effect Any one of the at least one anti-scarring agents may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring agent to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, substantially non-releasing.

The total amount of anti-scarring agent made available on, in or near the suture may be in an amount ranging from about 0.01 μg (micrograms) to about 2500 mg (milligrams). Generally, the anti-scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The total surface amount of anti-scarring agent on, in or near the suture may be in an amount ranging from less than 0.01 μg to about 100 μg per mm² of suture surface area. Generally, the anti-scarring agent may be in the amount ranging from less than 0.01 μg/mm²; or from 0.01 μg/mm² to about 10 μg/mm²; or from 10 μg/mm² to about 50 μg/mm²; or from 50 μg/mm² to about 100 μg/mm².

The anti-scarring agent that is on, in or near the suture may be released from the composition in a time period that may be measured from the time of implantation, which ranges from about less than 1 day to about 180 days. Generally, the release time may also be from about less than 1 day to about 7 days; from 7 days to about 14 days, from 14 days to about 28 days; from 28 days to about 56 days; from 56 days to about 90 days; from 90 days to about 180 days

The amount of anti-scarring agent released from the suture or composition as a function of time may be determined based on the in vitro release characteristics of the agent from the composition. The in vitro release rate may be determined by placing the anti-scarring agent within the composition or suture in an appropriate buffer such as 0.1 M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring agent per day may range from about 0.01 μg (micrograms) to about 2500 mg (milligrams). Generally, the anti-scarring agent may be released in a day in the amount ranging from 0.01 μg to about 10 μg; or from 10 μg to about 1 mg; or from 1 mg to about 10 mg; or from 10 mg to about 100 mg, or from 100 mg to about 500 mg; or from 500 mg to about 2500 mg.

In one embodiment, the anti-scarring agent is made available to the susceptible tissue site in a programmed, sustained, and/or controlled manner that results in increased efficiency and/orefficacy. Further, the release rates may vary during either or both of the initial and subsequent release phases. There may also be additional phase(s) for release of the same substance(s) and/or different substance(s).

3. Sterilization

Further, sutures that comprise anti-scarring agents (as well therapeutic compositions useful in making, or using in combination of, the sutures) of the present invention should preferably be have a stable shelf-life for at least several months and capable of being produced and maintained under sterile conditions. The compositions or sutures may be sterile either by preparing them under aseptic environment and/or they may be terminally sterilized using methods available in the art. Many pharmaceuticals/medical devices are manufactured to be sterile and this criterion is defined by the USP XXII <1211>. The term “USP” refers to U.S. Pharmacopeia (see www usp org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Sterilization may also occur by terminally using gamma radiation or electron beam sterilization methods. A combination of these methods may also be used to prepare the compositions and sutures in the sterile form.

E. Using Sutures in Combination with Anti-Scarring Agents

Sutures used in combination with anti-scarring agents according to the present application may be useful in reducing, or reducing the risk of, excessive scarring (e.g., hypertrophic scars and keloids) and side effects resulted from excessive scarring (e.g., surgical adhesions). In certain embodiments, sutures themselves comprise anti-scarring agents, which are implanted into a tissue. In certain other embodiments, sutures are not first combined with anti-scarring agents, compositions comprising anti-scarring agents, or other compositions effective in reducing, or reducing the risk of, excessive scarring or associated side effects (e.g., polymer compositions). Instead, anti-scarring agents, compositions comprising anti-scarring agents, or other compositions effective in reducing excessive scarring or risk thereof (e.g., polymer compositions) are delivered separately to the site where the sutures have been, are being, or are to be implanted, to infiltrate such a site. Such delivery may be performed by the use of drug-delivery catheter or by injections or direct applications (e.g., at wound sites).

The hosts on which various applications using sutures in combination with anti-scarring agents are performed may be mammals. In certain embodiments, the hosts are humans In certain other embodiments, other mammals (including but not limited to a non-human primate, rodent, cat, dog, horse, pig, bovine, sheep, or goat) may be the hosts

The implantation or insertion of, or the stitching with, a suture (with or without an anti-scarring agent) may be performed using the suture itself (e.g., a suture with one or two pointing ends), a needle removably attached to the suture, or an insertion means (e.g., a hallow device in which the suture may be hosted during insertion and subsequently removed to leave the suture inside the host tissue) (see, e.g., U.S. Pat. No. 5,342,376).

For the specific applications described herein, any suitable techniques by which a suture is properly implanted or used known in the art may be used. Such techniques include alpha suture, zigzag suture, coil suture, “switch back” suture, “finger-trap” suture, Connell suture, everting suture, Halsted suture, horizontal mattress suture, Lembert suture, lock or lock-stitch, locking-stitch, purse-string suture, subcuticular suture, and vertical mattress suture (see, e.g., Medical Textiles 2004, Advances in Biomedical Textiles and Healthcare Products, Conference Proceedings, IFA/Expo 2004, Oct. 26-27, 2004, Pittsburgh, Pa., pp 262-80). Description of certain exemplary techniques for using self-retaining sutures may be found in U.S. Pat. Nos. 6,773,450, 6,241,747, and 6,599,310 and U.S. Patent Application Publication No. 2003/0074023.

Any of the aforementioned sutures may be used in combination of an anti-scarring agent. For example, sutures may be biodegradable (or absorbable) or non-biodegradable (or non-absorbable), plain or self-retaining (one-directional or bi-directional). In certain embodiments, the suture may be attached to a needle or another insertion device. In certain other embodiments, the suture may further comprise an anchor member at one end for securing the implantation of the suture in a soft or hard tissue. In certain other embodiments, the suture is a suture connector as described above.

In certain embodiments, self-retaining sutures that comprise anti-scarring agents or in combination with anti-scarring agents are used. Due to their retainers, such sutures allow for joining a tissue with another tissue or with a foreign element, or repositioning a tissue without the need for using various time-consuming and skill-demanding knotting devices or techniques. In addition, the anti-scarring agents on or in the sutures or the tissues surrounding or in contact with the sutures reduce excessive scarring or the risk thereof that may cause by the retainers on the sutures.

Any anti-scarring agent disclosed herein may be used in combination with a suture, either directly on the suture or indirectly by infiltrating a tissue surrounding or in contact with the suture.

Within certain embodiments, compositions for reducing, or reducing the risk of, excessive scarring (e.g., hypertrophic scars or keloids) or associated side effects (e.g., surgical adhesion) may release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Examples of fibrosis-inhibiting agents for use with sutures for treating or preventing excessive scarring (e.g., hypertrophic scars or keloids) or associated side effects (e.g., surgical adhesion) include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (eg. sulconizole) and (J) p38 MAP kinase inhibitors (eg. SB202190), as well as analogues and derivatives of the aforementioned

In certain embodiments, the anti-scarring agent is cerivastatin, terbinafine, radicicol, trichostatin A, mithramycin A, 5-fluorouracil, doxorubicin, mitoxantrone, etoposide, paclitaxel, rapamycin, halofuginone hydrobromide, pitavastatin, or pirarubicin.

### Using Anti-Scarring Sutures

In one aspect, the present invention provides a method for reducing or reducing the risk of, excessive scarring, comprising: implanting an anti-scarring suture in a tissue and thereby reducing excessive scarring or risk thereof surrounding the suture.

“Excessive scarring” refers to the formation of excessive dense fibrous connective tissue that forms over a healed wound or cut (i.e., excessive scar). Excessive scarring may interfere the normal functions of the tissue where excessive scar is located (e.g., causing contractures) or disfigure the tissue (e.g., skin, especially facial skin).

An anti-scarring compound, composition, or suture that “reduces excessive scarring” if there is a statistically significant reduction in excessive scarring in the presence of the compound, composition, or suture compared to in the absence of the compound, composition, or suture. Similarly, a procedure or a method that “reduces excessive scarring” if there is a statistically significant reduction in excessive scarring if the procedure or method is performed compared to when the procedure or method is not performed.

An anti-scarring compound, composition, or suture that “reduces the risk of excessive scarring” if there is a statistically significant reduction in the likelihood that excessive scarring will occur in the presence of the compound, composition, or suture compared to in the absence of the compound, composition, or suture. Similarly, a procedure or a method that “reduces the risk of excessive scarring” if there is a statistically significant reduction in the likelihood that excessive scarring will occur if the procedure or method is performed compared to when the procedure or method is not performed

Anti-scarring sutures may be needed to close a wound (including those resulted from injuries or sugeries), to join tissues together, to attach a foreign element to a tissue, or to perform a tissue repositioning surgery (e.g., face lifts, neck lifts, brow lifts, thigh lifts, and breast lifts). Accordingly, in certain embodiments, excessive scarring that may be reduced by the use of anti-scarring sutures is at a wound closure site, a site of attachment of a foreign element to a tissue, or a site of a tissue repositioning surgery.

In certain embodiments, a method for reducing, or reducing the risk of, excessive scarring resulting from closing a wound is provided, comprising: closing the wound with a suture that comprises an anti-scarring agent.

In certain embodiments, a method for reducing, or reducing the risk of, excessive scarring resulting from joining tissues together is provided, comprising: joining the tissues together with a suture that comprises an anti-scarring agent.

In certain embodiments, a method for reducing, or reducing the risk of, excessive scarring resulting from attaching a foreign element to a tissue is provided, comprising: attaching the foreign element to the tissue with a suture that comprises an anti-scarring agent.

A “foreign element” refers to a tissue, organ, device, prostheses, or the like that is not originally part of the body of a patient.

In certain embodiments, a method for reducing, or reducing the risk of, excessive scarring resulting from a tissue repositioning surgery is provided, comprising: using a suture that comprises an anti-scarring agent in the tissue repositioning surgery.

In one aspect, a method for reducing, or reducing the risk of, suture granulomas is provided, comprising closing a wound with a suture that comprises an anti-scarring agent.

The drug dose (ie, the amount of anti-scarring agents) administered will depend on a variety of factors, including the type of formulation, the type of condition being treated, the surface area and design of the sutures. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than, to 50%, 20%, 10%, 5%, or even less than 1% of the concentration typically used in a single systemic dose application. In certain aspects, the anti-scarring agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In certain embodiments, the drug is released in effective concentrations for a period ranging from 1-90 days.

Anti-fibrosing agents, used alone or in combination, should be administered under the following dosing guidelines. The total amount (dose) of anti-scarring agent can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of suture surface to which the agent is applied may be in the range of about 0.01 μg/mm²-1 μg/mm², or 1 μg/mm²-10 μg/mm², or 10 μg/mm²-250 μg/mm², or 250 μg/mm²-500 μg/mm².

Provided below are exemplary dosage ranges for various anti-scarring agents that can be used in conjunction with sutures in accordance with the invention: A) Cell cycle inhibitors including doxorubicin and mitoxantrone. Doxorubicin analogues and derivatives thereof: total dose not to exceed 25 mg (range of 0.1 μg to 25 mg), preferred 1 μg to 5 mg The dose per unit area of suture surface to be 0.01 μg-100 μg per mm², preferred dose of 0.1 μg/mm²-10 μg/mm². Minimum concentration of 10⁻⁸-10⁻⁴ M of doxorubicin is to be maintained on the suture surface for the required duration of therapeutic effect. Mitoxantrone and analogues and derivatives thereof: total dose not to exceed 5 mg (range of 0.01 μg to 5 mg); preferred 0 1 μg to 1 mg. The dose per unit area of the suture of 0.01 μg-20 μg per mm²; preferred dose of 0.05 μg/mm²-μg/mm². Minimum concentration of 10⁻⁸-10⁴ M of mitoxantrone is to be maintained on the suture surface for the required duration of therapeutic effect. B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the suture of 0.1 μg-10 μg per mm²; preferred dose of 0.25 μg/mm²-5 μg/mm². Minimum concentration of 10⁻⁸-10⁻⁴ M of paclitaxel is to be maintained on the suture surface for the required duration of therapeutic effect. (C) Cell cycle inhibitors such as podophyllotoxins (e.g., etoposide): total dose not to exceed 10 mg (range of 0.1 μg to 10 mg); preferred 1 μg to 3 mg. The dose per unit area of the suture of 0.1 μg-10 μg per mm²; preferred dose of 0.25 μg/mm²-5 μg/mm². Minimum concentration of 10⁻⁸-10⁻⁴ M of etoposide is to be maintained on the suture surface for the required duration of therapeutic effect. (D) Immunomodulators including sirolimus and everolimus. Sirolimus (i.e., rapamycin, RAPAMUNE): Total dose not to exceed 10 mg (range of 0.1 μg to 10 mg); preferred 10 μg to 1 mg. The dose per unit area of the suture of 0.1 μg-100 μg per mm²; preferred dose of 0.5 μg/mm²-10 μg/mm². Minimum concentration of 10⁻⁸-10⁻⁴ M is to be maintained on the suture surface for the required duration of therapeutic effect. Everolimus and derivatives and analogues thereof: Total dose should not exceed 10 mg (range of 0.1 μg to 10 mg); preferred 10 μg to 1 mg. The dose per unit area of the suture of 0.1 μg-100 μg per mm² of surface area; preferred dose of 0.3 μg/mm²-10 μg/mm². Minimum concentration of 10⁻⁸-10⁻⁴ M of everolimus is to be maintained on the suture surface for the required duration of therapeutic effect (E) Heat shock protein 90 antagonists (e.g geldanamycin) and analogues and derivatives thereof, total dose not to exceed 20 mg (range of 0.1 μg to 20 mg), preferred 1 μg to 5 mg. The dose per unit area of the suture of 0.1 μg-10 μg per mm², preferred dose of 0.25 μg/mm²-5 μg/mm². Minimum concentration of 10⁻⁸-10⁻⁴ M of geldanamycin is to be maintained on the suture surface for the required duration of therapeutic effect. (F) HMGCoA reductase inhibitors (e.g., simvastatin) and analogues and derivatives thereof, total dose not to exceed 200 mg (range of 10.0 μg to 200 mg); preferred 10 μg to 200 mg. The dose per unit area of the suture of 1.0 μg-200 μg per mm²; preferred dose of 2.5 μg/mm²-200 μg/mm². Minimum concentration of 10⁻⁸-10⁻³ M of simvastatin is to be maintained on the suture surface for the required duration of therapeutic effect. (G) Inosine monophosphate dehydrogenase inhibitors (e.g., mycophenolic acid, 1-alpha-25 dihydroxy vitamin D₃) and analogues and derivatives thereof: total dose not to exceed 500 mg (range of 10.0 μg to 500 mg); preferred 10 μg to 300 mg. The dose per unit area of the suture of 1.0 μg-200 μg per mm²; preferred dose of 2.5 μg/mm²-200 μg/mm². Minimum concentration of 10⁻⁸-10⁻³ M of mycophenolic acid is to be maintained on the suture surface for the required duration of therapeutic effect. (H) NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the suture of 1.0 μg-100 μg per mm²; preferred dose of 2.5 μg/mm²-50 μg/mm². Minimum concentration of 10⁻⁸-10⁻⁴ M of Bay 11-7082 is to be maintained on the suture surface for the required duration of therapeutic effect. (I) Antimycotic agents (e.g., sulconizole) and analogues and derivatives thereof: total dose not to exceed 500 mg (range of 10.0 μg to 500 mg); preferred 10 μg to 300 mg. The dose per unit area of the suture of 1.0 μg-200 μg per mm²; preferred dose of 2.5 μg/mm²-200 μg/mm². Minimum concentration of 10⁻⁸-10⁻³ M of sulconizole is to be maintained on the suture surface for the required duration of therapeutic effect. (J) p38 MAP Kinase Inhibitors (e.g., SB202190) and analogues and derivatives thereof total dose not to exceed 500 mg (range of 10 0 μg to 500 mg); preferred 10 μg to 300 mg The dose per unit area of the suture of 1.0 μg-200 μg per mm², preferred dose of 2 5 μg/mm²-200 mg/mm² Minimum concentration of 10⁻⁸-10⁻³ M of SB202190 is to be maintained on the suture surface for the required duration of therapeutic effect. (K) anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the suture of 0.1 μg-10 μg per mm²; preferred dose of 0.25 μg/mm² 5 μg/mm² Minimum concentration of 10⁻⁸-10⁻⁴ M of halofuginone bromide is to be maintained on the suture surface for the required duration of therapeutic effect.

In addition to those described above (e.g., sirolimus, everolimus, and tacrolimus), several other examples of immunomodulators and appropriate dosages ranges for use with sutures include the following: (A) Biolimus and derivatives and analogues thereof: Total dose should not exceed 10 mg (range of 0.1 μg to 10 mg); preferred 10 μg to 1 mg. The dose per unit area of the suture of 0.1 μg-100 μg per mm² of surface area; preferred dose of 0.3 μg/mm²-10 μg/mm². Minimum concentration of 10⁻⁸-10⁻⁴ M of everolimus is to be maintained on the suture surface for the required duration of therapeutic effect. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area of the suture; preferred dose of 0.3 μg/mm²-10 μg/mm². Minimum concentration of 10⁻⁸-10⁻⁴ M of tresperimus is to be maintained on the suture surface for the required duration of therapeutic effect. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area of the suture; preferred dose of 0.3 μg/mm²-10 μg/mm². Minimum concentration of 10⁻⁸-10⁻⁴ M of auranofin is to be maintained on the suture surface for the required duration of therapeutic effect. (D) 27-0-Demethylrapamycin and derivatives and analogues thereof. Total dose should not exceed 10 mg (range of 0.1 μg to 10 mg), preferred 10 μg to 1 mg. The dose per unit area of the suture of 0.1 μg-100 μg per mm² of surface area, preferred dose of 0.3 μg/mm²-10 μg/mm². Minimum concentration of 10⁻⁸-10⁻⁴ M of 27-0-Demethylrapamycin is to be maintained on the suture surface for the required duration of therapeutic effect. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area of the suture; preferred dose of 0.3 μg/mm²-10 μg/mm². Minimum concentration of 10⁻⁸-10⁻⁴ M of gusperimus is to be maintained on the suture surface for the required duration of therapeutic effect. (F) Pimecrolimus and derivatives and analogues thereof: Total dose should not exceed 10 mg (range of 0.1 μg to 10 mg); preferred 10 μg to 1 mg. The dose per unit area of the suture of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the suture surface for the required duration of therapeutic effect and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area of the suture; preferred dose of 0.3 μg/mm²-10 μg/mm². Minimum concentration of 10⁻⁸-10⁻⁴ M of ABT-578 is to be maintained on the suture surface for the required duration of therapeutic effect.

Additional exemplary dosage ranges for various anti-scarring agents that can be used in conjunction with sutures in accordance with the invention include: (A) Angiogenesis inhibitors including Alphastatin, ZD-6474, IDN-5390, SB-2723005, ABT-518, combretastatin, and anecortane, analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (B) mTOR inhibitors including AP-23573, analogues and derivatives thereof: total dose not to exceed 200 mg (range of 0.1 μg to 200 mg); preferred 1 μg to 100 mg Dose per unit area of the suture of 0.01 μg-100 μg per mm²; preferred dose of 0.1 μg/mm²-20 μg/mm². Minimum concentration of 10⁻⁸-10⁴ M of agent is to be maintained on the suture surface for the required duration of therapeutic effect. (C) Tubulin antagonists including synthadotin, analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (D) Epithilones including ixabepilone and analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (E) Kinesin Antagonists including SB-715992 and analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (F) TNF alpha antagonists including Etanercept, Humicade, Adalimumab and analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (G) AKT inhibitor including erucylphosphocholine and analogues and derivatives thereof: total dose not to exceed 200 mg (range of 0.1 μg to 200 mg); preferred 1 μg to 100 mg. Dose per unit area of the suture of 0.01 μg-100 μg per mm², preferred dose of 0.1 μg/mm²-20 μg/mm² Minimum concentration of 10⁻⁸-10⁻⁴ M of agent is to be maintained on the suture surface for the required duration of therapeutic effect. (H) FGF Inhibitors including IDN-5390 and analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (I) Collagenase Antagonists including S-0885 and analogues and derivatives thereof: total dose not to exceed 500 mg (range of 0.1 μg to 500 mg); preferred 1 μg to 500 mg. Dose per unit area of the suture of 0.01 μg-200 μg per mm²; preferred dose of 0.1 μg/mm²-100 μg/mm². Minimum concentration of 10⁻⁸-10⁻³ M of agent is to be maintained on the suture surface for the required duration of therapeutic effect. (J) NF KAPPA B Inhibitors including BXT-51072 and analogues and derivatives thereof: total dose not to exceed 200 mg (range of 0.1 μg to 200 mg); preferred 1 μg to 100 mg. Dose per unit area of the suture of 0.01 μg-100 μg per mm²; preferred dose of 0.1 μg/mm²-20 μg/mm². Minimum concentration of 10⁻⁸-10⁻⁴ M of agent is to be maintained on the suture surface for the required duration of therapeutic effect. (K) Elongation Factor-1 alpha inhibitors including aplidine and analogues and derivatives thereof: total dose not to exceed 500 mg (range of 0.1 μg to 500 mg); preferred 1 μg to 500 mg. Dose per unit area of 0.01 μg-200 μg per mm²; preferred dose of 0.1 μg/mm²-100 μg/mm². Minimum concentration of 10⁻⁸-10⁻⁴ M of agent is to be maintained on the suture surface for the required duration of therapeutic effect. (L) Tyrosine kinase inhibitors including Gefitinib and analogues and derivatives thereof: total dose not to exceed 500 mg (range of 0.1 μg to 500 mg); preferred 1 μg to 100 mg. Dose per unit area of suture of 0.01 μg-200 μg per mm²; preferred dose of 0.1 μg/mm²-100 μg/mm². Minimum concentration of 10⁻⁸-10⁻⁴ M of agent is to be maintained on the suture surface for the required duration of therapeutic effect.

Exemplary uses of anti-scarring sutures (ie, in reducing, or reducing the risk of, a keloid, a hypertrophic scar, surgical adhesion, or suture granulomas) are described in more detail below.

### Hypertrophic Scars or Keloids

In one aspect, a method for reducing a hypertrophic scar or ketoid resulting from introduction (used interchangeably with “implantation”) of sutures into the skin is provided, comprising: implanting a suture that comprises an anti-scarring agent or a composition thereof into the skin.

Hypertrophic scars and keloids are an overgrowth of dense fibrous tissue that is the result of an excessive fibroproliferative wound healing process. Hypertrophic scars and keloids usually develop after healing of a skin injury. Briefly, healing of wounds and scar formation occurs in three phases: inflammation, proliferation, and maturation. The first phase, inflammation, occurs in response to an injury that is severe enough to break the skin. During this phase, which lasts 3 to 4 days, blood and tissue fluid form an adhesive coagulum and fibrinous network that serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months.

If too much connective tissue is produced and the wound remains persistently cellular, the scar may become red and raised. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds With hypertrophic scars, some degree of maturation occurs and gradual improvement occurs In the case of keloids however, an actual tumor is produced which can become quite large Spontaneous improvement in such cases rarely occurs.

Keloids and hypertrophic scars located at most sites are primarily of cosmetic concern; however, some keloids or hypertrophic scars can cause contractures, which may result in a loss of function if overlying a joint, or they can cause significant disfigurement if located on the face. Both keloids and hypertrophic scars can be painful or pruritic.

Anti-scarring agents or anti-scarring compositions as described above, may be combined with sutures as described herein to prevent the progression of these lesions. Exemplary anti-scarring agents especially useful in reducing, or reducing the risk of (i.e., preventing) hypertropic scars or keloids include halofuginone, paclitaxel, rapamycin, and terbinafine.

Surgical Adhesion

In one aspect, a method for reducing, or reducing the risk of, surgical adhesion is provided, comprising: performing a surgery using a suture that comprises an anti-scarring agent.

Surgical adhesion or risk thereof that may be reduced includes any adhesion resulting from a surgery where sutures are used. Such surgery includes, but is not limited to, abdominal surgery, gynecological surgery, and hysterectomies.

In certain embodiments, sutures that comprise an anti-scarring agent may release a sufficient amount of the anti-scarring agent to inhibit the excess scarring at their implantation site. Such inhibition may reduce, or reduce the risk of, surgical adhesion between the tissue at the suture implantation site and a nearby tissue or between different portions of the same tissue at the suturing site.

Suture Granulomas

In one aspect, a method for reducing, or reducing the risk of, suture granulomas is provided, comprising closing a wound with a suture that comprises an anti-scarring agent.

Suture granuloma, commonly known as a stitch abscess, is a fairly common benign complication seen after surgery. Suture material is a foreign body that causes local irritation and tissue necrosis. Suture granuloma can occur many years after the primary surgical procedure. At skin or subcutaneous level, the granuloma presents as a chronic intermittent indolent infection with a burrow sinus, with no fever or signs of systemic infection. The intra-abdominal presence of foreign material is an important cause of adhesion formation. Suture granulomas mimic neoplasms in clinical appearance. Suture granuloma can occur in the bronchial stump after lung resection, in the lung parenchyma after segmentectomy or as a paravesical mass or abscess after inguinal hernia repair. The paravesical abscess granuloma causes urinary discomfort, swelling, tenderness and microscopic hematuria.

In certain embodiments, the anti-scarring agents or compositions on the anti-scarring sutures described herein may reduce, or reduce the risk of, local irritation, tissue necrosis, and complications associated thereof due to the presence of suture material in the tissue.

Infiltrating Suture-Contacting Tissues with Anti-Scarring Agents or Compositions

In one aspect, a method for reducing, or reducing the risk of, excessive scarring (e.g., hypertropic scars or keloids) or side effects thereof (e.g., surgical adhesion) is provided, comprising: infiltrating a tissue at which a suture has been, is being, or to be, implanted with an effective amount of an anti-scarring agent or a composition comprising an anti-scarring agent.

In another aspect, a method for reducing, or reducing the risk of, suture granulomas is provided, comprising infiltrating a tissue at which a suture has been, is being, or to be, implanted with an effective amount of an anti-scarring agent or a composition comprising an anti-scarring agent.

In certain embodiments, polymers or polymer compositions effective in reducing scarring, irritation, or tissue necrosis or as an adhesion barrier may be used to infiltrate the tissue where a suture has been, is being, or to be implanted or to be combined with a suture before its implantation. Such polymers may be used alone or with an anti-scarring agent.

The infiltration of polymers, polymer compositions, or compositions that comprise an anti-scarring agent or another therapeutic agent can be accomplished in several ways including: (a) topical application of the agent into the site where the suture will be placed (particularly useful for this embodiment is the use of polymeric carriers which release an anti-scarring agent or another therapeutic agent over a period ranging from several hours to several weeks. Compositions that can be used for this application include, e.g., fluids, microspheres, pastes, gels, microparticulates, sprays, aerosols, solid implants and other formulations which release an anti-scarring agent or another therapeutic agent into the region where the suture will be implanted); (b) microparticulate forms of the therapeutic agent are also useful for directed delivery into the implantation site; (c) sprayable collagen-containing formulations such as COSTASIS and crosslinked derivatized poly(ethylene glycol) collagen compositions (described, e.g., in U.S. Pat. Nos. 5,874,500 and 5,565,519 and referred to herein as “CT3” (both from Angiotech Pharmaceuticals, Inc., Canada), either alone, or loaded with a therapeutic agent, applied to the implantation site; (d) sprayable PEG-containing formulations such as COSEAL or ADHIBIT (Angiotech Pharmaceuticals, Inc.), SPRAYGEL or DURASEAL (both from Confluent Surgical, Inc., Boston, Mass.), either alone, or loaded with a therapeutic agent, applied to the implantation site; (e) fibrin-containing formulations such as FLOSEAL or TISSEEL (both from Baxter Healthcare Corporation, Fremont, Calif.), applied to the implantation site; (f) hyaluronic acid-containing formulations such as RESTYLANE or PERLANE (both from Q-Med AB, Sweden), HYLAFORM (Inamed Corporation (Santa Barbara, Calif.)), SYNVISC (Biomatrix, Inc., Ridgefield, N.J.), SEPRAFILM or SEPRACOAT (both from Genzyme Corporation, Cambridge, Mass.) loaded with a therapeutic agent applied to the implantation site; (g) polymeric gels for surgical implantation such as REPEL (Life Medical Sciences, Inc., Princeton, N.J.) or FLOGEL (Baxter Healthcare Corporation) loaded with a therapeutic agent applied to the implantation site; (h) orthopedic “cements” used to hold prostheses and tissues in place with a therapeutic agent applied to the implantation site; (i) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND II (Veterniary Products Laboratories, Phoenix, Ariz.), VETBOND (3M Company, St. Paul, Minn.), HISTOACRYL BLUE (Davis & Geck, St. Louis, Mo.) and ORABASE SMOOTHE-N-SEAL Liquid Protectant (Colgate-Palmolive Company, New York, N.Y.) loaded with a therapeutic agent, applied to the implantation site; and/or (j) protein-based sealants or adhesives such as BIOGLUE (Cryolife, Inc.) and TISSUEBOND (TissueMed, Ltd.) loaded with a therapeutic agent, applied to the implantation site.

In one embodiment, polymers that useful for infiltrating a tissue at the suture implantation site may be those that can form a covalent bond with the tissue to which it is applied. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In this embodiment, the 4 armed NHS-derivatized polyethylene glycol is dissolved in an acidic solution (pH about 2-3) and is then co-applied to the tissue using a basic buffer (pH >about 8) The antifibrosisfibrosis-inhibiting agent(s) may be incorporated directly into the 4 armed NHS-derivatized polyethylene glycol, the acidic solution, or the basic buffer.

In another embodiment, a fibrosis-inhibiting agent may be incorporated into a secondary carrier that may then be incorporated into the 4 armed NHS-derivatized polyethylene glycol, the acidic solution and/or the basic buffer. The secondary carriers may include microparticles and/or microspheres that are made from degradable polymers. The degradable polymers may include polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X (where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

In another embodiment, a tissue reactive polymer may be applied initially and then a fibrosis-inhibiting agent may then be applied to the coated tissue. The fibrosis-inhibiting agent may be applied directly to the tissue or it may be incorporated into a secondary carrier. The secondary carriers may include microspheres (as described above), microparticles (as described above), gels (eg., hyaluronic acid, carboxymethyl cellulose, dextran, poly(ethylene oxide)-poly(propylene oxide) block copolymers as well as blends, association complexes and crosslinked compositions thereof) and films (degradable polyesters, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, and block copolymers of the form X—Y, Y—X—Y, R—(Y—X)ₙ, R—(X—Y)ₙ and X—Y—X where X in a polyalkylene oxide (e.g., poly(ethylene glycol, poly(propylene glycol) and block copolymers of poly(ethylene oxide) and poly(propylene oxide) (e.g., PLURONIC and PLURONIC R series of polymers from BASF Corporation, Mount Olive, N.J.) and Y is a biodegradable polyester, where the polyester may comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator, hyaluronic acid, carboxymethyl cellulose, dextran, poly(ethylene oxide)-poly(propylene oxide) block copolymers as well as blends, association complexes and crosslinked compositions thereof.

In one embodiment, a polymeric matrix which can be used to help prevent the formation of fibrous tissue that leads to hypertropic scars, keloids, or surgical adhesions or prevent tissue irritation or necrosis, either alone or in combination with a fibrosis inhibiting agent/composition, is formed from reactants comprising either one or both of pentaerythritol poly(ethylene glycol)ether tetra-sulfhydryl (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue.

Other examples of polymeric compositions that can be infiltrated into the suture implantation site with or without an additional fibrosis-inhibiting (and/or an anti-infective) therapeutic agent for the reduction or prevention of excessive scarring and its resulting hypertropic scars, keloids, or surgical adhesions, include a variety of commercial products. For example, Confluent Surgical, Inc. makes their DURASEAL, which is a synthetic hydrogel designed to augment sutured dura closures following cranial surgical procedures. Products that are being developed by Confluent Surgical, Inc. are described in, for example, U.S. Pat. No. 6,379,373. FzioMed, Inc. (San Luis Obispo, Calif.) makes OXIPLEXISP Gel that is being sold as an adhesion barrier for spine surgery. OXIPLEX/SP Gel is being used for the reduction of pain and radiculopathy in laminectomy, laminotomy and discectomy surgeries. Products being developed by FzioMed, Inc. are described in, for example, U.S. Pat. Nos. 6,566,345 and 6,017,301. Anika Therapeutics, Inc. (Woburn, Mass.) is developing INCERT-S for the prevention of internal adhesions or scarring following spinal surgery INCERT-S is part of a potential family of bioabsorbable, chemically modified hyaluronic acid therapies Products being developed by Anika Therapeutics, Inc are described in, for example, U.S. Pat. Nos. 6,548,081; 6,537,979; 6,096,727; 6,013,679; 5,502,081 and 5,356,883. Life Medical Sciences, Inc. (Little Silver, N.J.) is developing RELIEVE as a bio-resorbable polymer designed to prevent or reduce the formation of adhesions that can follow spinal surgery. Products being developed by Life Medical Sciences, Inc. are described in, for example, U.S. Pat. Nos. 6,696,499; 6,399,624; 6,211,249; 6,136,333 and 5,711,958. Wright Medical Technology, Inc. is selling the ADCON range of products which are dextran sulfate gels originally developed by Gliatech, Inc. (Beachwood, Ohio) to inhibit postsurgical peridural fibrosis that occurs in posterior lumbar laminectomy or laminotomy procedures where nerve routes are exposed. The ADCON range of products may be described in, for example, U.S. Pat. Nos. 6,417,173; 6,127,348; 6,083,930; 5,994,325 and 5,705,178.

Other commercially available materials that may be used alone or loaded with a therapeutic agent (e.g., a fibrosis-inhibiting agent and/or an anti-infective agent), applied to or infiltrated into a spinal or neurosurgical site (or to an implant surface) for the prevention of adhesions include: (a) sprayable collagen-containing formulations such as COSTASIS or CT3; (b) sprayable PEG-containing formulations such as COSEAL, ADHIBIT, FOCALSEAL, or SPRAYGEL; (c) fibrinogen-containing formulations such as FLOSEAL or TISSEAL (both from Baxter Healthcare Corporation, Fremont, Calif.); (d) hyaluronic acid-containing formulations such as RESTYLANE, PERLANE, HYLAFORM, SYNVISC, SEPRAFILM or SEPRACOAT; (e) polymeric gels for surgical implantation such as REPEL or FLOWGEL; (f) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT; (h) lipid based compositions such as ADSURF, and (j) film compositions such as INTERCEED (Ethicon, Inc., Somerville, N.J.) and HYDROSORB (MacroPore Biosurgery, Inc., San Diego, Calif./Medtronic Sofamor Danek, Memphis. Tenn.) It should be obvious to one of skill in the art that commercial compositions not specifically cited above as well as next-generation and/or subsequently-developed commercial products are to be anticipated and are suitable for use under the present invention.

In certain embodiments, topical and injectable compositions that include an anti-scarring agent and a polymeric carrier are provided for application on or into hypertrophic scars or keloids. Incorporation of a fibrosis-inhibiting agent into a topical formulation or an injectable formulation is one approach to treat this condition. Numerous polymeric and non-polymeric delivery systems for use in treating hypertrophic scars or keloids have been contemplated for prevention and reduction of these lesions. The topical formulation can be in the form of a solution, a suspension, an emulsion, a gel, an ointment, a cream, film or mesh. The injectable formulation can be in the form of a solution, a suspension, an emulsion or a gel. Polymeric and non-polymeric components that can be used to prepare these topical or injectable compositions are further described in, for example, WO 05/051452.

Any anti-scarring agents or compositions as described above may be used to infiltrate a tissue at which a suture has been, is being, or to be implanted to reduce hypertrophic scars, keloids, surgical adhesion, suture granulomas, or the risk thereof. Anti-scarring agents especially useful in reducing, or reducing the risk of, hypertropic scars or keloids include halofuginone, paclitaxel, rapamycin, and terbinafine.

The total amount (dose) of anti-scarring agent(s) useful for infiltrating a tissue at a suture implantation site may be in the range of about 0.01 μg-10 μg, or 10 μg-10 mg, or 10 mg-250 mg, or 250 mg-1000 mg, or 1000 mg-2500 mg. The dose (amount) of anti-scarring agent(s) per unit area of tissue surface to which the agent is applied may be in the range of about 0.01 μg/mm²-1 μg/mm², or 1 μg/mm²-10 μg/mm², or 10 μg/mm²-250 μg/mm², or 250 μg/mm²-500 μg/mm².

It should also be readily evident to one of skill in the art that any of the previously described anti-scarring agents, or derivatives and analogues thereof, can be utilized to create variations of the above compositions without deviating from the spirit and scope of the Invention. It should also be apparent that the agent can be utilized in a composition with or without polymer carrier and that altering the carrier does not deviate from the scope of this invention.

The invention having been described, the following examples are intended to illustrate, and not limit, the invention.

### EXAMPLES

### Example 1

### Dip Coating a Suture

Poly(lactide-co-glycolide) [30/70] (PLG) is dissolved in 10 ml dichloromethane to produce a solution that has a polymer concentration of approximately 40 mg/mL. Paclitaxel is added to the PLG solution to produce a final paclitaxel concentration of 3 mg/mL. A monofilament polydioxanone suture (size 3-0) is cleaned by immersing the filter in isopropanol for 30 minutes and then rinsing 3 times with isopropanol. The suture is air-dried. The suture is dip coated by completely immersing the cleaned suture into the PLG-paclitaxel solution. The suture is then removed from the solution and is air-dried. This process may be repeated until the desired paclitaxel dose is achieved. The suture is then dried under vacuum. Other fibrosis-inhibiting agents that may be coated onto a suture using this procedure include halofuginone, rapamycin, everolimus, and pimecerolimus.

### Example 2

### Spray Coating a Suture

A 2% solution of poly(glycolide-co-caprolactone) [10/90] {PGC} is prepared using dichmoromethane as the solvent. Paclitaxel is added to the PGC solution to produce a final paclitaxel concentration of 3 mg/mL. The PGC-paclitaxel solution is then transferred to the reservoir of an artist's airbrush tool. A monofilament polydioxanone suture (size 3-0) is cleaned by immersing the suture into isopropanol for 30 minutes and then rinsing 3 times with isopropanol. The suture is air-dried. Using a crocodile clip, the suture is suspended in the air and is spray coated from several angles to ensure complete coating of the suture. Once the coating is dry to the touch, the suture is removed from the clip and the uncoated portion is spray coated. The suture is then air dried and/or vacuum dried to remove the solvent. This process may be repeated until the desired paclitaxel dose is achieved. The suture is then dried under vacuum. Other fibrosis-inhibiting agents that may be coated onto a suture using this procedure include halofuginone, rapamycin, everolimus, and pimecerolimus.

### Example 3

### Application of a Second Coating to a Suture

Poly(lactide-co-glycolide) [30/70] (PLG) is dissolved in 10 ml dichloromethane to produce a solution that has a polymer concentration of approximately 40 mg/mL. Halofuginone is added to the PLG solution to produce a final halofuginone concentration of 3 mg/mL. A monofilament polydioxanone suture (size 3-0) is cleaned by immersing the suture into isopropanol for 30 minutes and then rinsing 3 times with isopropanol. The filter is air-dried. The suture is dip coated by completely immersing the cleaned filter into the PLG-halofuginone solution. The suture is then removed from the solution and is air dried. This process may be repeated until the desired halofuginone dose is achieved. The suture is then dried under vacuum to remove the residual solvent. The suture is then dipped into an aqueous solution of sodium hyaluronate [HA] (mw approximately 1-1.5×10⁶ kDa, 10 mg/mL). The water is removed by air-drying at 37° C. The process is repeated until the desired amount of HA is coated onto the suture. The suture is then dried under vacuum. Other fibrosis-inhibiting agents that may be coated onto a suture using this procedure include paclitaxel, rapamycin, everolimus, and pimecerolimus

### Example 4

### Coating Containing Two Bioactive Agents for a Suture

poly(glycolide-co-caprolactone) [10/90] {PGC} is dissolved in 10 ml dichloromethane to produce a solution that has a polymer concentration of approximately 40 mg/mL. Paclitaxel is added to the PGC solution to produce a final paclitaxel concentration of 3 mg/mL. Heparin-benzalkonium chloride is then added to the PCG solution to achieve a final concentration of 1 mg/ml. A monofilament polypropylene suture (size 2-0) is cleaned by immersing the suture into isopropanol for 30 minutes and then rinsing 3 times with isopropanol. The suture is air-dried. The suture is dip coated by completely immersing the cleaned suture into the PGC-paclitaxel solution. The suture is the removed from the solution and is air-dried. This process may be repeated until the desired paclitaxel dose is achieved. The suture is then dried under vacuum. Other fibrosis-inhibiting agents that may be coated onto a suture using this procedure include halofuginone, rapamycin, everolimus, and pimecerolimus.

### Example 5

### Two Coating Layers Containing Two Different Bioactive Agents for a Suture

poly(glycolide-co-caprolactone) [10/90] {PGC} is dissolved in 10 ml dichloromethane to produce a solution that has a polymer concentration of approximately 40 mg/mL. Rapamycin is added to the PGC solution to produce a final Rapamycin concentration of 3 mg/mL. A monofilament polydioxanone suture (size 3-0) is cleaned by immersing the suture into isopropanol for 30 minutes and then rinsing 3 times with isopropanol. The suture is air-dried. The suture is dip coated by completely immersing the cleaned suture into the PGC-rapamycin solution. The suture is the removed from the solution and is air-dried. This process may be repeated until the desired rapamycin dose is achieved. The suture is then dried under vacuum to remove the residual solvent. The suture is then dipped into an aqueous solution of sodium hyaluronate [HA] (mw approximately 1-1.5×10⁶ kDa, 10 mg/mL) that contains 1 mg/ml heparin. The water is removed by air drying at 37° C. The process is repeated until the desired amount of HA is coated onto the suture. The suture is then dried under vacuum. Other fibrosis-inhibiting agents that may be coated onto a suture using this procedure include halofuginone, paclitaxel, everolimus, and pimecerolimus.

### Example 6

### Drug Incorporation into a Suture

A solution of halofuginone is prepared by dissolving 70 mg halofuginone in 10 mL water/ethanol (1:1) in a 20 mL glass scintillation vial. A polypropylene suture (2-0) is immersed in the solution. The solution is placed in an ultrasonic bath (Fisher) for 1 min. The suture is removed using a pair of tweezers. The suture is air dried for 3 hours after which it is dried under vacuum for 24 hours. Other fibrosis-inhibiting agents that may be coated onto a suture using this procedure include rapamycin, paclitaxel, everolimus, and pimecerolimus.

### Example 7

### Drug Incorporation into a Suture

Five 15 mL solutions of paclitaxel at 5 mg/ml are prepared in methanol in a 20 mL scintillation vial. A polypropylene suture (2-0) is immersed in each of the paclitaxel solutions. The sutures are removed from the paclitaxel solutions at 30 minutes, 1 hour, 2 hours, 6 hours and 24 hours. The sutures are air dried and then dried under vacuum for 24 hours. Other fibrosis-inhibiting agents that may be coated onto a suture using this procedure include rapamycin, halofuginone, everolimus, and pimecerolimus.

### Example 8

### Drug Incorporation into a Suture

Five 15 mL solution of paclitaxel (5 mg/mL) and 5-fluorouracil (4 mg/mL) are prepared in methanol in a 20 mL scintillation vial. A polypropylene suture (2-0) is then immersed in each of the paclitaxel solutions. The sutures are removed from the paclitaxel solutions at 30 minutes, 1 hour, 2 hours, 6 hours and 24 hours The sutures are air dried and then dried under vacuum for 24 hours Other fibrosis-inhibiting agents that may be coated onto a suture using this procedure include halofuginone, rapamycin, everolimus, and pimecerolimus.

### Example 9

### Screening Assay for Assessing the Effect of Various Compounds on Nitric Oxide Production by Macrophages

The murine macrophage cell line RAW 264.7 was trypsinized to remove cells from flasks and plated in individual wells of a 6-well plate. Approximately 2×10⁶ cells were plated in 2 ml of media containing 5% heat-inactivated fetal bovine serum (FBS). RAW 264.7 cells were incubated at 37° C. for 1.5 hours to allow adherence to plastic. Mitoxantrone was prepared in DMSO at a concentration of 10⁻² M and serially diluted 10-fold to give a range of stock concentrations (10⁻⁸ M to 10⁻² M). Media was then removed and cells were incubated in 1 ng/ml of recombinant murine IFNγ and 5 ng/ml of LPS with or without mitoxantrone in fresh media containing 5% FBS. Mitoxantrone was added to cells by directly adding mitoxantrone DMSO stock solutions, prepared earlier, at a 1/1000 dilution, to each well. Plates containing IFNγ, LPS plus or minus mitoxantrone were incubated at 37° C. for 24 hours (Chem. Ber. (1879) 12: 426; J. AOAC (1977) 60-594; Ann. Rev. Biochem. (1994) 63:175).

At the end of the 24-hour period, supernatants were collected from the cells and assayed for the production of nitrites. Each sample was tested in triplicate by aliquoting 50 μL of supernatant in a 96-well plate and adding 50 μL of Greiss Reagent A (0.5 g sulfanilamide, 1.5 ml H₃PO₄, 48.5 ml ddH₂O) and 50 μL of Greiss Reagent B (0.05 g N-(1-naphthyl)-ethylenediamine, 1.5 ml H₃PO₄, 48.5 ml ddH₂O). Optical density was read immediately on microplate spectrophotometer at 562 nm absorbance. Absorbance over triplicate wells was averaged after subtracting background and concentration values were obtained from the nitrite standard curve (1 μM to 2 mM) Inhibitory concentration of 50% (CO₅₀) was determined by comparing average nitrite concentration to the positive control (cell stimulated with IFNγ and LPS) An average of n=4 replicate experiments was used to determine IC₅₀ values for mitoxantrone (see FIG. 1 (IC₅₀=927 nM)). The IC₅₀ values for the following additional compounds were determined using this assay: IC₅₀ (nM): paclitaxel, 7; CNI-1493, 249; halofuginone, 12; geldanamycin, 51; anisomycin, 68; 17-AAG, 840; epirubicin hydrochloride, 769.

### Example 10

### Screening Assay for Assessing the Effect of Various Anti-Scarring Agents on TNF-Alpha Production by Macrophages

The human macrophage cell line, THP-1 was plated in a 12-well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. Bay 11-7082 was prepared in DMSO at a concentration of 10⁻² M and serially diluted 10-fold to give a range of stock concentrations (10⁻⁸ M to 10⁻² M) (J. Immunol. (2000) 165:411-418; J. Immunol. (2000) 164:4804-4811; J. Immunol. Meth. (2000) 235 (1-2):33-40).

THP-1 cells were stimulated to produce TNFα by the addition of 1 mg/ml opsonized zymosan. Bay 11-7082 was added to THP-1 cells by directly adding DMSO stock solutions, prepared earlier, at a 1/1000 dilution, to each well. Each drug concentration was tested in triplicate wells. Plates were incubated at 37° C. for 24 hours.

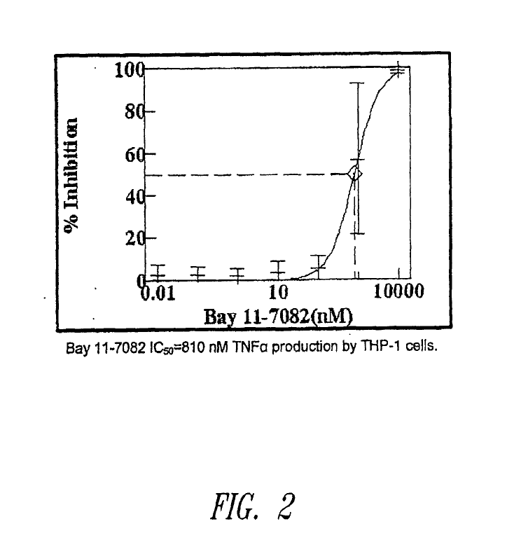

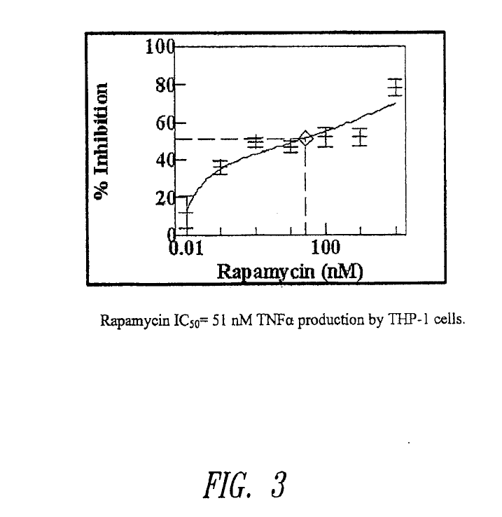

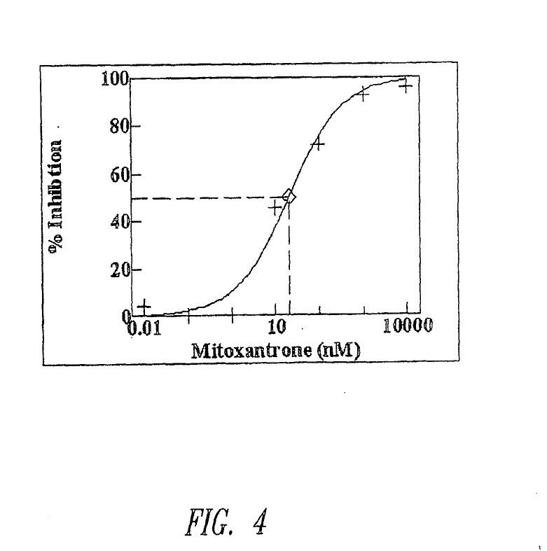

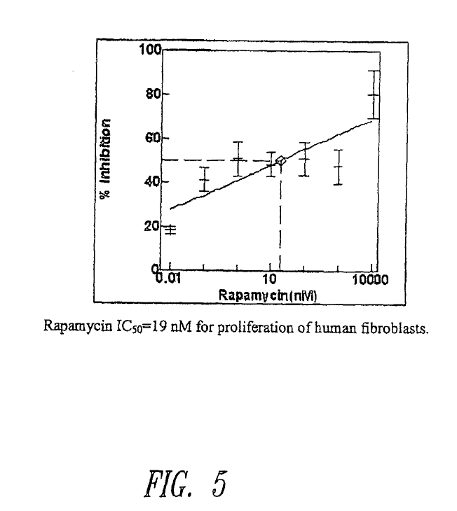

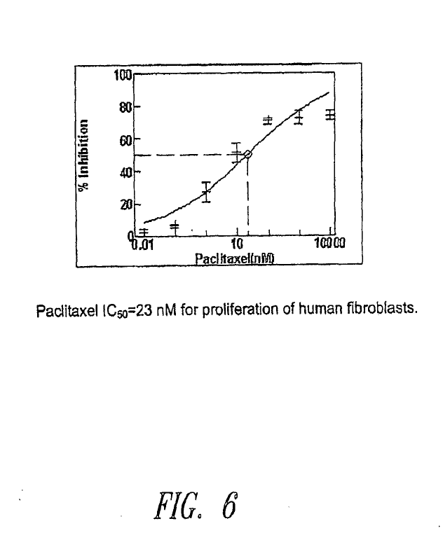

After a 24-hour stimulation, supernatants were collected to quantify TNFα production. TNFα concentrations in the supernatants were determined by ELISA using recombinant human TNFα to obtain a standard curve A 96-well MaxiSorb plate was coated with 100 μL of anti-human TNFα Capture Antibody diluted in Coating Buffer (0.1M sodium carbonate pH 9 5) overnight at 4° C. The dilution of Capture Antibody used was lot-specific and was determined empirically. Capture antibody was then aspirated and the plate washed 3 times with Wash Buffer (PBS, 0.05% TWEEN-20) Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/8 and 1/16; (b) recombinant human TNFα was prepared at 500 pg/ml and serially diluted to yield as standard curve of 7.8 pg/ml to 500 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human TNFα detection antibody+avidin-HRP) for 1 hour at room temperature. Following this incubation, the plates were washed 7 times and 100 μL of Substrate Solution (tetramethylbenzidine, H₂O₂) was added to plates and incubated for 30 minutes at room temperature. Stop Solution (2 NH₂SO₄) was then added to the wells and a yellow color reaction was read at 450 nm with A correction at 570 nm. Mean absorbance was determined from triplicate data readings and the mean background was subtracted. TNFα concentration values were obtained from the standard curve. Inhibitory concentration of 50% (IC₅₀) was determined by comparing average TNFα concentration to the positive control (THP-1 cells stimulated with opsonized zymosan). An average of n=4 replicate experiments was used to determine IC₅₀ values for Bay 11-7082 (see FIG. 2; IC₅₀=810 nM) and rapamycin (IC₅₀=51 nM; FIG. 3). The IC₅₀ values for the following additional compounds were determined using this assay: IC₅₀ (nM): geldanamycin, 14; mycophenolic acid, 756; mofetil, 792; chlorpromazine, 6; CNI-1493, 0.15; SKF 86002, 831; 15-deoxy prostaglandin J2, 742; fascaplycin, 701, podophyllotoxin, 75, mithramycin, 570, daunorubicin, 195, celastrol, 87, chromomycin A3, 394, vinorelbine, 605, vinblastine, 65.

### Example 11

### Surgical Adhesions Model to Assess Fibrosis Inhibiting Agents in Rats

The rat caecal sidewall model is used to as to assess the anti-fibrotic capacity of formulations in vivo. Sprague Dawley rats are anesthetized with halothane. Using aseptic precautions, the abdomen is opened via a midline incision. The caecum is exposed and lifted out of the abdominal cavity. Dorsal and ventral aspects of the caecum are successively scraped a total of 45 times over the terminal 1.5 cm using a #10 scalpel blade. Blade angle and pressure are controlled to produce punctate bleeding while avoiding severe tissue damage. The left side of the abdomen is retracted and everted to expose a section of the peritoneal wall that lies proximal to the caecum. The superficial layer of muscle (transverses abdominis) is excised over an area of 1×2 cm², leaving behind torn fibers from the second layer of muscle (internal oblique muscle). Abraded surfaces are tamponaded until bleeding stops. The abraded caecum is then positioned over the sidewall wound and attached by two sutures. The formulation is applied over both sides of the abraded caecum and over the abraded peritoneal sidewall. A further two sutures are placed to attach the caecum to the injured sidewall by a total of 4 sutures and the abdominal incision is closed in two layers. After 7 days, animals are evaluated post mortem with the extent and severity of adhesions being scored both quantitatively and qualitatively.

### Example 12

### Surgical Adhesions Model to Assess Fibrosis Inhibiting Agents in Rabbits

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 13

### Screening Assay for Assessing the Effect of Compounds on Cell Proliferation

A screening assay was conducted on numerous compounds to determining their effect on cell proliferation (In vitro toxicol. (1990) 3: 219; Biotech. Histochem. (1993) 68: 29; Anal. Biochem. (1993) 213: 426).

Fibroblasts at 70-90% confluency are trypsinized, replated at 600 cells/well in media in 96-well plates and allowed to attachment overnight. Compound is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and drug substance are incubated at 37° C. for 72 hours.

To terminate the assay, the media is removed by gentle aspiration. A 1/400 dilution of CYQUANT 400×GR™ dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Inhibitory concentration of 50% (IC₅₀) is determined by taking the average of triplicate wells and comparing average relative fluorescence units to the DMSO control. An average of n=4 replicate experiments is used to determine IC₅₀ values.

The following compounds had an IC₅₀ value between 0.1 nM and 10 nM; Dolastatin 15; Staurosporine, Streptomyces sp.; Streptokinase from Streptococcus hemolyticus; Vinblastine sulfate salt; Vinblastine sulfate salt; Josamycin; Geldanamycin from Streptomyces hygroscopicus; Actinomycin D, D actinomycin, Actinomycin IV, Dactinomycin, Actinomycin C1; Thapsigargin; Vincristine sulfate salt; Pirarubicin; Camptothecin, Camptothecine; 7-Ethyl-10-hydroxycamptothecin; Theophylline; Vincamine; Aclarubicin; Gemcitabine Hydrochloride, dFdC, dFdCyd, Warfarin, 3-(a-Acetonylbenzyl)-4-hydroxycoumarin sodium salt, 4-Hydroxy-3-(3-oxo-1-phenylbutyl)coumarin, 4-Hydroxy-3-(3-oxo-1-phenylbutyl)coumarin; Idarubicin hydrochloride, DMDR, IMI-30, Idamycin; 17-DMAG, Geldanamycin Analog, KOS-1022; Hesperetin; Nogalamycin from Streptomyces nogalater; Vinorelbine ditartrate salt, 5-Noranhydrovinoblastine tartrate, KW-2307, Navelbine tartrate, NVB; Triptolide, Tripterygium wilfordii, PG490; Nortriptyline Hydrochloride; Zotarolimus, and ABT578.

The following compounds had an IC₅₀ value between 10 nM and 100 nM: Gemfibrozil; Tubercidin; Tris(hydroxymethyl)aminomethane; Mithramycin A, Piicamycin, Aureolic acid; Pirfenidone; Candesartan, CV-11974; Trichostatin A from Streptomyces sp.; 10-Hydroxycamptothecin; Homoharringtonine; Radicicol, Humicola fuscoatra; Paclitaxel; Epirubicin Hydrochloride; Triflusal; Herbimycin A from Streptomyces hygroscopicus; Cephalomannine; Taxol B; Teniposide; 17-AAG; Uracil, 2,4-Pyrimidinediol, 2,4-Dihydroxypyrimidine; Epothilone D, KOS-862; Halofuginone Hydrobromide (HBr); Topotecan, Hydrochloride; Terbinafine; Daunorubicin hydrochloride, Daunomycin hydrochloride; Bleomycin sulfate from Streptomyces verticillus, Blexane, Bleo, Blenoxane; Raltitrexed, Tomudex; Brefeldin A, Ascotoxin, acid lambda-lactone; Torasemide; BM-531, N-tert-Butyl-N-[(2-cyclohexylamino-5-nitrobenzene)sulfonyl]urea CPKC 412A, Midostaurin, N-Benzoylstaurosporine, PKC412A; Dacarbazine, DTIC Cytarabine, Cytosine beta-D-arabinoside; Cytochalasin A Helminthosporium dematioideum; Bay11-7085; and Mizoribine.

The following compounds had an IC₅₀ value between 100 nM and 600 nM: Mitomycin C; Famotidine; Cladribine, 2-Chloro-2-deoxyadenosine; Anisomycin from Streptomyces griseolus; Ro106-9920, D-erythro-Sphingosine, N,N-Dimethyl-; Bay41-8543; CEP2563; Pamidronate disodium; Auranofin, SKF 39162; Puromycin dihydrochloride from Streptomyces alboniger; Ancitabine hydrochloride, Cyclocytidine, Cyclo-C; Decitabine, 5-Aza-2-deoxycytidine, 2-Deoxy-5-azacytidine; Celastrol, Celastrus scandens, Tripterin; Podophyllotoxin; Rolipram; Oxaliplatin; Pitavastatin, Itavastatin Ca; Albendazole; Clarithromycin, 6-O-Methylerythromycin, A-56268, TE-031, Biaxin; 3-BAABE; Ammonium pyrrolidinedithiocarbamate, 1-Pyrrolidinecarbodithioic acid ammonium salt, Ammonium pyrrolidinecarbodithioate; Mitobronitol; Celecoxib; Quercetin dihydrate; Mycophenolic acid; Oxytetracycline hydrochloride; IMD-0354; Fluvastatin, XU 62-320na; CEP701; Formononetin, 7-Hydroxy-4-methoxyisoflavone, 7-Hydroxy-3-(4-methoxyphenyl)chromone; and Everolimus.

The following compounds had an IC₅₀ value between 600 nM and 1200 nM: Etoposide; Safingol, L-threo-Dihydrosphingosine; Deuterium oxide, Water-d2, Heavy water; L(−)-Perillyl alcohol; Cepharanthine; Myricetin, Cannabiscetin; Ciglitazone, Ciglitizone; 5-Azacytidine, Ladakamycin; Roxatidine acetate; Fascaplysin, Synthetic; Kaempferol; Irinotecan Hydrochloride Trihydrate, 1,4-Bipiperidine]-1-carboxylic acid; Penfluridol, R16341; Parthenolide; Mycophenolate mofetil, CellCept; Itraconazole, Sporanox, Oriconazole, R51211; Fasudil, HA-1077 dihydrochloride; Lovastatin, Mevinolin from Aspergillus sp., Monacolin K, 6a-Methylcompactin, Mevacor, 6-a-Methylcompactin; Pentamidine; Mevastatin, Compactin, ML-236B; and Spermine tetrahydrochloride.

The following compounds had an IC₅₀ value between 1200 nM and 10,000 nM: HU-210; PD169316; INO-1001; Salubrinal; Chlorambucil; Apigenin, 4,5,7-Trihydroxyflavone; L-165,041. PPM-18; Atorvastatin calcium, Floxuridine; NPC-15437 dihydrochloride; Miconazole; H-1152, H-1152P, ROCK Inhibitor; GW8510. BXT-51072; 6-Azauridine, 6-Azauracil riboside; LY333531, Ruboxistaurin, LY 333531; SB 220025; Doxifluridine, 5-DFUR, 5-dFUrd, Ro-21-9738; Cliostazol, OPC 13013, Pletaal, OPC 21; Curcumin, Diferuloylmethane, Diferulylmethane, Natural Yellow 3; SC12267; Rosuvastatin; Cerivastatin Na, Lipobay, Baycol; Dicumarol, 3,3-Methylenebis(4-hydroxycoumarin), Dicoumarin, Dicoumarol; 17a-Ethynylestradiot 3-cyclopentyl ether, Quinestrol; Wedelolactone; Melphalan, L-Phenylalanine mustard, L-PAM; CGP53353, CGP 53353, DAPH 2; Sphingosine-1-phosphate, D-Erythro-sphingosine-1-phosphate; 5-Bromo-2-deoxyuridine, Br-dU, BUdR; SB 239063; Hydralazine hydrochloride; Imidazole, Glyoxaline; Idebenone; Sertraline hydrochloride, Zoloft; Sildenafil Citrate; Atovaquone; Cloricromene; CGP74514A; Chromomycin A3 Streptomyces griseus; CEP751; Fludarabine des-phosphate, 9-b-D-Arabinofuranosyl-2-fluoroadenine, F-ara-A; Emodin, 6-Methyl-1,3,8-trihydroxyanthraquinone, Emodol, Frangula-emodin; Diflorasone diacetate; AG1433, SU1433; Butoconazole Nitrate; Halobetasol Propionate, BMY 30056, CGP 14458, Ulobetasol propionate, Ultravate; WW-85; Pravastatin; MK-886, L 663536; Rottlerin, Mallotoxin; Carmofur; Erbstatin analog, Methyl 2,5-dihydroxycinnamate; NS-398; BMS-345541; SP600125, JNK Inhibitor; Tamoxifen; Spermidine trihydrochloride; Pipobroman, A 1803, A-8103, Amedel, N,N-Bis(3-bromopropionyl)piperazine, NSC-25154, piperazine, 1,4-bis(3-bromo-1-oxopropyl)-(9CI), piperazine, 1,4-bis(3-bromopropionyl)-(8CI), Vercyte, WLN: T6N DNTJ AV2E DV2E,1,4-Bis(3-bromopropio; Econazole nitrate salt, 1-[2,4-dichloro-a-[(p-chlorobenzyl)oxy]phenethyl]imidazole; Enalapril; 15-Deoxyprostaglandin J2, 15-deoxy PGJ2,15-Deoxy-D12,14-prostaglandin J2,15-Deoxy-D12,14-PGJ2; LBM642; SCH-58261; Betulinic acid; Irsogladine; B-Nicotinamide adenine dinucleotide, reduced disodium salt hydrate, Diphosphopyridine nucleotide reduced disodium salt, Coenzyme I reduced disodium salt, B-NADH, B-DPNH; MMP-2/MMP-3 Inhibitor II; GW9662; Carmustine; Piperidine; Indole-3-carbinol, 13C, Indole 3-methanol Midecamycin from Streptomyces mycarofaciens, Leucomycin V 3,4B-dipropionate; C2 Ceramide. N-Acetyl-D-sphingosine, Maprotiline. PD98059, Fludarabine Phosphate, Benzoyl peroxide, RS 102895, Erdosteine, Sulfamerazine, and Losartan potassium.

### Example 14

### Evaluation of Paclitaxel Containing Mesh on Intimal Hyperplasia Development in a Rat Balloon Injury Carotid Artery Model as an Example to Evaluate Fibrosis Inhibiting Agents

A rat balloon injury carotid artery model was used to demonstrate the efficacy of a paclitaxel containing mesh system on the development of intimal hyperplasia fourteen days following placement.

Control Group

Wistar rats weighing 400-500 g were anesthetized with 1.5% halothane in oxygen and the left external carotid artery was exposed. An A 2 French FOGARTY balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml) and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

### Local Perivascular Paclitaxel Treatment

Immediately after injury of the left common carotid artery, a 1 cm long distal segment of the artery was exposed and treated with a 1×1 cm paclitaxel-containing mesh (345 μg paclitaxel in a 50:50 PLG coating on a 10 90 PLG mesh) The wound was then closed the animals were kept for 14 days.

### Histology and Immunohistochemistry

At the time of sacrifice, the animals were euthanized with carbon dioxide and pressure perfused at 100 mmHg with 10% phosphate buffered formaldehyde for 15 minutes. Both carotid arteries were harvested and left overnight in fixative. The fixed arteries were processed and embedded in paraffin wax. Serial cross-sections were cut at 3 μm thickness every 2 mm within and outside the implant region of the injured left carotid artery and at corresponding levels in the control right carotid artery. Cross-sections were stained with Mayer's hematoxylin-and-eosin for cell count and with Movat's pentachrome stains for morphometry analysis and for extracellular matrix composition assessment.

### Results

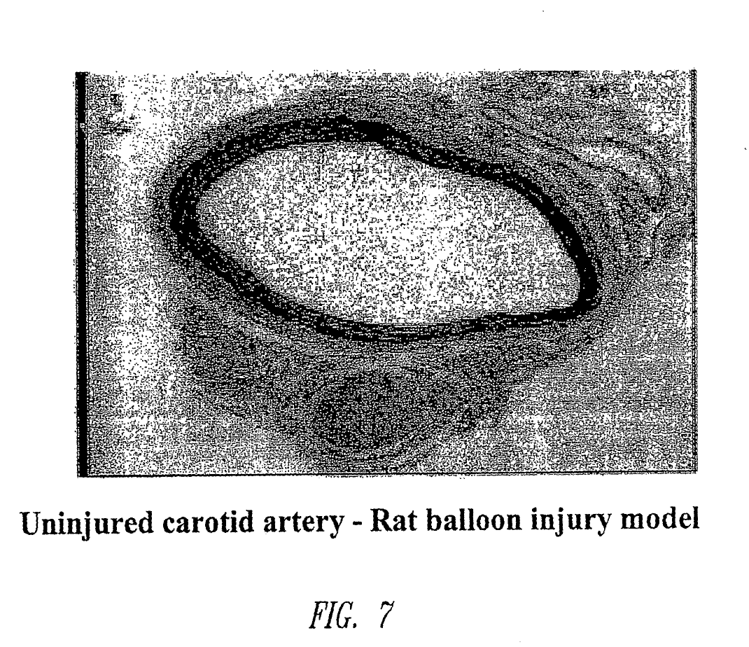

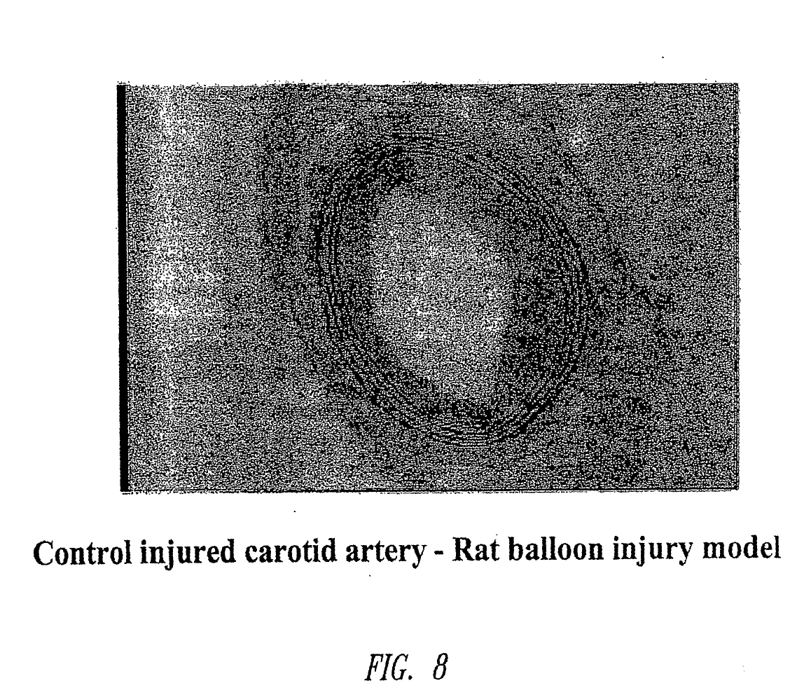

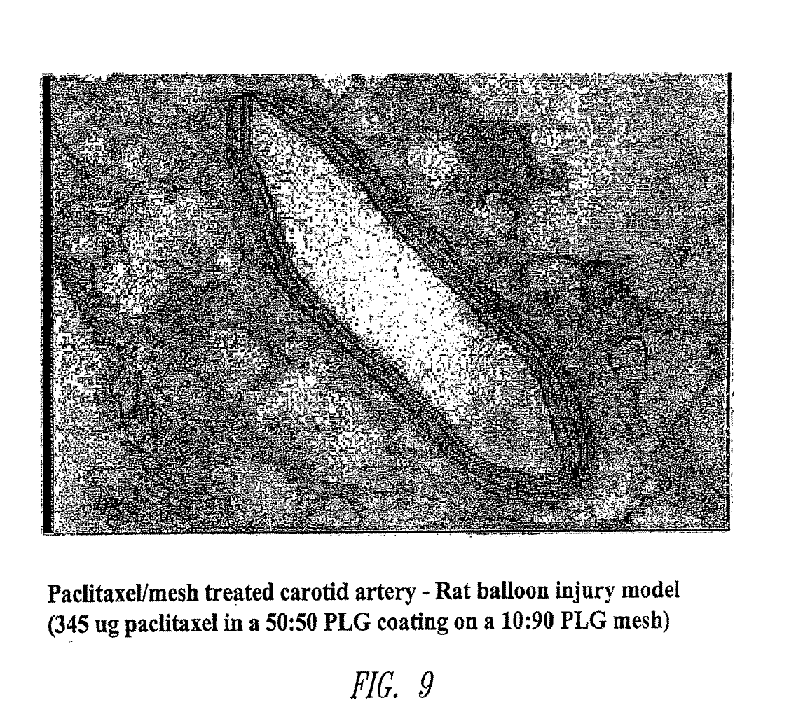

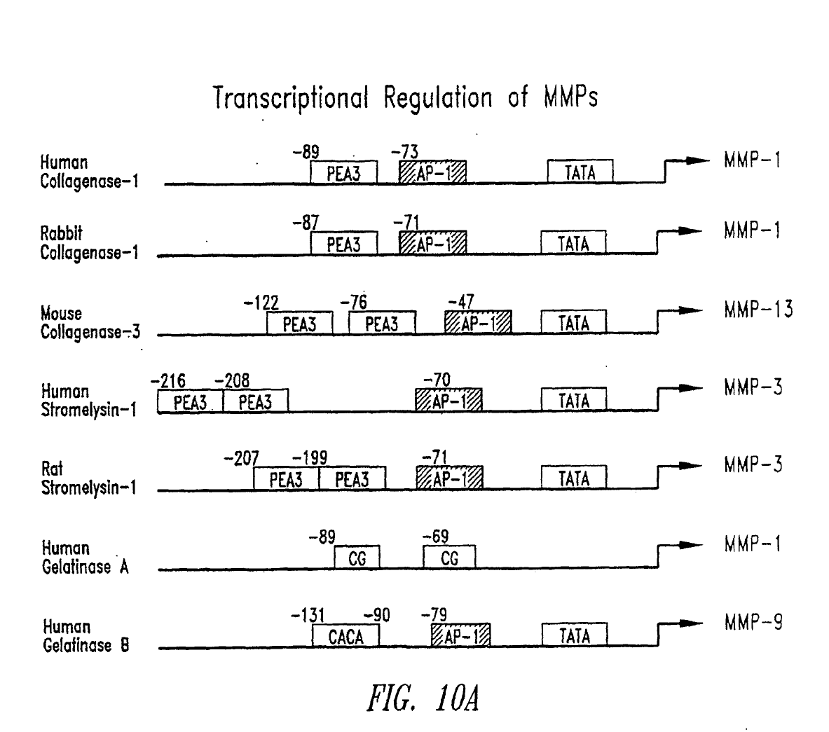

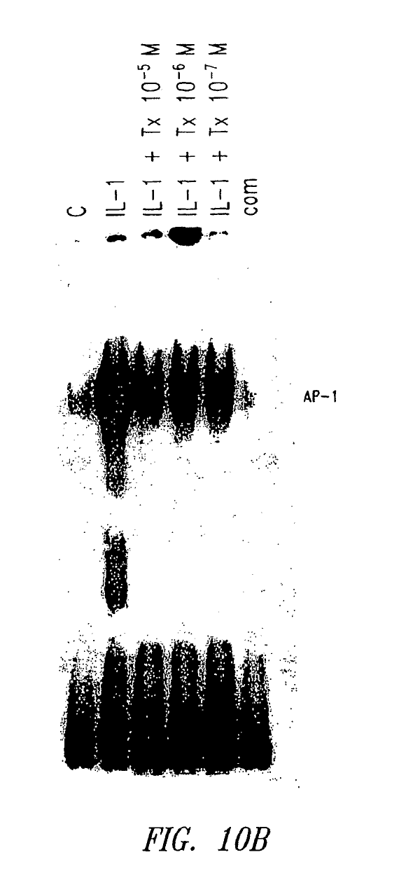

From FIGS. 7-9, it is evident that the perivascular delivery of paclitaxel using the paclitaxel mesh formulation resulted is a dramatic reduction in intimal hyperplasia.

### Example 15

### Effect of Paclitaxel and Other Anti-Microtubule Agents on Matrix Metalloproteinase Production

### A. Materials and Methods

1. IL-1 Stimulated AP-1 Transcriptional Activity is Inhibited by Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations Pacitaxel treatment decreased CAT activity in a concentration dependent manner (FIG. 1C; mean±SD) The data noted by an asterisk (*) have significance compared with IL-1-induced CAT activity according to a t-test, P<0.05 The results shown are representative of three independent experiments

2. Effect of Paclitaxel on IL-1 Induced AP-1 DNA Binding Activity, AP-1 DNA

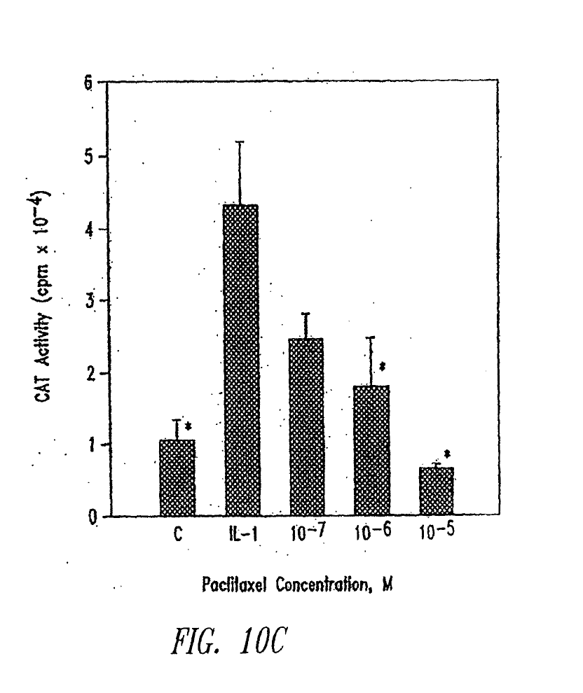

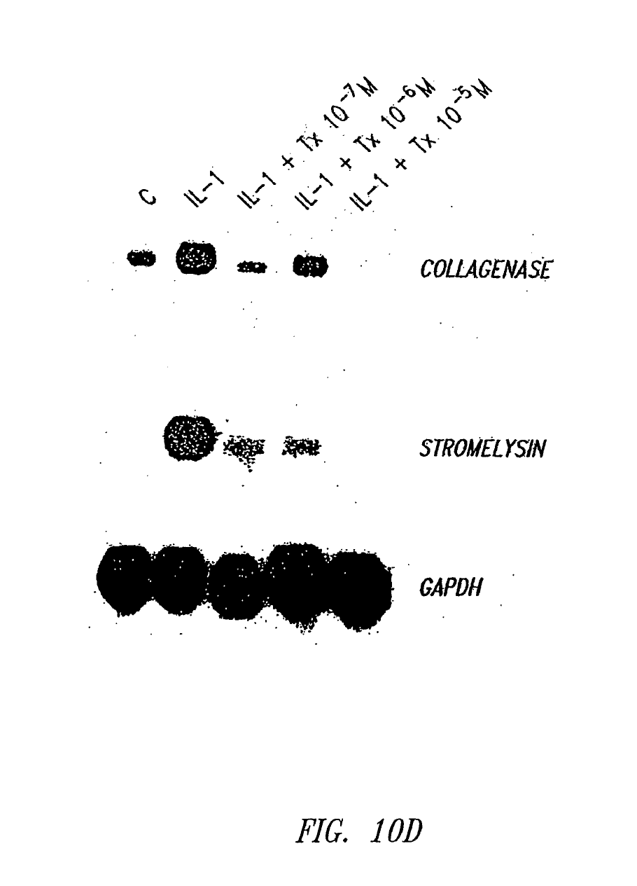

Binding activity was assayed with a radiolabeled human AP-1 sequence probe and gel mobility shift assay. Extracts from chondrocytes untreated or treated with various amounts of paclitaxet (10⁻⁷ to 10⁻⁵ M) followed by IL-1β (20 ng/ml) were incubated with excess probe on ice for 30 minutes, followed by non-denaturing gel electrophoresis (FIG. 10B). The “com” lane contains excess unlabeled AP-1 oligonucleotide. The results shown are representative of three independent experiments.

3. Effect of Paclitaxel on IL-1 Induced MMP-1 and MMP-3 mRNA Expression

Cells were treated with paclitaxel at various concentrations (10⁻⁷ to 10⁻⁵ M) for 24 hours, then treated with IL-1β (20 ng/ml) for additional 18 hours in the presence of paclitaxel. Total RNA was isolated, and the MMP-1 mRNA levels were determined by Northern blot analysis. The blots were subsequently stripped and re-probed with ³²P-radiolabeled rat GAPDH cDNA, which was used as a housekeeping gene. The results shown are representative of four independent experiments (FIG. 10D). Quantitation of collagenase-1 and stromelysin-expression mRNA levels were conducted. The MMP-1 and MMP-3 expression levels were normalized with GAPDH.

4. Effect of Other Anti-Microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel Denatured RNA samples (15 μg) were analyzed by gel electrophoresis in a 1% denatured gel, transferred to a nylon membrane and hydridized with the ³²P-labeled collagenase cDNA probe. ³²P-labeled glyceraldehyde phosphate dehydrase (GAPDH) cDNA was used as an internal standard to ensure roughly equal loading. The exposed films were scanned and quantitatively analyzed with IMAGEQUANT.

### F. Results

1. Promoters on the Family of Matrix Metalloproteinases

FIG. 10A shows that all matrix metalloproteinases contained the transcriptional elements AP-1 and PEA-3 with the exception of gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

2. Effect of Paclitaxel on AP-1 Transcriptional Activity

As demonstrated in FIG. 10B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrate that paclitaxel was a potent inhibitor of AP-1 activity in chondrocytes.

3. Effect of Paclitaxel on AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 10C, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

4. Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases, was examined. Briefly, as shown in FIG. 10D, IL-1 induction increased collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5. Effect of Other Anti-Microtubules on Collagenase Expression

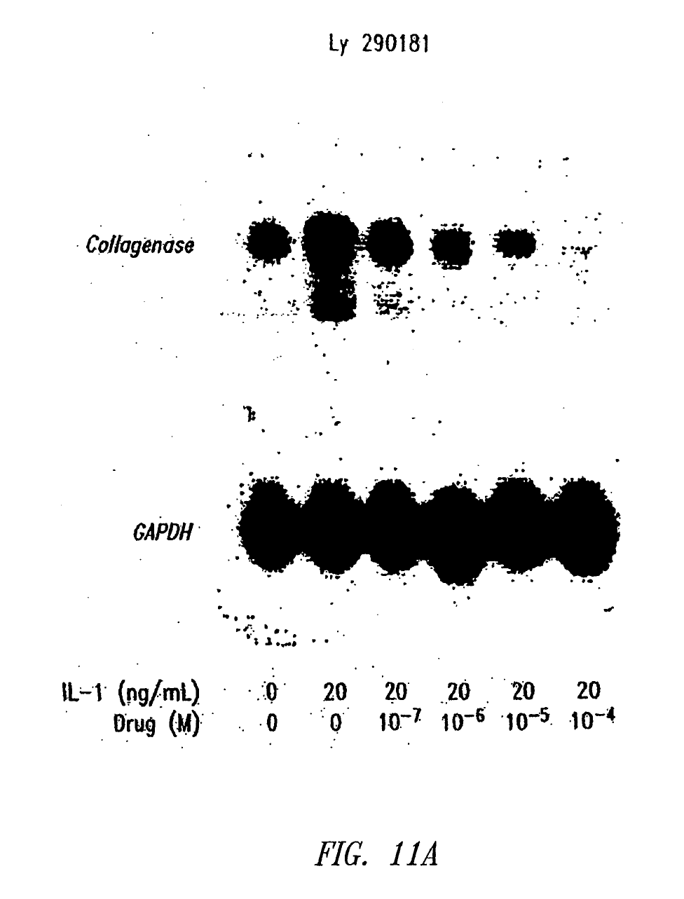

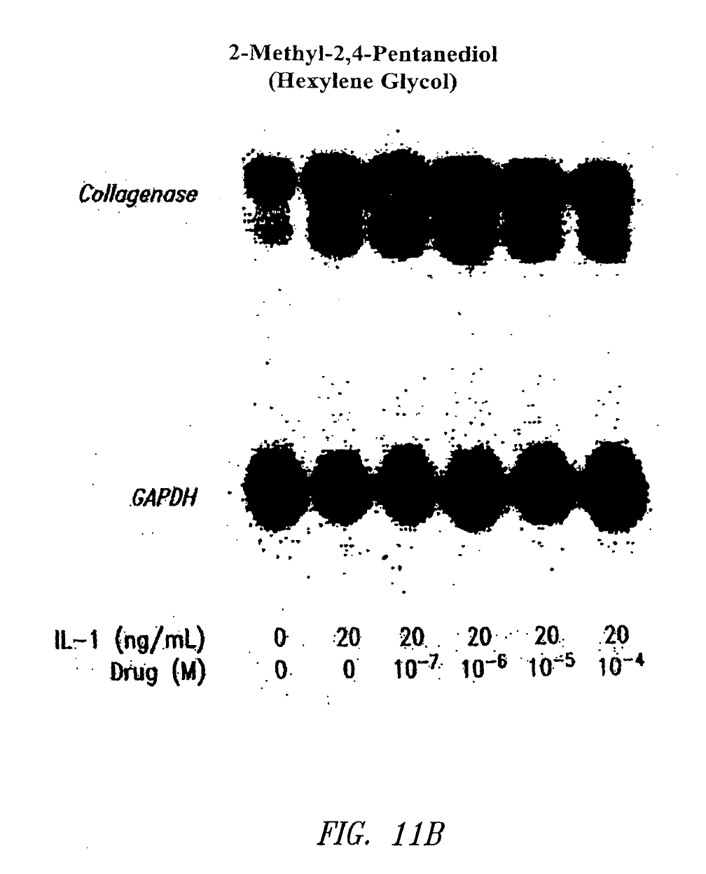

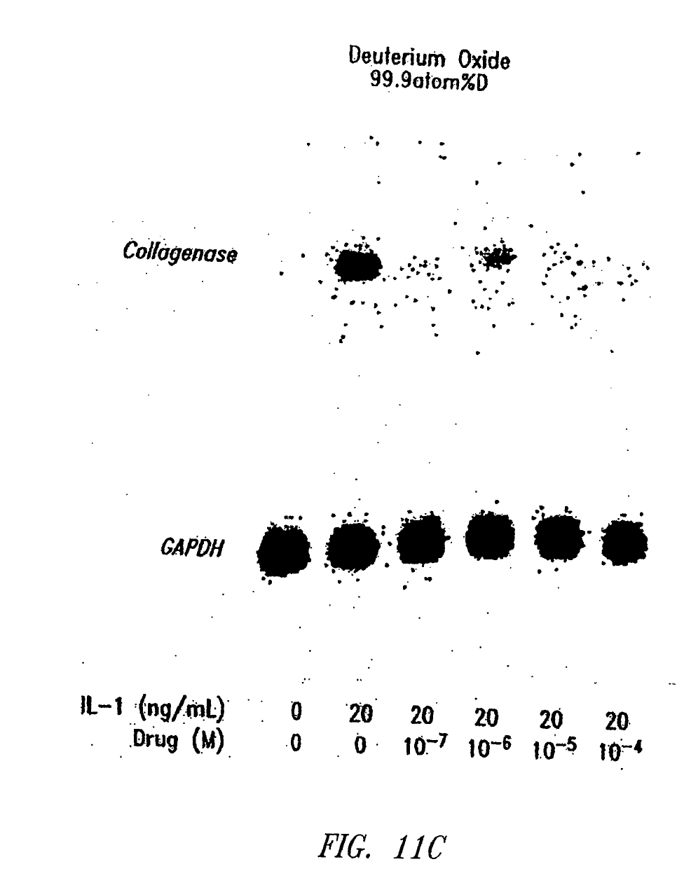

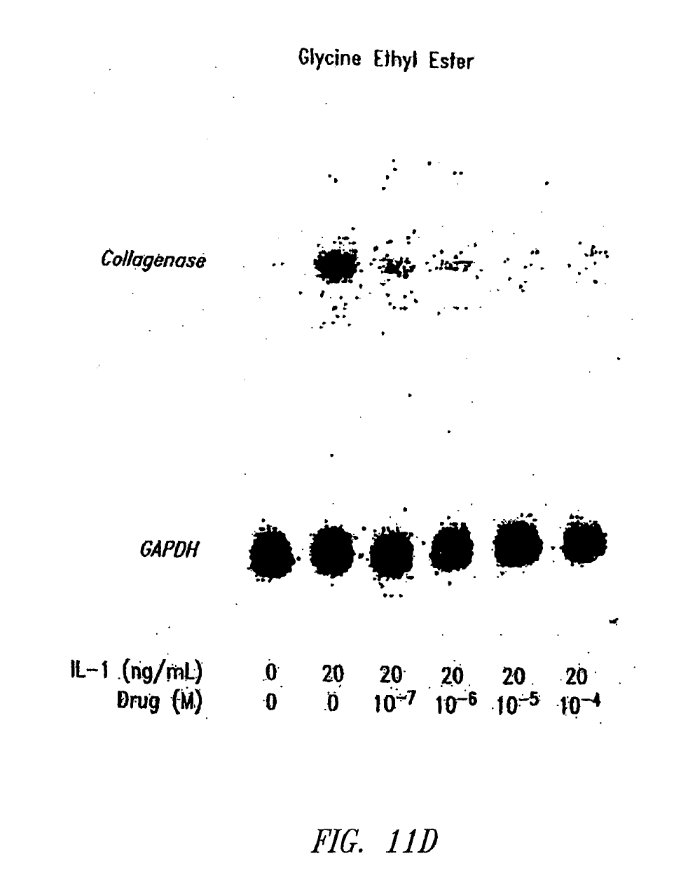

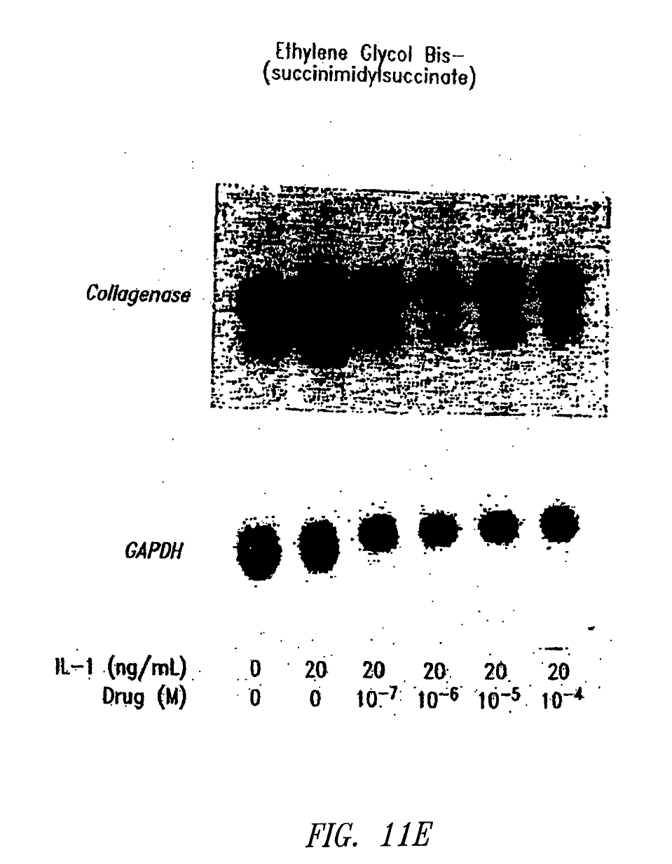

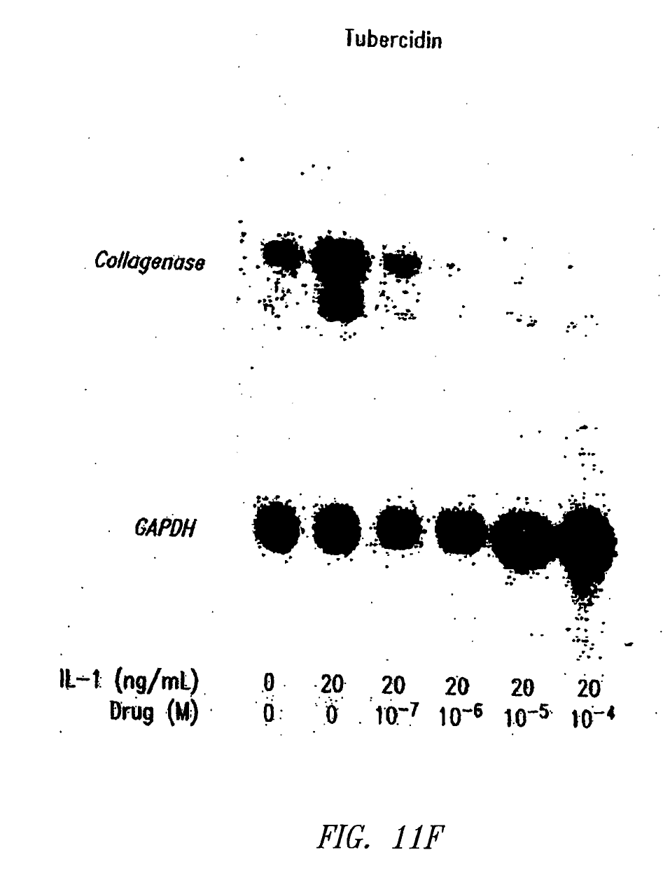

FIGS. 11A-H demonstrate that anti-microtubule agents inhibited collagenase expression. Expression of collagenase was stimulated by the addition of IL-1, which is a pro-inflammatory cytokine. Pre-incubation of chondrocytes with various anti-microtubule agents, specifically LY290181, hexylene glycol, deuterium oxide, glycine ethyl ester, ethylene glycol bis-(succinimidylsuccinate), tubercidin, AlF₃, and epothilone, all prevented IL-1-induced collagenase expression at concentrations as low as 1×10⁻⁷ M.

### G. Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases that are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin can have a beneficial effect in inflammatory diseases.

In addition to paclitaxel's inhibitory effect on collagenase expression, LY290181, hexylene glycol, deuterium oxide, glycine ethyl ester, AlF₃, tubercidin epothilone, and ethylene glycol bis-(succinimidylsuccinate), all prevented IL-1-induced collagenase expression at concentrations as low as 1×10⁻⁷ M. Thus, anti-microtubule agents were shown to be capable of inhibiting the AP-1 pathway at varying concentrations.

### Example 16

### Inhibition of Angiogenesis by Paclitaxel

### B. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with Petri dish covers. These were then placed into an incubator at 90% relative humidity and 3% CO₂ and incubated for 3 days.

Paclitaxel (Sigma, St. Louis. MO) was mixed at concentrations of 0.25, 0.5, 1, 5, 10, 30 μg per 10 ul aliquot of 0.5% aqueous methylcellulose. Since paclitaxel is insoluble in water, glass beads were used to produce fine particles. Ten microliter aliquots of this solution were dried on Parafilm for 1 hour, forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2-day exposure (day 8 of incubation), the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8-day-old shell-less embryo were flooded with 2% glutaraldehyde in 0.1M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were post-fixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding. Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5-minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged non-invasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160×, which permitted the direct visualization of blood cells within the capillaries; thereby blood flow in areas of interest may be easily assessed and recorded For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 1
Avascular Gradient

| | |
|---|---|
| 0 | normal vascularity |
| 1 | lacking some microvascular movement |
| 2* | small avascular zone approximately 2 mm in diameter |
| 3* | avascularity extending beyond the disk (6 mm in diameter) |

*indicates a positive antiangiogenesis response

The dose-dependent, experimental data of the effects of paclitaxel at different concentrations are shown in Table 2.

TABLE 2

| Agent | Delivery Vehicle | Concentration | Inhibition/n |
|---|---|---|---|
| paclitaxel | methylcellulose (10 μl) | 0.25 μg | 2/11 |
| | methylcellulose (10 μl) | 0.5 μg | 6/11 |
| | methylcellulose (10 μl) | 1 μg | 6/15 |
| | methylcellulose (10 μl) | 5 μg | 20/27 |
| | methylcellulose (10 μl) | 10 μg | 16/21 |
| | methylcellulose (10 μl) | 30 μg | 31/31 |

Typically paclitaxel-treated CAMs are also seen with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone that was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

### Example 17

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media and diluted to a concentration of 2-2.5×10⁵ cells/ml in migration media. Migration media consists of phenol red free Dulbecco's Modified Eagle Medium (DMEM) containing 0.35% human serum albumin. A 100 μL volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/ml in a total volume of 150 μL. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

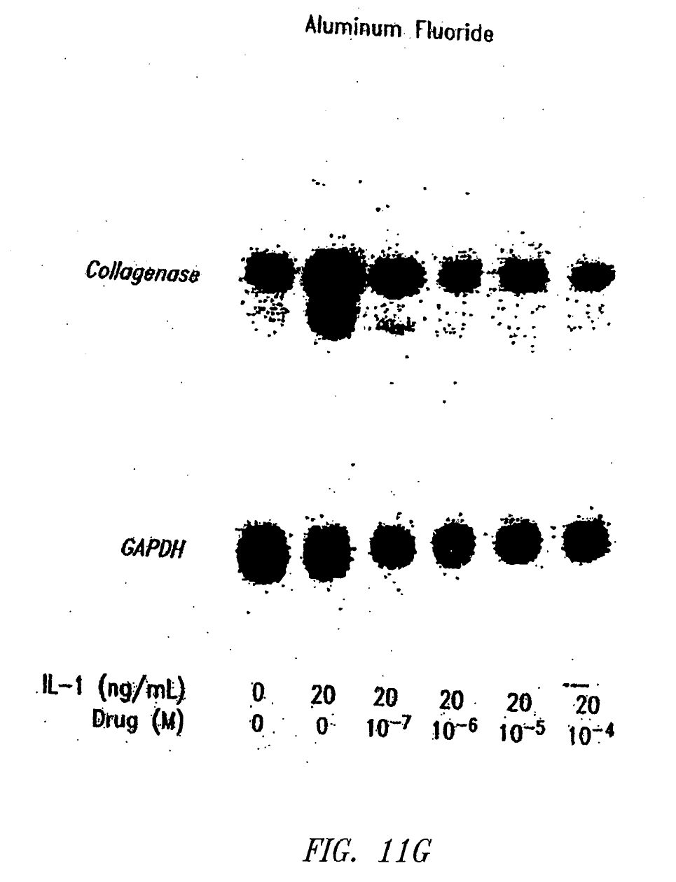

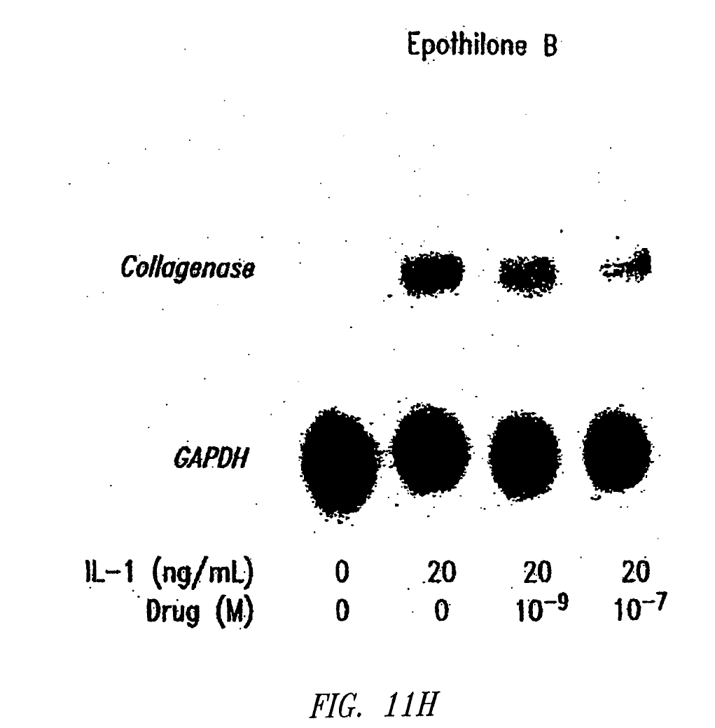

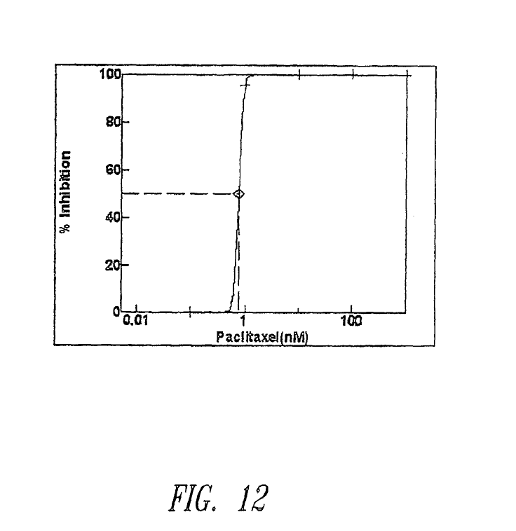

At the end of the 4-hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well Inhibitory concentration of 50% (IC₅₀) was determined by comparing the average number of cells migrating in the presence of paclitaxel to the positive control (smooth muscle cell chemotaxis in response to rhPDGF-BB) See FIG. 12 (IC₅₀=0 76 nM). References Biotechniques (2000) 29:81; J. Immunol. Methods (2001) 254:85

### Example 18

### Screening Assay for Assessing the Effect of Various Compounds on IL-1β Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-1β by the addition of 1 mg/ml opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

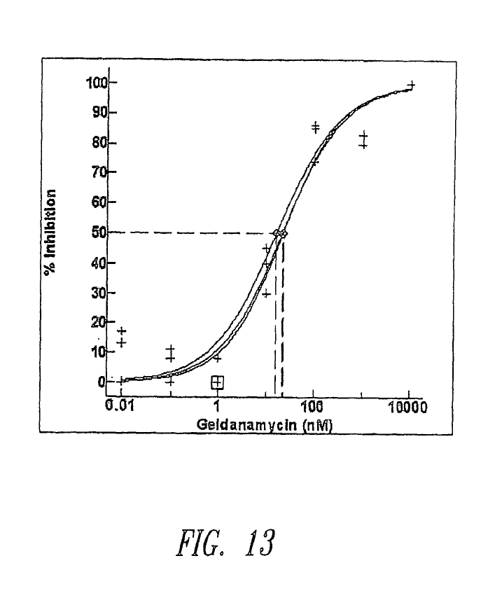

After a 24-hour stimulation, supernatants were collected to quantify IL-1β production. IL-1β concentrations in the supernatants were determined by ELISA using recombinant human IL-1β to obtain a standard curve. A 96-well MaxiSorb plate was coated with 100 μL of anti-human IL-1 Capture Antibody diluted in Coating Buffer (0.1M Sodium carbonate pH 9.5) overnight at 4° C. The dilution of Capture Antibody used was lot-specific and was determined empirically Capture antibody was then aspirated and the plate washed 3 times with Wash Buffer (PBS, 0 05% TWEEN-20) Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/4 and 1/8, (b) recombinant human IL-1β was prepared at 1000 pg/ml and serially diluted to yield as standard curve of 15.6 pg/ml to 1000 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human IL-1β detection antibody+avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 NH₂SO₄) was then added to the wells and a yellow color reaction was read at 450 nm with λ correction at 570 nm. Mean absorbance was determined from triplicate data readings and the mean background was subtracted. IL-1β concentration values were obtained from the standard curve. Inhibitory concentration of 50% (IC₅₀) was determined by comparing average IL-1β concentration to the positive control (THP-1 cells stimulated with opsonized zymosan). An average of n=4 replicate experiments was used to determine IC₅₀ values for geldanamycin (IC₅₀=20 nM). See FIG. 13. The IC₅₀ values for the following additional compounds were determined using this assay: IC₅₀ (nM): mycophenolic acid 2888 nM); anisomycin, 127; rapamycin, 0.48; halofuginone, 919; IDN-6556, 642; epirubicin hydrochloride, 774; topotemay, 509; fascaplycin, 425; daunorubicin, 517; celastrol, 23; oxalipatin, 107; chromomycin A3, 148.

References: J. Immunol. (2000) 165:411-418; J. Immunol. (2000) 164: 4804-4811; J. Immunol. Meth. (2000) 235 (1-2):33-40.

### Example 19

### Screening Assay for Assessing the Effect of Various Compounds on IL-8 Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g, resuspended in 4 ml of human serum for a final concentration of 5 mg/ml, and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-8 by the addition of 1 mg/ml opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

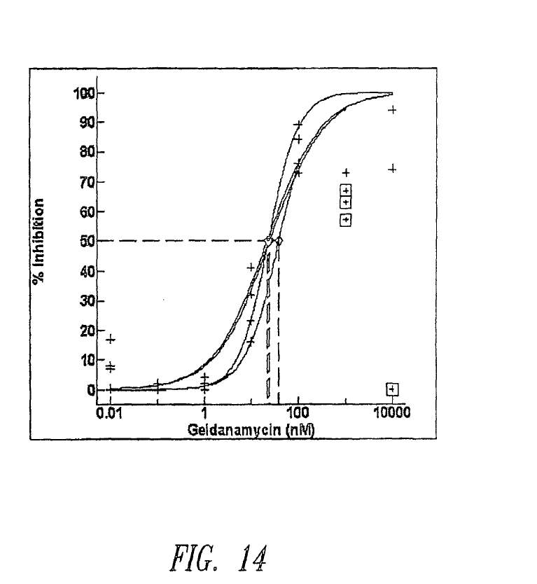

After a 24-hour stimulation, supernatants were collected to quantify IL-8 production. IL-8 concentrations in the supernatants were determined by ELISA using recombinant human IL-8 to obtain a standard curve. A 96-well MAXISORB plate was coated with 100 μL of anti-human IL-8 Capture Antibody diluted in Coating Buffer (0.1M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human IL-8 was prepared at 200 pg/ml and serially diluted to yield as standard curve of 3.1 pg/ml to 200 pg/ml Sample supernatants and standards were assayed in triplicate and were incubated at room temperature for 2 hours after addition to the plate coated with Capture Antibody The plates were washed 5 times and incubated with 100 μL of Working Detector (biotinylated anti-human IL-8 detection antibody+avidin-HRP) for 1 hour at room temperature. Following this incubation, the plates were washed 7 times and 100 μL of Substrate Solution (Tetramethylbenzidine, H₂O₂) was added to plates and incubated for 30 minutes at room temperature. Stop Solution (2 NH₂SO₄) was then added to the wells and a yellow color reaction was read at 450 nm with A correction at 570 nm. Mean absorbance was determined from triplicate data readings and the mean background was subtracted. IL-8 concentration values were obtained from the standard curve. Inhibitory concentration of 50% (IC₅₀) was determined by comparing average IL-8 concentration to the positive control (THP-1 cells stimulated with opsonized zymosan). An average of n=4 replicate experiments was used to determine IC₅₀ values for geldanamycin (IC₅₀=27 nM). See FIG. 14. The IC₅₀ values for the following additional compounds were determined using this assay: IC₅₀ (nM): 17-AAG, 56; mycophenolic acid, 549; resveratrol, 507; rapamycin, 4; 41; SP600125, 344; halofuginone, 641; D-mannose-6-phosphate, 220; epirubicin hydrochloride, 654; topotemay, 257; mithramycin, 33; daunorubicin, 421; celastrol, 490; chromomycin A3, 36

References: J. Immunol. (2000)165:411-418; J. Immunol. (2000) 164:480-44811; J. Immunol. Meth. (2000) 235 (1-2):33-40.

### Example 20

### Screening Assay for Assessing the Effect of Various Compounds on MCP-1 Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce MCP-1 by the addition of 1 mg/ml opsonized zymosan. Eldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

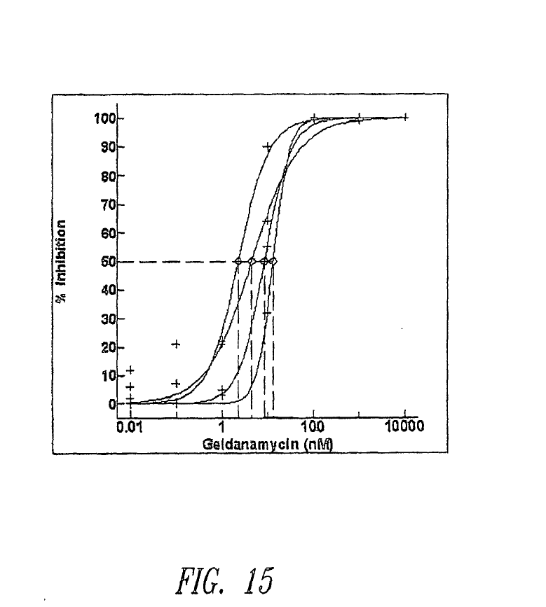

After a 24-hour stimulation, supernatants were collected to quantify MCP-1 production. MCP-1 concentrations in the supernatants were determined by ELISA using recombinant human MCP-1 to obtain a standard curve. A 96-well MaxiSorb plate was coated with 100 μL of anti-human MCP-1 Capture Antibody diluted in Coating Buffer (0.1M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μL/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human MCP-1 was prepared at 500 pg/ml and serially diluted to yield as standard curve of 7.8 pg/ml to 500 pg/ml. Sample supernatants and standards were assayed in triplicate and were incubated at room temperature for 2 hours after addition to the plate coated with Capture Antibody. The plates were washed 5 times and incubated with 100 μL of Working Detector (biotinylated anti-human MCP-1 detection antibody+avidin-HRP) for 1 hour at room temperature. Following this incubation, the plates were washed 7 times and 100 μL of Substrate Solution (tetramethylbenzidine, H₂O₂) was added to plates and incubated for 30 minutes at room temperature. Stop Solution (2 NH₂SO₄) was then added to the wells and a yellow color reaction was read at 450 nm with A correction at 570 nm. Mean absorbance was determined from triplicate data readings and the mean background was subtracted. MCP-1 concentration values were obtained from the standard curve. Inhibitory concentration of 50% (IC₅₀) was determined by comparing average MCP-1 concentration to the positive control (THP-1 cells stimulated with opsonized zymosan). An average of n=4 replicate experiments was used to determine IC₅₀ values for geldanamycin (IC₅₀=7 nM). See FIG. 15. The IC₅₀ values for the following additional compounds were determined using this assay: IC₅₀ (nM): 17-AAG, 135; anisomycin, 71; mycophenolic acid, 764; mofetil, 217; mitoxantrone, 62; chlorpromazine, 0.011; 1-α-25 dihydroxy vitamin D₃, 1; Bay 58-2667, 216; 15-deoxy prostaglandin J2, 724; rapamycin, 0.05; CNI-1493, 0.02; BXT-51072, 683; halofuginone, 9; CYC 202, 306; topotemay, 514; fascaplycin, 215; podophyllotoxin, 28; gemcitabine, 50; puromycin, 161; mithramycin, 18; daunorubicin, 570; celastrol, 421; chromomycin A3, 37; vinorelbine, 69; tubercidin, 56; vinblastine, 19; vincristine, 16.

References: J. Immunol. (2000) 165:411-418; J. Immunol. (2000) 164: 4804-4811; J. Immunol. Meth. (2000) 235 (1-2):33-40

### Example 21

### Screening Assay for Assessing the Effect of Paclitaxel on Cell Proliferation

Smooth muscle cells at 70-90% confluency were trypsinized, replated at 600 cells/well in media in 96-well plates and allowed to attachment overnight. Paclitaxel was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μL/well. Each drug concentration was tested in triplicate wells. Plates containing cells and paclitaxel were incubated at 37° C. for 72 hours.

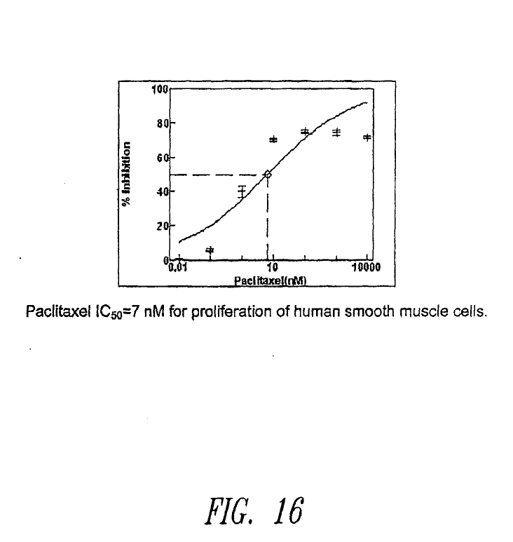

To terminate the assay, the media was removed by gentle aspiration A 1/400 dilution of CYQUANT 400×GR dye indicator (Molecular Probes; Eugene, Oreg.) was added to 1× Cell Lysis buffer, and 200 μL of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=3 replicate experiments was used to determine IC₅₀ values. See FIG. 16 (IC₅₀=7 nM). The IC₅₀ values for the following additional compounds were determined using this assay: IC₅₀ (nM): mycophenolic acid, 579; mofetil, 463; doxorubicin, 64; mitoxantrone, 1; geldanamycin, 5; anisomycin, 276; 17-AAG, 47; cytarabine, 85; halofuginone, 81; mitomycin C, 53; etoposide, 320; cladribine, 137; lovastatin, 978; epirubicin hydrochloride, 19; topotemay, 51; fascaplysin, 510; podophyllotoxin, 21; cytochalasin A, 221; gemcitabine, 9; puromycin, 384; mithramycin, 19; daunorubicin, 50; celastrol, 493; chromomycin A3, 12; vinorelbine, 15; idarubicin, 38; nogalamycin, 49; itraconazole, 795; 17-DMAG, 17; epothilone D, 5; tubercidin, 30; vinblastine, 3; vincristine, 9.

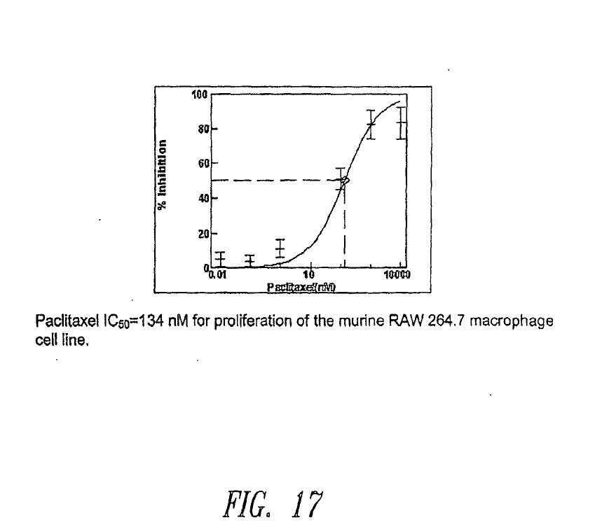

This assay also may be used assess the effect of compounds on proliferation of fibroblasts and murine macrophage cell line RAW 264.7. The results of the assay for assessing the effect of paclitaxel on proliferation of murine RAW 264.7 macrophage cell line were shown in FIG. 17 (IC₅₀=134 nM).

Reference: In vitro toxicol. (1990) 3:219; Biotech. Histochem. (1993) 68:29; Anal. Biochem. (1993) 213:426.

### Example 22

### Perivascular Administration of Paclitaxel to Assess Inhibition of Fibrosis

WISTAR rats weighing 250-300 g are anesthetized by the intramuscular Injection of Innovar (0.33 ml/kg). Once sedated, they are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 French Fogarty balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

Pactitaxel (33%) in ethelyne vinyl acetate (EVA) is then injected in a circumferential fashion around the common carotid artery in ten rats. EVA alone is injected around the common carotid artery in ten additional rats. (The paclitaxel may also be coated onto an EVA film, which is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

### Example 23

### MIC Determination by Microtitre Broth Dilution Method

### A. MIC Assay of Various Gram Negative and Positive Bacteria

MIC assays were conducted essentially as described by Amsterdam, D 1996, “Susceptibility testing of antimicrobials in liquid media”, p. 52-111, in Loman, V, ed. Antibiotics in laboratory medicine, 4th ed Williams and Wilkins, Baltimore, Md. Briefly, a variety of compounds were tested for antibacterial activity against isolates of P. aeruginosa, K. pneumoniae, E. coli, S. epidermidus and S. aureus in the MIC (minimum inhibitory concentration assay under aerobic conditions using 96 well polystyrene microtitre plates (Falcon 1177), and Mueller Hinton broth at 37° C. incubated for 24 h. (MHB was used for most testing except C721 (S. pyogenes), which used Todd Hewitt broth, and Haemophilus influenzae, which used Haemophilus test medium (HTM)) Tests were conducted in triplicate. The results are provided below in Table 1.

TABLE 1
MINIMUM INHIBITORY CONCENTRATIONS OF THERAPEUTIC AGENTS
AGAINST VARIOUS GRAM NEGATIVE AND POSITIVE BACTERIA

| | Bactrial Strain | | | | | |
|---|---|---|---|---|---|---|
| | P. aeruginosa | K. pneumoniae | E. coli | S. aureus | | |
| | PAE/K799 | ATCC13883 | UB1005 | ATCC25923 | S. epidermidis | S. pyogenes |
| | H187 | C238 | C498 | C622 | C621 | C721 |
| | Wt | wt | wt | wt | wt | wt |
| Drug | Gram− | Gram− | Gram− | Gram+ | Gram+ | Gram+ |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5-fluorouracil | 10⁻⁵ | 10⁻⁶ | 10⁻⁶ | 10⁻⁷ | 10⁻⁷ | 10⁻⁴ |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2- | N | N | N | N | N | N |
| mercaptopurine | | | | | | |
| 6- | N | N | N | N | N | N |
| mercaptopurine | | | | | | |
| Cytarabine | N | N | N | N | N | N |

Activities are in Molar concentrations
Wt = wild type
N = No activity

### H. MIC of Antibiotic-Resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea and cytarabine were tested for antibacterial activity against clinical isolates of a methicillin resistant S. aureus and a vancomycin resistant pediocoocus clinical isolate in an MIC assay as described above. Compounds which showed inhibition of growth (MIC value of <1.0×10−3) included: mitoxantrone (both strains), methotrexate (vancomycin resistant pediococcus), 5-fluorouracil (both strains), etoposide (both strains), and 2-mercaptopurine (vancomycin resistant pediococcus).

### Example 24

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1 L HPLC grade water is added and the solution is stirred until all the salts are dissolved. If required, the pH of the solution is adjusted to pH 7.4±0.2 using either 0.1 N NaOH or 0.1 N phosphoric acid.

### Example 25

### Release Study to Determine Release Profile of a Therapeutic Agent from a Polymeric Composition

The release profile of a therapeutic agent from a polymeric composition can be determined according to the following procedure.

### Release and Extraction

A sample is placed in a 16×125 mm screw capped culture tube 16 ml release buffer (Example 35) is added to the tube. The samples are placed on a rotating wheel (30 rpm) in a 37° C. oven. At the various time intervals (2 h, 5 h, 8 h, 24 h and then daily), the sample tubes are taken from the oven, placed in a rack and the caps are removed in a fume hood. As much of the release buffer as possible is removed from the tube and placed in a second culture tube. 16 ml of release media is then added to the sample containing tube using an Oxford pipettor bottle. The samples are capped with a new PTFE lined cap. All samples are returned to the rotating wheel device in the oven.

Using a p1000 pipettor (PIPETMAN) and a clean pipette tip, remove and discard 1 ml of release media from each sample. Add 1 ml of dichloromethane to each sample using an oxford pipettor bottle. Cap each sample tube with the respective PTFE lined screw cap. Hand shake each sample vigorously for 5 seconds. Place samples on the labquake rotator and rotate for 15 min. Centrifuge samples at 1500 rpm for 10 minutes. Transfer the sample tubes to a fume hood and uncap. Remove most of the supernatant (aqueous phase) using a Pasteur pipette and vacuum system. Remove the final portion of the supernatant with a glass syringe. Transfer sample tubes to the pierce drying system, set the heating block to 1.5 (45° C.) and turn on the system. Dry all samples on the pierce drying system under a stream of nitrogen gas (approximately 45 min.). Re-cap the sample tubes, place in a plastic bag, label bag with date and time of sample, and store at −20° C. (freezer) until analysis.

### External Standard Preparation

Paclitaxel (GMP grade) from Hauser Chemical Research, Inc. is be used as reference standard for this assay. Paclitaxel (100 mg) is be accurately weighed, quantitatively transferred and made up to volume with ACN in a 100 ml volumetric flask (1 mg/ml). Transfer 5 ml of this standard solution, using a volumetric pipette, to a 100 ml volumetric flask and make up to volume with ACN (50 μg/ml) Serial dilutions (5 ml qs ad 10 ml with ACN) will be used to prepare 25, 12.5, 6.25, 3 13, 1 56, 0 781 and 0.391 μg/ml solutions respectively On the day of HPLC analysis of samples, place an aliquot (˜100 μl) of each standard into separate autosampler vials using small volume inserts and transfer to the HPLC.

### Control and System Suitability Sample Preparation

Paclitaxel and 7-epi-taxel from Hauser Chemical Research, Inc. is used as control standards for this assay. Accurately weigh and quantitatively transfer 25 mg 7-Epi-taxel to a 25 ml volumetric flask and make up to volume with ACN (1 mg/ml). Transfer 5 ml of this standard solution, using a volumetric pipette, to a 100 ml flask and make up to volume with ACN (50 μg/ml 7-Epi-taxel). A 50/50 mixture of paclitaxel standard (25 μg/ml) and 7-epi-taxol standard (25 μg/ml) is used as the control and the system suitability samples. Prepare by adding a 5 ml aliquot of each paclitaxel dissolved in ACN (50 μg/ml paclitaxel) and 7-Epi-taxel dissolved in ACN (50 μg/ml 7-Epi-taxel) into the same culture tube. Cap and shake. Refrigerate until ready to use. On the day of HPLC analysis of samples, place an aliquot (˜150 μl) into two separate autosampler vials with small volume inserts and transfer to the HPLC. One sample is used for the system suitability. The other sample is used as the control sample.

### Sample Reconstitution

Remove samples to be analyzed from the freezer, place in a fume hood, and allow tubes to come to room temperature. Uncap and add 1 ml of water/acetonitrile (50/50) to each tube with an Oxford pipettor. Recap sample tubes and vortex for 60 s. Centrifuge sample tubes at 1500 rpm for 15 min. In a fume hood, transfer approximately 500 μl of each sample to a separate HPLC autosampler vial with a clean Pasteur pipette Cap each autosampler vial and transfer to the HPLC. Dispose of the sample tube and Pasteur pipette.

### HPLC Analysis

The following chromatographic conditions are used for paclitaxel analysis:

| | |
|---|---|
| Stationary | ODS |
| Phase | (Hypersil ODS, Hewlett Packard, 125 × 4 mm ID, 5 μm) |
| Guard Column | Hypersil ODS Guard column |
| Mobile Phase | Acetonitrile(ACN)/Water(H₂O) 45/55 |
| Flow Rate | 1.0 ml/min |
| Injection | 10 μL |
| Volume | |
| Detection | Ultraviolet at 232 nm |
| Run Time | 15 min |
| Column | 28.0° C. |
| Temperature | |

Inject the acetonitrile sample five times at the beginning to ensure equilibration. Inject the control sample five times after the acetonitrile sample, once following the standard curve samples, once following every ten samples throughout the set of samples, and once at the end of the sample set to verify system performance. Chromatograph the standard curve samples by injecting once at the start of each set of samples.

### Data Analysis

Integrate paclitaxel peak areas for all standards, control samples and release samples using HP ChemStation Batch Mode and generate a Batch Report saved in xls format. Use Excel to evaluate data from the Batch Report. Calculate the control sample peak area standard deviation (Excel: descriptive statistics) and % coefficient of variation (100× standard deviation/mean). Calculate the amount of paclitaxel injected (μg) for each standard curve sample based on the concentration prepared and a 10 μL injection Calculate the slope and intercept of the standard curve (peak area versus amount of paclitaxel injected) using Excel: regression analysis Calculate the amount of paclitaxel in each of the release samples injected. Establish the amount of paclitaxel (μg) released per 16 ml sample using the formula. The amount of paclitaxel released over time is established using the amount of paclitaxel per sample and the time the sample is taken.

### Example 26

### Formulation of a Drug in a Vehicle Comprising a Triblock Copolymer

Paclitaxel was incorporated into a formulation comprising a triblock copolymer and a diluent (described below) by dissolving the paclitaxel in the diluent with stirring at ambient temperature for at least two hours, then adding the triblock copolymer, again with stirring for at least 2 hours. Longer periods of time were used to add triblock copolymer at higher concentrations. For example, the addition of 33% triblock copolymer was accomplished by stirring for at least 15 hours (overnight). The diluent was PEG 300 NF or PEG 400 derivatized by end addition of trimethylene carbonate 90%/glycolide 10% in a ratio of 400:100. The triblock copolymer was an ABA copolymer with blocks A containing polymerized trimethylene carbonate (90%) and glycolide (10%), having a total molecular weight of about 900 g/mol and the B block containing PEG 400. Paclitaxel was effectively incorporated into this formulation at a concentration of 0.015 to 0.45 mg/ml. The amount of triblock copolymer in the formulation was varied from 2.3 to 50% w/w using PEG 400 as the diluent. The product was sterilized by exposure to about 2.5 kGy of gamma radiation.

### Example 27

### Formulation of a Drug in a Co-Solvent Vehicle

Paclitaxel was incorporated into a formulation comprising water and PEG 300 NF. The paclitaxel was first dissolved in a 90.10 mixture of PEG 300 NF:water by stirring at ambient temperature for at least two hours. Once the drug was dissolved, the composition was combined with equal parts of a 50:50 mixture of PEG 300 NF:water. The final composition was paclitaxel dissolved in a mixture of 70:30 PEG 300 NF:water. Paclitaxel was incorporated at concentrations of 0.45 to 4.5 mg/ml. The composition was passed through a 0.22 μm filter to render it sterile.

### Example 28

### Spinal Surgical Adhesions Model to Assess Fibrosis Inhibiting Agents in Rabbits

Extensive scar formation and adhesions often occur after lumbar spine surgery involving the vertebrae. The dense and thick fibrous tissue adherent to the spine and adjacent muscles must be removed by surgery. Unfortunately, fibrous adhesions usually reform after the secondary surgery. Adhesions are formed by proliferation and migration of fibroblasts from the surrounding tissue at the site of surgery. These cells are responsible for the healing response after tissue injury. Once they have migrated to the wound they lay down proteins such as collagen to repair the injured tissue. Overproliferation and secretion by these cells induce local obstruction, compression and contraction of the surrounding tissues with accompanying side effects.

The rabbit laminectomy spinal adhesion model described herein is used to investigate spinal adhesion prevention by local slow release of antifibrotic drugs.

Five to six animals are included in each experimental group to allow for meaningful statistical analysis. Formulations with various concentrations of antifibrotic drugs are tested against control animals to assess inhibition of adhesion formation.

Rabbits are anesthetized with an IM injection of ketamine/zylazine An endotracheal tube is inserted for maintenance of anesthesia with halothane The animal is placed prone on the operating table on top of a heating pad and the skin over the lower half of the back is shaved and prepared for sterile surgery. A longitudinal midline skin incision is made from L-1 to L-5 and down the lumbosacral fascia. The fascia is incised to expose the tips of the spinous processes. The paraspinous muscles are dissected and retracted from the spinous process and lamina of L-4. A laminectomy is performed at L-4 by removal of the spinal process with careful bilateral excision of the laminae, thus creating a small 5×10 mm laminectomy defect. Hemostasis is obtained with Gelfoam. The test formulations are applied to the injury site and the wound is closed in layers with Vicryl sutures. The animals are placed in an incubator until recovery from anesthesia and then returned to their cage.

Two weeks after surgery, the animals are anesthetized using procedures similar to those described above. The animals are euthanized with Euthanyl. After a skin incision, the laminectomy site is analyzed by dissection and the amount of adhesion is scored using scoring systems published in the scientific literature for this type of injury.

### Example 29

### Tendon Surgical Adhesions Model to Assess Fibrosis Inhibiting Agents in Rabbits

This model is used to investigate whether adhesion of the tendons can be prevented by local slow release of drugs known to inhibit fibrosis. Polymeric formulations are loaded with drugs and implanted around injured tendons in rabbits. In animals without fibrosis-inhibiting formulations, adhesions develop within 3 weeks of flexor tendon injury if immobilization of the tendon is maintained during that period An advantage of rabbits is that their tendon anatomy and cellular behaviour during tendon healing are similar to those in man except for the rate of healing that is much faster in rabbits

Rabbits are anesthetized and the skin over the right hindlimb is shaved and prepared for sterile surgery Sterile surgery is performed aided by an operating microscope. A longitudinal midline skin incision is made on the volvar aspect of the proximal phalange in digits 2 and 4. The synovial sheath of the tendons is carefully exposed and incised transversally to access the flexor digitorum profundus distal to the flexor digitorum superficialis bifurcation. Tendon injury is performed by gently lifting the flexor digitorum profundus with curved forceps and incising transversally through half of its substance. The formulation containing the test drug is applied around the tendons in the sheath of one of the two digits randomly selected. The other digit is left untreated and is used as a control. The sheath is then repaired with 6-0 nylon suture. An immobilizing 6-0 nylon suture is inserted through the transverse metacarpal ligament into the tendon/sheath complex to immobilize the tendon and the sheath as a single unit to encourage adhesion formation. The wound is closed with 4-0 interrupted sutures. A bandage is applied around the hindpaw to further augment immobilization of the digits and ensure comfort and ambulation of the animals. The animals are recovered and returned to their cage.

Three weeks after surgery, the animals are anesthetized. After a skin incision, the tissue plane around the synovial sheath is dissected and the tendon-sheath complex harvested en block and transferred in 10% phosphate buffered formaldehyde for histopathology analysis. The animals are then euthanized. After paraffin embedding, serial 5-um thin cross-sections are cut every 2 mm through the sheath and tendon complex. Sections are stained with H&E and Movat's stains to evaluate adhesion growth. Each slide is digitized using a computer connected to a digital microscope camera (Nikon Micropublisher cooled camera). Morphometry analysis is then performed using image analysis software (ImagePro). Thickness and area of adhesion defined as the substance obliterating the synovial space are measured and compared between formulation-treated and control animals

### Example 30

### Screening Assay for Assessing the Effect of PDGF on Smooth Muscle Cell Migration

An in vitro assay was described for determining whether a substance stimulates cell (e.g., fibroblast) migration. Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media, and diluted to a concentration of 2-2.5×10⁵ cells/ml in migration media. Migration media consisted of phenol red free Dulbecco's Modified Eagle Medium (DMEM) containing 0.35% human serum albumin. A 100 μl volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB), was added at a concentration of 10 ng/ml in a total volume of 150 μL. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

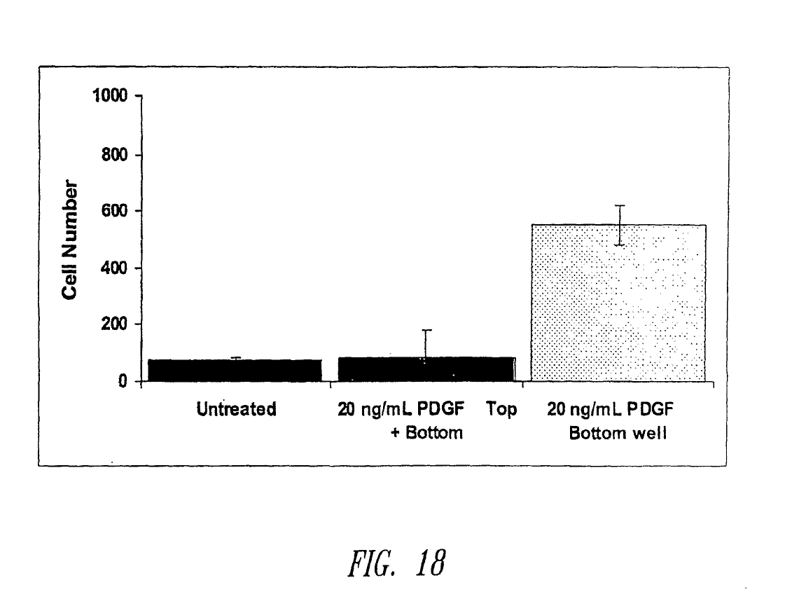

At the end of the 4-hour period, cells in the top chamber were discarded, and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant), and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). The results of a representative assay were shown in FIG. 18. (See also references: Biotechniques 29:81 (2000), J. Immunol. Methods 254:85 (2001)).

### Example 31

### Wound Healing Model to Assess Scar Inhibition by Drug-Eluting Sutures

The purpose of this study is to evaluate the cosmesis of closing skin wounds with sutures containing a scar prevention drug compared to standard techniques using the same suture without a drug. This study is done in miniature swine, a well-accepted model for tissue approximation studies because of its morphological and functional similarities to human tissues.

Three miniature, white, male Gottingen pigs (25-30 kg) are used in this study. The animals are fasted overnight prior to surgery. Anaesthesia is induced by an intramuscular injection of Telazol (6.6 mg/kg), Xylazine (2 mg/kg) and Glycopyrrolate (0.3 mL). These animals receive a preoperative intramuscular dose of Excenel antibiotic (50 mg/pig). Each pig is then intubated, induced to a surgical plane, and maintained by inhalation of 2% isoflurane. The animal's back and sides are shaved and depiliated with Nair. The skin is prepped with iodophor scrub, isopropyl alcohol, and iodophor solution and wiped with sterile gauze. An IV of lactated ringers (5 cc/kg/hr) is placed in an ear vein to maintain hydration. The respiratory rate, heart rate, blood oxygen saturation and body temperature is monitored throughout the procedure. The animals are kept warm with a heating pad under the animal. Following surgical preparation 12 pairs of full thickness skin incisions are made on the dorsolateral aspects of each pig. Each incision is 4 cm in length and full-thickness in depth. Using aseptic technique, a scalpel with No. 10 blade is used to make the full-thickness skin incisions Bleeding is controlled by pressure with dry gauze and bipolar cautery when required.

One incision of each pair is closed with a drug-eluting suture and the other with the same suture without drug. On each animal, 6 pairs of incisions are closed with barbed sutures and 6 are closed with knotted sutures. All incisions are closed with a continuous running dermal suture. Each wound is supported by applying wound closure tapes across the incision. The wounds are covered with gauze pads and the entire torso of the pig wrapped circumferentially with porous elastic tape. The animals are recovered and given food and water ad libitum. They are monitored frequently for any signs of complications. Dressings are adjusted, repaired, or changed as needed to keep the wounds protected for 2 weeks until removed.

All pigs are euthanized at 6 weeks post surgery for evaluation. All wounds are photographed with a digital camera. Quality of wound healing is assessed by a plastic surgeon not associated with the study who is blinded to the type of suture used for closure. Cosmesis is assessed by two different quantitative assessment tools: the visual analog scale (VAS) and the wound evaluation scale (WES).

### WES

Each wound is assigned a cosmetic score (0-6) based on its appearance. Each wound is assessed by 6 parameters and scored as either 0 or 1. A score of 6 represents acceptable cosmesis, while a score of 5 or less represents a suboptimal result.

The 6 parameters are as follows:

| | | |
|---|---|---|
| Stepoff of borders (edges not on same plane) | Yes (0) | No (1) |
| Contour irregularities (wrinkled skin near wound | Yes (0) | No (1) |
| Margin separation (gap between sides) | Yes (0) | No (1) |
| Edge inversion (wound not properly everted) | Yes (0) | No (1) |
| Excession distortion (swelling/edema/infection) | Yes (0) | No (1) |
| Overall appearance unacceptable | Yes (0) | No (1) |

### VAS

The visual analog scale is a 100 point scale where 0 is the worst scar ever observed and 100 is the perfect scar. The healing is assessed for rate of healing by an experienced plastic surgeon using this score range. In addition, quantitive histomorphology is performed in each tissue section cut at 5 mm intervals along the 4 cm length of the incision. Scar is defined as dermal tissue containing collagen (positive trichrome stain) arranged in a dense configuration. The width and thickness of the scar tissue are measured on microscopic images of each sectioned and stained specimen after being digitized for image analysis. The scar area for a given animal is expressed as the summation of all area determinations divided by the number of tissue sections evaluated. (References: Hollander J E, Singer A J, Valentine S, Henry M C. Wound registry: development and validation. Ann Emerg Med 25: 675-685, 1995; Quinn J V, Drzewiecki A E, Stiell I G, Elmslie T J. Appearance scales to measure cosmetic outcomes of healed lacerations. Amer J Emerg Med 13(2): 229-231, 1995)

### Example 32

### Capsular Formation Model for Assessing Inhibition of Fibrosis

Male Wistar rats weighing 300 g or more were used for this procedure. The rats were anaesthetized by placing them in a box filled with 5% isoflurane. Once an appropriate level of general anaesthesia was achieved, the rats were weighed and their back was carefully shaved and cleaned with chlorhexidine and surgical grade iodine. The rats were then transferred to the surgical table and placed in a prone position on a towel and draped in a sterile fashion. Anaesthesia was maintained by the use of a nose cone at a dose of 1.5-2% isoflurane. Buprenorphine (0.01 mg/kg) was administered subcutaneously in the flank, and was sufficient to provide effective analgesia throughout the post-surgical recovery period. Each rat also received an intramuscular injection of duplocillin (7500 U/kg) in the contralateral flank. Using full aseptic technique, a two centimetre incision was made with a scalpel and a No. 15 blade along the dorsal midline just below the caudal edge of the scapula Careful blunt dissection was performed laterally on each side of this incision to create an intramuscular tunnel, at least two centimetres long, between the latissimus dorsi muscle and the fascia of the underlying skeletal musculature just large enough to accommodate a 1 cm×0 3 cm silicon disk implant. The disk was inserted in the pocket at the lateral end of the tunnel to ensure that the medial edge of the implant was placed in a position one centimeter from the incision site, and two centimetres from the contralateral disk on the other side of the vertebral column. Only a single midline incision, therefore, was required to insert both disks, and allowed sufficient distance from the disk to the incision site and between each disk so that capsular formation around each disc was not affected by the presence of the other disks, or by the healing process at the surgical incision. This surgical procedure was repeated with another incision placed two centimetres caudal to the initial incision, so that each rat was implanted with four disks, two on each side of the vertebral column with all the disks two centimetres from each other. The drug under investigation was instilled into three of the pockets and a control vehicle solution was introduced into the other pocket. The spatial position of the implants was coded and varied equally between the two groups to control for the tissue response and perfusion differences. In addition, control rats underwent the same surgical procedure, but without administration of drug into any of the 4 implant sites The pockets were carefully sutured with 5-0 Prolene monofilament sutures, to ensure that no leakage of drug or migration of the disc occurred. Each incision was closed by suturing the fascia with 5-0 Prolene sutures. The skin was closed with 3-0 Prolene sutures and Op-site dressing was sprayed over the wound. The concentration of isoflurane gradually was reduced and oxygen administered by the nose cone. The subjects were placed in a clean cage, under a heat lamp, and covered with towels to maintain body temperature during recovery. Following recovery, rats were returned to the housing room and provided with food and water ad libitum. Each rat was examined daily with particular attention to wound appearance, occurrence of soft-tissue swelling, clinical signs of sepsis, and ability to move and thrive Weight was measured daily until sacrifice

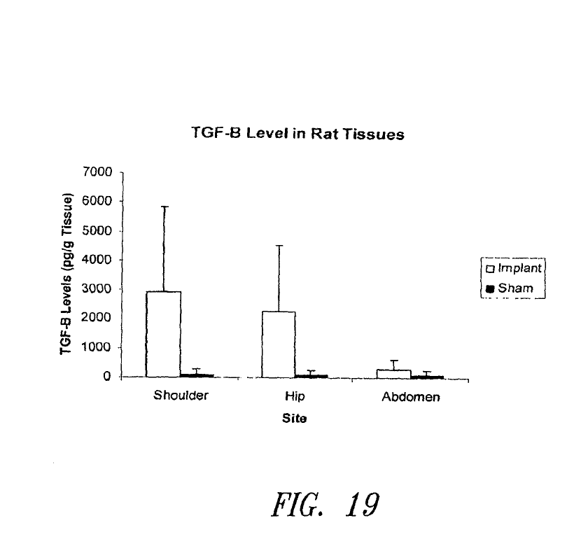

The animals were then euthanized by CO₂ inhalation After allowing sufficient time for the tissue to be cut without bleeding, the implants and the surrounding tissue were excised, and the implant carefully removed from the pocket so as to maintain tissue architecture. The tissue from two of the treated implant sites (one shoulder disk and one hip disk), as well as the site where no drug was added, was processed for the presence of TGF-β. This inflammatory cytokine had been shown to act as a potent stimulatory signal for connective tissue formation in fibrotic conditions where the levels peaked at 7 to 10 days. In addition, high levels of TGF-β were found in contracted capsules. The tissue from the remaining implant site was fixed in 10% buffered formalin for eventual histological processing and examination. Efficacy of the therapeutic agent was determined by a reduction in the level of TGF-β in the treated sites, compared to the site where no drug was added and to the levels observed in the tissues of the implant sites in the untreated control rats. In addition, histological examination of implant tissues from the treated and control rats was used to quantitate the degree of fibrotic capsule formation around the implant by the use of standard morphometric analysis (see, FIG. 19) (References: Border W. A. & Noble N. A. (1994) Transforming growth factor beta in tissue fibrosis. N. Engl. J. Med. 331, 1286-1292; Eltze E, Bettendorf O, Rody A, Jackisch C, Herchenroder, Bocker W, Pfleiderer B. (2003) Influence of local complications on capsule formation around model implants in a rat model. J. Biomed. Mater. Res. A. 64, 12-19; Gad S. A. (2002) Implantation Biology and Studies; in Safety Evaluation of Medical Devices, 2^{nd} edition, Marcel Dekker, Inc, New York, USA, pg 269-292).

### Example 33

### Suture Barbing

For examples 1 to 8, the suture that is used can be either a self-retaining suture (prepared according to U.S. Pat. No. 6,848,152) or a standard suture. For the cases where a standard suture is used for the coating processes, the coated suture is then transformed into a self-retaining suture according to U.S. Pat. No. 6,848,152.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-35. (canceled)

36. An anti-scarring suture, comprising: a self-retaining suture and an anti-scarring agent.

37. The anti-scarring suture of claim 36, wherein the anti-scarring agent is an anti-microbial agent, an anti-angiogenic agent, an anti-inflammatory agent, a heat shock protein 90 (HSP90) antagonist, a histamine receptor antagonist, an anesthetic, a protein, a peptide, an anti-thrombotic agent, an anti-platelet agent, or a steroid.

38. The anti-scarring suture of claim 37, wherein the anti-microbial agent is an anti-mycotic agent.

39. The anti-scarring suture of claim 38, wherein the anti-mycotic agent is terbinafine, staurosporine, itraconazole, or miconazole.

40. The anti-scarring suture of claim 37, wherein the anti-microbial agent is an anthracycline, fluoropyrimidine, folic acid antagonist, or podophylotoxin.

41. The anti-scarring suture of claim 37, wherein the anti-angiogenic agent is paclitaxel.

42. The anti-scarring suture of claim 37, wherein the HSP90 antagonist is geldanamycin.

43. The anti-scarring suture of claim 37, wherein the histamine receptor antagonist is famotidine.

44. The anti-scarring suture of claim 36, further comprising a polymer.

45. The anti-scarring suture of claim 44, wherein the polymer is a polymeric carrier of the anti-scarring agent.

46. A method for making the anti-scarring suture of claim 36, comprising: combining a self-retaining suture with an anti-scarring agent or a composition comprising an anti-scarring agent.

47. A method for reducing, or reducing the risk of, excessive scarring, comprising implanting the anti-scarring suture of claim 36 into a tissue.

48. The method of claim 47, wherein the excessive scarring is at a wound closure site, a site of attachment of a foreign element to a tissue, or a site of a tissue repositioning surgery.

49. The method of claim 47, wherein the excessive scarring results in a keloid or hypertrophic scar.

50. The method of claim 47, wherein the excessive scarring results in surgical adhesion.

51. A method for reducing, or reducing the risk of, excessive scarring, comprising infiltrating a tissue at which a self-retaining suture has been, is being, or to be, implanted with an effective amount of an anti-scarring agent or a composition comprising an anti-scarring agent.

52. The method of claim 51, wherein the excessive scarring is at a wound closure site, a site of attachment of a foreign element to a tissue, or a site of a tissue repositioning surgery.

53. The method of claim 51, wherein the excessive scarring results in a keloid or hypertrophic scar.

54. The method of claim 51, wherein the excessive scarring results in surgical adhesion.
